(19) United States
(12) Patent Application Publication
(10) Pub. No.: US 2026/0115305 A1
(43) Pub. Date: Apr. 30, 2026

(54) LINKERS, CONJUGATES AND APPLICATIONS THEREOF

(71) Applicants: GENEQUANTUM HEALTHCARE (SUZHOU) CO., LTD., Suzhou (CN); GENEQUANTUM MEDICINE (SUZHOU) CO., LTD., Suzhou (CN)

(72) Inventors: Gang QIN, Suzhou (CN); Mingyu HU, Suzhou (CN); Zhaoxiong CAI, Suzhou (CN)

(21) Appl. No.: 18/994,258

(22) PCT Filed: Jul. 14, 2023
(86) PCT No.: PCT/CN2023/107455

§ 371 Date: Jan. 14, 2025

(30) Foreign Application Priority Data
Jul. 15, 2022 (WO) PCT/CN2022/106025

(51) Int. Cl. A61K 47/68; A61P 35/00; C07K 16/28; C07K 16/30; C07K 16/32

(52) U.S. Cl. CPC A61K 47/68037; A61K 47/6849; A61K 47/6855; A61K 47/6889; A61P 35/00; C07K 16/2863; C07K 16/30; C07K 16/32

(57) ABSTRACT

The present disclosure relates to the biopharmaceutical field, in particular, to a linker for preparing targeting molecule-drug conjugates, and the corresponding conjugates, the preparing process and use thereof.

### CROSS REFERENCE TO RELATED APPLICATION

This application claims priority to the following application: International Application No. PCT/CN2022/106025, filed on Jul. 15, 2022.

### TECHNICAL FIELD

The present disclosure relates to the biopharmaceutical field, in particular, to a linker for preparing targeting molecule-drug conjugates, and the corresponding conjugates, the preparing process and use thereof.

### BACKGROUND

HER2 was found to be overexpressed in several cancer types, including breast cancer and gastric cancer, and proved to be a promising target for cancer therapies. Multiple HER2 targeting therapeutic modalities has been approved, including HER2 tyrosine kinase inhibitors (Lapatinib, Tucatinib), therapeutic HER2 antibodies (Herceptin, Perjeta), and HER2 targeting ADC (Kadcyla, Enhertu). These therapeutic agents have significantly improved the survival of HER2 positive breast cancer and gastric cancer patients. Especially, Enhertu not only showed great efficacy in HER2 high patients, but also demonstrated sign of efficacy in HER2 medium/low patients, which may potentially benefit more HER2 expressing cancer patients. Albeit the great efficacy, Enhertu caused more than 10% of interstitial lung disease, which limited its usage in part of patients.

Enhertu, as well as the other commercially available ADCs and most of the ADCs in clinical trials, are prepared by chemical conjugation, using a thiosuccinimide structure (thiosuccinimide linkage) to conjugate the small molecule drug with the targeting antibody or protein. The thiosuccinimide structure is formed by the reaction of a thiol group and a maleimide. However, the thiosuccinimide linkage is not stable. In organisms, reverse Michael addition or exchange with other thiol groups leads to the fall-off of the cytotoxin from the ADC and off-target toxicity, which reduces the safety and limits the clinical application.

TROP2 is a transmembrane glycoprotein encoded by the Tacstd2 gene. It is an intracellular calcium signal transducer and is overexpressed in a variety of tumors. IMMU-132 (also known as SG) and DS-1062 are the famous ant-trop2 ADCs in clinical. Although SG has shown good anti-tumor effect in clinical trials, it has the same side effects as toxic payload (SN-38), including bone marrow suppression and gastrointestinal toxicity. As for DS1062, in the first-phase clinical trial, 48% patients had adverse events above Grade 3, and 8% patients had interstitial lung disease. The recently published phase III clinical data of DS-1062 in the treatment of advanced non-small cell lung cancer shows that DS-1062 does not show better safety. In this clinical trial, some Grade 5 adverse events were observed. Since the linker of DS1062 and IMMU-132 both uses the maleimide group, which is unstable in the blood, its potential toxin shedding may be one of the reasons for the poor safety above.

The protein encoded by FGFR3 gene is a member of the Fibroblast growth factor receptor (FGFR) family and can bind to acidic and basic Fibroblast growth factor (FGF) and plays an important role in bone development and maintenance. It may be an important target for ADC.

A novel branched linker has been developed by the present inventors, which contains no maleimide group and is conjugated to the antibody at a specific site through an amino acid at a tail, thus avoiding the potential instability in blood. Meanwhile, the branched linker has good assembling ability, and can be adapted to a variety of different payloads and commercialized linker-payload fragments conveniently and environment-friendly. The formed linker-payload structures are easy to be separated and purified, which is beneficial for CMC development in a later stage. In addition, by conjugating different kinds of linkers to different antibodies, the stability, compatibility of the linkers and the excellent biological activity of the ADCs formed by the linkers are verified.

Also provided are some conjugates which have better molecular stability and better antitumor efficacy compared to benchmarker, e.g. Enhertu. Additionally, the conjugate may have good physicochemical properties, good pharmacokinetic properties and good safety. Furtherly, high modular design makes it easy to assemble with multiple drugs.

### SUMMARY

In a first aspect, provided is a compound of formula (I):

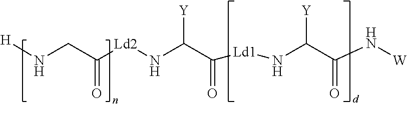

wherein,
W is hydrogen, LKb or —C₂H₄-(PEG)ₜ-(CO)NH₂;
Y is LKa-LKb;
each LKa is independently selected from

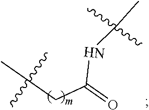 [SMILES: *CC(=O)N*]

each LKb is independently L²-L¹-B;
each B is independently a terminal group R¹⁰, or a combination of 1) a self-immolative spacer Sp1; 2) a bond, or one of or a combination of two or more of the bivalent groups selected from: —CR¹R²—, C₁₋₁₀ alkylene, C₄₋₁₀ cycloalkylene, C₄₋₁₀ heterocyclylene and —(CO)—; and 3) a terminal group R¹⁰;
R¹⁰ is hydrogen, or a group which can leave when reacting with a group in the payload;
each L¹ is independently Cleavable sequence 1 comprising an amino acid sequence which can be cleaved by enzyme, and Cleavable sequence 1 comprises 1-10 amino acids;
each L² is independently a bond; or a C₂₋₂₀ alkylene wherein one or more —CH₂— structures in the alkylene is optionally replaced by —CR³R⁴—, —O—, —(CO)—, —S(═O)₂—, —NR⁵—, —N^{⊕}R⁶R⁷—, C₄₋₁₀ cycloalkylene, C₄₋₁₀ heterocyclylene, phenylene; wherein the cycloalkylene, heterocyclylene and phenylene are each independently unsubstituted or substituted with at least one substituent selected from halogen, —C₁₋₁₀ alkyl, —C₁₋₁₀haloalkyl, —C₁₋₁₀ alkylene—NH—R⁸ and -C₁₋₁₀ alkylene-O—R⁹;
Ld2 and each Ld1 are independently a bond; or selected from —NH—C₁₋₂₀ alkylene-(CO)—, —NH—(PEG)ᵢ-(CO)—, or are a natural amino acid or oligomeric natural amino acids having a degree of polymerization of 2-10 independently unsubstituted or substituted with —(CO)-(PEG)ⱼ-OR¹¹ on the side chain;
(PEG)ₜ-, -(PEG)ᵢ- and -(PEG)ⱼ- are each a PEG fragment, which comprises the denoted number of consecutive —(O—C₂H₄)— structure units or consecutive —(C₂H₄—O)— structure units, with an optional additional C₁₋₁₀ alkylene at one terminal;
R¹, R², R³, R⁴, R⁵, R⁶, R⁷, R⁸, R⁹ are each independently selected from hydrogen, halogen, —C₁₋₁₀ alkyl, —C₁₋₁₀ haloalkyl, C₄₋₁₀ cycloalkylene; or
R¹ and R² together with the carbon atom to which they are attached form a 3-6 membered cycloalkyl group; or
R³ and R⁴ together with the carbon atom to which they are attached form a 3-6 membered cycloalkyl group;
R¹¹ is C₁₋₁₀ alkyl;
m is any integer of 1 to 5;
n is any integer of 2 to 20;
d is any integer of 1 to 6, particularly 1, 2, 3;
each i is independently an integer of 0-100, preferably 0 to 20; preferably each i is independently an integer of 0 to 12; more preferably 0 to 8; particularly 4;
each j is independently an integer of 1-100, preferably 1 to 20; preferably each j is independently an integer of 1 to 12; more preferably 8 to 12; particularly 8 or 12;
each t is independently an integer of 1-100, preferably 1 to 20; preferably each t is independently an integer of 1 to 12; more preferably 8 to 12; particularly 8 or 12.

In a second aspect, provided is a compound having the structure of formula (II)

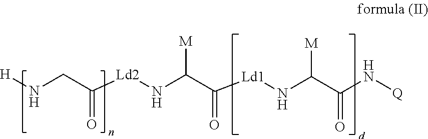

Wherein
Q is hydrogen, —C₂H₄-(PEG)ₜ-(CO)NH₂ or LKb-P;
M is LKa-LKb-P;
P is a payload which is linked to the B moiety or L¹ moiety of the compound of formula (I);
n, d, Ld1, Ld2, t, LKa and LKb are as defined in formula (I).

In a third aspect, provided is a conjugate having the structure of formula (III):

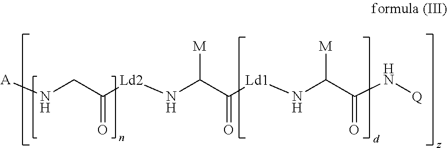

wherein,
Q is hydrogen, —C₂H₄-(PEG)ₜ-(CO)NH₂ or LKb-P;
n, d, t, Ld1 and Ld2 are as defined in formula (I);
M is LKa-LKb-P;
P is a payload which is linked to the B moiety or L¹ moiety of the compound of formula (I);
A is a targeting molecule;
z is an integer of 1 to 20.

In a fourth aspect, provided is an intermediate compound having the structure of formula (IV) for the preparation of formula (I):

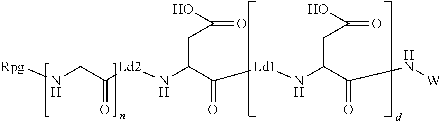

wherein,
Rpg is selected from hydrogen, or a protecting group, preferably selected from acetyl, trifluoroacetyl, t-butoxycarbonyl (Boc), benzyloxycarbonyl (Cbz) and 9-fluorenylmethylenoxycarbonyl (Fmoc);
W, Ld1, Ld2, n and d are as defined in formula (I).

In a fifth aspect, provided is use of the conjugate of the present disclosure or the pharmaceutical composition thereof in the manufacture of a medicament for treating a disease; wherein the disease is a tumor or an autoimmune disease.

### BRIEF DESCRIPTION OF THE DRAWINGS

FIG. 1 shows the efficacy of ADCs in the SK-BR-3 and MDA-MB-468 co-culture cell line.

FIG. 2 shows the efficacy of conjugate ADC-2 and conjugate ADC-1 in JIMT-1 CDX model in vivo.

FIG. 3 shows the efficacy of ADC-2, ADC-3 and Enhertu in JIMT-1 CDX model in vivo.

FIG. 4 shows the efficacy of conjugate ADC-2 and conjugate ADC-1 in NCI-N87 CDX model in vivo.

FIG. 5 shows the efficacy of conjugate ADC-5, ADC-6 and ADC-7 on the proliferation of TROP2-positive tumor cells BxPC-3.

FIG. 6 shows the results of internalization assay of conjugate ADC-5 and ADC-6.

FIG. 7 shows the efficacy of conjugate ADC-5 and ADC-6 on tumor growth in TROP2 positive NCI-N87 CDX mouse model.

FIG. 8 shows the efficacy of conjugate ADC-5 and ADC-6 on tumor growth in TROP2 positive FaDu CDX mouse model.

FIG. 9 shows the efficacy of conjugate ADC-8, ADC-9 and ADC-10 on tumor growth in FGFR3 positive RT112/84CDX mouse model.

FIG. 10 shows the efficacy of conjugate ADC-8, ADC-9 and ADC-10 on tumor growth in FGFR3 positive RT4CDX mouse model.

### DETAILED DESCRIPTION

The specific embodiments are provided below to illustrate technical contents of the present disclosure. Those skilled in the art can easily understand other advantages and effects of the present disclosure through the contents disclosed in the specification. The present disclosure can also be implemented or applied through other different specific embodiments. Various modifications and variations can be made by those skilled in the art without departing from the spirit of the present disclosure.

### Definitions

Unless otherwise defined hereinafter, all technical and scientific terms used herein have the same meaning as commonly understood by those skilled in the art. The techniques used herein refer to those that are generally understood in the art, including the variants and equivalent substitutions that are obvious to those skilled in the art. While the following terms are believed to be readily comprehensible by those skilled in the art, the following definitions are set forth to better illustrate the present disclosure. When a trade name is present herein, it refers to the corresponding commodity or the active ingredient thereof. All patents, published patent applications and publications cited herein are hereby incorporated by reference.

When a certain amount, concentration, or other value or parameter is set forth in the form of a range, a preferred range, or a preferred upper limit or a preferred lower limit, it should be understood that it is equivalent to specifically revealing any range formed by combining any upper limit or preferred value with any lower limit or preferred value, regardless of whether the said range is explicitly recited. Unless otherwise stated, the numerical ranges listed herein are intended to include the endpoints of the range and all integers and fractions (decimals) within the range. For example, the expression “i is an integer of 0 to 20” means that i is any integer of 0 to 20, for example, i can be 0, 1, 2, 3, 4, 5, 6, 7, 8, 9, 10, 11, 12, 13, 14, 15, 16, 17, 18, 19 or 20. Other similar expressions such as d, m, n, j and k should also be understood in a similar manner.

Unless the context clearly dictates otherwise, singular forms like “a” and “the” include the plural forms. The expression “one or more” or “at least one” may mean 1, 2, 3, 4, 5, 6, 7, 8, 9 or more.

The terms “about” and “approximately”, when used in connection with a numerical variable, generally mean that the value of the variable and all values of the variable are within experimental error (for example, within a 95% confidence interval for the mean) or within ±10% of a specified value, or a wider range.

The term “optional” or “optionally” means the event described subsequent thereto may, but not necessarily happen, and the description includes the cases wherein said event or circumstance happens or does not happen.

The expressions “comprising”, “including”, “containing” and “having” are open-ended, and do not exclude additional unrecited elements, steps, or ingredients. The expression “consisting of” excludes any element, step, or ingredient not designated. The expression “consisting essentially of” means that the scope is limited to the designated elements, steps or ingredients, plus elements, steps or ingredients that are optionally present that do not substantially affect the essential and novel characteristics of the claimed subject matter. It should be understood that the expression “comprising” encompasses the expressions “consisting essentially of” and “consisting of”.

The term “targeting molecule” refers to a molecule that has an affinity for a particular target (e.g., a receptor, a cell surface protein, a cytokine, a tumor specific antigen, etc.). A targeting molecule can deliver the payload to a specific site in vivo through targeted delivery. A targeting molecule can recognize one or more targets. The specific target sites are defined by the targets it recognizes. For example, a targeting molecule that targets a receptor can deliver a cytotoxin to a site containing a large number of the receptor. Examples of targeting molecules include, but are not limited to antibodies, antibody fragments, binding proteins for a given antigen, antibody mimics, scaffold proteins having affinity for a given target, ligands, and the like.

As used herein, the term “antibody” is used in a broad way and particularly includes intact monoclonal antibodies, polyclonal antibodies, monospecific antibodies, multispecific antibodies (e.g., bispecific antibodies), and antibody fragments, as long as they have the desired biological activity. The antibody may be of any subtype (such as IgG, IgE, IgM, IgD, and IgA) or subclass, and may be derived from any suitable species. In some embodiments, the antibody is of human or murine origin. The antibody may also be a fully human antibody, humanized antibody or chimeric antibody prepared by recombinant methods. In some embodiments, the antibody can be engineered, for example, the introduction of a ligase-specific recognition sequence at its terminals.

Monoclonal antibodies are used herein to refer to antibodies obtained from a substantially homogeneous population of antibodies, i.e., the individual antibodies constituting the population are identical except for a small number of possible natural mutations. Monoclonal antibodies are highly specific for a single antigenic site. The word “monoclonal” refers to that the characteristics of the antibody are derived from a substantially homogeneous population of antibodies and are not to be construed as requiring some particular methods to produce the antibody.

An intact antibody or full-length antibody essentially comprises the antigen-binding variable region(s) as well as the light chain constant region(s) (CL) and heavy chain constant region(s) (CH), which could include CH1, CH2, CH3 and CH4, depending on the subtype of the antibody. An antigen-binding variable region (also known as a fragment variable region, Fv fragment) typically comprises a light chain variable region (VL) and a heavy chain variable region (VH). A constant region can be a constant region with a native sequence (such as a constant region with a human native sequence) or an amino acid sequence variant thereof. The variable region recognizes and interacts with the target antigen. The constant region can be recognized by and interact with the immune system.

An antibody fragment may comprise a portion of an intact antibody, preferably its antigen binding region or variable region. Examples of antibody fragments include Fab, Fab′, F(ab′)2, Fd fragment consisting of VH and CH1 domains, Fv fragment, single-domain antibody (dAb) fragment, and isolated complementarity determining region (CDR). The Fab fragment is an antibody fragment obtained by papain digestion of a full-length immunoglobulin, or a fragment having the same structure produced by, for example, recombinant expression. A Fab fragment comprises a light chain (comprising a VL and a CL) and another chain, wherein the said other chain comprises a variable domain of the heavy chain (VH) and a constant region domain of the heavy chain (CH1). The F(ab′)2 fragment is an antibody fragment obtained by pepsin digestion of an immunoglobulin at pH 4.0-4.5, or a fragment having the same structure produced by, for example, recombinant expression. The F(ab′)2 fragment essentially comprises two Fab fragments, wherein each heavy chain portion comprises a few additional amino acids, including the cysteines that form disulfide bonds connecting the two fragments. A Fab′ fragment is a fragment comprising one half of a F(ab′)2 fragment (one heavy chain and one light chain). The antibody fragment may comprise a plurality of chains joined together, for example, via a disulfide bond and/or via a peptide linker. Examples of antibody fragments also include single-chain Fv (scFv), Fv, dsFv, diabody, Fd and Fd′ fragments, and other fragments, including modified fragments. An antibody fragment typically comprises at least or about 50 amino acids, and typically at least or about 200 amino acids. An antigen-binding fragment can include any antibody fragment that, when inserted into an antibody framework (e.g., by substitution of the corresponding region), can result in an antibody that immunospecifically binds to the antigen.

Antibodies according to the present disclosure can be prepared using techniques well known in the art, such as the following techniques or a combination thereof: recombinant techniques, phage display techniques, synthetic techniques, or other techniques known in the art. For example, a genetically engineered recombinant antibody (or antibody mimic) can be expressed by a suitable culture system (e.g., E. coli or mammalian cells). The engineering can refer to, for example, the introduction of a ligase-specific recognition sequence at its terminals.

HER2 refers to human epidermal growth factor receptor-2, which belongs to the epidermal growth factor (EGFR) receptor tyrosine kinase family. In the present application, the terms ErbB2 and HER2 have the same meaning and can be used interchangeably.

TROP2 is a transmembrane glycoprotein encoded by the Tacstd2 gene. It is an intracellular calcium signal transducer and is overexpressed in a variety of tumors.

The protein encoded by FGFR3 gene is a member of the Fibroblast growth factor receptor (FGFR) family and can bind to acidic and basic Fibroblast growth factor (FGF) and play an important role in bone development and maintenance.

As used herein, the term “targeting molecule-drug conjugate” is referred to as “conjugate”. Examples of conjugates include, but are not limited to, antibody-drug conjugates.

A small molecule compound refers to a molecule with a size comparable to that of an organic molecule commonly used in medicine. The term does not encompass biological macromolecules (e.g., proteins, nucleic acids, etc.), but encompasses low molecular weight peptides or derivatives thereof, such as dipeptides, tripeptides, tetrapeptides, pentapeptides, and the like. Typically, the molecular weight of the small molecule compound can be, for example, about 100 to about 2000 Da, about 200 to about 1000 Da, about 200 to about 900 Da, about 200 to about 800 Da, about 200 to about 700 Da, about 200 to about 600 Da, about 200 to about 500 Da.

Cytotoxin refers to a substance that inhibits or prevents the expression activity of a cell, cellular function, and/or causes destruction of cells. The cytotoxins currently used in ADCs are more toxic than chemotherapeutic drugs. Examples of cytotoxins include, but are not limited to, drugs that target the following targets: microtubule cytoskeleton, DNA, RNA, kinesin-mediated protein transport, regulation of apoptosis. The drug that targets microtubule cytoskeleton may be, for example, a microtubule-stabilizing agent or a tubulin polymerization inhibitor. Examples of microtubule-stabilizing agents include but are not limited to taxanes. Examples of tubulin polymerization inhibitors include but are not limited to maytansinoids, auristatins, vinblastines, colchicines, and dolastatins. The DNA-targeting drug can be, for example, a drug that directly disrupts the DNA structure or a topoisomerase inhibitor. Examples of drugs that directly disrupt DNA structure include but are not limited to DNA double strand breakers, DNA alkylating agents, DNA intercalators. The DNA double strand breakers can be, for example, an enediyne antibiotic, including but not limited to dynemicin, esperamicin, neocarzinostatin, uncialamycin, and the like. The DNA alkylating agent may be, for example, a DNA bis-alkylator (i.e. DNA-cross linker) or a DNA mono-alkylator. Examples of DNA alkylating agents include but are not limited to pyrrolo[2,1-c][1,4]benzodiazepine (PBD) dimer, 1-(chloromethyl)-2,3-dihydrogen-1H-benzo[e]indole (CBI) dimer, CBI-PBD heterodimer, dihydroindolobenzodiazepine (IGN) dimer, duocarmycin-like compound, and the like. Examples of topoisomerase inhibitors include but are not limited to exatecan and derivatives thereof (such as DX8951f, DXd-(1) and DXd-(2), the structures of which are depicted below), camptothecins and anthracyclines. The RNA-targeting drug may be, for example, a drug that inhibits splicing, and examples thereof include but are not limited to pladienolide. Drugs that target kinesin-mediated protein transport can be, for example, mitotic kinesin inhibitors including, but not limited to, kinesin spindle protein (KSP) inhibitors.

A spacer is a structure that is located between different structural modules and can spatially separate the structural modules. The definition of spacer is not limited by whether it has a certain function or whether it can be cleaved or degraded in vivo. Examples of spacers include but are not limited to amino acids and non-amino acid structures, wherein non-amino acid structures can be, but are not limited to, amino acid derivatives or analogues. “Spacer sequence” refers to an amino acid sequence serving as a spacer, and examples thereof include but are not limited to a single amino acid, a sequence containing a plurality of amino acids, for example, a sequence containing two amino acids such as GA, etc., or, for example, GGGGS, GGGGSGGGGS, GGGGSGGGGSGGGGS, etc. Self-immolative spacers are covalent assemblies tailored to correlate the cleavage of two chemical bonds after activation of a protective part in a precursor: Upon stimulation, the protective moiety (such as a cleavable sequence) is removed, which generates a cascade of disassembling reactions leading to the temporally sequential release of smaller molecules. Examples of self-immolative spacers include but not limited to PABC (p-benzyloxycarbonylaniline), acetal, heteroacetal and the combination thereof.

The term “alkyl” refers to a straight or branched saturated aliphatic hydrocarbon group consisting of carbon atoms and hydrogen atoms, which is connected to the rest of the molecule through a single bond. The alkyl group may contain 1 to 20 carbon atoms, referring to C₁-C₂₀ alkyl group, for example, C₁-C₄ alkyl group, C₁-C₃ alkyl group, C₁-C₂ alkyl, C₃ alkyl, C₄ alkyl, C₃-C₆ alkyl. Non-limiting examples of alkyl groups include but are not limited to methyl, ethyl, propyl, butyl, pentyl, hexyl, isopropyl, isobutyl, sec-butyl, tert-butyl, isopentyl, 2-methylbutyl, 1-methylbutyl, 1-ethylpropyl, 1,2-dimethylpropyl, neopentyl, 1,1-dimethylpropyl, 4-methylpentyl, 3-methylpentyl, 2-methylpentyl, 1-methylpentyl, 2-ethylbutyl, 1-ethylbutyl, 3,3-dimethylbutyl, 2,2-dimethyl butyl, 1,1-dimethylbutyl, 2,3-dimethylbutyl, 1,3-dimethylbutyl or 1,2-dimethylbutyl, or their isomers. A bivalent radical refers to a group obtained from the corresponding monovalent radical by removing one hydrogen atom from a carbon atom with free valence electron(s). A bivalent radical have two connecting sites which are connected to the rest of the molecule. For example, an “alkylene” or an “alkylidene” refers to a saturated divalent hydrocarbon group, either straight or branched. Examples of alkylene groups include but are not limited to methylene (—CH₂—), ethylene (—C₂H₄—), propylene (—C₃H₆—), butylene (—C₄H₈—), pentylene (—C₅H₁₀—), hexylene (—C₆H₁₂—), 1-methylethylene (—CH(CH₃)CH₂—), 2-methylethylene (—CH₂CH(CH₃)—), methylpropylene, ethylpropylene, and the like.

As used herein, when a group is combined with another group, the connection of the groups may be linear or branched, provided that a chemically stable structure is formed. The structure formed by such a combination can be connected to other moieties of the molecule via any suitable atom in the structure, preferably via a designated chemical bond. For example, when two or more of the bivalent groups selected from: —CR¹R²—, C₁₋₁₀ alkylene, C₄₋₁₀ cycloalkylene, C₄₋₁₀ heterocyclylene and —(CO)— are combined together to form a combination, the two or more of the bivalent groups may form a linear connection with each other, such as —CR¹R²-C₁₋₁₀ alkylene-(CO)—, —CR¹R²-C₄₋₁₀ cycloalkylene-(CO)—, —CR¹R²-C₄₋₁₀ cycloalkylene-C₁₋₁₀ alkylene-(CO)—, —CR¹R²-CR^{1′}R^{2′}—(CO)—, —CR¹R²-CR^{1′}R^{2′}-CR^{1″}R^{2″}—(CO)—, etc. The resulting bivalent structure can be further connected to other moieties of the molecule.

The term “protecting group” or “Pg” refers to a substituent that can be commonly employed to block or protect a certain functionality while reacting other functional groups on the compound. For example, an “amino-protecting group” is a substituent attached to an amino group that blocks or protects the amino functionality in the compound. Suitable amino-protecting groups include but are not limited to acetyl, trifluoroacetyl, t-butoxycarbonyl (Boc), benzyloxycarbonyl (Cbz) and 9-fluorenylmethylenoxycarbonyl (Fmoc). For a general description of protecting groups and their use, see T. W. Greene, Protective Groups in Organic Synthesis, John Wiley & Sons, New York, 1991.

As used herein, the expressions “antibody-conjugated drug” and “antibody-drug conjugate” has the same meaning.

### Compound of Formula (I)

In one aspect, provide is a compound of formula (I):

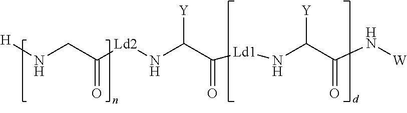

wherein,
W is hydrogen, LKb or —C₂H₄-(PEG)ₜ-(CO)NH₂;
Y is LKa-LKb;
each LKa is independently selected from

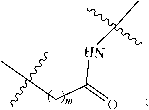 [SMILES: *CC(=O)N*]

each LKb is independently L²-L₁-B;
each B is independently a terminal group R¹⁰, or a combination of 1) a self-immolative spacer Sp1; 2) a bond, or one of or a combination of two or more of the bivalent groups selected from: —CR¹R²—, C₁₋₁₀ alkylene, C₄₋₁₀ cycloalkylene, C₄₋₁₀ heterocyclylene and —(CO)—; and 3) a terminal group R¹⁰;
R¹⁰ is hydrogen, or a group which can leave when reacting with a group in the payload;
each L¹ is independently Cleavable sequence 1 comprising an amino acid sequence which can be cleaved by enzyme, and Cleavable sequence 1 comprises 1-10 amino acids;
each L² is independently a bond; or a C₂₋₂₀ alkylene wherein one or more —CH₂— structures in the alkylene is optionally replaced by —CR³R⁴—, —O—, —(CO)—, —S(═O)₂—, —NR⁵—, —N^{⊕}R⁶R⁷—, C₄₋₁₀ cycloalkylene, C₄₋₁₀ heterocyclylene, phenylene; wherein the cycloalkylene, heterocyclylene and phenylene are each independently unsubstituted or substituted with at least one substituent selected from halogen, —C₁₋₁₀ alkyl, —C₁₋₁₀ haloalkyl, —C₁₋₁₀ alkylene—NH—R⁸ and -C₁₋₁₀ alkylene-O—R⁹;
Ld2 and each Ld1 are independently a bond; or selected from —NH—C₁₋₂₀ alkylene-(CO)—, —NH-(PEG)ᵢ-(CO)—, or are a natural amino acid or oligomeric natural amino acids having a degree of polymerization of 2-10 independently unsubstituted or substituted with —(CO)-(PEG)ⱼ-OR¹¹ on the side chain;
(PEG)ₜ-, -(PEG)ᵢ- and -(PEG)ⱼ- are each a PEG fragment, which comprises the denoted number of consecutive —(O—C₂H₄)— structure units or consecutive —(C₂H₄—O)— structure units, with an optional additional C₁₋₁₀ alkylene at one terminal;
R¹, R², R³, R⁴, R⁵, R⁶, R⁷, R⁸, R⁹ are each independently selected from hydrogen, halogen, —C₁₋₁₀ alkyl, —C₁₋₁₀haloalkyl, C₄₋₁₀ cycloalkylene; or
R¹ and R² together with the carbon atom to which they are attached form a 3-6 membered cycloalkyl group; or
R³ and R⁴ together with the carbon atom to which they are attached form a 3-6 membered cycloalkyl group;
R¹¹ is C₁₋₁₀ alkyl;
m is any integer of 1 to 5;
n is any integer of 2 to 20;
d is any integer of 1 to 6, particularly 1, 2, 3; each i is independently an integer of 0-100, preferably 0 to 20; preferably each i is independently an integer of 0 to 12; more preferably 0 to 8; particularly 4;
each j is independently an integer of 1-100, preferably 1 to 20; preferably each j is independently an integer of 1 to 12; more preferably 8 to 12; particularly 8 or 12;
each t is independently an integer of 1-100, preferably 1 to 20; preferably each t is independently an integer of 1 to 12; more preferably 8 to 12; particularly 8 or 12.

In one embodiment, LKa is selected from

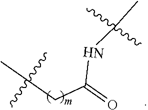

wherein m is an integer of 1-5.

In one embodiment, LKa is selected from

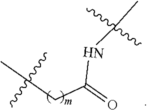

wherein m is an integer of 1-3.

In one embodiment, LKa is selected from

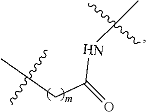

wherein m is 1 or 2.

In one embodiment, L² is selected from: —(C₂H₄—O)ₚ—(CH₂)₂(CO)—, p is an integer of 0 to 5; more preferably p is 0, 2 or 4; most preferably p is 2.

In one embodiment, the carbonyl group in each of the above structure of L² is connected to L¹, and the other linking site is connected to an amide.

In one embodiment, Ld2 and each Ld1 are independently a bond or

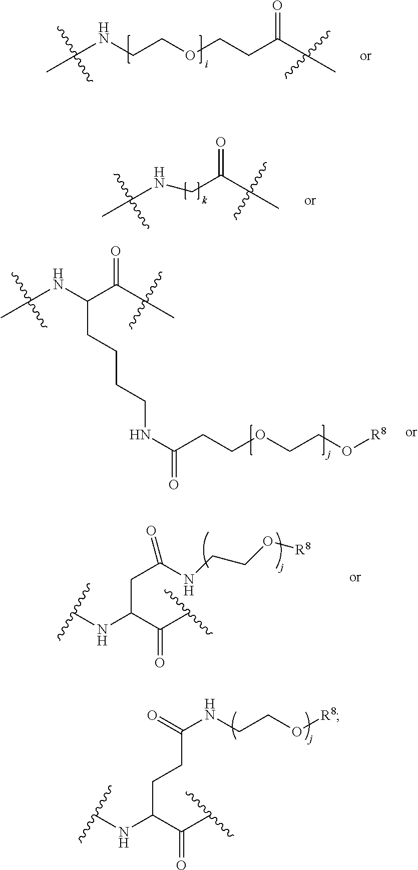

each i is independently an integer of 0-100;
each j and k are independently an integer of 1-100.

In one embodiment, Ld1 is

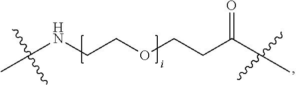

wherein i is an integer of 0-5; preferably i is 0-4; more preferably 0, 2 or 4; most preferably 4.

In one embodiment, Ld2 is a bond.

In one embodiment, Ld2 is a natural amino acid or oligomeric natural amino acids having a degree of polymerization of 2-10 independently unsubstituted or substituted with —(CO)-(PEG)ⱼ-OR¹¹ on the side chain;
(PEG)ⱼ- is a PEG fragment, which comprises the denoted number of consecutive —(O—C₂H₄)— structure units or consecutive —(C₂H₄—O)— structure units, with an optional additional C₁₋₁₀ alkylene at one terminal.

In one embodiment, each i is independently an integer of 0-20, each j and k are independently an integer of 1-20. In one embodiment, each i is independently 0-12, each j and k are independently an integer of 1-12.

In one embodiment, each i is independently an integer of 0-8; particularly 4.

In one embodiment, each j is independently an integer of 8 to 12; particularly 8 or 12.

In one embodiment, each k is independently an integer of 1 to 7; particularly 1, or 3 or 5.

In one embodiment, Ld2 and each d1 are independently a bond; or a C₁₋₂₀ alkylene with an amino and a carbonyl at the two terminals respectively, or a PEG fragment of a certain length (denoted as -(PEG)ᵢ-) with an amino and a carbonyl at the two terminals respectively, or one or more natural amino acids independently unsubstituted or substituted with a PEG fragment of a certain length (denoted as -(PEG)ⱼ-) on the side chain.

In one embodiment, -(PEG)ᵢ- comprises —(O—C₂H₄)ᵢ- or —(C₂H₄—O)ᵢ—, and an optional additional C₁₋₁₀ alkylene at one terminal; -(PEG)ⱼ-, comprises —(O—C₂H₄)ⱼ- or —(C₂H₄—O)ⱼ—, and an optional additional C₁₋₁₀ alkylene at one terminal. In a very specific -(PEG)ᵢ- comprises -C₂H₄-(O—C₂H₄)ᵢ— or —(C₂H₄—O)ᵢ-C₂H₄—; in a very specific -(PEG)ⱼ- comprises -C₂H₄-(O—C₂H₄)ⱼ— or —(C₂H₄—O)ⱼ-C₂H₄—.

In one embodiment, Ld2 is a Lysine substituted with —(CO)-(PEG)ⱼ-OR¹¹ on the side chain.

In a specific embodiment, Ld2 is

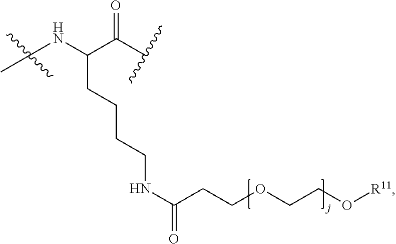

wherein j is an integer of 1-100; preferably 1-20; more preferably 1-12; most preferably 8-12; particularly 8 or 12.

It is to be understood that when there are two or more Ld1, B, L² or L¹ structures in the molecule, the structure of each Ld1, B, L² or L¹ is selected independently. When there are two or more R^{x} (x being 1, 2, 3, 4, 5, 6, 7, 8, 9, etc.) in the molecule, each R^{x} is selected independently. In some embodiments, the “x”s in the molecule are denoted with or without additional apostrophe (′) or apostrophes (such as ″, ′″, ″″, etc.), for example R, R^{1′}, R^{1″}, R^{1′″}, R^{2′}, R^{2″}, R^{2′″}, etc, wherein each R^{X}, with or without additional apostrophe or apostrophes, are selected independently. The other R^{x}s such as R³, R⁴, R⁵, R⁶, R⁷, R⁸, R⁹, and “Ld1”s, “B”s, “L²”s and “L¹”s should be understood in a similar way.

In one embodiment, Cleavable sequence 1 is selected from Gly-Gly-Phe-Gly, Phe-Lys, Val-Cit, Val-Lys, Gly-Phe-Leu-Gly, Ala-Leu-Ala-Leu, Ala-Ala-Ala and the combination thereof; preferably, Cleavable sequence 1 is Gly-Gly-Phe-Gly or Val-Cit.

In one embodiment, R¹⁰ is hydrogen, hydroxy, or

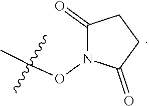 [SMILES: *ON1C(=O)CCC1=O]

In one embodiment, R¹¹ is C₁₋₆ alkyl, preferably methyl.

In one embodiment, W is hydrogen.

In one embodiment, W is —C₂H₄-(PEG)ₜ-(CO)NH₂, wherein t is independently an integer of 1-100, preferably 1 to 20; preferably each t is independently an integer of 1 to 12; more preferably 8 to 12; particularly 8 or 12.

In a specific embodiment, t is 12.

In one embodiment, n is an integer of 2 to 5, especially 3.

In one embodiment, d is any integer of 1 to 4, preferably 1.

In another aspect, provided is an intermediate compound having the structure of formula (IV) for the preparation of formula (I):

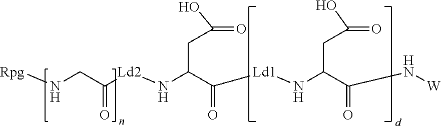

wherein,
Rpg is selected from hydrogen, or a protecting group, preferably selected from acetyl, trifluoroacetyl, t-butoxycarbonyl (Boc), benzyloxycarbonyl (Cbz) and 9-fluorenylmethylenoxycarbonyl (Fmoc);
W, Ld1, Ld2, n and d are as defined in formula (I).

In one embodiment, Pg is 9-fluorenylmethylenoxycarbonyl (Fmoc).

In one embodiment, Ld2 is

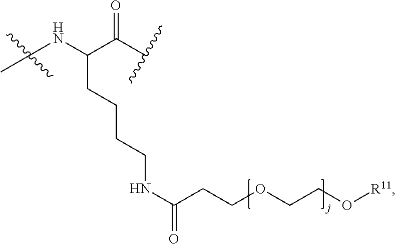

wherein j is an integer of 1-100; preferably 1-20; more preferably 1-12; most preferably 8-12; particularly 8 or 12. In one embodiment, R¹¹ is methyl.

In one embodiment, Ld1 is

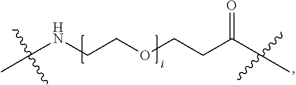

wherein i is an integer of 0-5; preferably i is 0-4; more preferably 0, 2 or 4; most preferably 4.

In one embodiment, W is hydrogen.

In one embodiment, n is 3.

In one embodiment, the formula (IV) having the following structure:

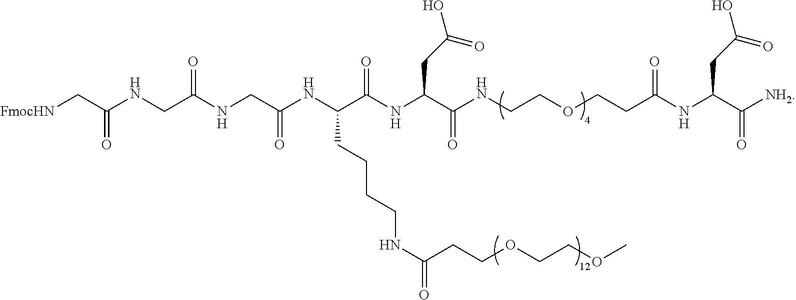

### Moiety Comprising Recognition Sequence of the Ligase Acceptor or Donor Substrate

In one embodiment, the Gₙ moiety of the compound of formula (I) is a recognition sequence of a ligase acceptor substrate, which facilitates enzyme-catalyzed coupling of compound of formula (I) with the targeting molecule under the catalysis of the ligase. The targeting molecule optionally modified and comprises the corresponding recognition sequence of a ligase acceptor substrate.

In one embodiment, the ligase is a transpeptidase. In one embodiment, the ligase is selected from the group consisting of a natural transpeptidase, an unnatural transpeptidase, variants thereof, and the combination thereof. Unnatural transpeptidase enzymes can be, but are not limited to, those obtained by engineering of natural transpeptidase. In a preferred embodiment, the ligase is selected from the group consisting of a natural Sortase, an unnatural Sortase, and the combination thereof. The species of natural Sortase include Sortase A, Sortase B, Sortase C, Sortase D, Sortase L. plantarum, etc. (detailed description can be found in US20110321183A1, which is incorporated herein by reference). The type of ligase corresponds to the ligase recognition sequence and is thereby used to achieve specific conjugation between different molecules or structural fragments.

In some embodiments, the ligase is a Sortase selected from Sortase A, Sortase B, Sortase C, Sortase D and Sortase L. plantarum. In these embodiments, the recognition sequence of the ligase acceptor substrate is selected from the group consisting of oligomeric glycine, oligomeric alanine, and a mixture of oligomeric glycine/alanine having a degree of polymerization of 3-10. In a particular embodiment, the recognition sequence of the ligase acceptor substrate is Gn, wherein G is glycine (Gly), and n is an integer of 2 to 10.

In another particular embodiment, the ligase is Sortase A from Staphylococcus aureus. Accordingly, the ligase recognition sequence may be a typical recognition sequence of the enzyme as LPXTG. In yet another particular embodiment, the recognition sequence of the ligase donor substrate is LPXTGJ, and the recognition sequence of the ligase acceptor substrate is Gn, wherein X can be any single amino acid that is natural or unnatural; J is absent, or is an amino acid fragment comprising 1-10 amino acids, optionally labeled. In one embodiment, J is absent. In yet another embodiment, J is an amino acid fragment comprising 1-10 amino acids, wherein each amino acid is independently any natural or unnatural amino acid. In another embodiment, J is Gₘ, wherein m is an integer of 1 to 10. In yet another particular embodiment, the recognition sequence of the ligase donor substrate is LPETG. In another particular embodiment, the recognition sequence of the ligase donor substrate is LPETGG.

In one embodiment, the ligase is Sortase B from Staphylococcus aureus and the corresponding donor substrate recognition sequence can be NPQTN. In another embodiment, the ligase is Sortase B from Bacillus anthracis and the corresponding donor substrate recognition sequence can be NPKTG.

In yet another embodiment, the ligase is Sortase A from Streptococcus pyogenes and the corresponding donor substrate recognition sequence can be LPXTGJ, wherein J is as defined above. In another embodiment, the ligase is Sortase subfamily 5 from Streptomyces coelicolor, and the corresponding donor substrate recognition sequence can be LAXTG.

In yet another embodiment, the ligase is Sortase A from Lactobacillus plantarum and the corresponding donor substrate recognition sequence can be LPQTSEQ.

The ligase recognition sequence can also be other totally new recognition sequence for transpeptidase optimized by manual screening.

### Moiety Comprising Reactive Group

Reactive Group for Connection with Payload

In one embodiment, B is a terminal group R¹⁰, and the Cleavable sequence 1 in L¹ is connected to the payload. In such case, B is absent in the resulting molecule of the connection of Cleavable sequence 1 with the payload. In one embodiment, B is used for connection to the payload. For connection with the payload, the compound of formula (I) comprises a reactive group. In one embodiment, B in the compound of formula (I) is connected to the payload through an amide bond or an ester bond or an ether bond. In one embodiment, the reactive group in B in formula (I) is independently a reactive group for condensation reaction, nucleophilic addition or electrophilic addition (such as reactive C═O moiety, reactive C═C—C═O moiety, amino group, amine group, hydroxy group or thiol group), or a reactive group for substitution reaction (such as a leaving group attached to an O, C, N or S atom). In one embodiment, the reactive group in B is independently selected from carboxyl group, active ester, aldehyde group, amino group, amine group, hydroxy group and thiol group. In a specific embodiment, the reactive group in B which is used to connect to the payload is independently selected from amino group, amine group, hydroxy group, thiol group, carboxyl group and active ester.

In one embodiment, the reactive group in B is independently amino group, amine group or hydroxy group, which reacts with corresponding groups (such as carboxyl group, sulfonic acid group, phosphoryl group with free -OH end, active ester, acid chloride or isocyanate group) in the payload. In another embodiment, the reactive group in B is independently carboxyl group or active ester, which reacts with corresponding groups (such as amino group, amine group or hydroxy group) in the payload.

In one embodiment, the reactive group in B is independently amino group, hydroxy group or thiol group, which reacts with corresponding groups (such as halogen, hydroxy group, aldehyde group) in the payload. In another embodiment, the reactive group in B is independently hydroxy group, which reacts with corresponding groups (such as halogen or hydroxy group) in the payload.

In one embodiment, each B is independently a terminal group R¹⁰, or a combination of 1) a self-immolative spacer Sp1; 2) a bond, or one of or a combination of two or more of the bivalent groups selected from: -CR¹R²—, C₁₋₁₀ alkylene, C₄₋₁₀ cycloalkylene, C₄₋₁₀ heterocyclylene and —(CO)—; and 3) a terminal group R¹⁰.

In one embodiment, Sp1 is selected from PABC, acetal, heteroacetal and the combination thereof.

In one embodiment, Sp1 is acetal, heteroacetal or PABC. In one embodiment, the heteroacetal is selected from N,O-heteroacetal. In one embodiment, Sp1 is —O—CH₂—U—, or —NH—CH₂—U— wherein the —O— or the —NH— is connected to Cleavable sequence 1, wherein U is absent or U is O, S or N, preferably O or S.

In one embodiment, Sp1 is PABC.

In one embodiment, B is R¹⁰, —NH—CH₂—U—R¹⁰ or is —NH—CH₂—U—(CR¹R²)_{g}—(CO)—R¹⁰, wherein U is absent or U is O, S or N, g is 1; preferably U is O or S.

In one embodiment, B is R¹⁰.

In one embodiment, B is —NH—CH₂—O-R¹⁰.

In one embodiment, B is —NH—CH₂—O—(CR¹R²)_{g}—(CO)—R¹⁰, g is an integer of 1 to 10, preferably 1.

In one embodiment, B is —NH—CH₂—R¹⁰.

In one embodiment, R¹ is hydrogen.

In one embodiment, R² is hydrogen.

In one embodiment, R¹⁰ is hydrogen, hydroxy or

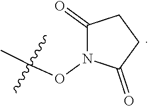 [SMILES: *ON1C(=O)CCC1=O]

In one embodiment, R¹⁰ is hydrogen. In one embodiment, R¹⁰ is hydroxy or

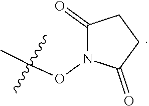 [SMILES: *ON1C(=O)CCC1=O]

In one embodiment, R¹⁰ represents the part of structure which would not appear in the product molecule resulting from the reaction of B with the payload.

### Specific Embodiment of the Formula (I) Compound

In one embodiment, each LKa is

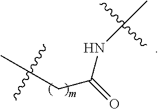

In one embodiment, formula (I) has the structure of formula (I-1)

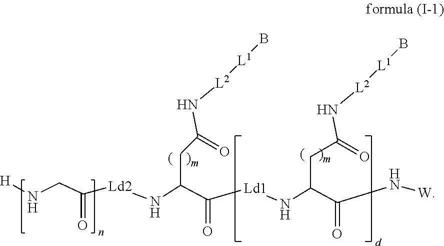

In one embodiment, Ld2 is

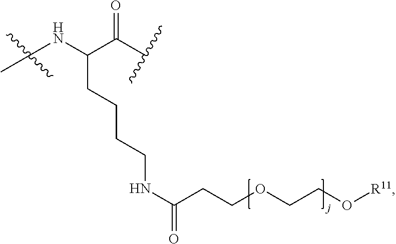

d is 1, W is hydrogen. In one embodiment, the compound of formula (I-1) is as follows:

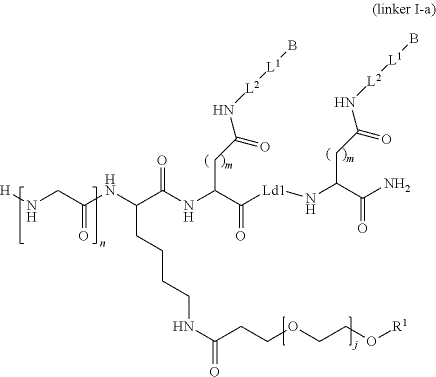

In one embodiment, Ld2 is a bond, d is 1, W is —C₂H₄-(PEG)-(CO)NH₂. In one embodiment, the compound of formula (I-1) is as follows:

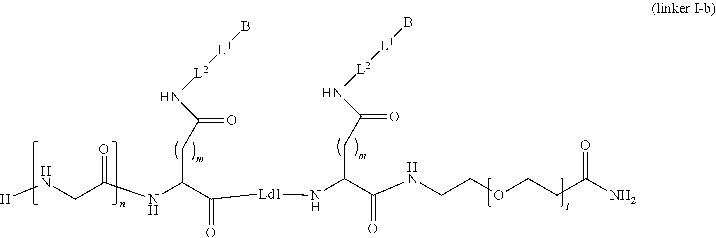

In one embodiment, Ld1 is

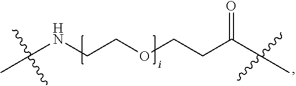

i is 4, n is 3, m is 1, j is 12, L² is —(C₂H₄—O)ₚ—(CH₂)₂(CO)—, p is 2, L¹ is Gly-Gly-Phe-Gly, B is —NH—CH₂—U-R¹⁰ or -R¹⁰ or —NH—CH₂-U-(CR¹R²)_{g}—(CO)—R¹⁰, U is O, g is 1, R¹¹ is methyl. In one embodiment, linker I-a has the structure of:

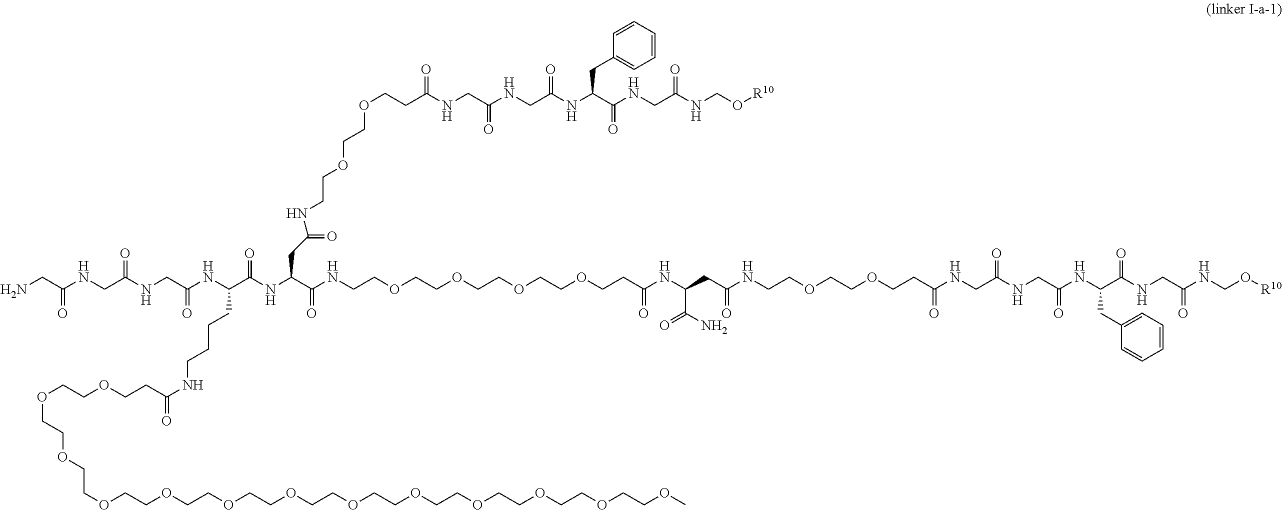

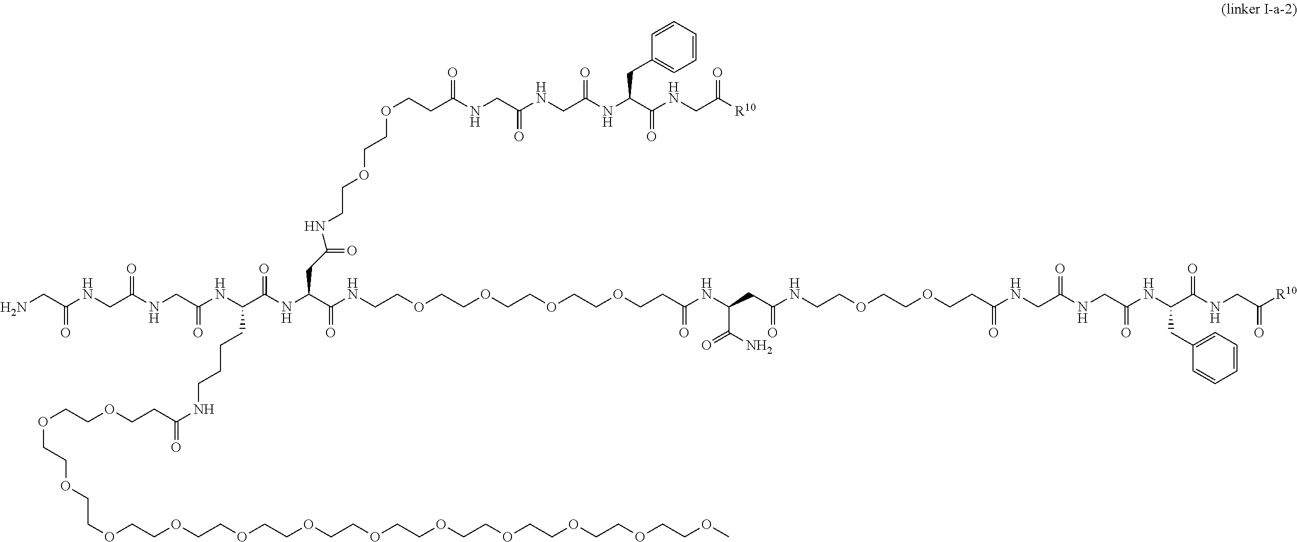

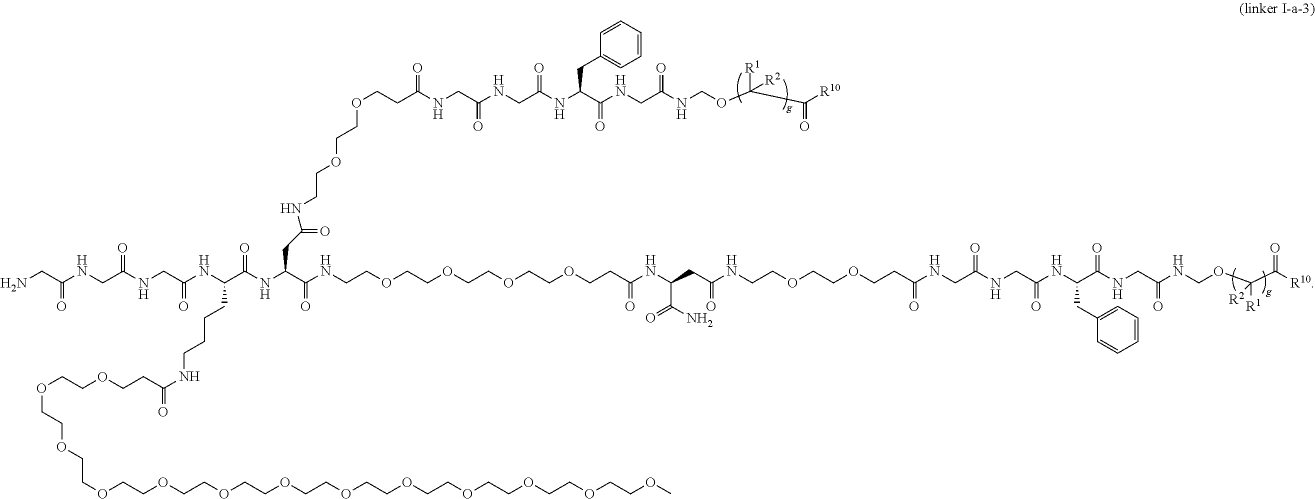

In one embodiment, Ld1 is

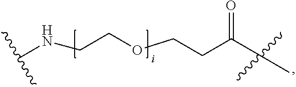

i is 4, n is 3, m is 2, j is 12, L² is —(C₂H₄—O)ₚ—(CH₂)₂(CO)—, p is 2, L¹ is Gly-Gly-Phe-Gly, B is —NH—CH₂—U-R¹⁰ or —R¹⁰ or -NH-CH₂-U—(CR¹R²)_{g}—(CO)—R¹⁰, U is O, g is 1, R¹¹ is methyl. In one embodiment, linker I-a has the structure of:

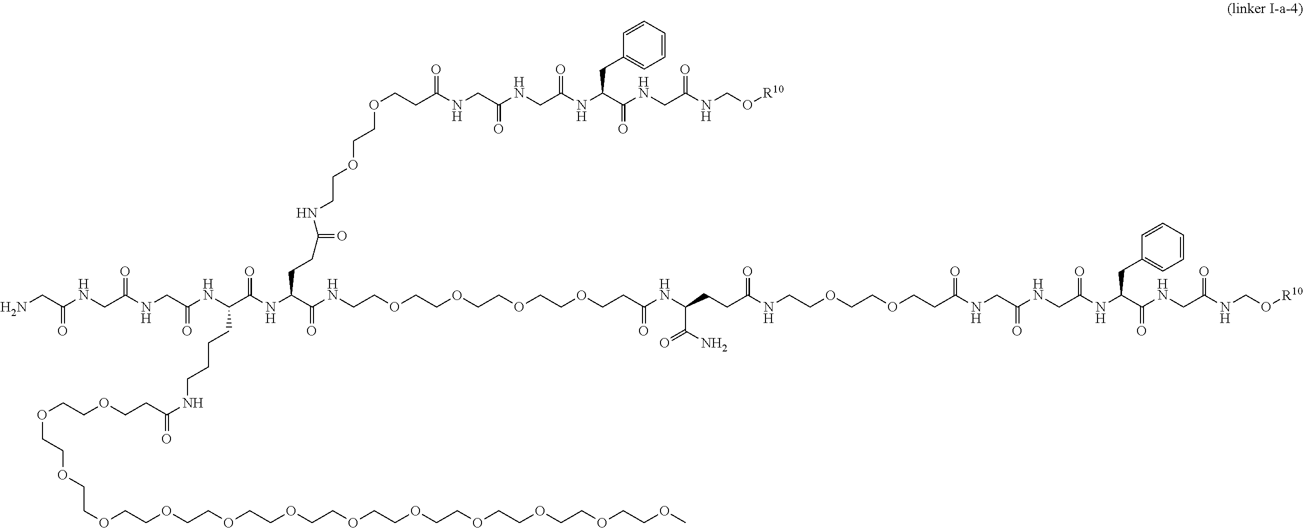

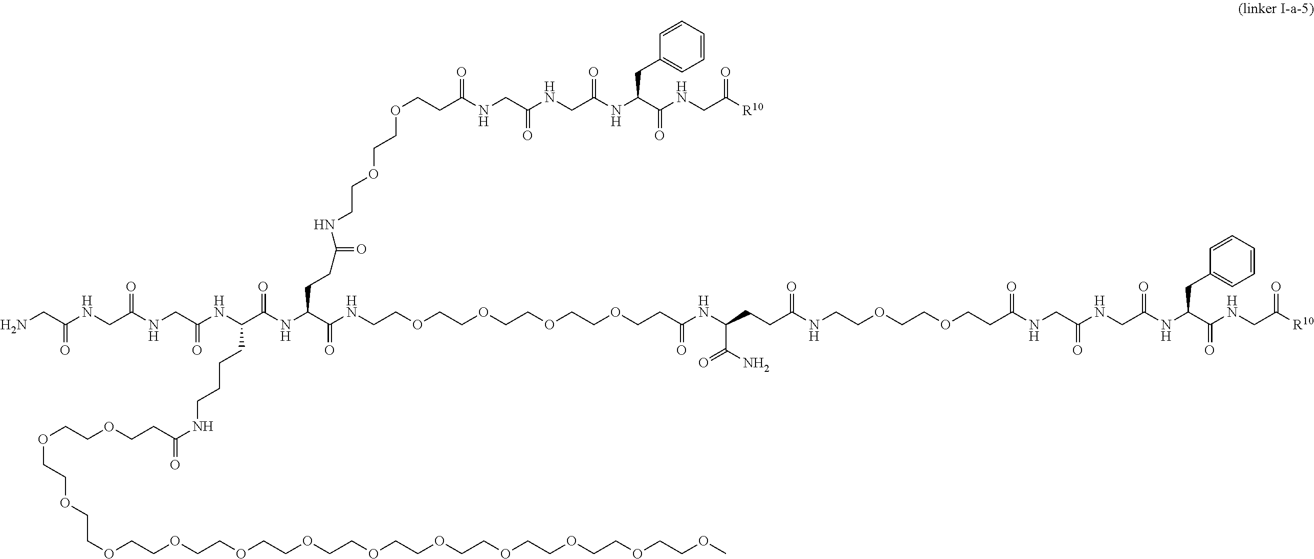

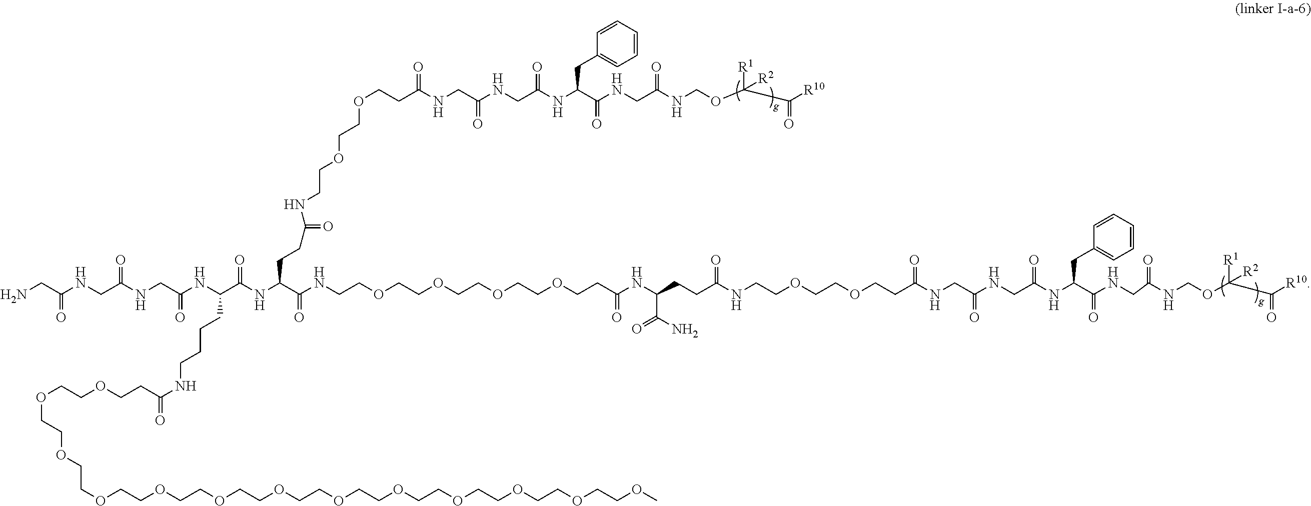

In one embodiment, Ld1 is

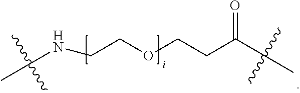

i is 4, n is 3, m is 2, j is 12, L² is —(C₂H₄—O)ₚ—(CH₂)₂(CO)—, p is 2, L¹ is Gly-Gly-Phe-Gly, B is —NH-CH₂-U-R¹⁰ or —R¹⁰ or PABC-NH-CH₂-U-(CR¹R²)_{g}—(CO)—R¹⁰, U is O, g is 1, R¹¹ is methyl. In one embodiment, linker I-a has the structure of:

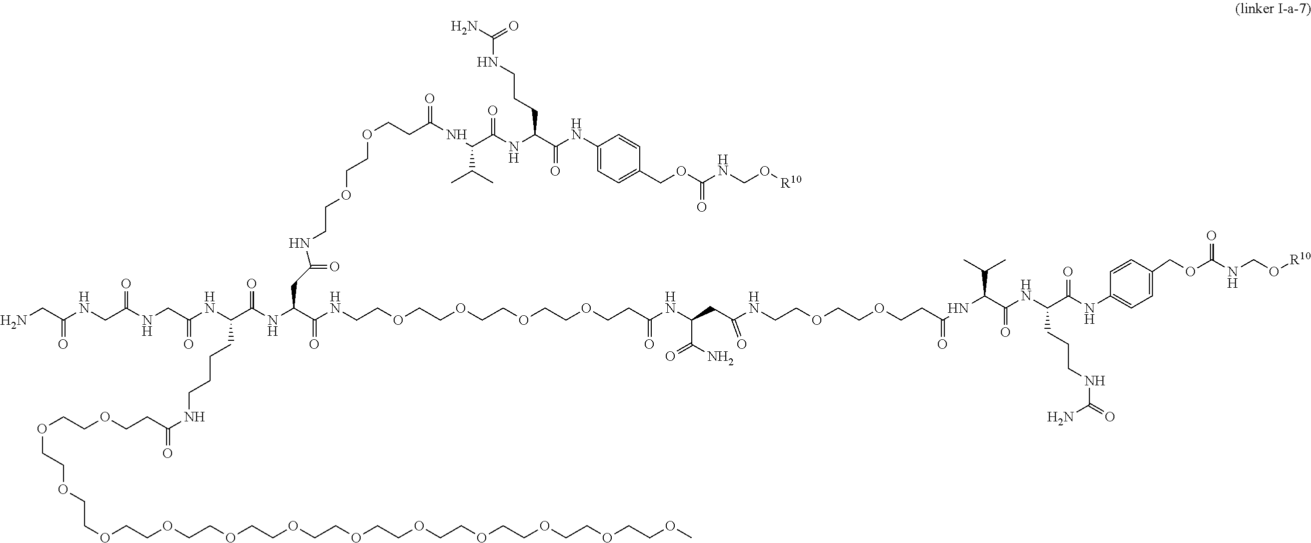

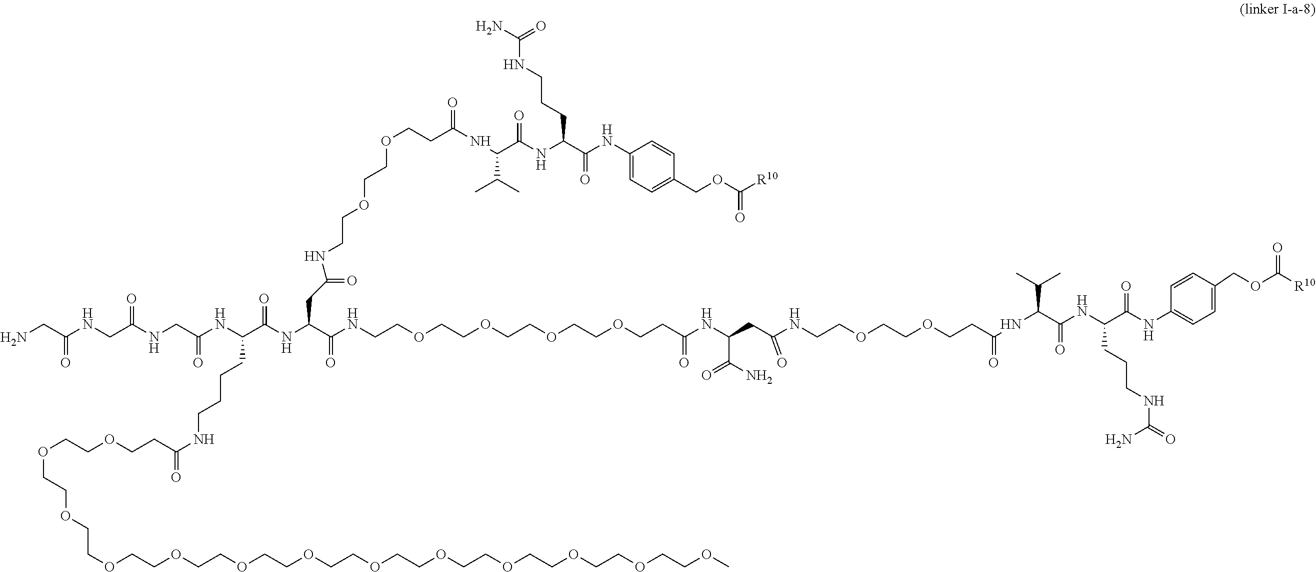

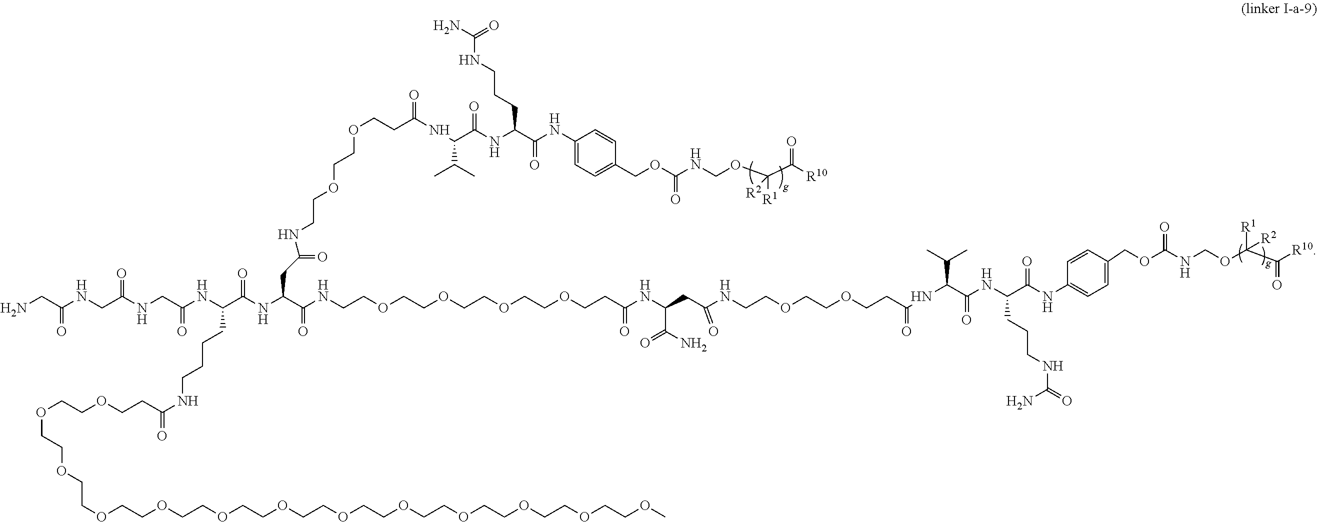

In one embodiment, Ld1 is

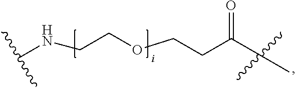

i is 4, n is 3, m is 2, j is 12, L² is —(C₂H₄—O)ₚ—(CH₂)₂(CO)—, p is 2, L¹ is Val-Cit, B is PABC—NH—CH₂-U—R¹⁰ or PABC-R¹⁰ or PABC—NH—CH₂—U-(CR¹R²)_{g}—(CO)—R¹⁰, U is O, g is 1, R¹¹ is methyl. In one embodiment, linker I-a has the structure of:

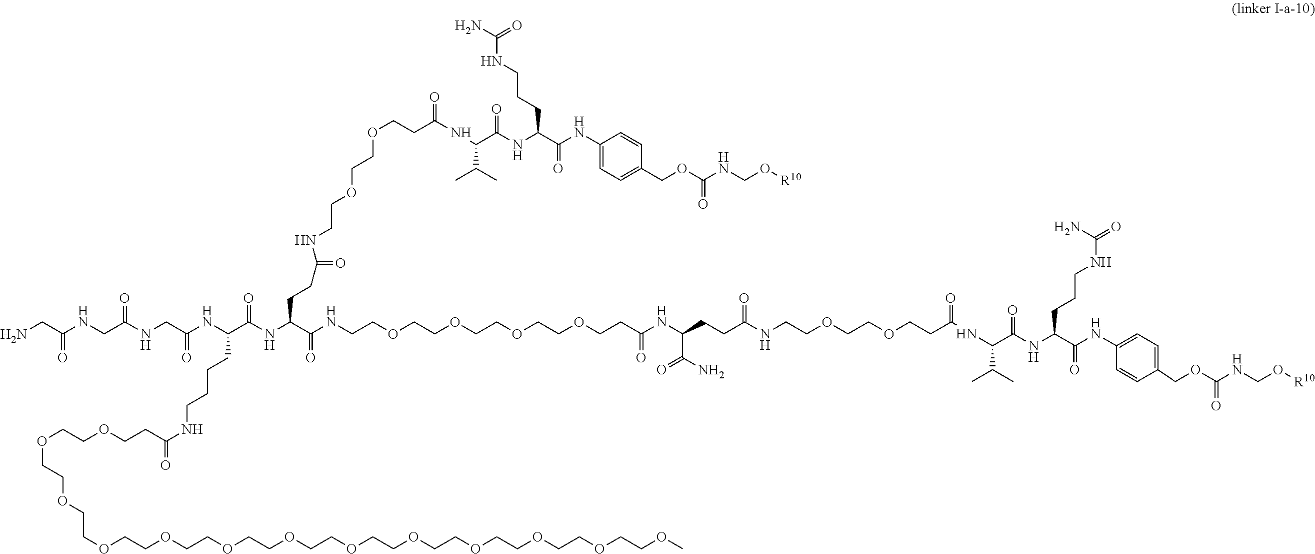

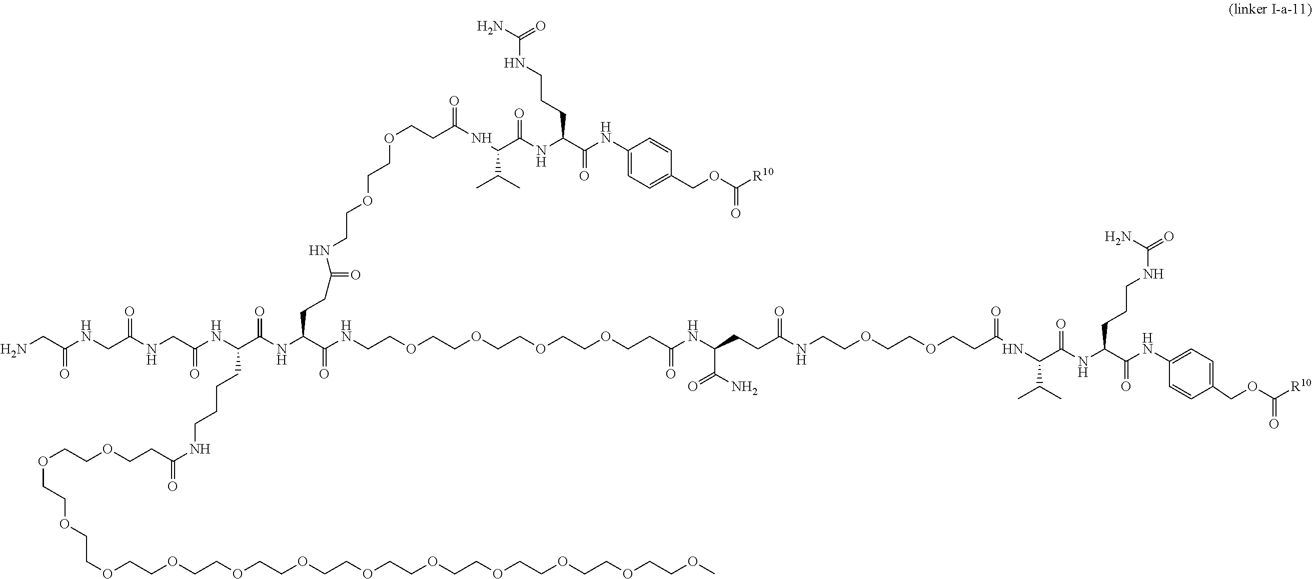

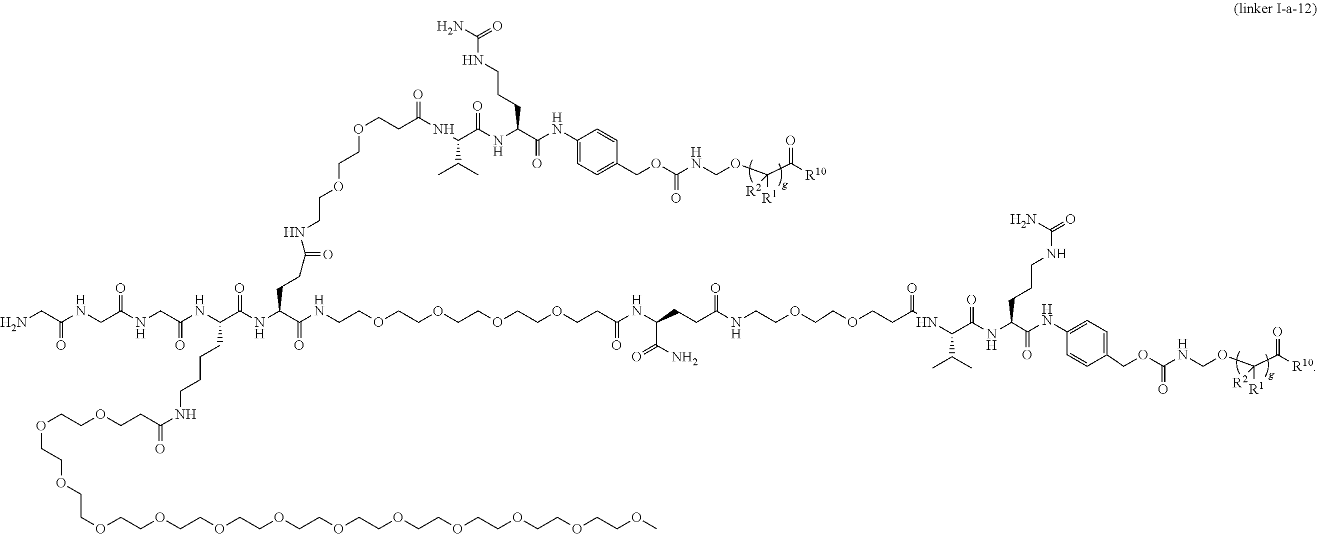

In one embodiment, Ld1 is

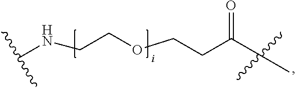

i is 4, n is 3, m is 1, t is 12, L² is —(C₂H₄—O)ₚ—(CH₂)₂(CO)—, p is 2, L¹ is Gly-Gly-Phe-Gly, B is —NH—CH₂—U—R¹⁰ or —R¹⁰ or —NH—CH₂—U-(CR¹R²)_{g}—(CO)—R¹⁰, U is O, g is 1. In one embodiment, linker I-b has the structure of:

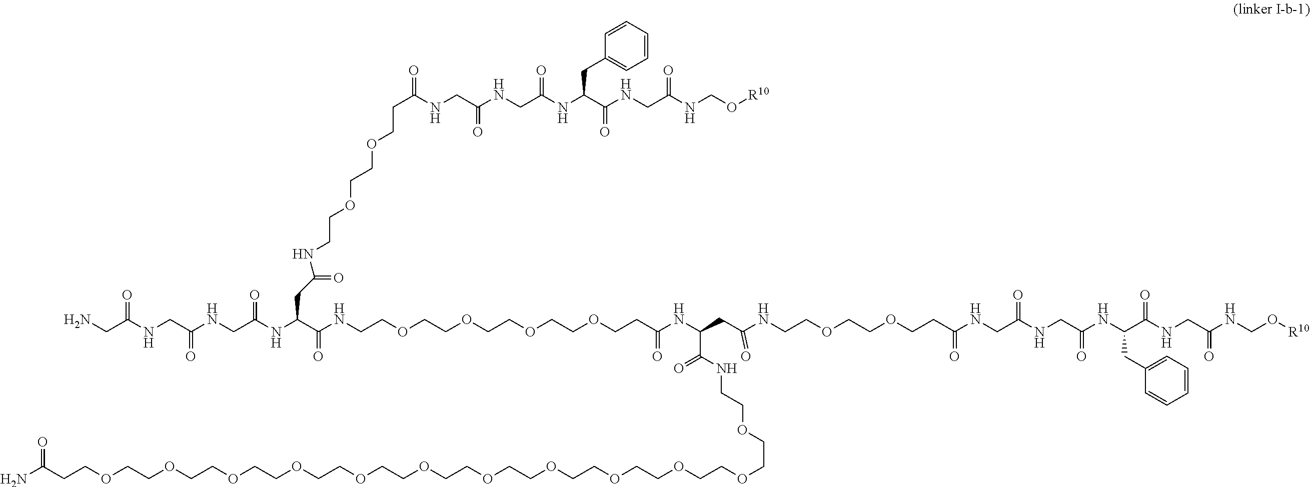

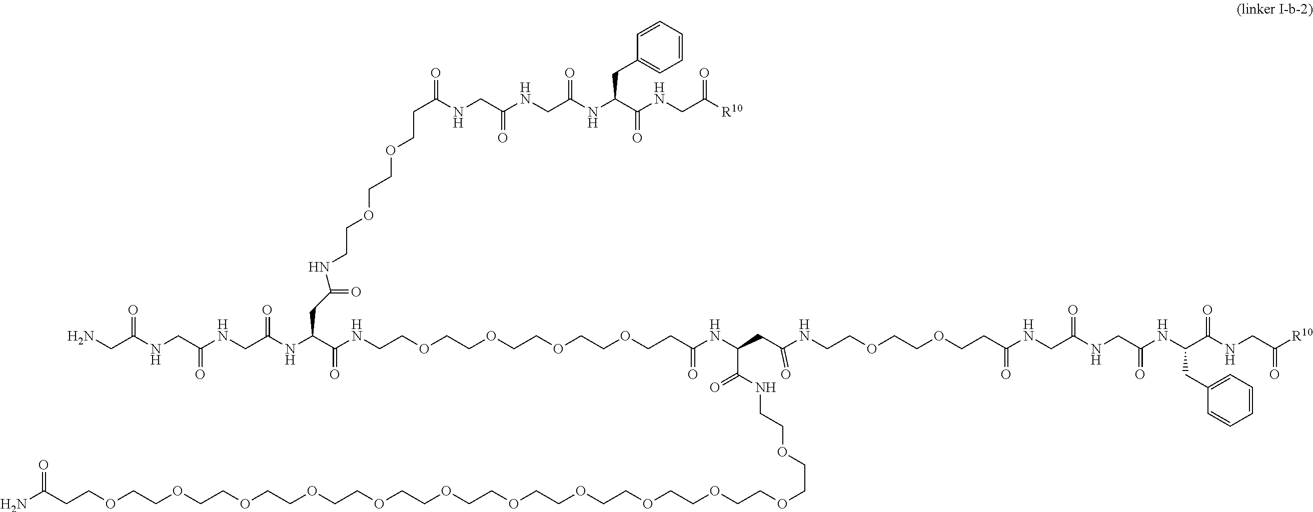

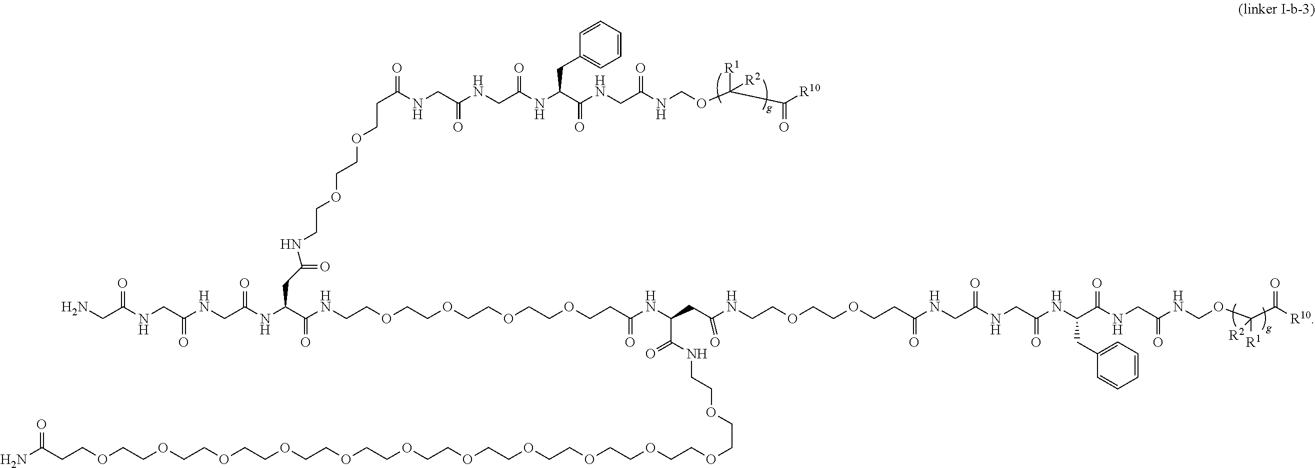

In one embodiment, Ld1 is

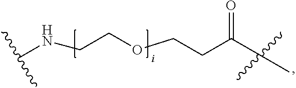

i is 4, n is 3, m is 2, t is 12, L² is —(C₂H₄—O)ₚ—(CH₂)₂(CO)—, p is 2, L¹ is Gly-Gly-Phe-Gly, B is —NH—CH₂—U-R¹⁰ or -R¹⁰ or —NH-CH₂-U—(CR¹R²)_{g}—(CO)—R¹⁰, U is O, g is 1. In one embodiment, linker I-b has the structure of:

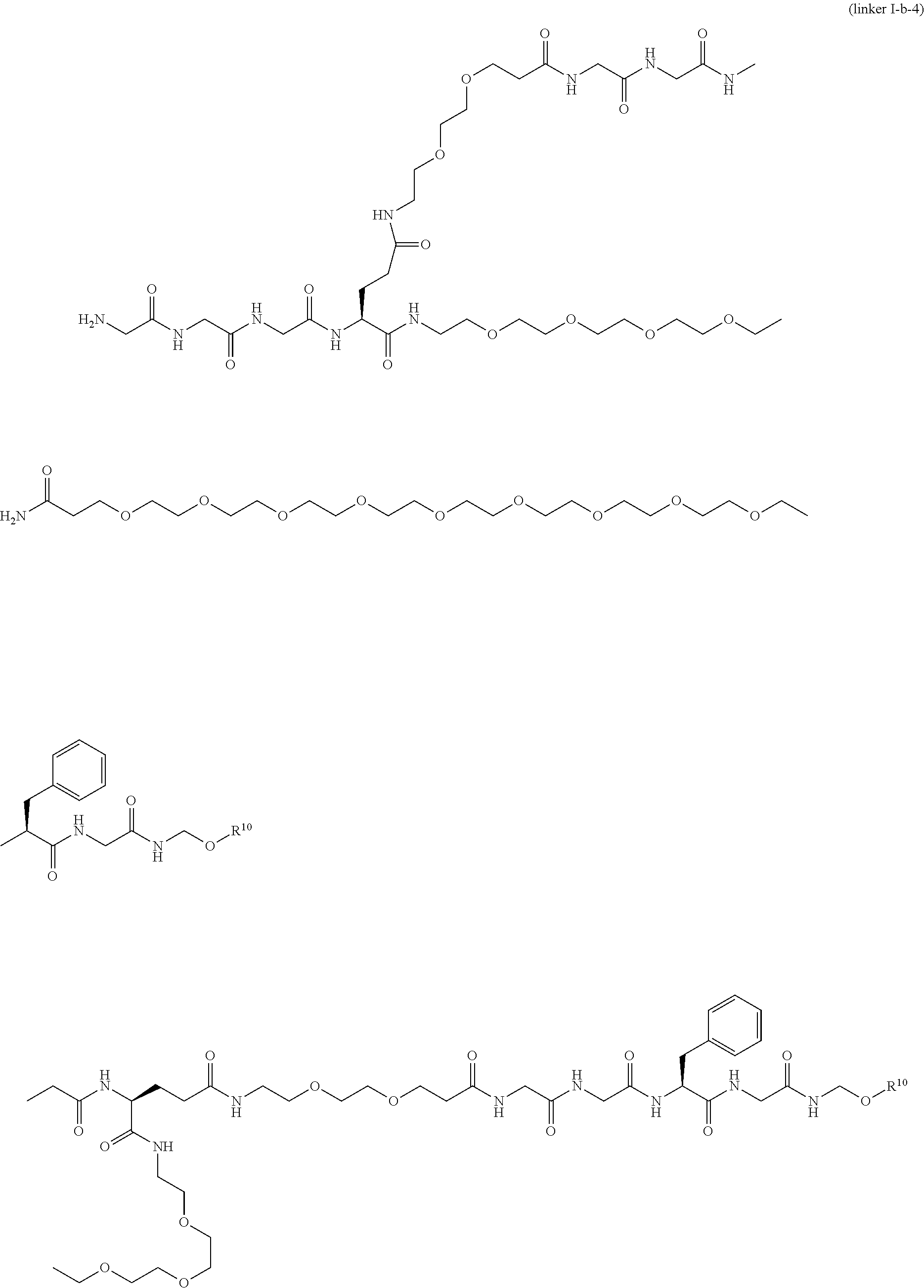

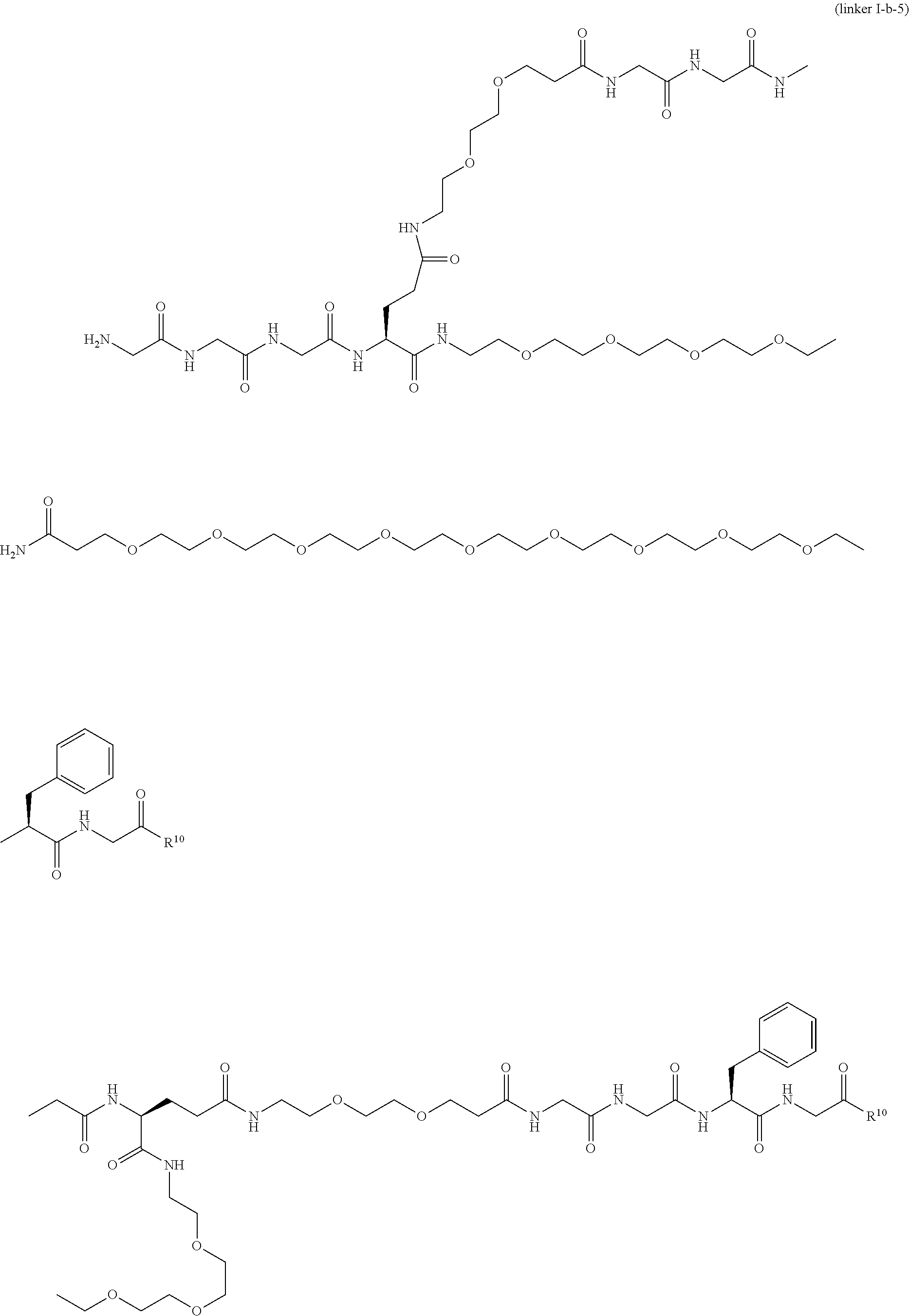

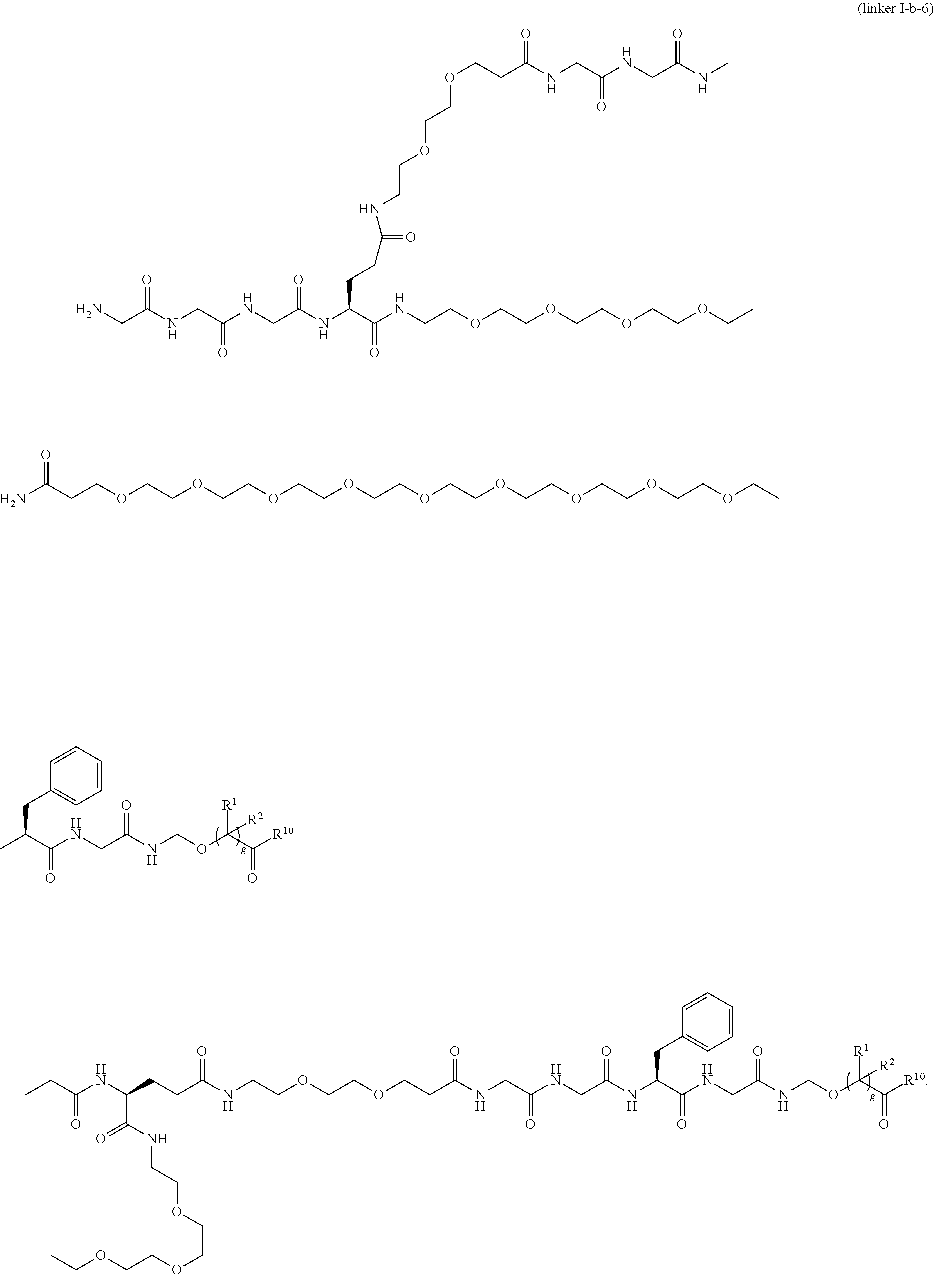

In one embodiment, Ld1 is

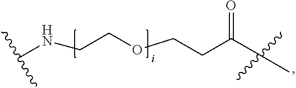

i is 4, n is 3, m is 1, t is 12, L² is —(C₂H₄-O), —(CH₂)₂(CO)—, p is 2, L¹ is Val-Cit, B is PABC—NH—CH₂—U-R¹⁰ or PABC-R¹⁰ or PABC—NH—CH₂-U—(CR¹R²)_{g}—(CO)—R¹⁰, U is O, g is 1. In one embodiment, linker I-b has the structure of:

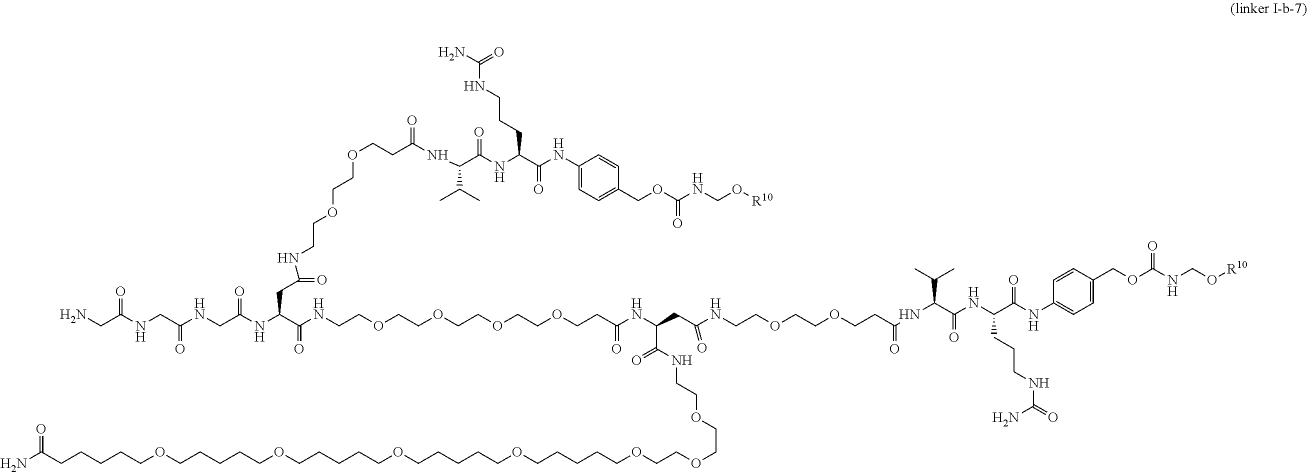

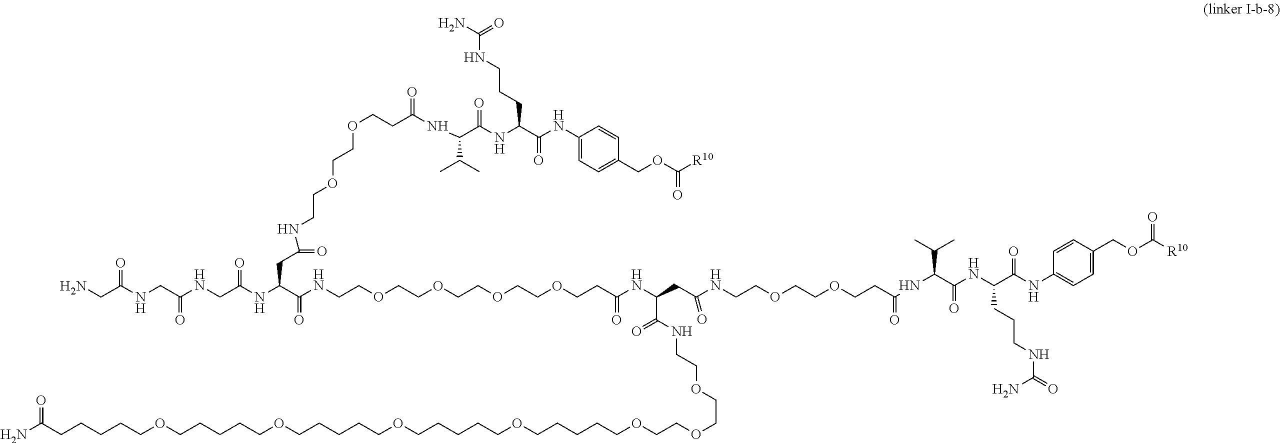

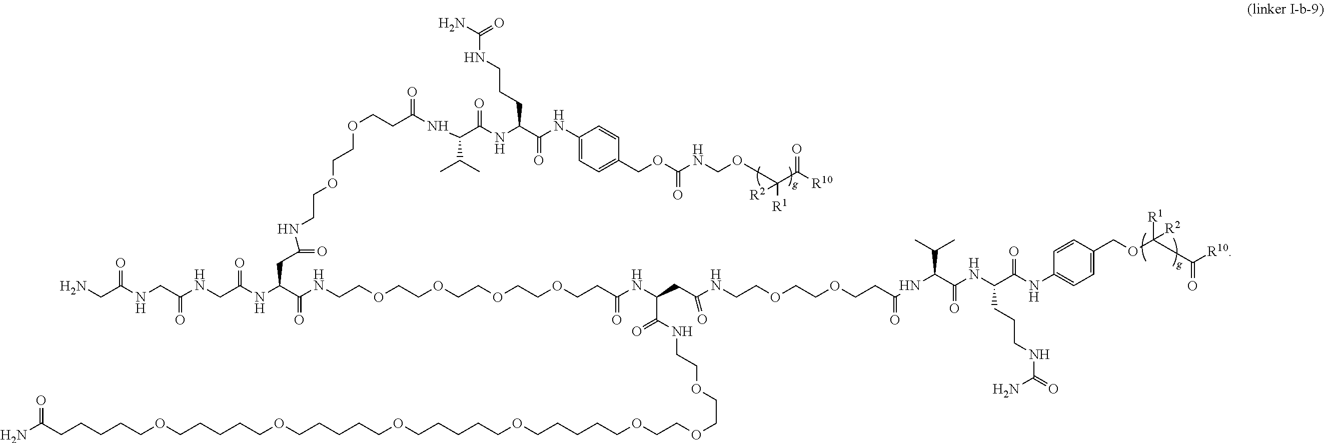

In one embodiment, Ld1 is

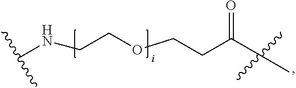

i is 4, n is 3, m is 2, t is 12, L² is —(C₂H₄—O)ₚ—(CH₂)₂(CO)—, p is 2, L¹ is Val-Cit, B is PABC—NH-CH2-U—R¹⁰ or PABC-R¹⁰ or PABC—NH—CH₂-U—(CR¹R²)_{g}—(CO)—R¹⁰, U is O, g is 1. In one embodiment, linker I-b has the structure of:

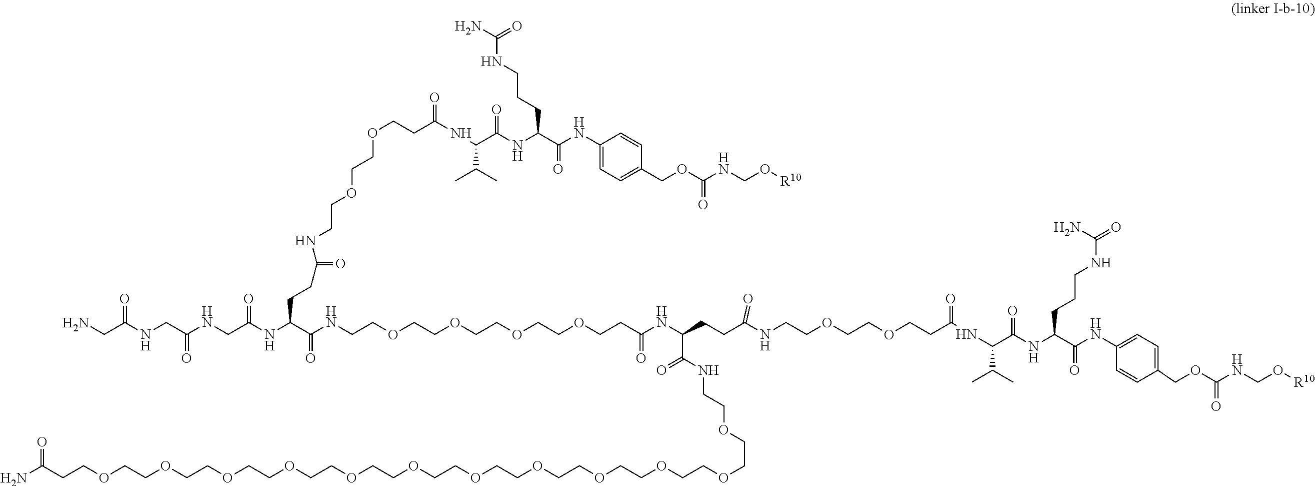

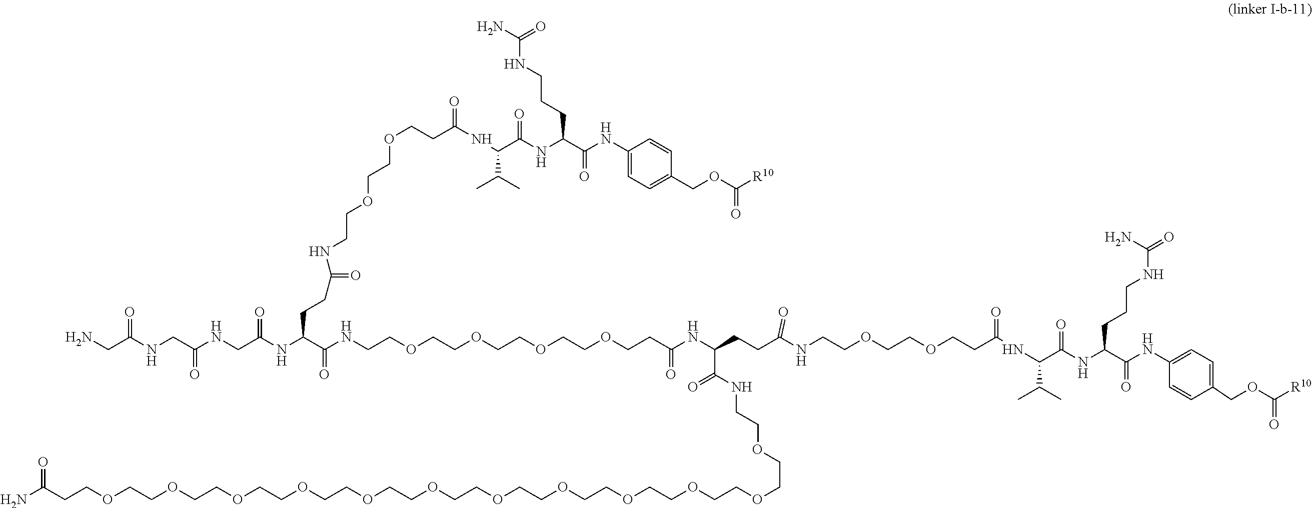

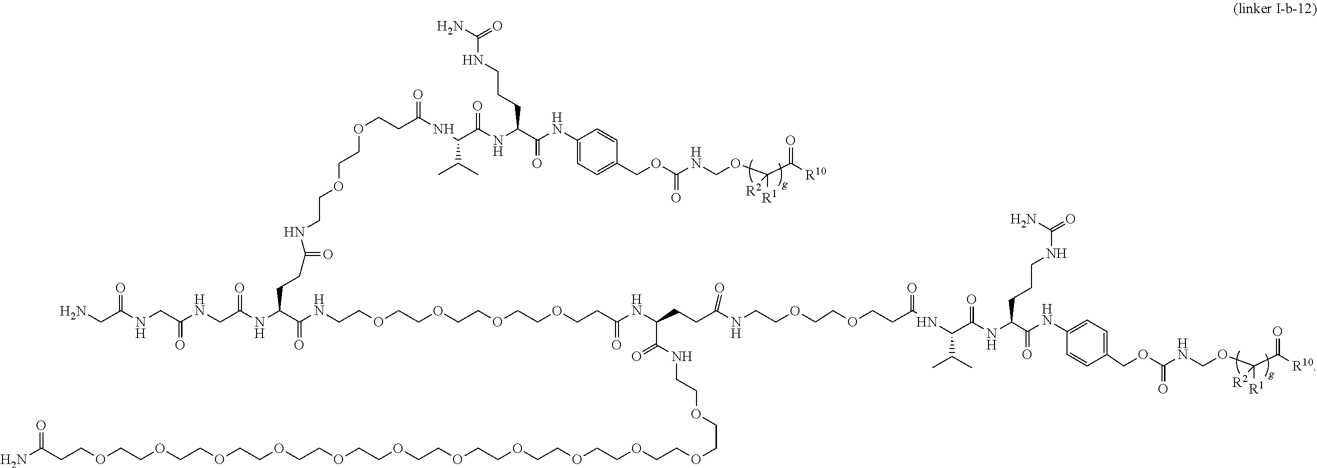

### Payload-Bearing Formula (I) Compound

The reactive group comprised by B is covalently conjugated with a payload containing another reactive group to give a payload-bearing formula (I) compound.

In yet another aspect, provided is a compound having the structure of formula (II)

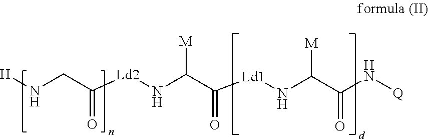

Wherein
Q is hydrogen, —C₂H₄-(PEG)ₜ-(CO)NH₂ or LKb-P;
M is LKa-LKb-P;
P is a payload which is linked to the B moiety or L¹ moiety of the compound of formula (I);
n, d, Ld1, Ld2, t, LKa and LKb are as defined in formula (I).

### Payload

In the present disclosure, the payload may be selected from the group consisting of small molecule compounds, nucleic acids and analogues, tracer molecules (including fluorescent molecules, etc.), short peptides, polypeptides, peptidomimetics, and proteins. In one embodiment, the payload is selected from the group consisting of small molecule compounds, nucleic acid molecules, and tracer molecules. In a preferred embodiment, the payload is selected from small molecule compounds. In a more preferred embodiment, the payload is selected from the group consisting of cytotoxin and fragments thereof.

In one embodiment, the cytotoxin is selected from the group consisting of drugs that target microtubule cytoskeleton. In a preferred embodiment, the cytotoxin is selected from the group consisting of taxanes, maytansinoids, auristatins, epothilones, combretastatin A-4 phosphate, combretastatin A-4 and derivatives thereof, indol-sulfonamides, vinblastines such as vinblastine, vincristine, vindesine, vinorelbine, vinflunine, vinglycinate, anhydrovinblastine, dolastatin 10 and analogues, halichondrin B and eribulin, indole-3-oxoacetamide, podophyllotoxins, 7-diethylamino-3-(2′-benzoxazolyl)-coumarin (DBC), discodermolide, laulimalide. In another embodiment, the cytotoxin is selected from the group consisting of DNA topoisomerase inhibitors such as camptothecins and derivatives thereof, mitoxantrone, mitoguazone. In a preferred embodiment, the cytotoxin is selected from the group consisting of nitrogen mustards such as chlorambucil, chlornaphazine, cholophosphamide, estramustine, ifosfamide, mechlorethamine, mechlorethamine oxide hydrochloride, melphalan, novembichin, phenamet, phenesterine, prednimustine, trofosfamide, uracil mustard. In yet another preferred embodiment, the cytotoxin is selected from the group consisting of nitrosoureas such as carmustine, flubenzuron, formoterol, lomustine, nimustine, ramustine. In one embodiment, the cytotoxin is selected from the group consisting of aziridines. In a preferred embodiment, the cytotoxin is selected from the group consisting of benzodopa, carboquone, meturedepa, and uredepa. In one embodiment, the cytotoxin is selected from the group consisting of an anti-tumor antibiotic. In a preferred embodiment, the cytotoxin is selected from the group consisting of enediyne antibiotics. In a more preferred embodiment, the cytotoxin is selected from the group consisting of dynemicin, esperamicin, neocarzinostatin, and aclacinomycin. In another preferred embodiment, the cytotoxin is selected from the group consisting of actinomycin, antramycin, bleomycins, actinomycin C, carabicin, carminomycin, and cardinophyllin, carminomycin, actinomycin D, daunorubicin, detorubicin, adriamycin, epirubicin, esorubicin, idarubicin, marcellomycin, mitomycins, nogalamycin, olivomycin, peplomycin, porfiromycin, puromycin, ferric adriamycin, rodorubicin, rufocromomycin, streptozocin, zinostatin, zorubicin. In yet another preferred embodiment, the cytotoxin is selected from the group consisting of trichothecene. In a more preferred embodiment, the cytotoxin is selected from the group consisting of T-2 toxin, verracurin A, bacillocporin A, and anguidine. In one embodiment, the cytotoxin is selected from the group consisting of an anti-tumor amino acid derivatives. In a preferred embodiment, the cytotoxin is selected from the group consisting of ubenimex, azaserine, 6-diazo-5-oxo-L-norleucine. In another embodiment, the cytotoxin is selected from the group consisting of folic acid analogues. In a preferred embodiment, the cytotoxin is selected from the group consisting of dimethyl folic acid, methotrexate, pteropterin, trimetrexate, and edatrexate. In one embodiment, the cytotoxin is selected from the group consisting of purine analogues. In a preferred embodiment, the cytotoxin is selected from the group consisting of fludarabine, 6-mercaptopurine, tiamiprine, thioguanine. In yet another embodiment, the cytotoxin is selected from pyrimidine analogues. In a preferred embodiment, the cytotoxin is selected from the group consisting of ancitabine, gemcitabine, enocitabine, azacitidine, 6-azauridine, carmofur, cytarabine, dideoxyuridine, doxifluridine, floxuridine. In one embodiment, the cytotoxin is selected from the group consisting of androgens. In a preferred embodiment, the cytotoxin is selected from the group consisting of calusterone, dromostanolone propionate, epitiostanol, mepitiostane, testolactone. In another embodiment, the cytotoxin is selected from the group consisting of anti-adrenals. In a preferred embodiment, the cytotoxin is selected from the group consisting of aminoglutethimide, mitotane, and trilostane. In one embodiment, the cytotoxin is selected from the group consisting of anti-androgens. In a preferred embodiment, the cytotoxin is selected from the group consisting of flutamide, nilutamide, bicalutamide, leuprorelin acetate, and goserelin. In yet another embodiment, the cytotoxin is selected from the group consisting of a protein kinase inhibitor and a proteasome inhibitor. In another embodiment, the cytotoxin is selected from the group consisting of vinblastines, colchicines, taxanes, auristatins, maytansinoids, calicheamicin, doxonubicin, duocarmucin, SN-38, cryptophycin analogue, deruxtecan, duocarmazine, calicheamicin, centanamycin, dolastansine, and pyrrolobenzodiazepine (PBD). In a particular embodiment, the cytotoxin is selected from the group consisting of vinblastines, colchicines, taxanes, auristatins, and maytansinoids.

In a particular embodiment, the cytotoxin is exatecan or a derivative thereof, such as DX8951f and the like.

In another particular embodiment, the cytotoxin is an maytansinoid, such as DM1 and the like.

In a particular embodiment, the cytotoxin is an auristatin, such as MMAE (monomethyl auristatin E), MMAF (monomethyl auristatin F), MMAD (monomethyl auristatin D) and the like. The synthesis and structure of austenitic compounds are described in US20060229253, the entire disclosure of which is incorporated herein by reference.

The payload contains a reactive group which can react with the reactive group in the compound of formula (I) and thus covalently conjugate the payload with the compound of formula (I). Compounds that do not contain reactive groups require appropriate derivatization to give the payload.

In one embodiment, the cytotoxin is a compound of the following formula (i)

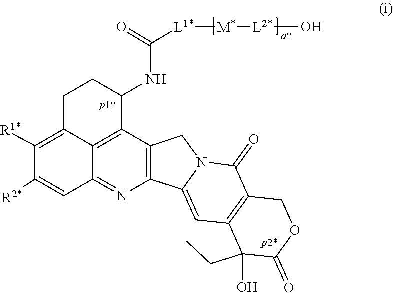

wherein,
a* is 0 or 1;
the carbon atoms marked with p1* and p2* each is asymmetric center, and the asymmetric center is S configured, R configured or racemic;
L^{1*} is selected from C₁₋₆ alkylene, which is unsubstituted or substituted with one substituent selected from halogen, —OH and -NH₂;
M* is —CH₂—, —NH— or —O—;
L^{2*} is C₁₋₃ alkylene;
R^{1*} and R^{2*} are each independently selected from hydrogen, C₁₋₆ alkyl, halogen and C₁₋₆ alkoxy.

In a particular embodiment, the cytotoxin is a compound of the following formula (i′)

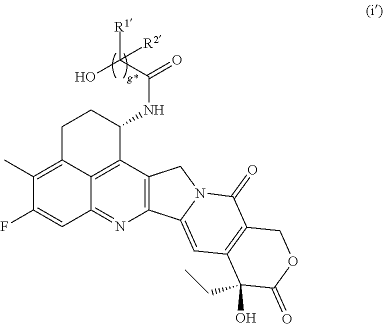

wherein, g* is any integer of 1 to 6;
R^{1′} and R^{2′} are each independently selected from hydrogen, halogen, —C₁₋₁₀ alkyl, —C₁₋₁₀ haloalkyl, C₄₋₁₀ cycloalkylene; or
R^{1′} and R^{2′} together with the carbon atom to which they are attached form a 3-6 membered cycloalkyl group.

In one embodiment, g* is any integer of 1 to 3, preferably 1.

In one embodiment, R^{1′} is hydrogen.

In one embodiment, R^{2′} is hydrogen.

In one embodiment, L^{1*} is selected from C₁₋₆linear alkylene, C₁₋₆branched alkylene, C₃₋₆ cyclic alkylene and C₃₋₄ cyclic alkyl-C₁₋₂ linear alkylene group, which are each independently unsubstituted or substituted with one substituent selected from halogen, —OH and —NH₂. In one embodiment, L^{1*} is selected from C₁₋₄ alkylene, which is unsubstituted or substituted with one substituent selected from halogen, —OH and —NH₂. In a preferred embodiment, L^{1*} is selected from —CH₂—, —C₂H₄—,

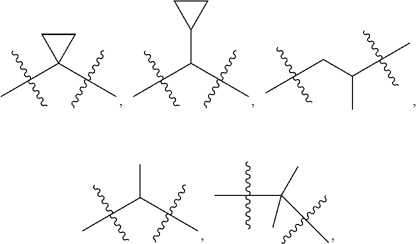

which are each independently unsubstituted or substituted with at least one substituent selected from halogen, —OH and —NH₂. In a preferred embodiment, L^{1*} is selected from —CH₂—,

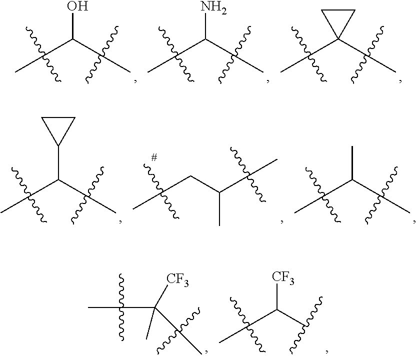

wherein “#” marks the position attached to carbonyl. In a more preferred embodiment, L^{1*} is selected from —CH₂—,

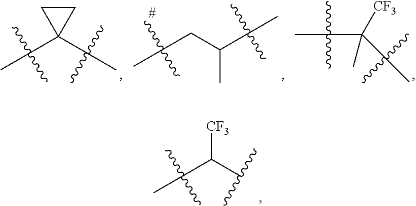

wherein “#” marks the position attached to carbonyl. In a particular embodiment, L^{1*} is selected from —CH₂—,

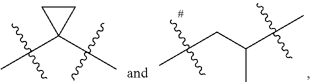

wherein “#” marks the position attached to carbonyl. In a preferred embodiment, the halogen is selected from F, Cl and Br, especially F.

In one embodiment, a* is 1, M* is —CH₂—, —NH— or —O—; and L^{2*} is —C₂H₄—. In another embodiment, a* is 1, M* is —CH₂—, and L^{2*} is —CH₂—. In one embodiment, a* is 0.

In one embodiment, the carbon atom marked with p1* is S configured or racemic, preferably S configured. In another embodiment, the carbon atom marked with p2* is S configured or racemic, preferably S configured.

In one embodiment, R^{1*} and R^{2*} are each independently selected from hydrogen, C₁₋₃ alkyl, halogen and C₁₋₃ alkoxy. In a preferred embodiment, R^{1*} and R^{2*} are each independently selected from CH₃—, F, Cl, Br and CH₃O—. In one embodiment, R^{1*} is selected from CH₃- and Cl. In another embodiment, R²* is F.

In one embodiment, a* is 0, L^{1*} is selected from —CH₂—,

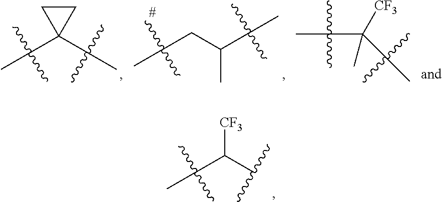

wherein “#” marks the position attached to carbonyl. In one embodiment, a* is 1, L^{1*} is

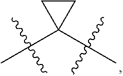 [SMILES: *C1(*)CC1]

M* is O, and L^{2*} is —C₂H₄—.

In one embodiment, a* is 0, R^{1*} is Cl, R^{2*} is F, and L^{1*} is selected from —CH₂—,

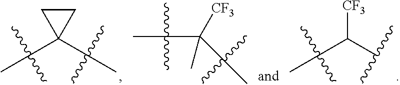

In one embodiment, a* is 0, R^{1*} is CH₃—, R^{2*} is F, and L^{1*} is selected from

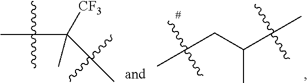

wherein “#” marks the position attached to carbonyl.

In one embodiment, a* is 1, R^{1*} is CH₃—, R^{2*} is F, L^{1*} is

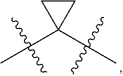 [SMILES: *C1(*)CC1]

M is O, and L^{2*} is —C₂H₄—.

In one embodiment, the cytotoxin is selected from:

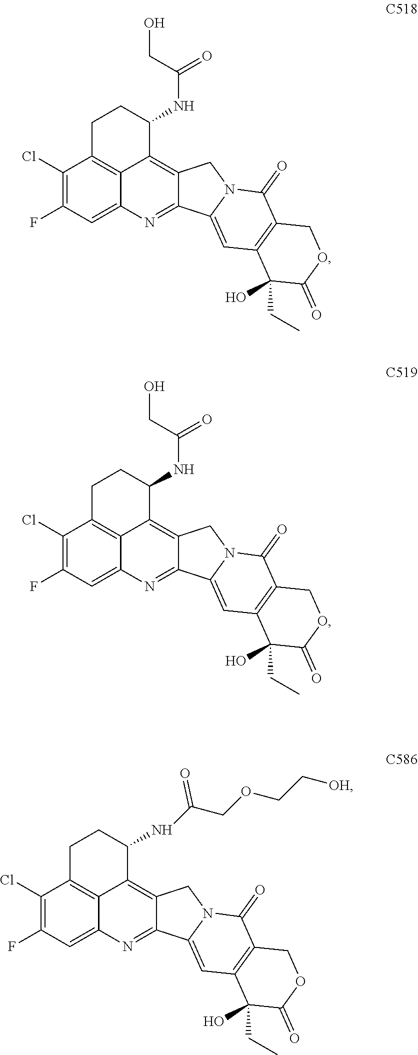 [SMILES: CC[C@@]1(O)C(=O)OCc2c1cc1n(c2=O)Cc2c-1nc1cc(F)c(Cl)c3c1c2[C@@H](NC(=O)CO)CC3 ; CC[C@@]1(O)C(=O)OCc2c1cc1n(c2=O)Cc2c-1nc1cc(F)c(Cl)c3c1c2[C@H](NC(=O)CO)CC3 ; CC[C@@]1(O)C(=O)OCc2c1cc1n(c2=O)Cc2c-1nc1cc(F)c(Cl)c3c1c2[C@@H](NC(=O)COCCO)CC3]

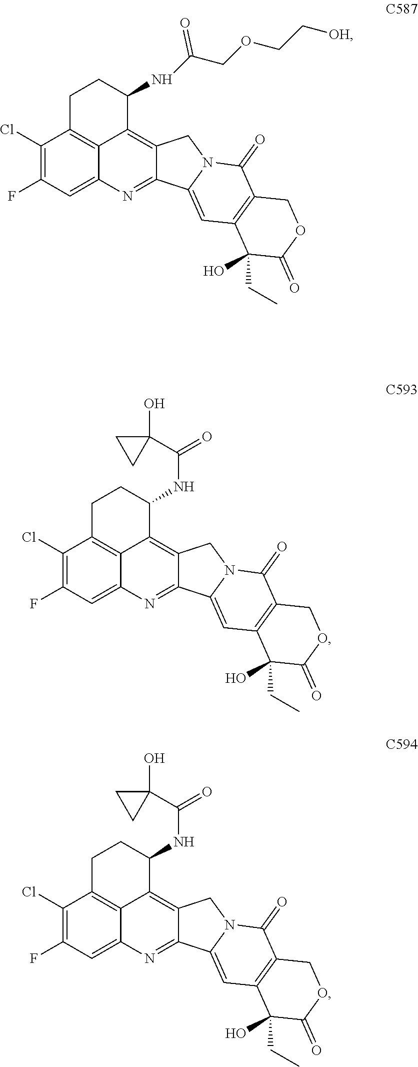 [SMILES: CC[C@@]1(O)C(=O)OCc2c1cc1n(c2=O)Cc2c-1nc1cc(F)c(Cl)c3c1c2[C@H](NC(=O)COCCO)CC3 ; CC[C@@]1(O)C(=O)OCc2c1cc1n(c2=O)Cc2c-1nc1cc(F)c(Cl)c3c1c2[C@@H](NC(=O)C1(O)CC1)CC3 ; CC[C@@]1(O)C(=O)OCc2c1cc1n(c2=O)Cc2c-1nc1cc(F)c(Cl)c3c1c2[C@H](NC(=O)C1(O)CC1)CC3]

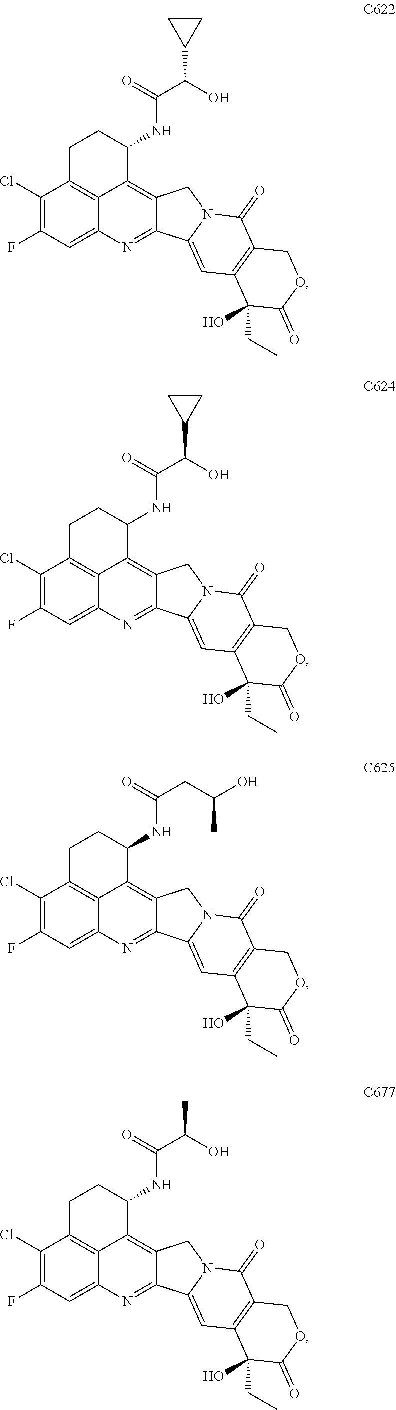 [SMILES: CC[C@@]1(O)C(=O)OCc2c1cc1n(c2=O)Cc2c-1nc1cc(F)c(Cl)c3c1c2[C@@H](NC(=O)[C@@H](O)C1CC1)CC3 ; CC[C@@]1(O)C(=O)OCc2c1cc1n(c2=O)Cc2c-1nc1cc(F)c(Cl)c3c1c2C(NC(=O)[C@H](O)C1CC1)CC3 ; CC[C@@]1(O)C(=O)OCc2c1cc1n(c2=O)Cc2c-1nc1cc(F)c(Cl)c3c1c2[C@H](NC(=O)C[C@H](C)O)CC3 ; CC[C@@]1(O)C(=O)OCc2c1cc1n(c2=O)Cc2c-1nc1cc(F)c(Cl)c3c1c2[C@@H](NC(=O)[C@@H](C)O)CC3]

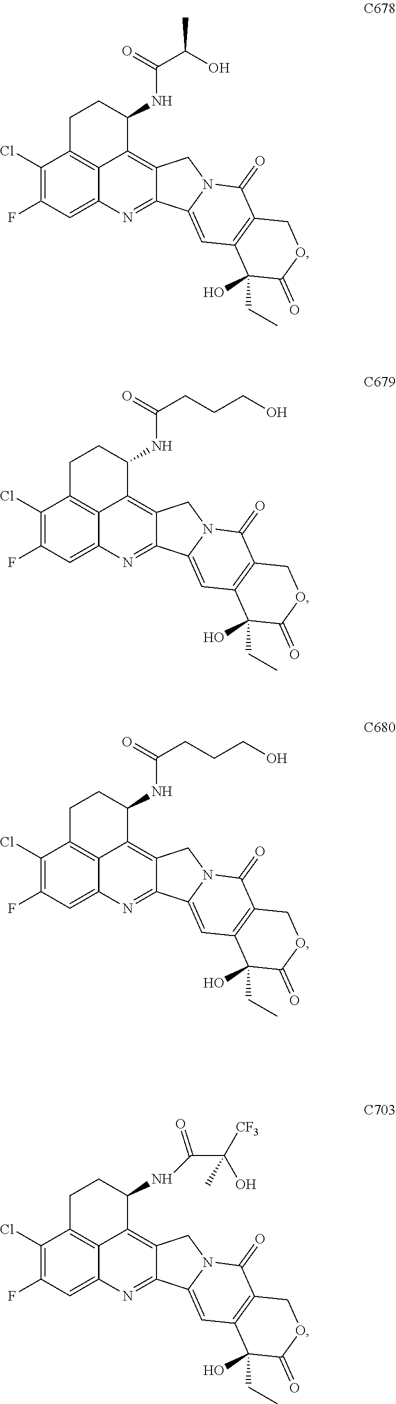 [SMILES: CC[C@@]1(O)C(=O)OCc2c1cc1n(c2=O)Cc2c-1nc1cc(F)c(Cl)c3c1c2[C@H](NC(=O)[C@@H](C)O)CC3 ; CC[C@@]1(O)C(=O)OCc2c1cc1n(c2=O)Cc2c-1nc1cc(F)c(Cl)c3c1c2[C@@H](NC(=O)CCCO)CC3 ; CC[C@@]1(O)C(=O)OCc2c1cc1n(c2=O)Cc2c-1nc1cc(F)c(Cl)c3c1c2[C@H](NC(=O)CCCO)CC3 ; CC[C@@]1(O)C(=O)OCc2c1cc1n(c2=O)Cc2c-1nc1cc(F)c(Cl)c3c1c2[C@H](NC(=O)[C@](C)(O)C(F)(F)F)CC3]

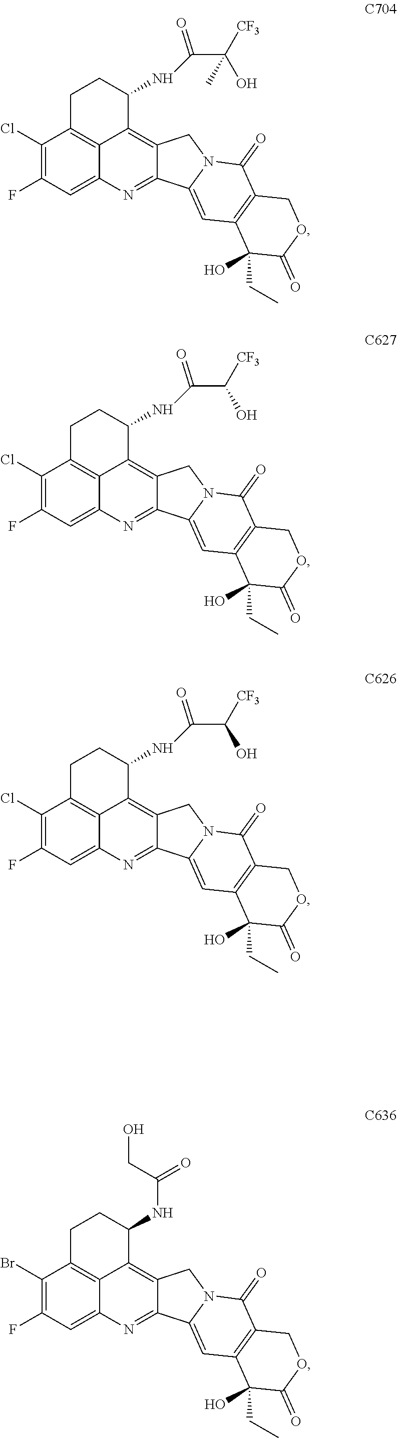 [SMILES: CC[C@@]1(O)C(=O)OCc2c1cc1n(c2=O)Cc2c-1nc1cc(F)c(Cl)c3c1c2[C@@H](NC(=O)[C@](C)(O)C(F)(F)F)CC3 ; CC[C@@]1(O)C(=O)OCc2c1cc1n(c2=O)Cc2c-1nc1cc(F)c(Cl)c3c1c2[C@@H](NC(=O)[C@@H](O)C(F)(F)F)CC3 ; CC[C@@]1(O)C(=O)OCc2c1cc1n(c2=O)Cc2c-1nc1cc(F)c(Cl)c3c1c2[C@@H](NC(=O)[C@H](O)C(F)(F)F)CC3 ; CC[C@@]1(O)C(=O)OCc2c1cc1n(c2=O)Cc2c-1nc1cc(F)c(Br)c3c1c2[C@H](NC(=O)CO)CC3]

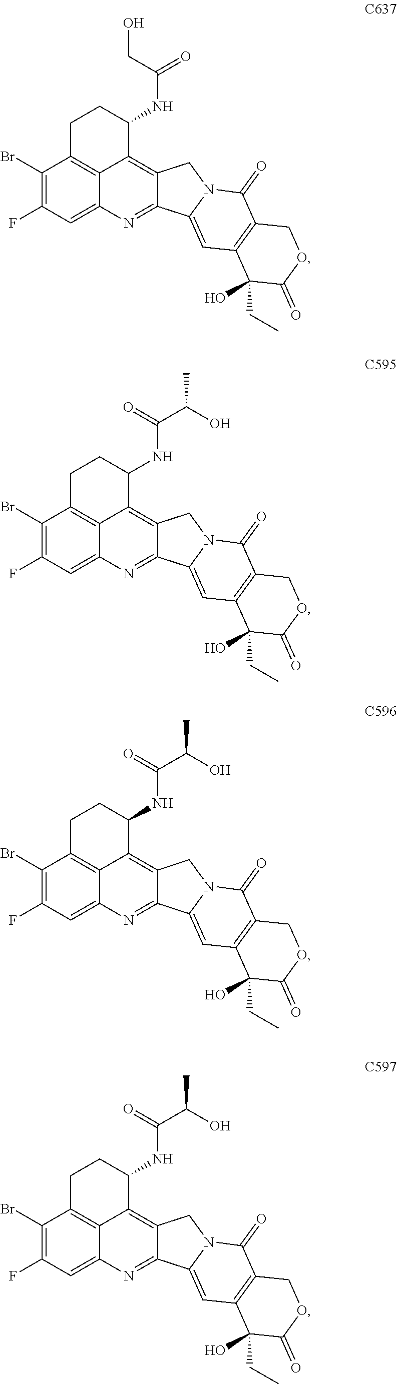 [SMILES: CC[C@@]1(O)C(=O)OCc2c1cc1n(c2=O)Cc2c-1nc1cc(F)c(Br)c3c1c2[C@@H](NC(=O)CO)CC3 ; CC[C@@]1(O)C(=O)OCc2c1cc1n(c2=O)Cc2c-1nc1cc(F)c(Br)c3c1c2C(NC(=O)[C@H](C)O)CC3 ; CC[C@@]1(O)C(=O)OCc2c1cc1n(c2=O)Cc2c-1nc1cc(F)c(Br)c3c1c2[C@H](NC(=O)[C@@H](C)O)CC3 ; CC[C@@]1(O)C(=O)OCc2c1cc1n(c2=O)Cc2c-1nc1cc(F)c(Br)c3c1c2[C@@H](NC(=O)[C@@H](C)O)CC3]

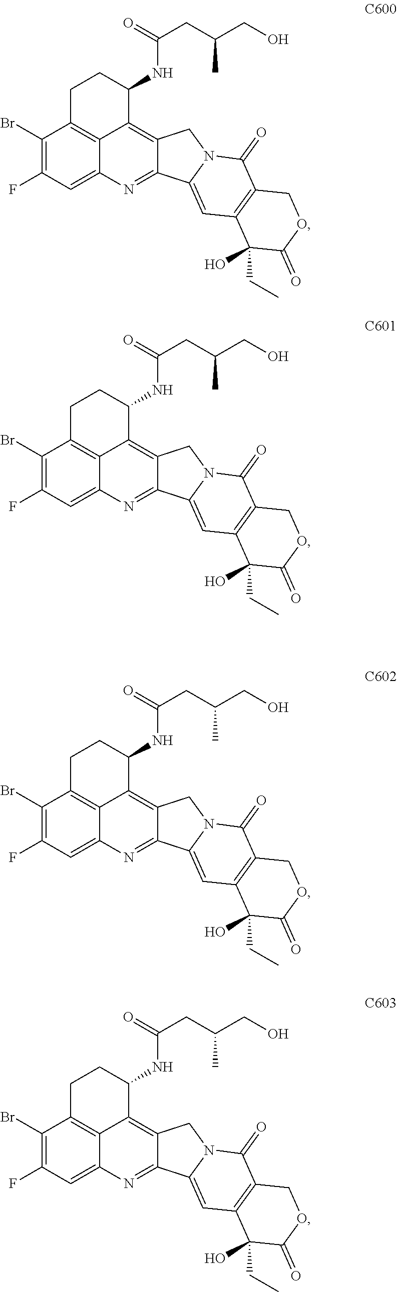 [SMILES: CC[C@@]1(O)C(=O)OCc2c1cc1n(c2=O)Cc2c-1nc1cc(F)c(Br)c3c1c2[C@H](NC(=O)C[C@H](C)CO)CC3 ; CC[C@@]1(O)C(=O)OCc2c1cc1n(c2=O)Cc2c-1nc1cc(F)c(Br)c3c1c2[C@@H](NC(=O)C[C@H](C)CO)CC3 ; CC[C@@]1(O)C(=O)OCc2c1cc1n(c2=O)Cc2c-1nc1cc(F)c(Br)c3c1c2[C@H](NC(=O)C[C@@H](C)CO)CC3 ; CC[C@@]1(O)C(=O)OCc2c1cc1n(c2=O)Cc2c-1nc1cc(F)c(Br)c3c1c2[C@@H](NC(=O)C[C@@H](C)CO)CC3]

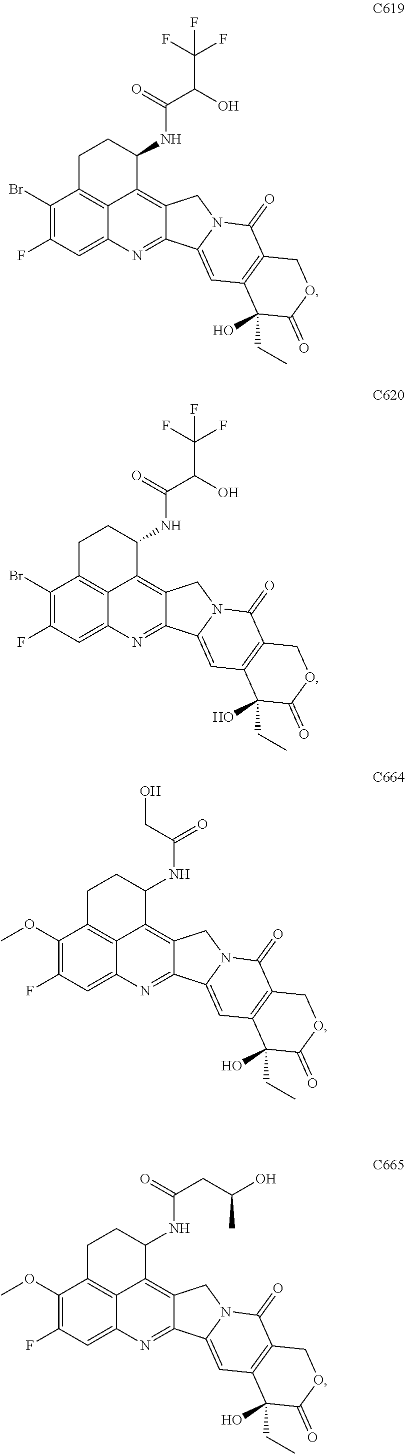 [SMILES: CC[C@@]1(O)C(=O)OCc2c1cc1n(c2=O)Cc2c-1nc1cc(F)c(Br)c3c1c2[C@H](NC(=O)C(O)C(F)(F)F)CC3 ; CC[C@@]1(O)C(=O)OCc2c1cc1n(c2=O)Cc2c-1nc1cc(F)c(Br)c3c1c2[C@@H](NC(=O)C(O)C(F)(F)F)CC3 ; CC[C@@]1(O)C(=O)OCc2c1cc1n(c2=O)Cc2c-1nc1cc(F)c(OC)c3c1c2C(NC(=O)CO)CC3 ; CC[C@@]1(O)C(=O)OCc2c1cc1n(c2=O)Cc2c-1nc1cc(F)c(OC)c3c1c2C(NC(=O)C[C@H](C)O)CC3]

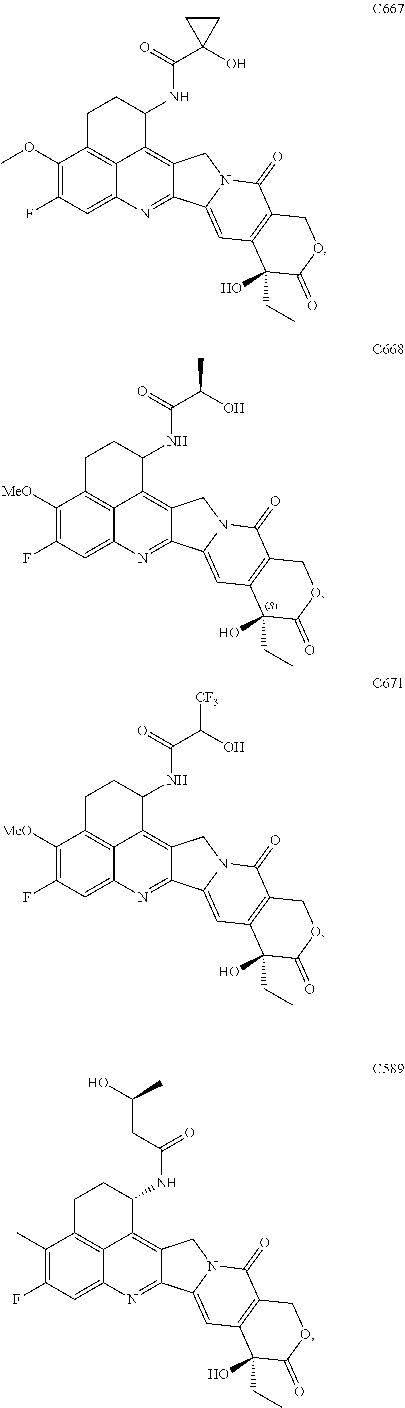

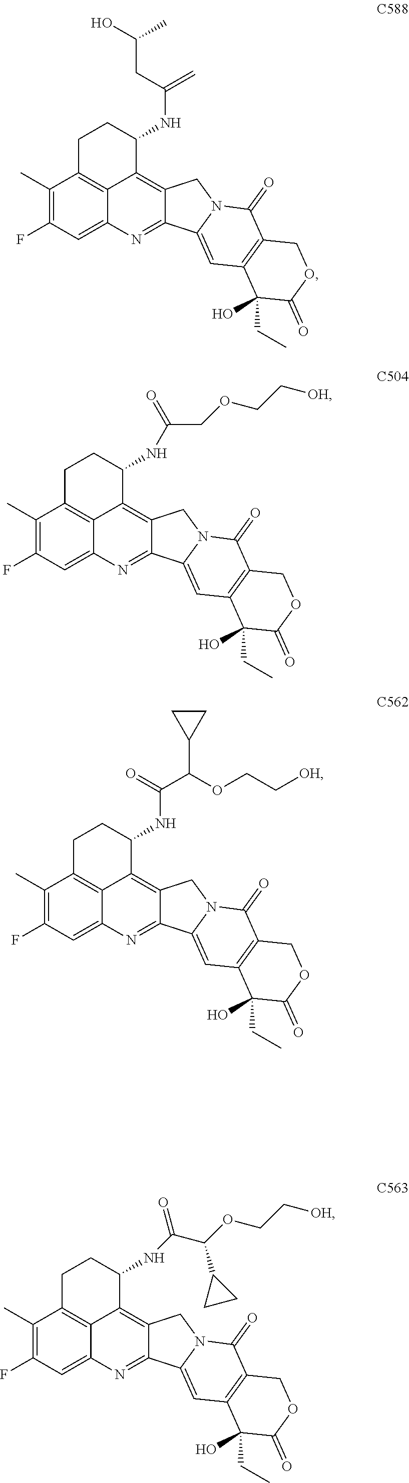

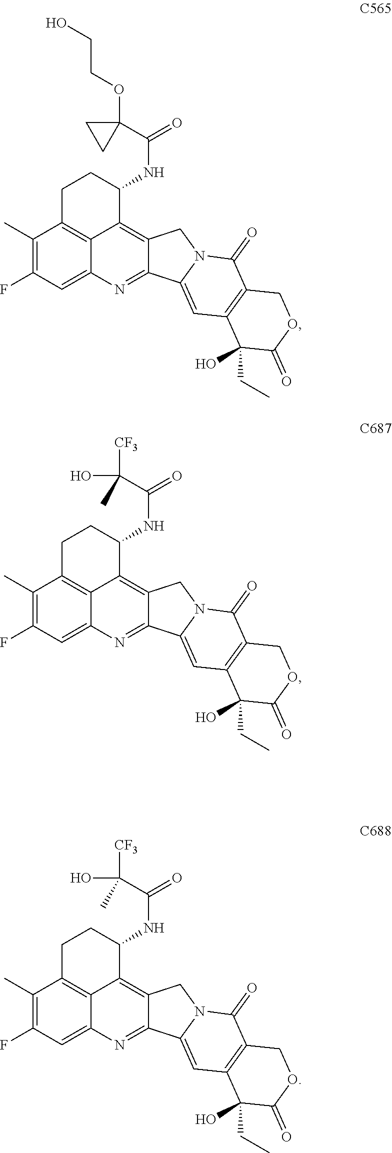 [SMILES: CC[C@@]1(O)C(=O)OCc2c1cc1n(c2=O)Cc2c-1nc1cc(F)c(C)c3c1c2[C@@H](NC(=O)C1(OCCO)CC1)CC3 ; CC[C@@]1(O)C(=O)OCc2c1cc1n(c2=O)Cc2c-1nc1cc(F)c(C)c3c1c2[C@@H](NC(=O)[C@](C)(O)C(F)(F)F)CC3 ; CC[C@@]1(O)C(=O)OCc2c1cc1n(c2=O)Cc2c-1nc1cc(F)c(C)c3c1c2[C@@H](NC(=O)[C@@](C)(O)C(F)(F)F)CC3]

In one embodiment, the cytotoxin is selected from:

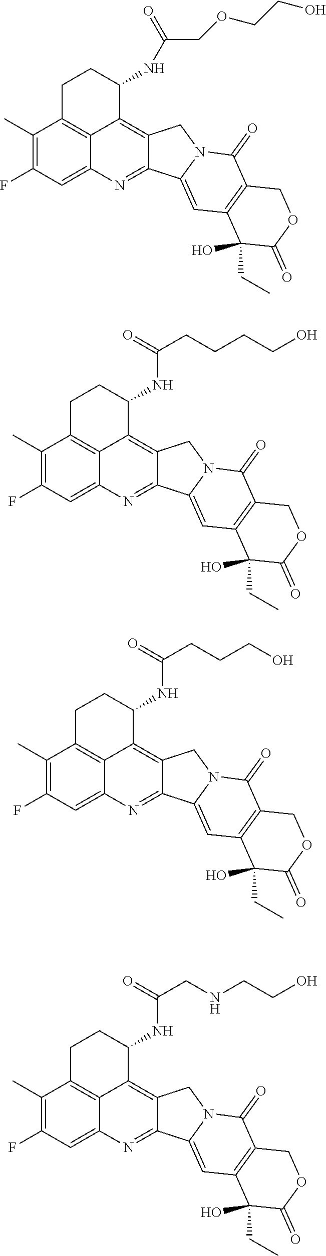

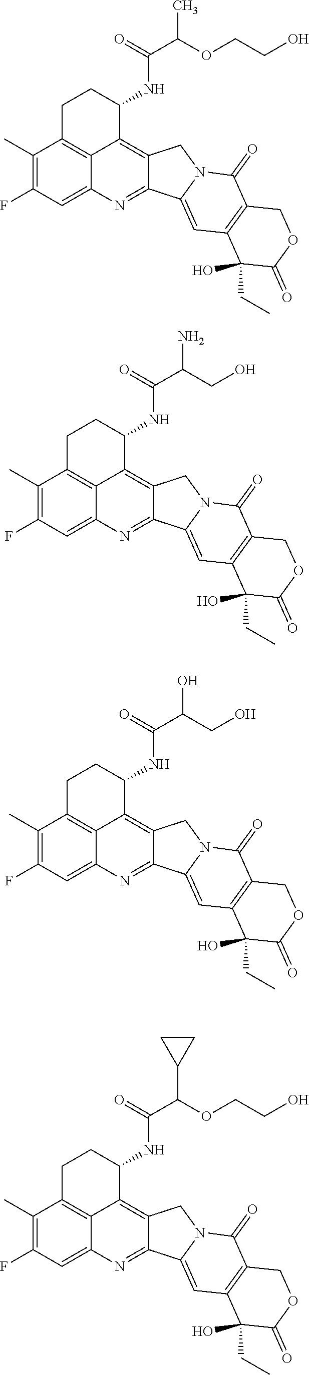 [SMILES: CC[C@@]1(O)C(=O)OCc2c1cc1n(c2=O)Cc2c-1nc1cc(F)c(C)c3c1c2[C@@H](NC(=O)C(C)OCCO)CC3 ; CC[C@@]1(O)C(=O)OCc2c1cc1n(c2=O)Cc2c-1nc1cc(F)c(C)c3c1c2[C@@H](NC(=O)C(N)CO)CC3 ; CC[C@@]1(O)C(=O)OCc2c1cc1n(c2=O)Cc2c-1nc1cc(F)c(C)c3c1c2[C@@H](NC(=O)C(O)CO)CC3 ; CC[C@@]1(O)C(=O)OCc2c1cc1n(c2=O)Cc2c-1nc1cc(F)c(C)c3c1c2[C@@H](NC(=O)C(OCCO)C1CC1)CC3]

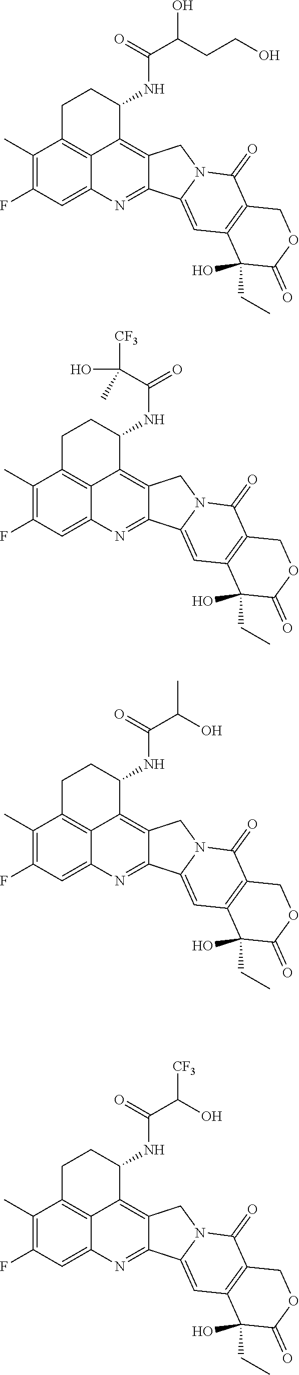 [SMILES: CC[C@@]1(O)C(=O)OCc2c1cc1n(c2=O)Cc2c-1nc1cc(F)c(C)c3c1c2[C@@H](NC(=O)C(O)CCO)CC3 ; CC[C@@]1(O)C(=O)OCc2c1cc1n(c2=O)Cc2c-1nc1cc(F)c(C)c3c1c2[C@@H](NC(=O)[C@@](C)(O)C(F)(F)F)CC3 ; CC[C@@]1(O)C(=O)OCc2c1cc1n(c2=O)Cc2c-1nc1cc(F)c(C)c3c1c2[C@@H](NC(=O)C(C)O)CC3 ; CC[C@@]1(O)C(=O)OCc2c1cc1n(c2=O)Cc2c-1nc1cc(F)c(C)c3c1c2[C@@H](NC(=O)C(O)C(F)(F)F)CC3]

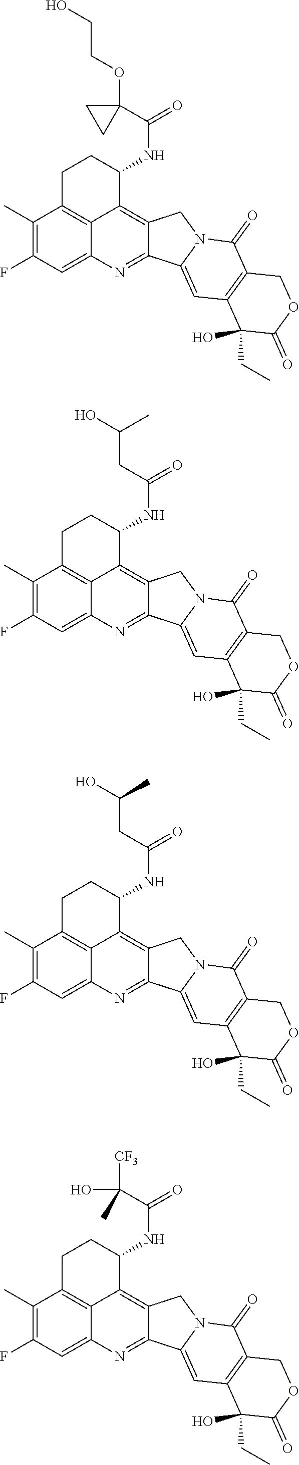 [SMILES: CC[C@@]1(O)C(=O)OCc2c1cc1n(c2=O)Cc2c-1nc1cc(F)c(C)c3c1c2[C@@H](NC(=O)C1(OCCO)CC1)CC3 ; CC[C@@]1(O)C(=O)OCc2c1cc1n(c2=O)Cc2c-1nc1cc(F)c(C)c3c1c2[C@@H](NC(=O)CC(C)O)CC3 ; CC[C@@]1(O)C(=O)OCc2c1cc1n(c2=O)Cc2c-1nc1cc(F)c(C)c3c1c2[C@@H](NC(=O)C[C@H](C)O)CC3 ; CC[C@@]1(O)C(=O)OCc2c1cc1n(c2=O)Cc2c-1nc1cc(F)c(C)c3c1c2[C@@H](NC(=O)[C@](C)(O)C(F)(F)F)CC3]

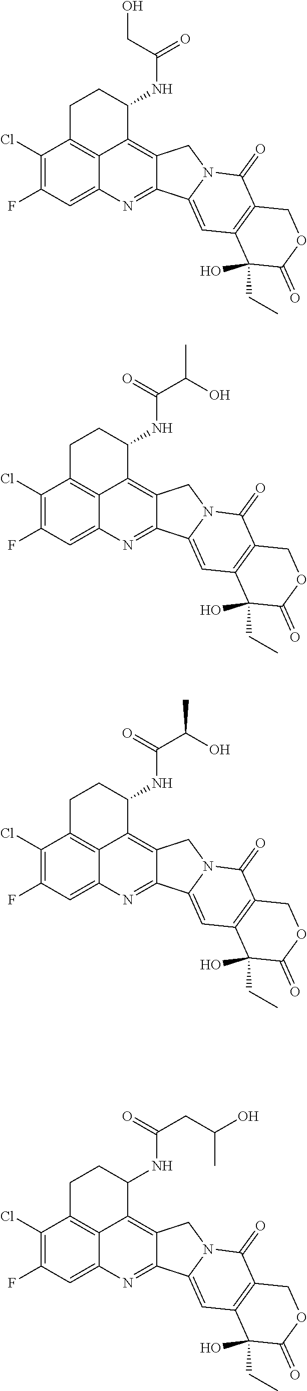 [SMILES: CC[C@@]1(O)C(=O)OCc2c1cc1n(c2=O)Cc2c-1nc1cc(F)c(Cl)c3c1c2[C@@H](NC(=O)CO)CC3 ; CC[C@@]1(O)C(=O)OCc2c1cc1n(c2=O)Cc2c-1nc1cc(F)c(Cl)c3c1c2[C@@H](NC(=O)C(C)O)CC3 ; CC[C@@]1(O)C(=O)OCc2c1cc1n(c2=O)Cc2c-1nc1cc(F)c(Cl)c3c1c2[C@@H](NC(=O)[C@@H](C)O)CC3 ; CC[C@@]1(O)C(=O)OCc2c1cc1n(c2=O)Cc2c-1nc1cc(F)c(Cl)c3c1c2C(NC(=O)CC(C)O)CC3]

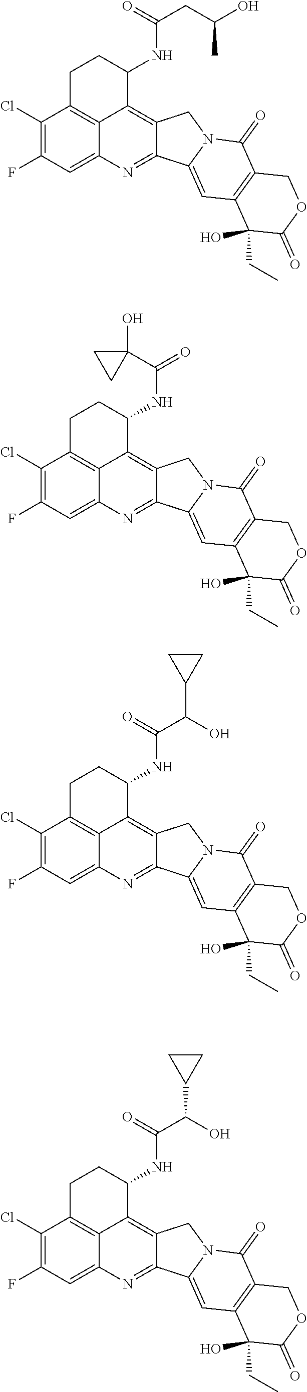 [SMILES: CC[C@@]1(O)C(=O)OCc2c1cc1n(c2=O)Cc2c-1nc1cc(F)c(Cl)c3c1c2C(NC(=O)C[C@H](C)O)CC3 ; CC[C@@]1(O)C(=O)OCc2c1cc1n(c2=O)Cc2c-1nc1cc(F)c(Cl)c3c1c2[C@@H](NC(=O)C1(O)CC1)CC3 ; CC[C@@]1(O)C(=O)OCc2c1cc1n(c2=O)Cc2c-1nc1cc(F)c(Cl)c3c1c2[C@@H](NC(=O)C(O)C1CC1)CC3 ; CC[C@@]1(O)C(=O)OCc2c1cc1n(c2=O)Cc2c-1nc1cc(F)c(Cl)c3c1c2[C@@H](NC(=O)[C@@H](O)C1CC1)CC3]

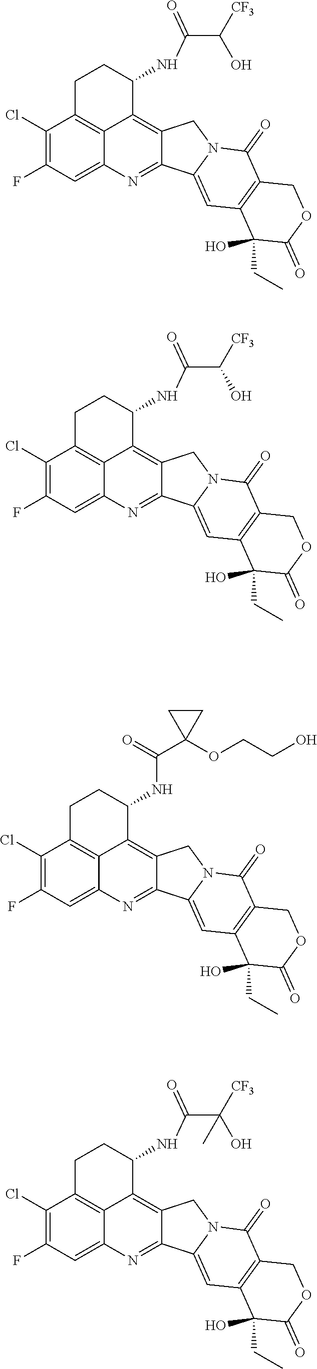 [SMILES: CC[C@@]1(O)C(=O)OCc2c1cc1n(c2=O)Cc2c-1nc1cc(F)c(Cl)c3c1c2[C@@H](NC(=O)C(O)C(F)(F)F)CC3 ; CC[C@@]1(O)C(=O)OCc2c1cc1n(c2=O)Cc2c-1nc1cc(F)c(Cl)c3c1c2[C@@H](NC(=O)[C@@H](O)C(F)(F)F)CC3 ; CC[C@@]1(O)C(=O)OCc2c1cc1n(c2=O)Cc2c-1nc1cc(F)c(Cl)c3c1c2[C@@H](NC(=O)C1(OCCO)CC1)CC3 ; CC[C@@]1(O)C(=O)OCc2c1cc1n(c2=O)Cc2c-1nc1cc(F)c(Cl)c3c1c2[C@@H](NC(=O)C(C)(O)C(F)(F)F)CC3]

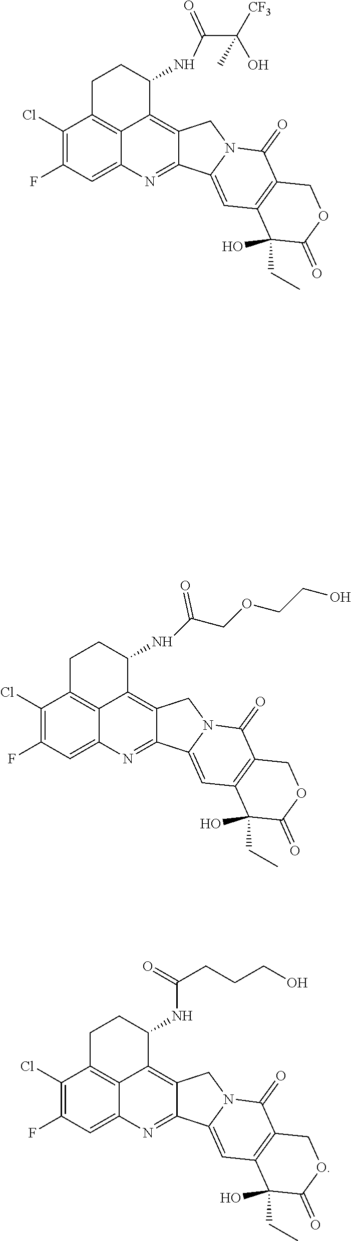 [SMILES: CC[C@@]1(O)C(=O)OCc2c1cc1n(c2=O)Cc2c-1nc1cc(F)c(Cl)c3c1c2[C@@H](NC(=O)[C@](C)(O)C(F)(F)F)CC3 ; CC[C@@]1(O)C(=O)OCc2c1cc1n(c2=O)Cc2c-1nc1cc(F)c(Cl)c3c1c2[C@@H](NC(=O)COCCO)CC3 ; CC[C@@]1(O)C(=O)OCc2c1cc1n(c2=O)Cc2c-1nc1cc(F)c(Cl)c3c1c2[C@@H](NC(=O)CCCO)CC3]

In a preferred embodiment, the cytotoxin is selected from:

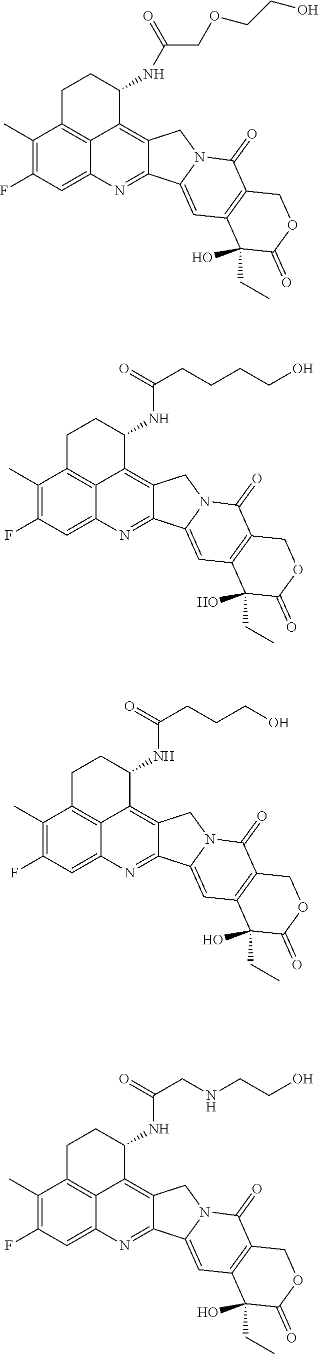 [SMILES: CC[C@@]1(O)C(=O)OCc2c1cc1n(c2=O)Cc2c-1nc1cc(F)c(C)c3c1c2[C@@H](NC(=O)COCCO)CC3 ; CC[C@@]1(O)C(=O)OCc2c1cc1n(c2=O)Cc2c-1nc1cc(F)c(C)c3c1c2[C@@H](NC(=O)CCCCO)CC3 ; CC[C@@]1(O)C(=O)OCc2c1cc1n(c2=O)Cc2c-1nc1cc(F)c(C)c3c1c2[C@@H](NC(=O)CCCO)CC3 ; CC[C@@]1(O)C(=O)OCc2c1cc1n(c2=O)Cc2c-1nc1cc(F)c(C)c3c1c2[C@@H](NC(=O)CNCCO)CC3]

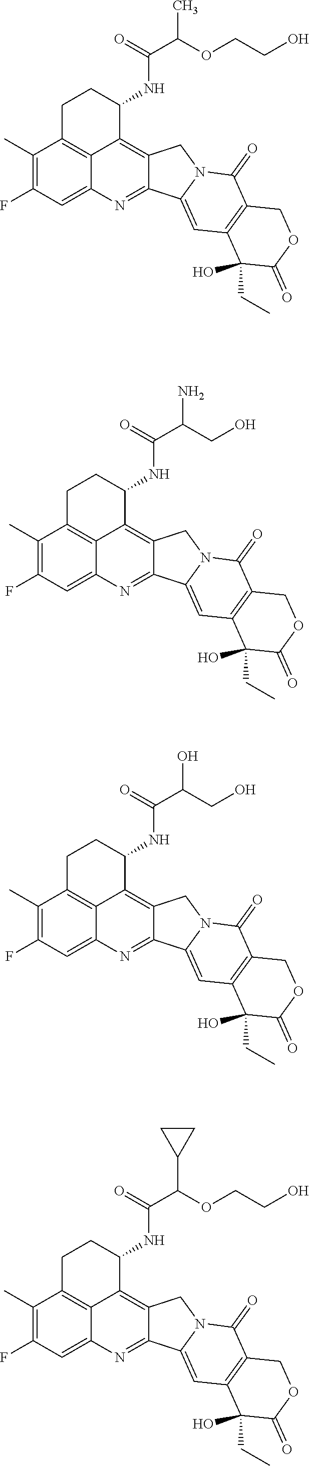 [SMILES: CC[C@@]1(O)C(=O)OCc2c1cc1n(c2=O)Cc2c-1nc1cc(F)c(C)c3c1c2[C@@H](NC(=O)C(C)OCCO)CC3 ; CC[C@@]1(O)C(=O)OCc2c1cc1n(c2=O)Cc2c-1nc1cc(F)c(C)c3c1c2[C@@H](NC(=O)C(N)CO)CC3 ; CC[C@@]1(O)C(=O)OCc2c1cc1n(c2=O)Cc2c-1nc1cc(F)c(C)c3c1c2[C@@H](NC(=O)C(O)CO)CC3 ; CC[C@@]1(O)C(=O)OCc2c1cc1n(c2=O)Cc2c-1nc1cc(F)c(C)c3c1c2[C@@H](NC(=O)C(OCCO)C1CC1)CC3]

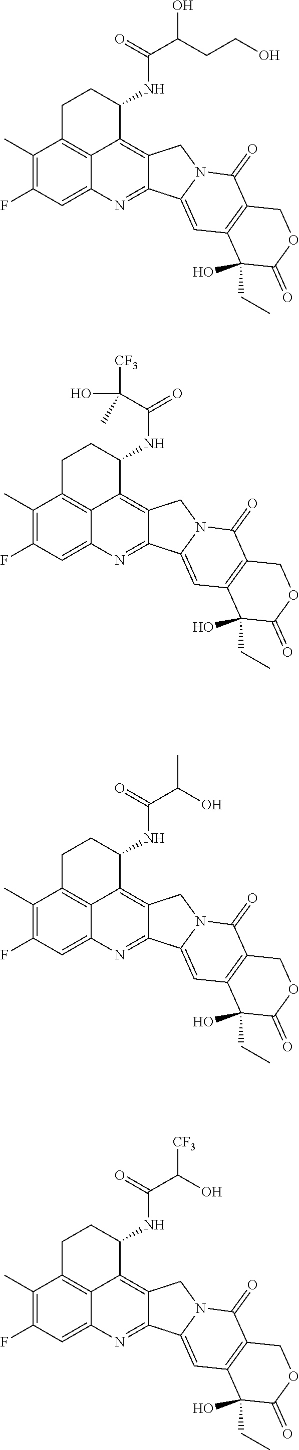 [SMILES: CC[C@@]1(O)C(=O)OCc2c1cc1n(c2=O)Cc2c-1nc1cc(F)c(C)c3c1c2[C@@H](NC(=O)C(O)CCO)CC3 ; CC[C@@]1(O)C(=O)OCc2c1cc1n(c2=O)Cc2c-1nc1cc(F)c(C)c3c1c2[C@@H](NC(=O)[C@@](C)(O)C(F)(F)F)CC3 ; CC[C@@]1(O)C(=O)OCc2c1cc1n(c2=O)Cc2c-1nc1cc(F)c(C)c3c1c2[C@@H](NC(=O)C(C)O)CC3 ; CC[C@@]1(O)C(=O)OCc2c1cc1n(c2=O)Cc2c-1nc1cc(F)c(C)c3c1c2[C@@H](NC(=O)C(O)C(F)(F)F)CC3]

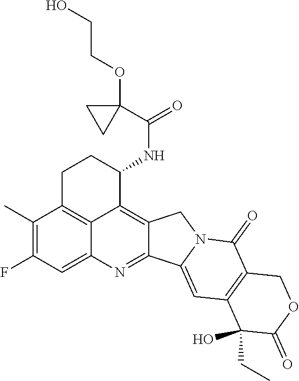 [SMILES: CC[C@@]1(O)C(=O)OCc2c1cc1n(c2=O)Cc2c-1nc1cc(F)c(C)c3c1c2[C@@H](NC(=O)C1(OCCO)CC1)CC3]

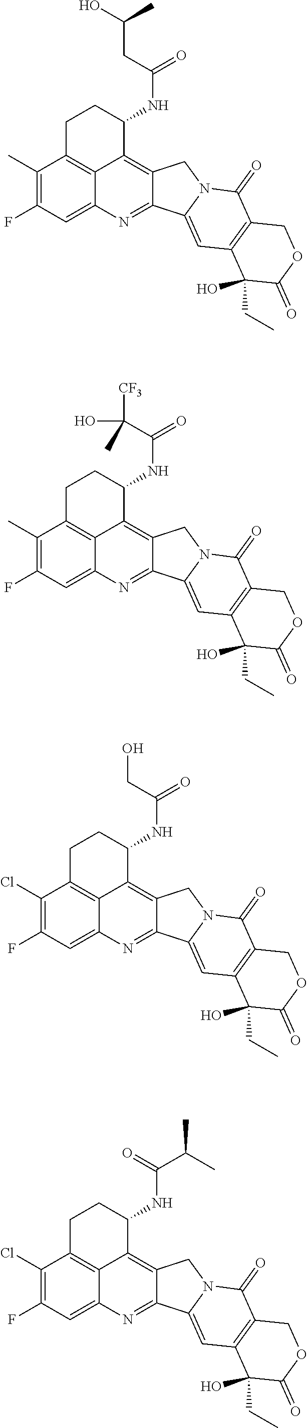

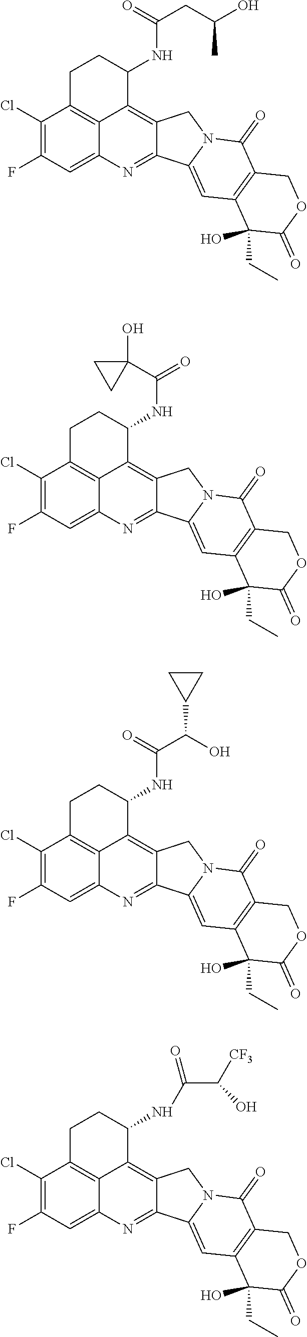 [SMILES: CC[C@@]1(O)C(=O)OCc2c1cc1n(c2=O)Cc2c-1nc1cc(F)c(Cl)c3c1c2C(NC(=O)C[C@H](C)O)CC3 ; CC[C@@]1(O)C(=O)OCc2c1cc1n(c2=O)Cc2c-1nc1cc(F)c(Cl)c3c1c2[C@@H](NC(=O)C1(O)CC1)CC3 ; CC[C@@]1(O)C(=O)OCc2c1cc1n(c2=O)Cc2c-1nc1cc(F)c(Cl)c3c1c2[C@@H](NC(=O)[C@@H](O)C1CC1)CC3 ; CC[C@@]1(O)C(=O)OCc2c1cc1n(c2=O)Cc2c-1nc1cc(F)c(Cl)c3c1c2[C@@H](NC(=O)[C@@H](O)C(F)(F)F)CC3]

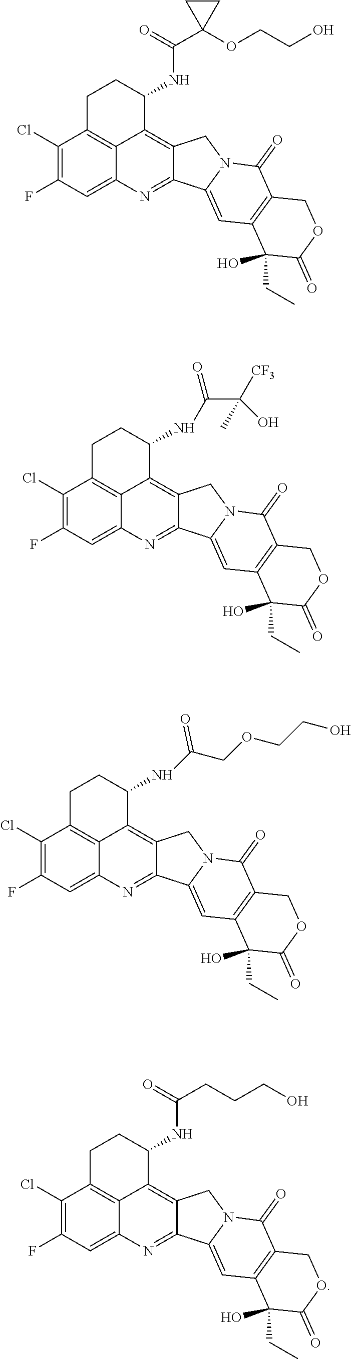 [SMILES: CC[C@@]1(O)C(=O)OCc2c1cc1n(c2=O)Cc2c-1nc1cc(F)c(Cl)c3c1c2[C@@H](NC(=O)C1(OCCO)CC1)CC3 ; CC[C@@]1(O)C(=O)OCc2c1cc1n(c2=O)Cc2c-1nc1cc(F)c(Cl)c3c1c2[C@@H](NC(=O)[C@](C)(O)C(F)(F)F)CC3 ; CC[C@@]1(O)C(=O)OCc2c1cc1n(c2=O)Cc2c-1nc1cc(F)c(Cl)c3c1c2[C@@H](NC(=O)COCCO)CC3 ; CC[C@@]1(O)C(=O)OCc2c1cc1n(c2=O)Cc2c-1nc1cc(F)c(Cl)c3c1c2[C@@H](NC(=O)CCCO)CC3]

In a referred embodiment the cytotoxin is selected from:

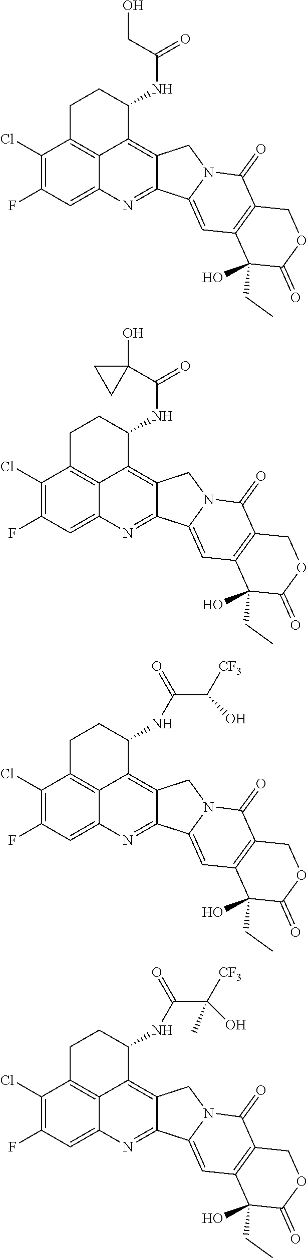

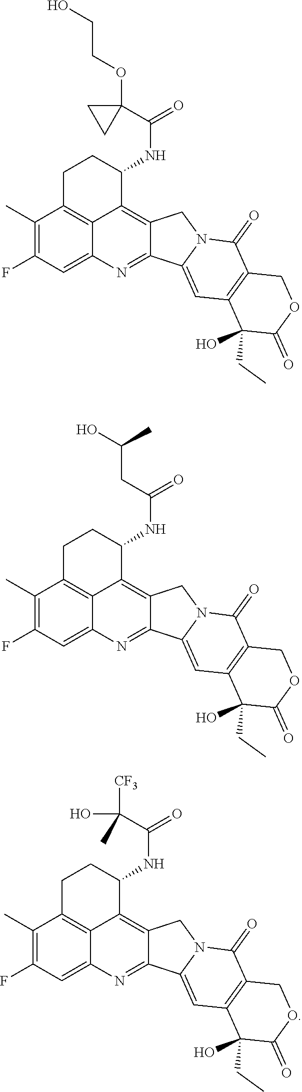 [SMILES: CC[C@@]1(O)C(=O)OCc2c1cc1n(c2=O)Cc2c-1nc1cc(F)c(C)c3c1c2[C@@H](NC(=O)C1(OCCO)CC1)CC3 ; CC[C@@]1(O)C(=O)OCc2c1cc1n(c2=O)Cc2c-1nc1cc(F)c(C)c3c1c2[C@@H](NC(=O)C[C@H](C)O)CC3 ; CC[C@@]1(O)C(=O)OCc2c1cc1n(c2=O)Cc2c-1nc1cc(F)c(C)c3c1c2[C@@H](NC(=O)[C@](C)(O)C(F)(F)F)CC3]

In a more preferred embodiment, the cytotoxin is selected from:

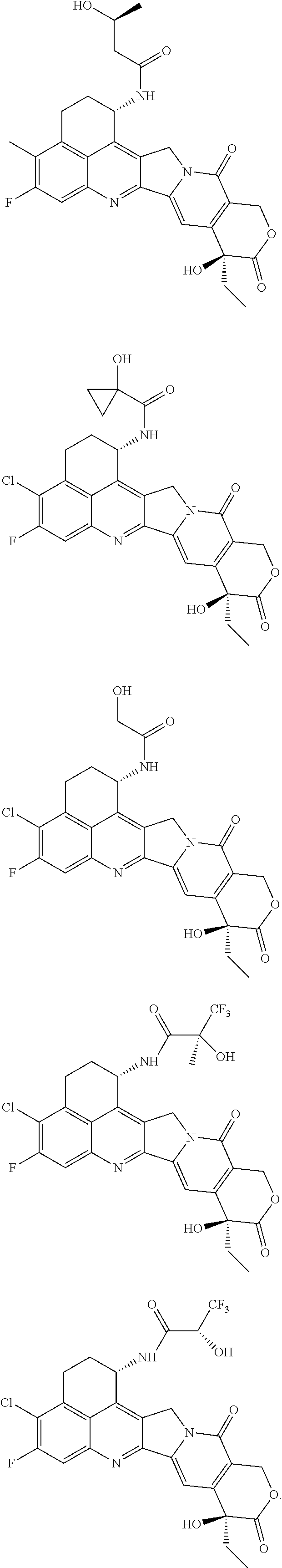 [SMILES: CC[C@@]1(O)C(=O)OCc2c1cc1n(c2=O)Cc2c-1nc1cc(F)c(C)c3c1c2[C@@H](NC(=O)C[C@H](C)O)CC3 ; CC[C@@]1(O)C(=O)OCc2c1cc1n(c2=O)Cc2c-1nc1cc(F)c(Cl)c3c1c2[C@@H](NC(=O)C1(O)CC1)CC3 ; CC[C@@]1(O)C(=O)OCc2c1cc1n(c2=O)Cc2c-1nc1cc(F)c(Cl)c3c1c2[C@@H](NC(=O)CO)CC3 ; CC[C@@]1(O)C(=O)OCc2c1cc1n(c2=O)Cc2c-1nc1cc(F)c(Cl)c3c1c2[C@@H](NC(=O)[C@](C)(O)C(F)(F)F)CC3 ; CC[C@@]1(O)C(=O)OCc2c1cc1n(c2=O)Cc2c-1nc1cc(F)c(Cl)c3c1c2[C@@H](NC(=O)[C@@H](O)C(F)(F)F)CC3]

In a particular embodiment the cytotoxin is selected from:

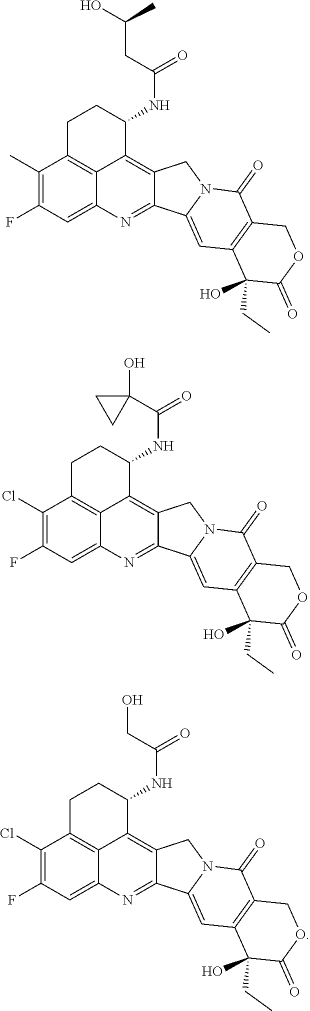 [SMILES: CC[C@@]1(O)C(=O)OCc2c1cc1n(c2=O)Cc2c-1nc1cc(F)c(C)c3c1c2[C@@H](NC(=O)C[C@H](C)O)CC3 ; CC[C@@]1(O)C(=O)OCc2c1cc1n(c2=O)Cc2c-1nc1cc(F)c(Cl)c3c1c2[C@@H](NC(=O)C1(O)CC1)CC3 ; CC[C@@]1(O)C(=O)OCc2c1cc1n(c2=O)Cc2c-1nc1cc(F)c(Cl)c3c1c2[C@@H](NC(=O)CO)CC3]

In one embodiment, the cytotoxin is selected from the following compounds; wherein the wavy bond shows the connection site for connection with the compound of formula (I).

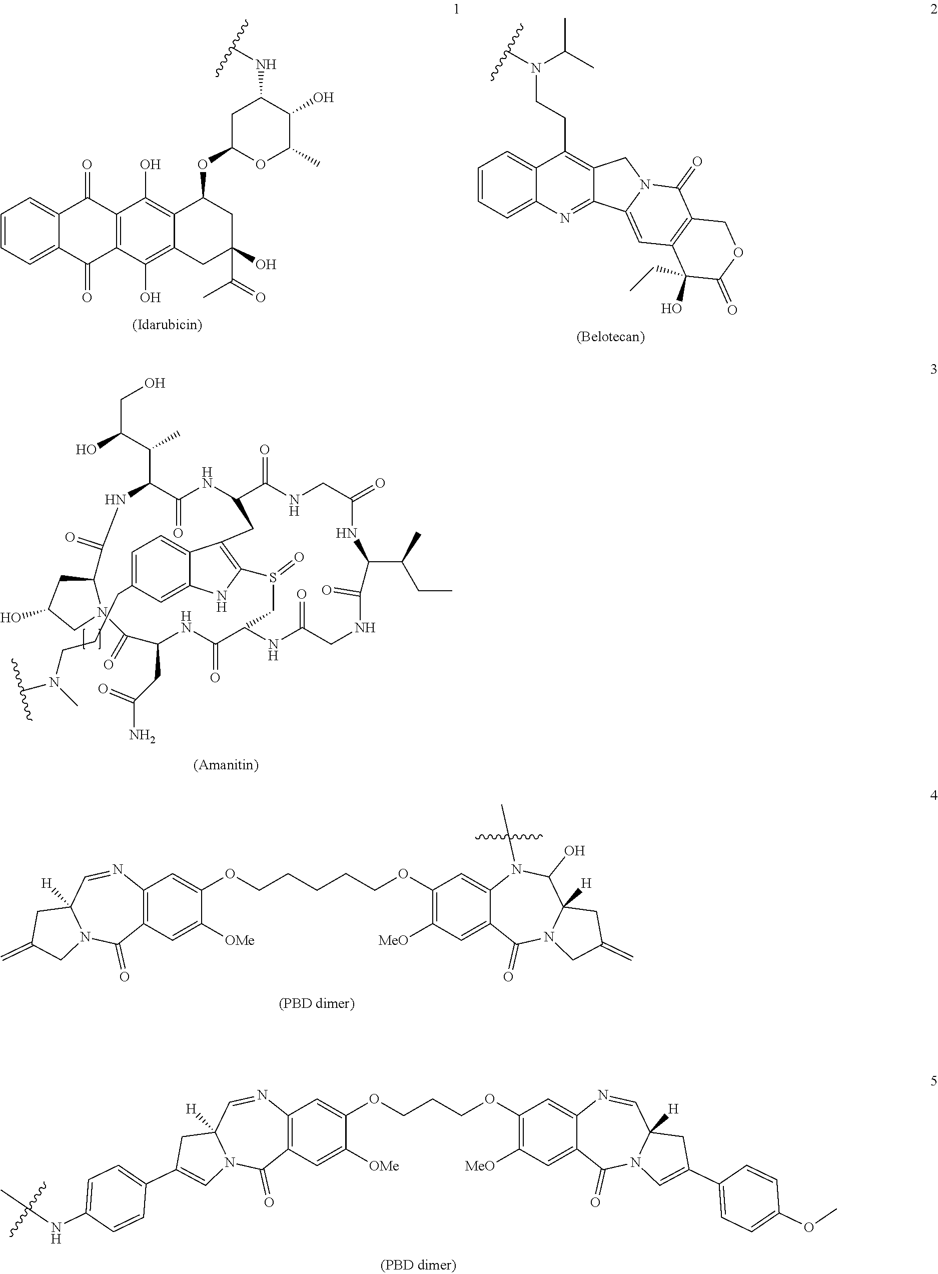

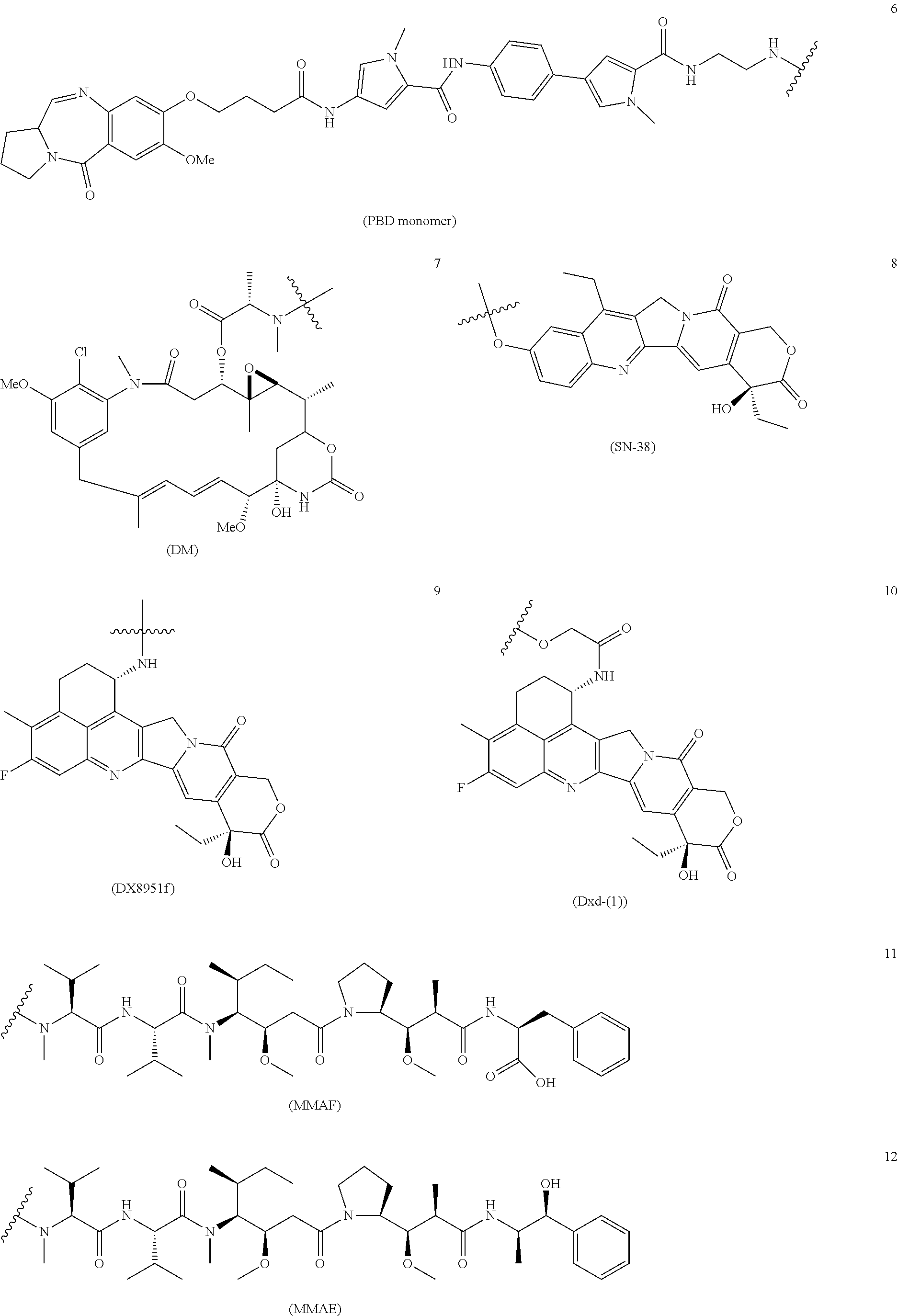

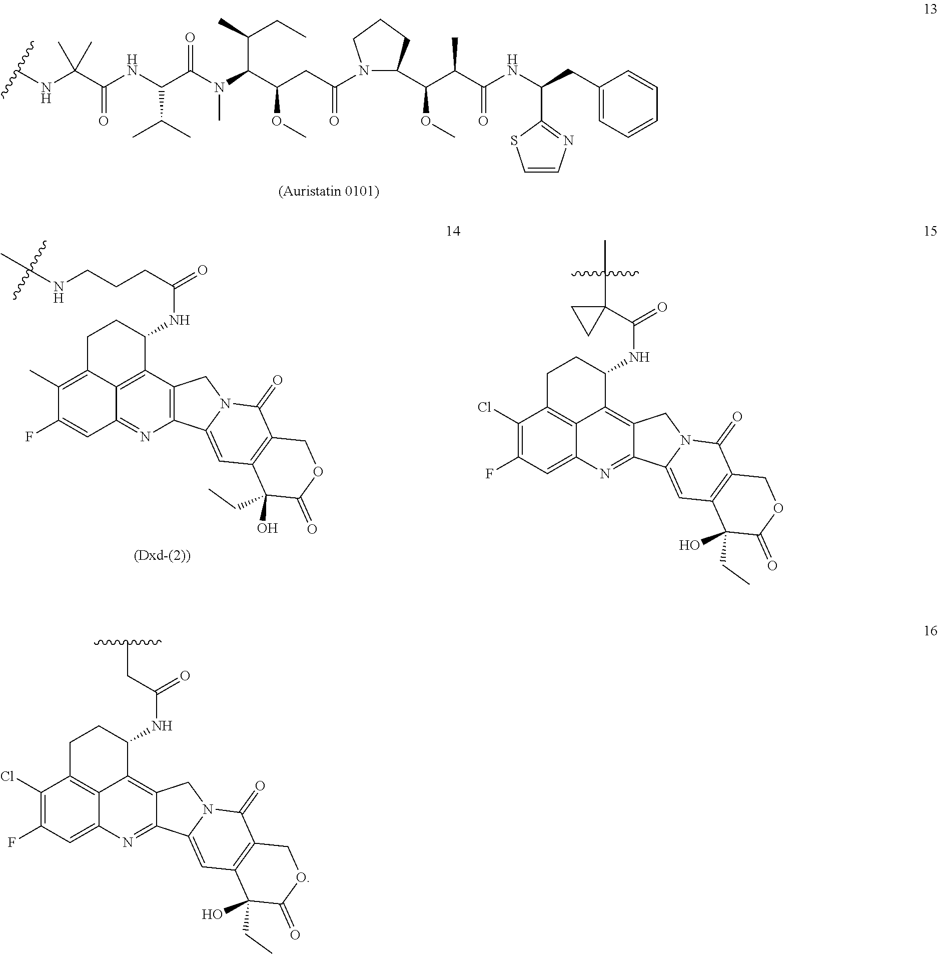

In some embodiments, the payload is selected from DX8951f (compound 9), DXd-(1) (compound 10), DXd-(2) (compound 14),

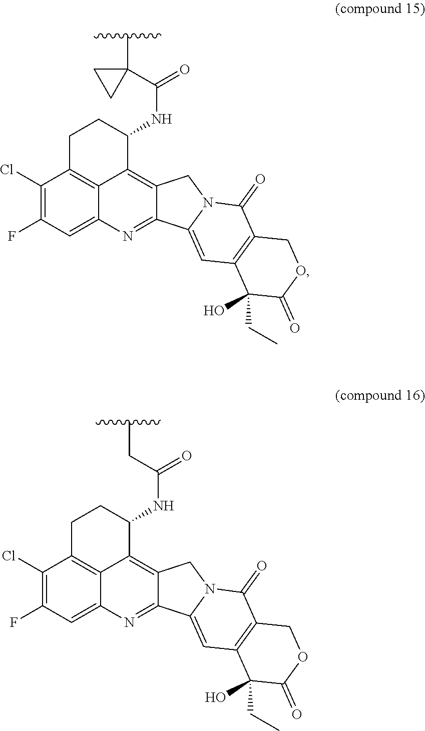 [SMILES: *C1(C(=O)N[C@H]2CCc3c(Cl)c(F)cc4nc5c(c2c34)Cn2c-5cc3c(c2=O)COC(=O)[C@]3(O)CC)CC1 ; *CC(=O)N[C@H]1CCc2c(Cl)c(F)cc3nc4c(c1c23)Cn1c-4cc2c(c1=O)COC(=O)[C@]2(O)CC]

preferably DX8951f, DXd-(1)

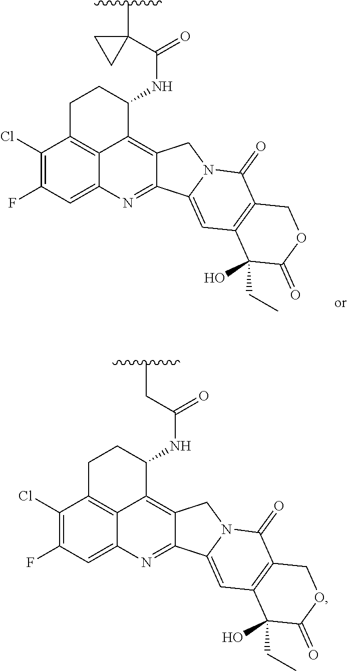 [SMILES: *C1(C(=O)N[C@H]2CCc3c(Cl)c(F)cc4nc5c(c2c34)Cn2c-5cc3c(c2=O)COC(=O)[C@]3(O)CC)CC1 ; *CC(=O)N[C@H]1CCc2c(Cl)c(F)cc3nc4c(c1c23)Cn1c-4cc2c(c1=O)COC(=O)[C@]2(O)CC]

more preferably DXd-(1),

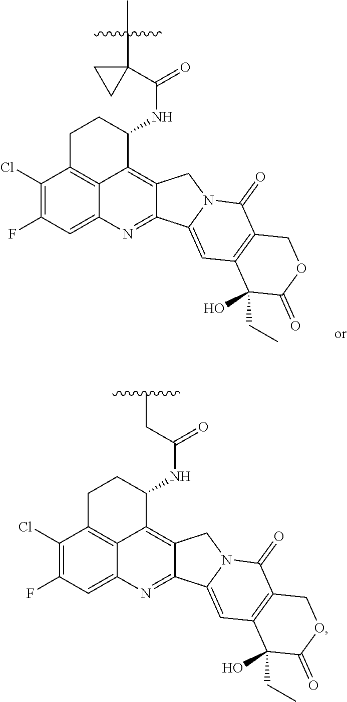 [SMILES: *C1(C(=O)N[C@H]2CCc3c(Cl)c(F)cc4nc5c(c2c34)Cn2c-5cc3c(c2=O)COC(=O)[C@]3(O)CC)CC1 ; *CC(=O)N[C@H]1CCc2c(Cl)c(F)cc3nc4c(c1c23)Cn1c-4cc2c(c1=O)COC(=O)[C@]2(O)CC]

most preferably

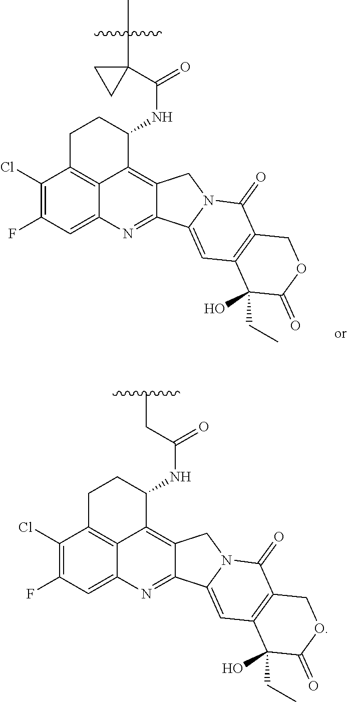 [SMILES: *C1(C(=O)N[C@H]2CCc3c(Cl)c(F)cc4nc5c(c2c34)Cn2c-5cc3c(c2=O)COC(=O)[C@]3(O)CC)CC1 ; *CC(=O)N[C@H]1CCc2c(Cl)c(F)cc3nc4c(c1c23)Cn1c-4cc2c(c1=O)COC(=O)[C@]2(O)CC]

### Preparation of the Payload-Bearing Formula (I) Compound

In one embodiment, the linking unit and the Payload are connected via reactive groups as defined above, using any reaction known in the art, including but not limit to condensation reaction, nucleophilic addition, electrophilic addition, etc.

In one embodiment, the payload is a cytotoxin. In one embodiment, the linking unit-payload intermediate (numbered as LBx) is as shown in the following table.

| Compound of | | Formula of | Values of m, n, i, j and t (when | |
|---|---|---|---|---|
| formula (II) | Linker | Linker | applicable)* | Payload |
| II-a-1 | I-a-1 | I-a | i is 4, n is 3, m is 1, j is 12 | Compound 15 |
| II-a-2 | I-a-2 | I-a | i is 4, n is 3, m is 1, j is 12 | Compound 15 |
| II-a-3 | I-a-3 | I-a | i is 4, n is 3, m is 1, j is 12 | Compound 15 |
| II-a-4 | I-a-4 | I-a | i is 4, n is 3, m is 2, j is 12 | Compound 15 |
| II-a-5 | I-a-5 | I-a | i is 4, n is 3, m is 2, j is 12 | Compound 15 |
| II-a-6 | I-a-6 | I-a | i is 4, n is 3, m is 2, j is 12 | Compound 15 |
| II-a-7 | I-a-7 | I-a | i is 4, n is 3, m is 1, j is 12 | Compound 15 |
| II-a-8 | I-a-8 | I-a | i is 4, n is 3, m is 1, j is 12 | Compound 15 |
| II-a-9 | I-a-9 | I-a | i is 4, n is 3, m is 1, j is 12 | Compound 15 |
| II-a-10 | I-a-10 | I-a | i is 4, n is 3, m is 2, j is 12 | Compound 15 |
| II-a-11 | I-a-11 | I-a | i is 4, n is 3, m is 2, j is 12 | Compound 15 |
| II-a-12 | I-a-12 | I-a | i is 4, n is 3, m is 2, j is 12 | Compound 15 |
| II-b-1 | I-b-1 | I-b | i is 4, n is 3, m is 1, t is 12 | Compound 15 |
| II-b-2 | I-b-2 | I-b | i is 4, n is 3, m is 1, t is 12 | Compound 15 |
| II-b-3 | I-b-3 | I-b | i is 4, n is 3, m is 1, t is 12 | Compound 15 |
| II-b-4 | I-b-4 | I-b | i is 4, n is 3, m is 2, t is 12 | Compound 15 |
| II-b-5 | I-b-5 | I-b | i is 4, n is 3, m is 2, t is 12 | Compound 15 |
| II-b-6 | I-b-6 | I-b | i is 4, n is 3, m is 2, t is 12 | Compound 15 |
| II-b-7 | I-b-7 | I-b | i is 4, n is 3, m is 1, t is 12 | Compound 15 |
| II-b-8 | I-b-8 | I-b | i is 4, n is 3, m is 1, t is 12 | Compound 15 |
| II-b-9 | I-b-9 | I-b | i is 4, n is 3, m is 1, t is 12 | Compound 15 |
| II-b-10 | I-b-10 | I-b | i is 4, n is 3, m is 2, t is 12 | Compound 15 |
| II-b-11 | I-b-11 | I-b | i is 4, n is 3, m is 2, t is 12 | Compound 15 |
| II-b-12 | I-b-12 | I-b | i is 4, n is 3, m is 2, t is 12 | Compound 15 |

### Conjugates and Preparation Thereof

Furthermore, the payload-bearing formula (I) compound which has the moiety comprising ligase recognition sequence can be conjugated with other molecules comprising a ligase recognition sequence, and can be thereby used in for example, the preparation of a targeting molecule-drug conjugate, such as an antibody-drug conjugate. Accordingly, in yet another aspect, provided is a conjugate which comprises a compound of formula (I), a targeting molecule, and a payload.

In yet another aspect, provided is a conjugate having the structure of formula (III):

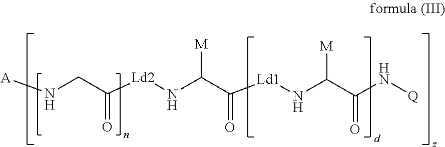

wherein,
Q is hydrogen, —C₂H₄-(PEG)ₜ-(CO)NH₂ or LKb-P;
n, d, t, Ld1 and Ld2 are as defined in formula (I);
M is LKa-LKb-P;
P is a payload which is linked to the B moiety or L¹ moiety of the compound of formula (I);
A is a targeting molecule;
z is an integer of 1 to 20.

In one embodiment, A is an anti-human monoclonal antibody connected to the rest of the conjugate through a modified heavy chain and/or light chain C-terminal, wherein the modified heavy chain and/or light chain C-terminal is modified to comprise Leu-Pro-Xaa-Thr, wherein Xaa is any natural or unnatural single amino acid. In one embodiment, z is 2. In a preferred embodiment, A is an anti-human monoclonal antibody connected to the rest of the conjugate through a modified heavy chain and/or light chain C-terminal, wherein the modified heavy chain and/or light chain C-terminal is modified to comprise Leu-Pro-Xaa-Thr, wherein Xaa is any natural or unnatural single amino acid, and z is 2.

### Targeting Molecule

In one embodiment, the targeting molecule is an antibody or an antigen binding fragment thereof.

In one embodiment, the targeting molecule is an anti-human HER2 antibody or antigen binding fragment thereof. Examples of anti-human HER2 antibodies include but are not limited to Pertuzumab and Trastuzumab. Pertuzumab binds to the second extracellular domain (ECD2) of HER2 and is approved for the treatment of HER2-positive breast cancer. Trastuzumab binds to the fourth extracellular domain (ECD4) of HER2 and is approved for the treatment of Her2-positive breast cancer and gastric cancer.

In a preferred embodiment, the anti-human HER2 antibody is one or more selected from engineered anti-HER2 antibodies based on Trastuzumab.

In one embodiment, the targeting molecule is an anti-human TROP2 antibody or antigen binding fragment thereof. Examples of anti-human TROP2 antibodies include but are not limited to Trodelvy's antibody (hRS7) and DS1062's antibody (Datopotamab).

In one embodiment, the targeting molecule is an anti-FGFR3 antibody or antigen binding fragment thereof.

In a preferred embodiment, the anti-human HER2 antibody is a recombinant antibody selected from monoclonal antibody, chimeric antibody, humanized antibody, antibody fragment, and antibody mimic. In a preferred embodiment, the anti-human Trop2 antibody is a recombinant antibody selected from monoclonal antibody, chimeric antibody, humanized antibody, antibody fragment, and antibody mimic. In a preferred embodiment, the anti-FGFR3 antibody is a recombinant antibody selected from monoclonal antibody, chimeric antibody, humanized antibody, antibody fragment, and antibody mimic. For the conjugation with the compound of formula (I), the targeting molecule of the present disclosure may comprise a modified moiety to connect with Gn in the compound of formula (I). In one embodiment, the antibody mimic is selected from scFv, minibody, diabody, nanobody. The introduction position of such modified moiety is not limited, for example, when the targeting molecule is an antibody, its introduction position can be, but not limited to, located at the C-terminal or the N-terminal of the heavy chain or light chain of the antibody.

In an alternative embodiment, a modified moiety for the conjugation with Gn in the compound of formula (I) can be introduced at a non-terminal position of the heavy chain or light chain of the antibody using, for example, chemical modification methods.

In one embodiment, the targeting molecule of the present disclosure is an antibody or antigen-binding fragment thereof, which may comprise terminal modification. A terminal modification refers to a modification at the C-terminal or N-terminal of the heavy chain or light chain of the antibody, which for example comprises a ligase recognition sequence. In another embodiment, the terminal modification may further comprise spacer Sp2 comprising 2-100 amino acids, wherein the antibody, Sp2 and the ligase recognition sequence are sequentially linked. In a preferred embodiment, Sp2 is a spacer sequence containing 2-20 amino acids. In a particular embodiment, Sp2 is a spacer sequence selected from GA, GGGGS, GGGGSGGGGS and GGGGSGGGGSGGGGS, especially GA.

In a preferred embodiment, the light chain of the antibody or antigen-binding fragment thereof includes 3 types: wild-type (LC); the C-terminus modified light chain (LCCT), which is modified by direct introduction of a ligase recognition sequence LPXTG and C-terminus modified light chain (LCCT_{L}), which is modified by introduction of short peptide spacers plus the ligase donor substrate recognition sequence LPXTG. The heavy chain of the antibody or antigen-binding fragment thereof includes 3 types: wild-type (HC); the C-terminus modified heavy chain (HCCT), which is modified by direct introduction of a ligase recognition sequence LPXTG; and C-terminus modified heavy chain (HCCT_{L}), which is modified by introduction of short peptide spacers plus the ligase donor substrate recognition sequence LPXTG. X can be any natural or non-natural single amino acid. When z in the compound of formula (III) is 1 or 2, the combination of the above heavy and light chains can form 8 preferred antibody molecules, see the amino acid sequence table.

The conjugates of the present disclosure can further comprise a payload. The payload is as described above.

Anti-HER2, TROP2, FGFR3 antibodies are listed for reference, however, without limitation, all the antibodies and be used to connect to the linker-payload. For example, anti-human monoclonal antibody can be connected to the rest of the conjugate (the linker-payload part) through a modified heavy chain and/or light chain C-terminal, wherein the modified heavy chain and/or light chain C-terminal is modified to comprise Leu-Pro-Xaa-Thr, wherein Xaa is any natural or unnatural single amino acid, and z is 2.

### Specific Embodiments for the Conjugate

In one embodiment, each LKa is

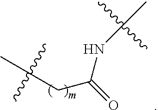

In one embodiment, formula (III) has the structure of formula (III-1):

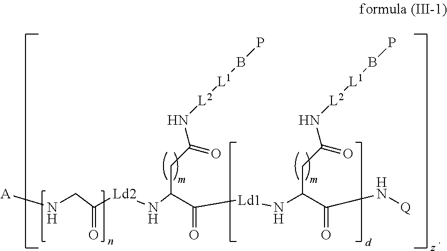

In one embodiment, Ld2 is

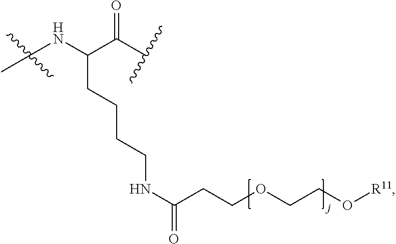

### Ld1 is

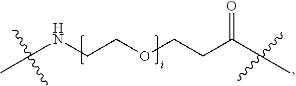

d is 1, Q is hydrogen. In one embodiment, the compound of formula (III-1) is as follows:

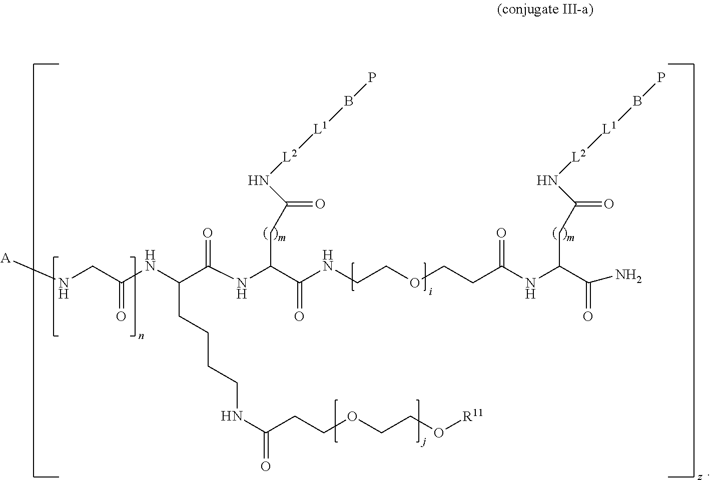

In one embodiment, Ld2 is a bond, Ld1 is

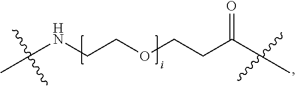

d is 1, Q is —C₂H₄—(PEG)ₜ-(CO)NH₂. In one embodiment, the compound of formula (III-1) is as follows:

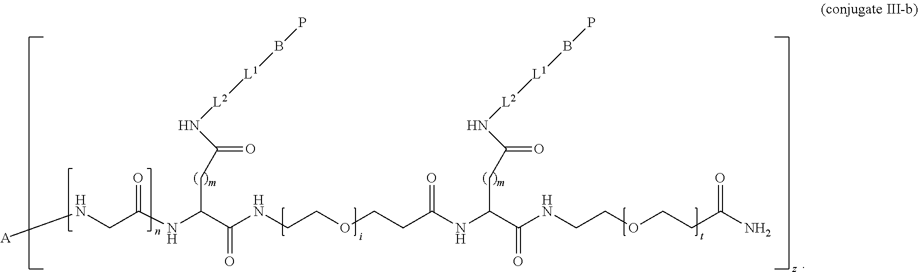

In one embodiment, z is 1 to 4. In one embodiment, z is 2 or 4.

In one embodiment, z is 2.

In one embodiment, z is 4.

In one embodiment, in conjugate III-a, III-b, z is 2 or 4.

In one embodiment, in conjugate III-a, III-b, z is 2.

In one embodiment, in conjugate III-a, III-b, z is 4.

In one embodiment, B in compound of formula (I) is a terminal group R¹⁰, and the Cleavable sequence 1 in L¹ is connected to the payload to form a compound of formula (II) wherein B is absent in the resulting molecule of the connection of Cleavable sequence 1 with the payload.

In one embodiment, i is 4, n is 3, m is 1, j is 12, L² is —(C₂H₄—O)ₚ—(CH₂)₂(CO)—, p is 2, L¹ is Gly-Gly-Phe-Gly, B is —NH—CH₂—U—R¹⁰ or —R or —NH—CH₂—U—(CR¹R²)_{g}—(CO)—R¹⁰, U is O, g is 1, R¹¹ is methyl. In one embodiment, conjugate III-a has the structure of:

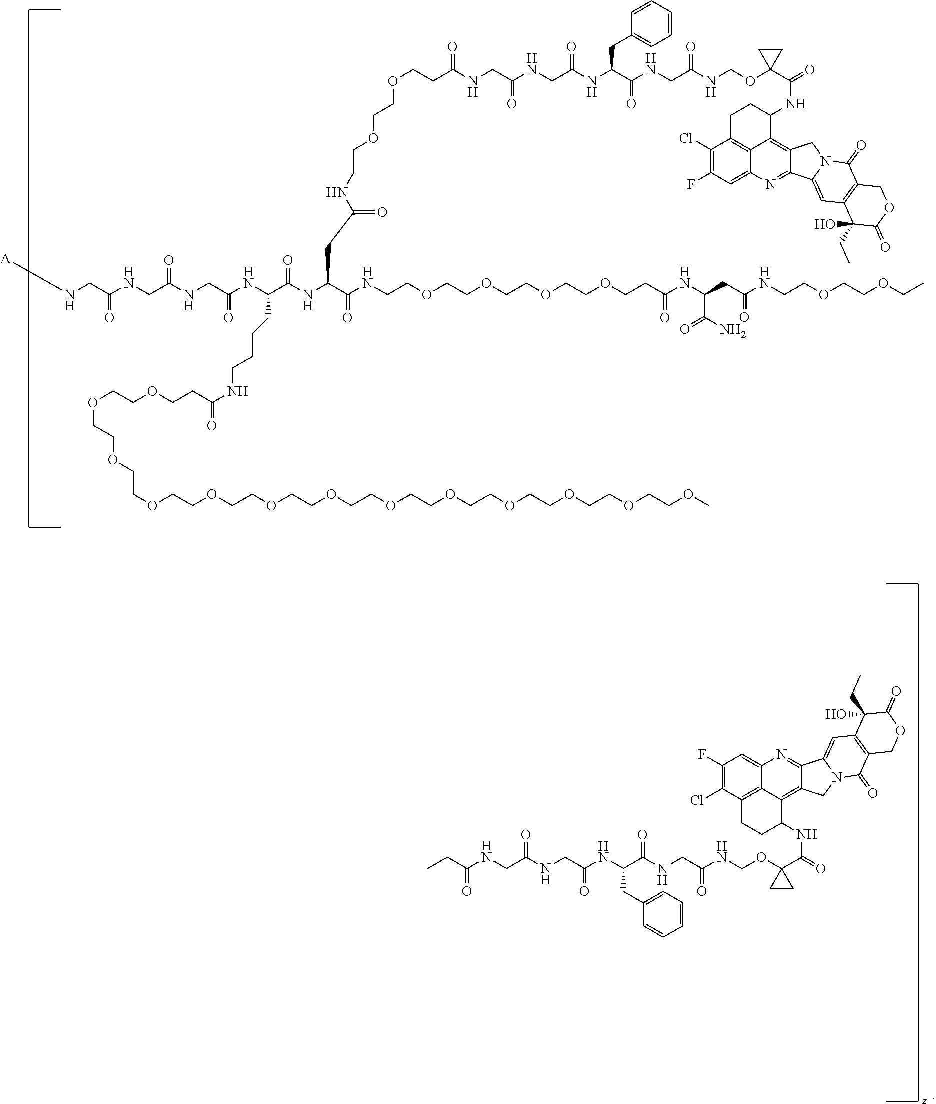

In one embodiment, conjugate III-a has the structure of:

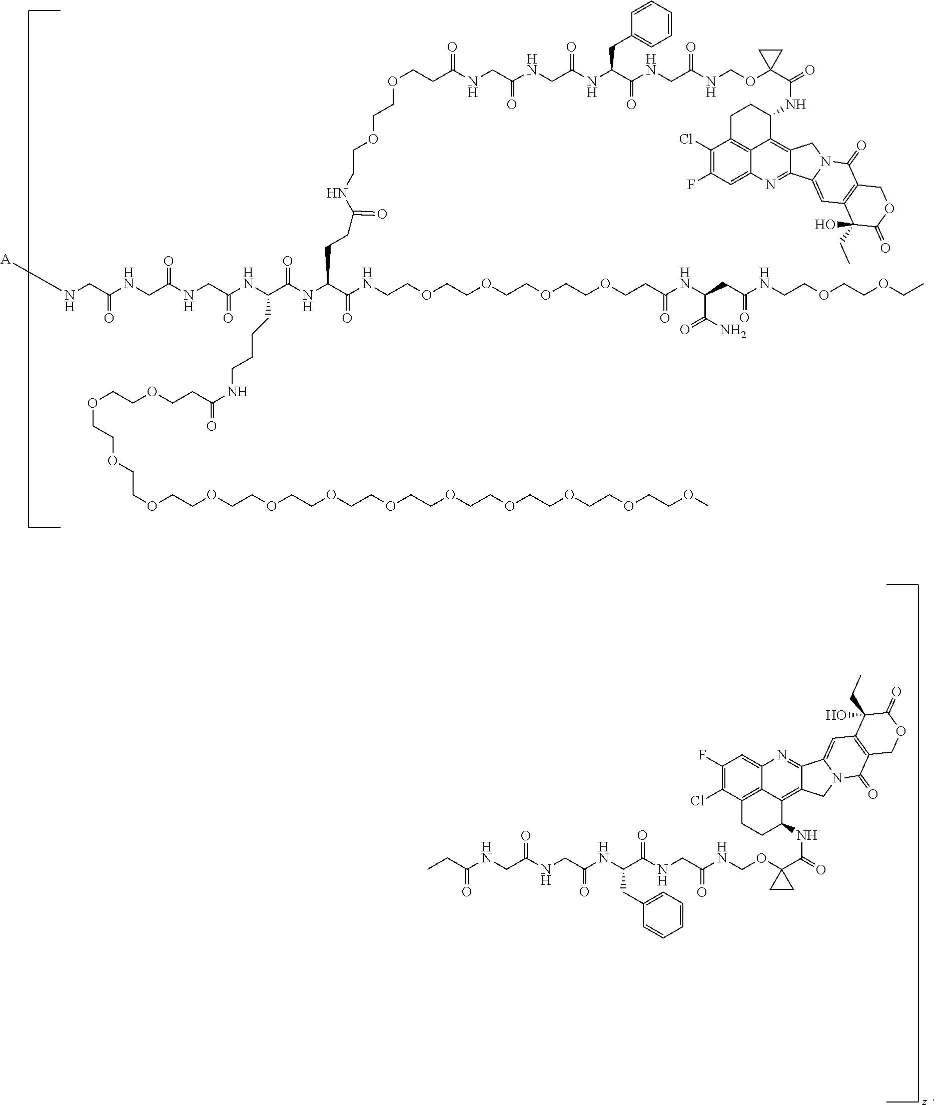

In one embodiment, i is 4, n is 3, m is 2, j is 12, L² is —(C₂H₄—O)ₚ—(CH₂)₂(CO)—, p is 2, L¹ is Gly-Gly-Phe-Gly, B is—NH—CH₂-U-R¹⁰ or —R¹⁰ or —NH—CH₂-U-(CR¹R²)_{g}—(CO)—R¹⁰, U is O, g is 1, R¹¹ is methyl. In one embodiment, the conjugate III-a has the structure of:

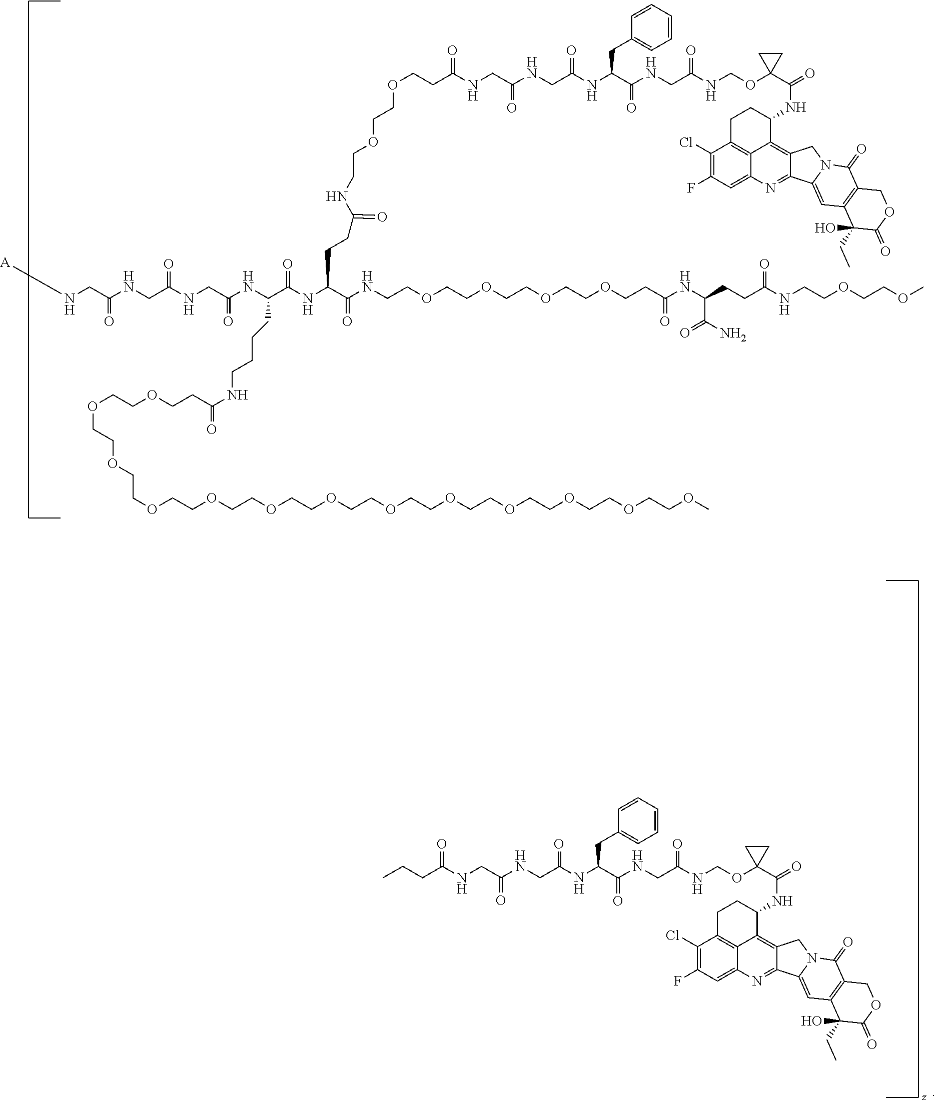

In one embodiment, the conjugate III-a has the structure of:

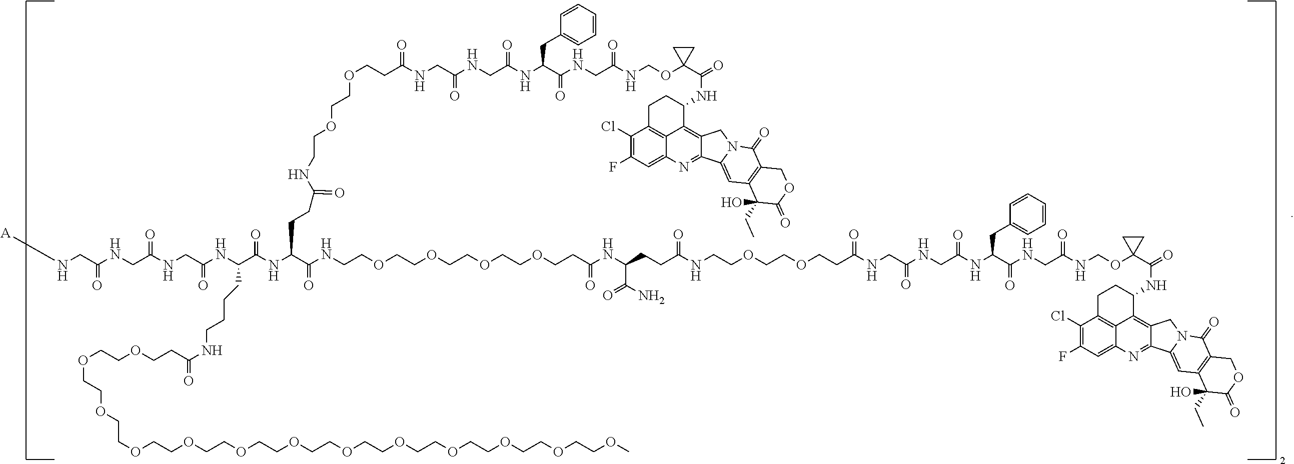

In one embodiment, i is 4, n is 3, m is 1, j is 12, L² is —(C₂H₄—O)ₚ—(CH₂)₂ (CO)—, p is 2, L¹ is Val-Cit, B is PABC—NH—CH₂-U-R¹⁰ or PABC-R¹⁰ or PABC—NH—CH₂-U—(CR¹R²)_{g}—(CO)—R¹⁰, U is O, g is 1, R¹¹ is methyl. In one embodiment, conjugate III-a has the structure of:

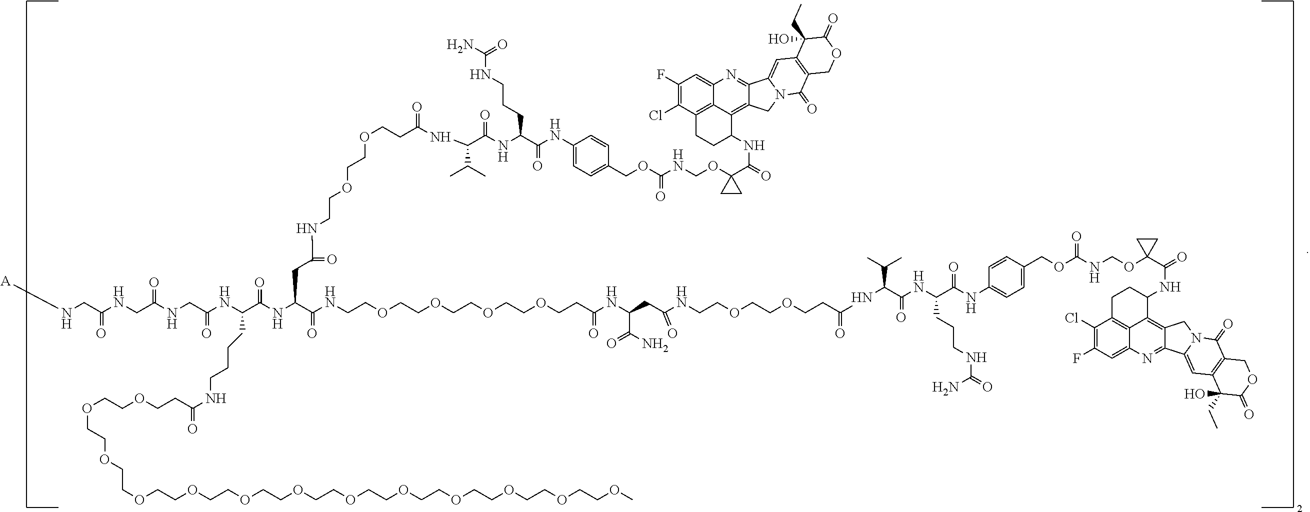

In one embodiment, conjugate III-a has the structure of:

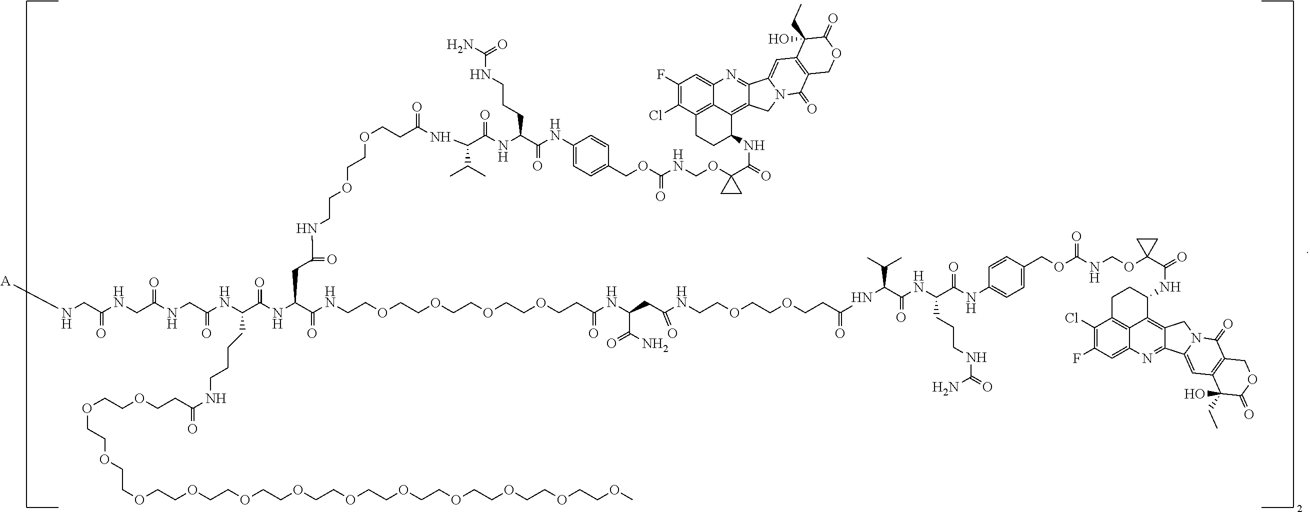

In one embodiment, i is 4, n is 3, m is 2, j is 12, L² is —(C₂H₄—O)ₚ—(CH₂)₂(CO)—, p is 2, L¹ is Val-Cit, B is PABC—NH—CH₂-U-R¹⁰ or PABC-R¹⁰ or PABC—NH—CH₂-U—(CR¹R²), (CO)—R¹⁰, U is O, g is 1, R¹¹ is methyl. In one embodiment, conjugate III-a has the structure of:

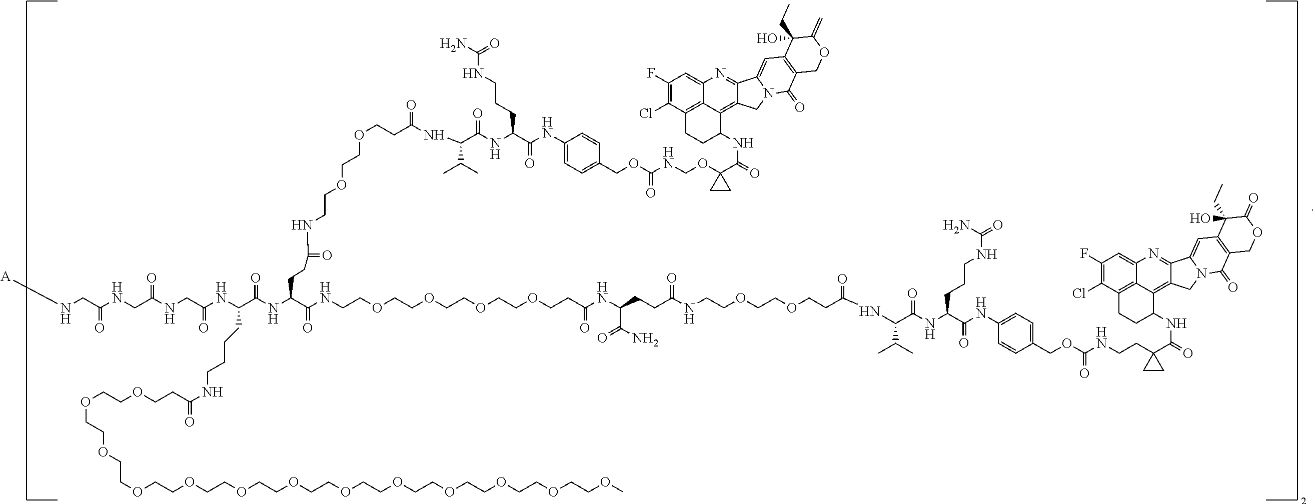

In one embodiment, conjugate III-a has the structure of:

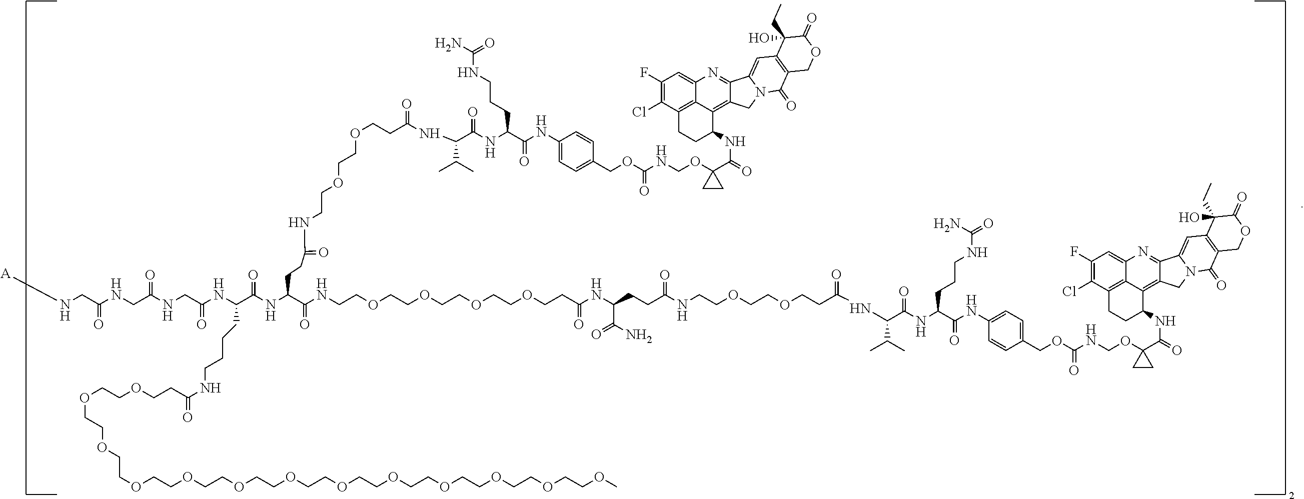

In one embodiment, i is 4, n is 3, m is 1, t is 12, L² is —(C₂H₄—O)ₚ—(CH₂)₂(CO)—, p is 2, L¹ is Gly-Gly-Phe-Gly, B is-NH-CH2-U-R¹⁰ or —R¹⁰ or —NH—CH₂-U—(CR¹R²)_{g}—(CO)—R¹⁰, U is O, g is 1. In one embodiment, conjugate III-b has the structure of:

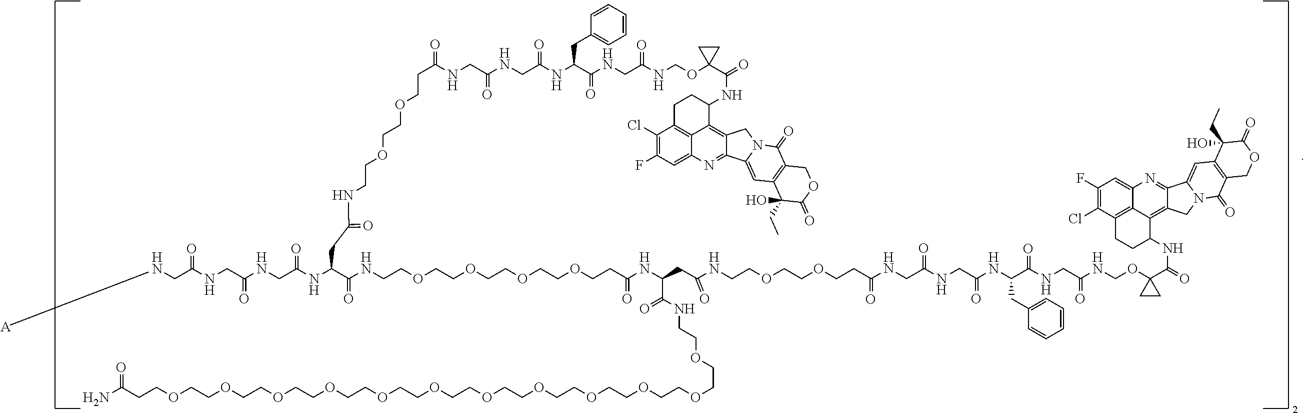

In one embodiment, conjugate III-b has the structure of:

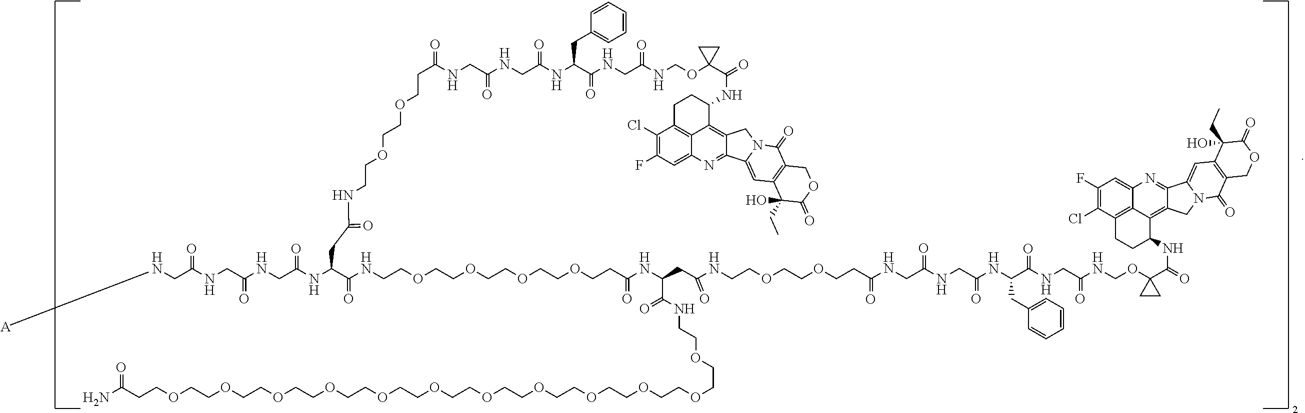

In one embodiment, i is 4, n is 3, m is 2, t is 12, L² is —(C₂H₄—O)ₚ—(CH₂)₂(CO)—, p is 2, L¹ is Gly-Gly-Phe-Gly, B is—NH—CH₂-U-R¹⁰ or —R¹⁰ or —NH—CH₂-U—(CR¹R²)_{g}—(CO)—R¹⁰, U is O, g is 1. In one embodiment, conjugate III-b has the structure of:

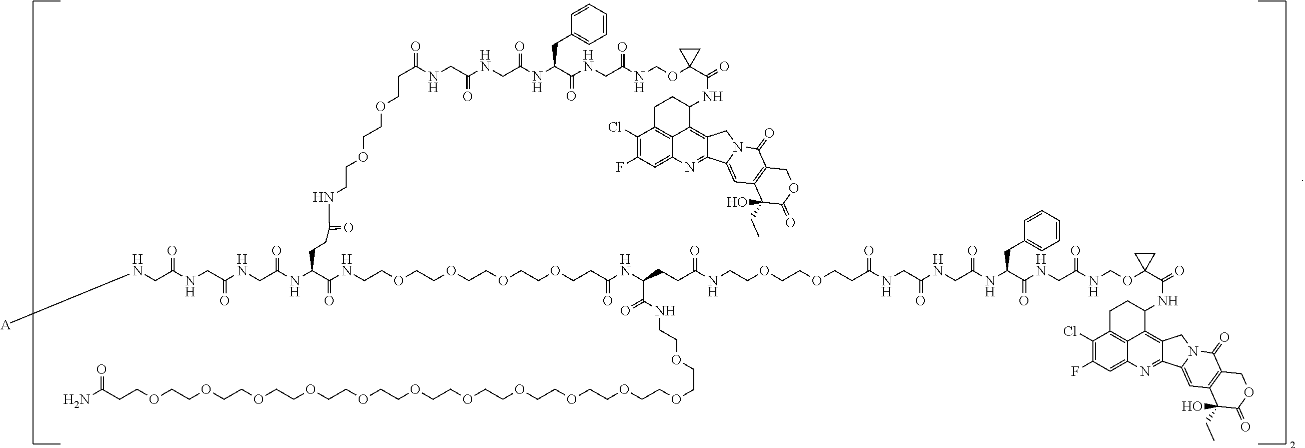

In one embodiment, conjugate III-b has the structure of:

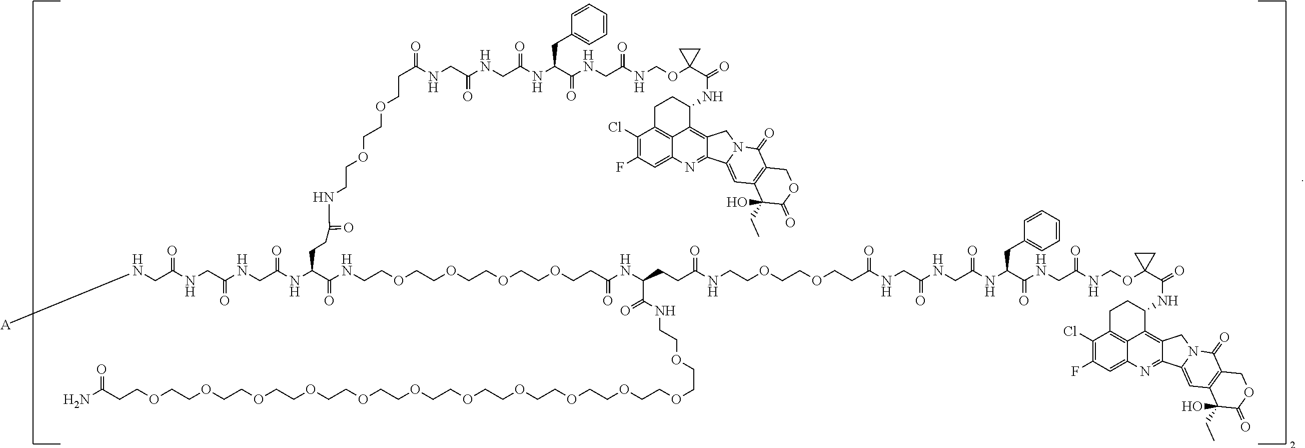

In one embodiment, i is 4, n is 3, m is 1, t is 12, L² is —(C₂H₄—O)ₚ—(CH₂)₂(CO)—, p is 2, L¹ is Val-Cit, B is PABC—NH—CH₂-U-R¹⁰ or PABC-R¹⁰ or PABC—NH—CH₂-U—(CR¹R²)_{g}—(CO)—R¹⁰, g is 1. In one embodiment, conjugate III-b has the structure of:

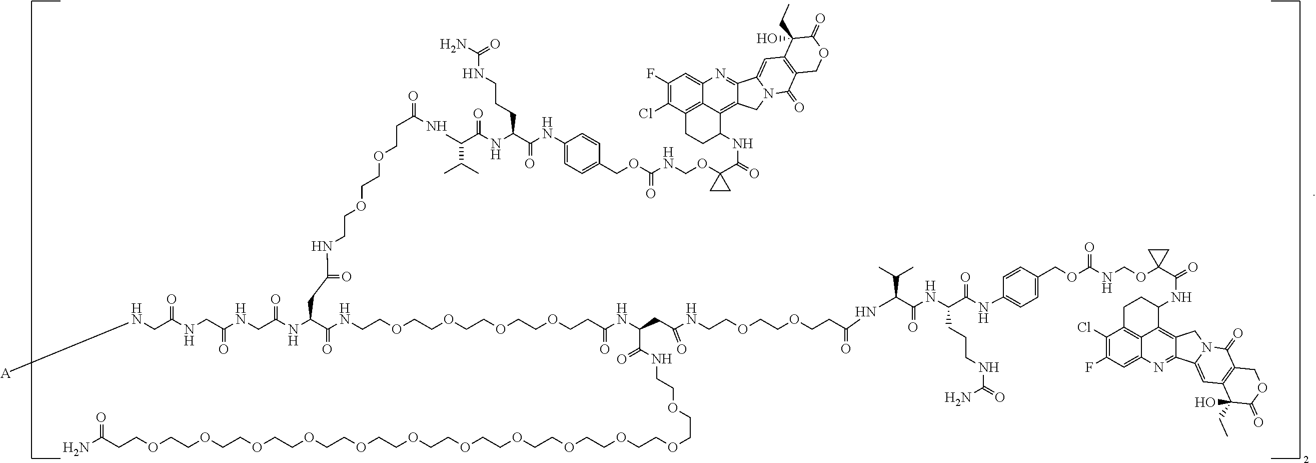

In one embodiment, conjugate III-b has the structure of:

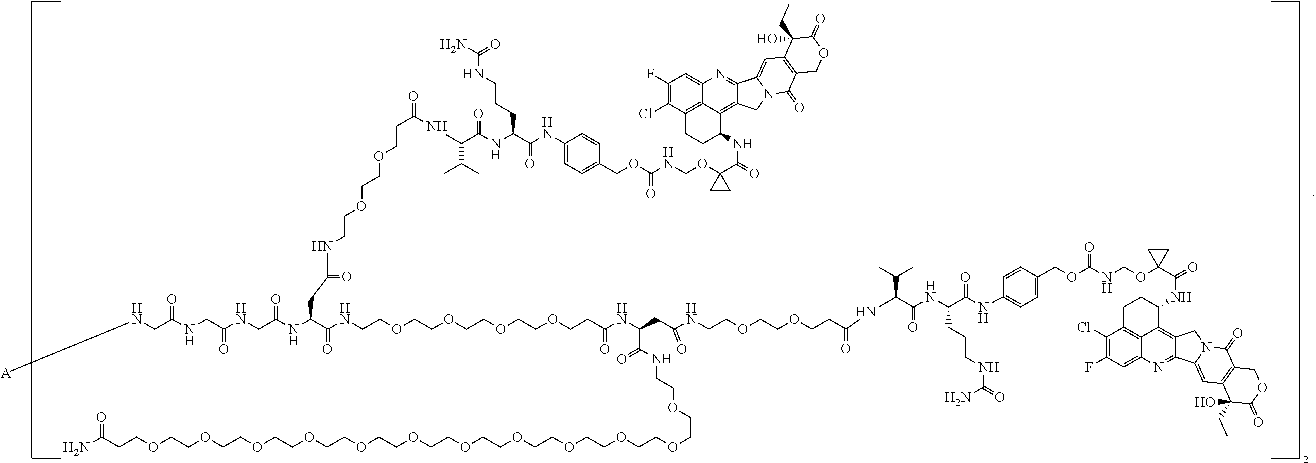

In one embodiment, i is 4, n is 3, m is 2, t is 12, L² is —(C₂H₄—O)ₚ—(CH₂)₂(CO)—, p is 2, L¹ is Val-Cit, B is PABC—NH—CH₂—U—R¹⁰ or PABC-R¹⁰ or PABC—NH—CH₂—U—(CR¹R²)_{g}—(CO)—R¹⁰, g is 1. In one embodiment, conjugate III-b has the structure of:

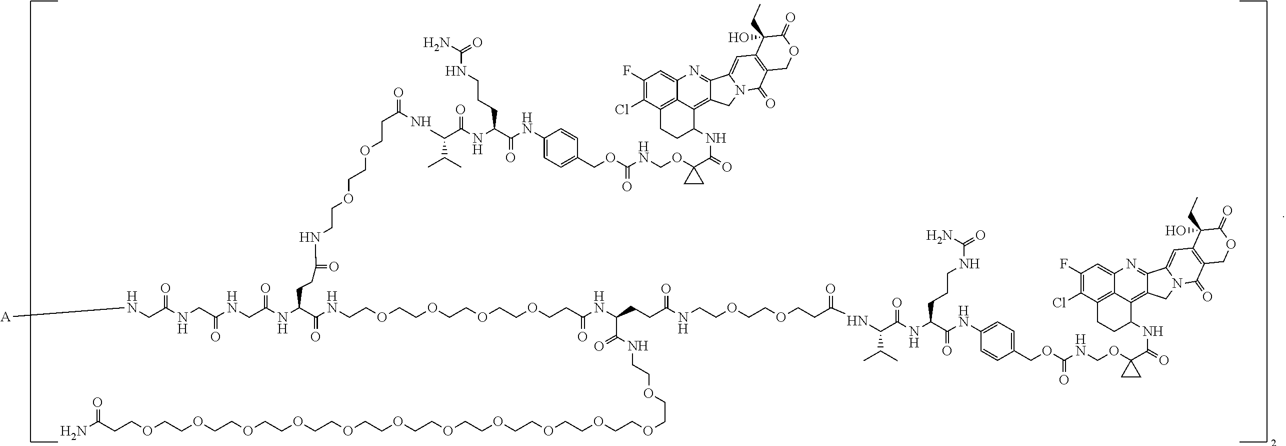

In one embodiment, conjugate III-b has the structure of:

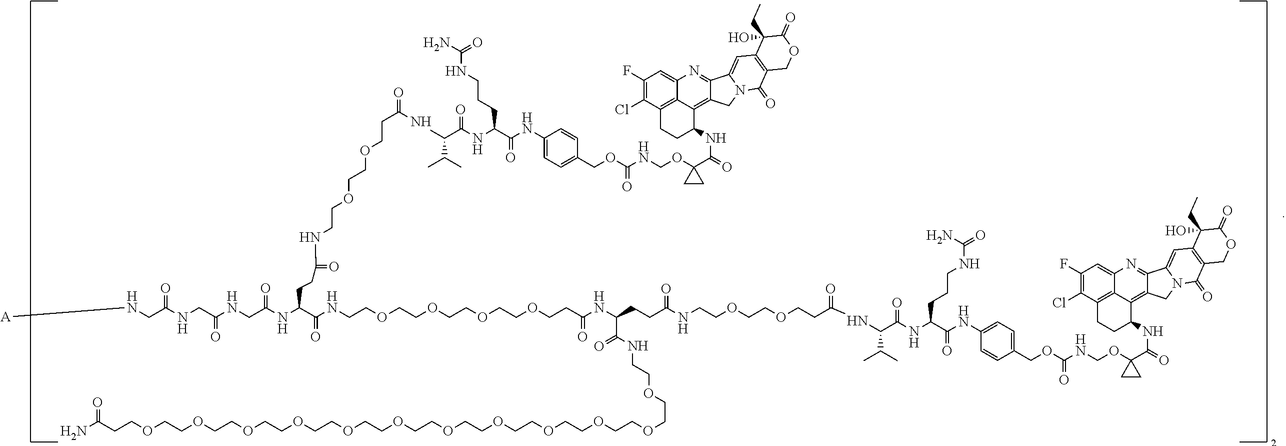

### Preparation of the Conjugate

The conjugates of the present disclosure can be prepared by any method known in the art. In some embodiments, the conjugate is prepared by the ligase-catalyzed site-specific conjugation of a targeting molecule and a payload-bearing formula (I) compound, wherein the targeting molecule is modified by a ligase recognition sequence. The method comprises step A and step B.

### Step A. Preparation of the Linking Unit-Payload Intermediate

In a preferred embodiment, B in the compound of formula (I) is covalently linked via a reactive group to a payload containing another reactive group.

The linking unit-payload intermediate prepared using the compound of formula (I) of the present disclosure has defined structure, defined composition and high purity, so that when the conjugation reaction with an antibody is conducted, fewer impurities are introduced or no other impurities are introduced. When such an intermediate is used for the ligase-catalyzed site-specific conjugation with a modified antibody containing a ligase recognition sequence, a homogeneous ADC with highly controllable quality is obtained.

### Step B. Linking the Targeting Molecule to the Payload-Bearing Formula (I) Compound

The targeting molecule of the present disclosure can be conjugated with the payload-bearing formula (I) compound (i.e., the compound of formula (II) by any method known in the art.

The targeting molecule and the payload-bearing formula (I) compound are linked to each other via the ligase-specific recognition sequences of the substrates. The recognition sequence depends on the particular ligase employed. In one embodiment, the targeting molecule is an antibody with recognition sequence-based terminal modifications introduced at the C-terminal of the light chain and/or the heavy chain, and the targeting molecule is conjugated with the compound of formula (II), under the catalysis of the wild type or optimized engineered ligase or any combination thereof, and under suitable catalytic reaction conditions.

In a specific embodiment, the ligase is Sortase A and the conjugation reaction can be represented by the following scheme:

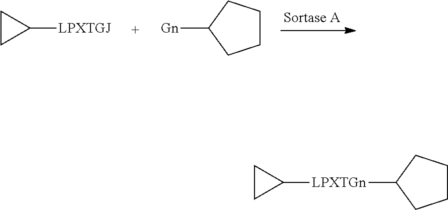

The triangle represents a portion of an antibody; and the pentagon represents a portion of a compound of formula (II). n, X and J are respectively as defined above. When conjugated with Gn, which is the corresponding recognition sequence of the acceptor substrate, the upstream peptide bond of the glycine in the LPXTGJ sequence is cleaved by Sortase A, and the resulting intermediate is linked to the free N-terminal of Gn to generate a new peptide bond. The resulting amino acid sequence is LPXTGn. The sequences Gn and LPXTGJ are as defined above.

### Metabolism of the Conjugate in a Physiological Environment

When a part or whole linker is cleaved in tumor cells, the antitumor compound moiety is released to exhibit the antitumor effect of the antitumor compound. As the linker is cleaved at a connecting position to drug, the antitumor compound is released in its intrinsic structure to exhibit its intrinsic antitumor effect.

In one embodiment, Cleavable sequence 1 (such as Gly-Gly-Phe-Gly) can be cleaved by lysosomal enzymes (such as cathepsin B and/or cathepsin L).

In one embodiment, Sp1 comprises a self-immolative spacer. In one embodiment, Sp1 comprises PABC, an acetal or a heteroacetal. In one embodiment, L¹ is Gly-Gly-Phe-Gly. In one embodiment, the linker comprises - Gly-Gly-Phe-Gly—NH—CH₂-O—. In one embodiment, -Gly-Gly-Phe-Gly—NH—CH₂—O- represents a combination of a restriction enzyme site and a self-immolative spacer, which would cleave in the cell and release the aimed molecule (such as the drug).

### Table of Specific Conjugates

In one embodiment, the payload is a cytotoxin or a fragment thereof. In one embodiment, the antibody is a modified Trastuzumab, preferably Ab0001-LCCT_{L}-HC (light chain SEQ ID NO: 1, heavy chain: SEQ ID NO: 2) or Ab0001-LCCT_{L}-HCCT_{L} (light chain SEQ ID NO: 3, heavy chain: SEQ ID NO: 4). The sequence of each of Ab0001-LCCT_{L}-HC and Ab0001-LCCT_{L}-HCCT_{L} is based on the amino acid sequence of Ab0001 (Trastuzumab), and GALPETGG was introduced at the C-terminal of the light chain (Ab0001-LCCT_{L}-HC) or at the C-terminal of the light chain and the heavy chain (Ab0001-LCCT_{L}-HCCT_{L}), wherein LPETGG is the recognition sequence of the ligase donor substrate, and GA is a spacer sequence. In Ab0001-LCCT_{L}-HCCT_{L}, the lysine at the C-terminal of the heavy chain of Ab0001 can be maintained as in SEQ ID NO: 4 or removed (resulting sequence not shown in the sequence list) before the GALPETGG is introduced. In one embodiment, the antibody-drug conjugate is as shown in the following table.

In one embodiment, the antibody is a Ab2 (light chain SEQ ID NO: 5, heavy chain: SEQ ID NO: 6).

In one embodiment, the antibody is a Ab3 (light chain SEQ ID NO: 7, heavy chain: SEQ ID NO: 8).

Nomenclature of the ADCs: the number in the parenthesis indicates the number of payload (drug) molecules that is intended to be connected to the antibody.

| | Linker-Payload | | A |
|---|---|---|---|
| ADC | formula | Payload | (targeting molecule) |
| III-a-1(2) | II-a-1 | compound 15 | Ab0001-LCCT_{L}-HC |
| III-a-1(4) | II-a-1 | compound 15 | Ab0001-LCCT_{L}-HCCT_{L} |
| ADC-5 | II-a-1 | compound 15 | Ab2 |
| ADC-8 | II-a-1 | compound 15 | Ab3 |

### Pharmaceutical Composition and Pharmaceutical Preparation

Another object of the disclosure is to provide a pharmaceutical composition comprising a prophylactically or therapeutically effective amount of a conjugate of the present disclosure, and at least one pharmaceutically acceptable carrier.

The pharmaceutical composition of the present disclosure may be administered in any manner as long as it achieves the effect of preventing, alleviating, preventing or curing the symptoms of a human or animal. For example, various suitable dosage forms can be prepared according to the administration route, especially injections such as lyophilized powder for injection, injection, or sterile powder for injection.

The term “pharmaceutically acceptable” means that when contacted with tissues of the patient within the scope of normal medical judgment, no undue toxicity, irritation or allergic reaction, etc. shall arise, having reasonable advantage-disadvantage ratios and effective for the intended use.

The term pharmaceutically acceptable carrier refers to those carrier materials which are pharmaceutically acceptable and which do not interfere with the bioactivities and properties of the conjugate. Examples of aqueous carriers include but are not limited to buffered saline, and the like. The pharmaceutically acceptable carrier also includes carrier materials which brings the composition close to physiological conditions, such as pH adjusting agents, buffering agents, toxicity adjusting agents and the like, and sodium acetate, sodium chloride, potassium chloride, calcium chloride, sodium lactate, and the like.

In one embodiment, the pharmaceutical composition of the present disclosure has a drug to antibody ratio (DAR) of an integer or non-integer of about 1 to about 20, such as about 1 to about 10, about 1 to about 8, about 1 to about 6, about 1 to about 4, about 1 to about 3, about 1 to about 2.5, about 1 to about 2. In a particular embodiment, the conjugate of the present disclosure has a DAR of about 2, about 4, about 6 or about 8.

### Treatment Method and Use

The conjugates of the present disclosure are useful for the treatment of tumors and/or autoimmune diseases. Tumors conjugate treatment include those characterized by specific tumor-associated antigens or cell surface receptors, and those will be recognized by the targeting molecule in the conjugate and can be killed by the payload/cytotoxin in the conjugate.

Accordingly, in yet another aspect, also provided is use of a conjugate of the present disclosure or a pharmaceutical composition of the present disclosure in the manufacture of a medicament for treating a disease, disorder or condition selected from a tumor or an autoimmune disease.

In another aspect, provided is a conjugate of the present disclosure or a pharmaceutical composition of the present disclosure for use in the treatment of a tumor or an autoimmune disease.

In a further aspect, provided is a method of treating a tumor or an autoimmune disease, the method comprising administering to an individual in need thereof an effective amount of a conjugate of the present disclosure or a pharmaceutical composition of the present disclosure.

In a preferred embodiment, the conjugate of the present disclosure formed by conjugation of the anti-human HER2 antibody and the small molecule cytotoxin can specifically bind to HER2 on the surface of the tumor cell and selectively kill the HER2-expressing tumor cells. In another preferred embodiment, provided is use of a conjugate of the present disclosure or a pharmaceutical composition of the present disclosure in the manufacture of a medicament for treating a disease, disorder or condition selected from HER2-positive tumors. In a more preferred embodiment, the disease, disorder or condition is selected from the group consisting of breast cancer, gastric cancer, lung cancer, ovarian cancer, urothelial cancer, and the like.

In a preferred embodiment, the conjugate of the present disclosure formed by conjugation of the anti-human TROP2 antibody and the small molecule cytotoxin can specifically bind to TROP2 on the surface of the tumor cell and selectively kill the TROP2-expressing tumor cells. In another preferred embodiment, provided is use of a conjugate (or an antibody) of the present disclosure or a pharmaceutical composition of the present disclosure in the manufacture of a medicament for treating a disease, disorder or condition selected from TROP2-positive tumors. In a more preferred embodiment, the disease, disorder or condition is TROP2-positive tumor. In one embodiment, the TROP2-positive tumor is selected from the group consisting of breast cancer, gastric cancer, lung cancer, ovarian cancer, urothelial cancer, and the like.

In a preferred embodiment, the conjugate of the present disclosure formed by conjugation of the anti-FGFR3 antibody and the small molecule cytotoxin can specifically bind to FGFR3 on the surface of the tumor cell and selectively kill the FGFR3-expressing tumor cells. In another preferred embodiment, provided is use of a conjugate of the present disclosure or a pharmaceutical composition of the present disclosure in the manufacture of a medicament for treating FGFR3-mediated disease. Specifically, an FGFR3-positive tumor, more specifically brain cancer, bladder cancer, urothelial cancer, cervical cancer, or intrahepatic cholangiocarcinoma. In some embodiments, the disease includes tumor overexpressing FGFR3 or tumor with FGFR3 gene mutation. In some embodiments, the disease is selected from the group consisting of fibrosarcoma, myxosarcoma, liposarcoma, chondrosarcoma, osteogenic sarcoma, chordoma, angiosarcoma, endotheliosarcoma, lymphangiosarcoma, lymphangioendothelial sarcoma, synovioma, mesothelioma, Ewing's tumor, leiomyosarcoma, rhabdomyosarcoma, colon cancer, pancreatic cancer, breast cancer, thyroid cancer, endometrial cancer, melanoma, ovarian cancer, prostate cancer, squamous cell carcinoma, basal cell carcinoma, adenocarcinoma, sweat gland carcinoma, sebaceous carcinoma, papillary carcinoma, papillary adenocarcinoma, cystadenocarcinoma, medullary carcinoma, bronchial carcinoma, renal cell carcinoma, liver cancer, bile duct cancer, choriocarcinoma, seminoma, embryonal carcinoma, Wilms' tumor, cervical cancer, testicular tumor, lung cancer, small cell lung cancer, bladder cancer, epithelial cancer, glioma, astrocytoma, medulloblastoma, craniopharyngioma, ependymoma, pinealoma, hemangioblastoma, acoustic neuroma, oligodendroglioma, meningioma, neuroblastoma and retinoblastoma. In some embodiments, the disease is selected from: brain cancer, bladder cancer, urothelial cancer, cervical cancer, or intrahepatic cholangiocarcinoma. In some embodiments, the disease is glioblastoma.

The dosage of the conjugate administered to the subject can be adjusted to a considerable extent. The dosage can vary according to the particular route of administration and the needs of the subject, and can be subjected to the judgment of the health care professional.

### Beneficial Effects

The present branched linker contains no maleimide group and is conjugated to the antibody at a specific site through an amino acid at a tail, and thus avoiding the potential instability in blood. Meanwhile, the branched linker has good assembling ability, and can be adapted to a variety of different payloads and commercialized linker-payload fragments conveniently and environment-friendly. The formed linker-payload structures are easy to be separated and purified, which is beneficial for CMC development in a later stage. In addition, by conjugating different kinds of linkers to different antibodies, the stability, compatibility of the linkers and the excellent biological activity of the ADCs formed by the linkers are verified.

The antibody-drug conjugate of the present disclosure uses specially designed linker-payload, and is more stable and can achieve great efficacy in lower DAR, and therefore can reduce side effects and increase the therapeutic index.

The present disclosure utilizes a linking unit with unique structure and uses a ligase to catalyze the conjugation of the targeting molecule and the payload. The conjugate of the present disclosure has good homogeneity, high activity and high selectivity. Furthermore, the toxicity of the linking unit-payload intermediate is much lower than that of the free payload, and thus the manufacture process of the drug is less detrimental, which is advantageous for industrial production.

The conjugates of the present disclosure achieve at least one of the following technical effects:
(1) High inhibitory activity against target cells, or strong killing effect on target cells.
(2) Good physicochemical properties (e.g., solubility, physical and/or chemical stability).
(3) Good pharmacokinetic properties (e.g., good stability in plasma, appropriate half-life and duration of action).
(4) Good safety (low toxicity on non-target normal cells or tissues, and/or fewer side effects, wider treatment window), etc.
(5) Highly modular design, simple assembly of multiple drugs.
(6) Low payload consumption, and low production cost.
(7) Few isomers, and easy for CMC.

### EXAMPLES

### Preparation Example

In order to more clearly illustrate the objects and technical solutions, the present disclosure is further described below with reference to specific examples. It is to be understood that the examples are not intended to limit the scope of the disclosure. The specific experimental methods which were not mentioned in the following examples were carried out according to conventional experimental method.

### Instruments, Materials and Reagents

Unless otherwise stated, the instruments and reagents used in the examples are commercially available. The reagents can be used directly without further purification. The intermediates used are either commercially available or synthetic.

MS: Thermo Fisher Q Exactive Plus, Water2795-Quattro micro triple quadrupole mass spectrometer

HPLC: Waters 2695, Agilent 1100, Agilent 1200

Semi-preparative HPLC: Lisure HP plus 50D

Flow Cytometry: CytoFLEX S

HIC-HPLC: Butyl-HIC; mobile phase A: 25 mM PB, 2M (NH₄)₂SO₄, pH 7.0; mobile phase B: 25 mM PB, pH 7.0; flow rate: 0.8 ml/min; acquisition time: 25 min; injection amount: 20 μg; column temperature: 25° C.; detection wavelength: 280 nn; sample chamber temperature: 8° C. SEC-HPLC: column: TSK-gel G3000 SWXL, TOSOH 7.8 mm ID×300 mm, 5 μm; mobile phase: 0.2 M KH₂PO₄, 0.25 M KCl, pH 6.2; flow rate: 0.5 ml/min; acquisition time: 30 min; injection volume: 50 μl; column temperature: 25° C.; detection wavelength; 280 nm; sample tray temperature: 8° C.

CHO was obtained from Thermo Fisher Scientific; Rink-amide-MBHA-resin were obtained from Nankai synthesis; SK-BR-3 was obtained from ATCC CAT #HTB-30; NCI-N87 cells was obtained from ATCC CAT #CRL-5822; MDA-MB-468 was obtained from ATCC CAT #HTB-132; JIMT-1 was obtained from Wuxi Apptech; antibody Trastuzumab is prepared according to the known sequence; optimized recombinant enzyme Sortase A derived from Staphylococcus aureus is prepared in E. coli.

### Example 1 Construction of Antibody Expression Vector, Antibody Expression, Purification and Identification

### 1.1 Production of the Modified Anti-Human HER2 Antibody Ab0001-LCCT_{L}-HC

The expression plasmids for antibody Ab0001-LCCT_{L}-HC (light chain SEQ ID NO: 1, heavy chain: SEQ ID NO: 2) were constructed as follows. The sequence of the antibody Ab0001-LCCT_{L}-HC: based on the amino acid sequence of Trastuzumab, and GALPETGG was introduced at the C-terminal of the light chain, wherein LPETGG is the recognition sequence of the ligase donor substrate, and GA is a spacer sequence. The plasmids were transfected into CHO cells and the cell population was established and screened for a highly expressed cell population, which was cultured with reference to the culture process of Trastuzumab in a 5-10 L reactor, and supernatant was collected.

### 1.2 the Purification of Antibody Ab0001-LCCT_{L}-HC

The purification of Ab0001-LCCT_{L}-HC was carried out in a standard process using the combination of MabSelect affinity chromatography and Sepharose S cation exchange chromatography, the purified products were dissolved in the original Trastuzumab drug buffer (5 mM histidine-HCl, 2% Trehalose, 0.009% Polysorbate 20, PH 6.0), and frozen in small aliquots.

### 1.3 the Quality Control of Antibody Ab0001-LCCT_{L}-HC

The purity of the above purified antibody Ab0001-LCCT_{L}-HC is 98.5% by SDS-PAGE; the content of high molecular weight polymer of the sample is less than 0.4% by SEC-HPLC; endotoxin content is less than 0.098 EU/mg.

### 1.4 Preparation of Other Modified Anti-Human Antibodies

According to a similar method, a terminal modification based on the ligase recognition sequence was introduced at the C-terminal of the light and/or heavy chain of the Trastuzumab, hRS7(Ab2) and Ab3, respectively, giving a modified antibody.

The modified anti-human antibodies are listed in Table 1. LPETGG in the terminal modification sequence is a recognition sequence of the ligase donor substrate, and GA is a spacer sequence.

TABLE 1
Modified anti-human antibodies

| | | Sequence |
|---|---|---|
| | | introduced at |
| | Sequence | the terminal |
| Ab0001-LCCT_{L}-HC light chain | SEQ ID NO: 1 | GALPETGG |
| Ab0001-LCCT_{L}-HC heavy chain | SEQ ID NO: 2 | -* |
| Ab0001-LCCT_{L}-HCCT_{L} light chain | SEQ ID NO: 3 | GALPETGG |
| Ab0001-LCCT_{L}-HCCT_{L} heavy chain | SEQ ID NO: 4 | GALPETGG |
| Ab2 light chain | SEQ ID NO: 5 | GALPETGG |
| Ab2 heavy chain | SEQ ID NO: 6 | -* |
| Ab3 light chain | SEQ ID NO: 7 | GALPETGG |
| Ab3 heavy chain | SEQ ID NO: 8 | -* |

*: “-” indicates no terminal modification

### Example 2 Preparation of Linker-Payload Intermediate

### 2.1 Preparation of Intermediate Mc-GGFG-Dxd

The intermediate Mc-GGFG-Dxd is commercially available or prepared following the procedures as described in EP2907824. This compound is used to prepare the Linker-Payload intermediate, and is also used to directly connect to the (optionally modified) antibody to prepare reference ADCs.

### 2.2 Preparation of Linker-Payload 1

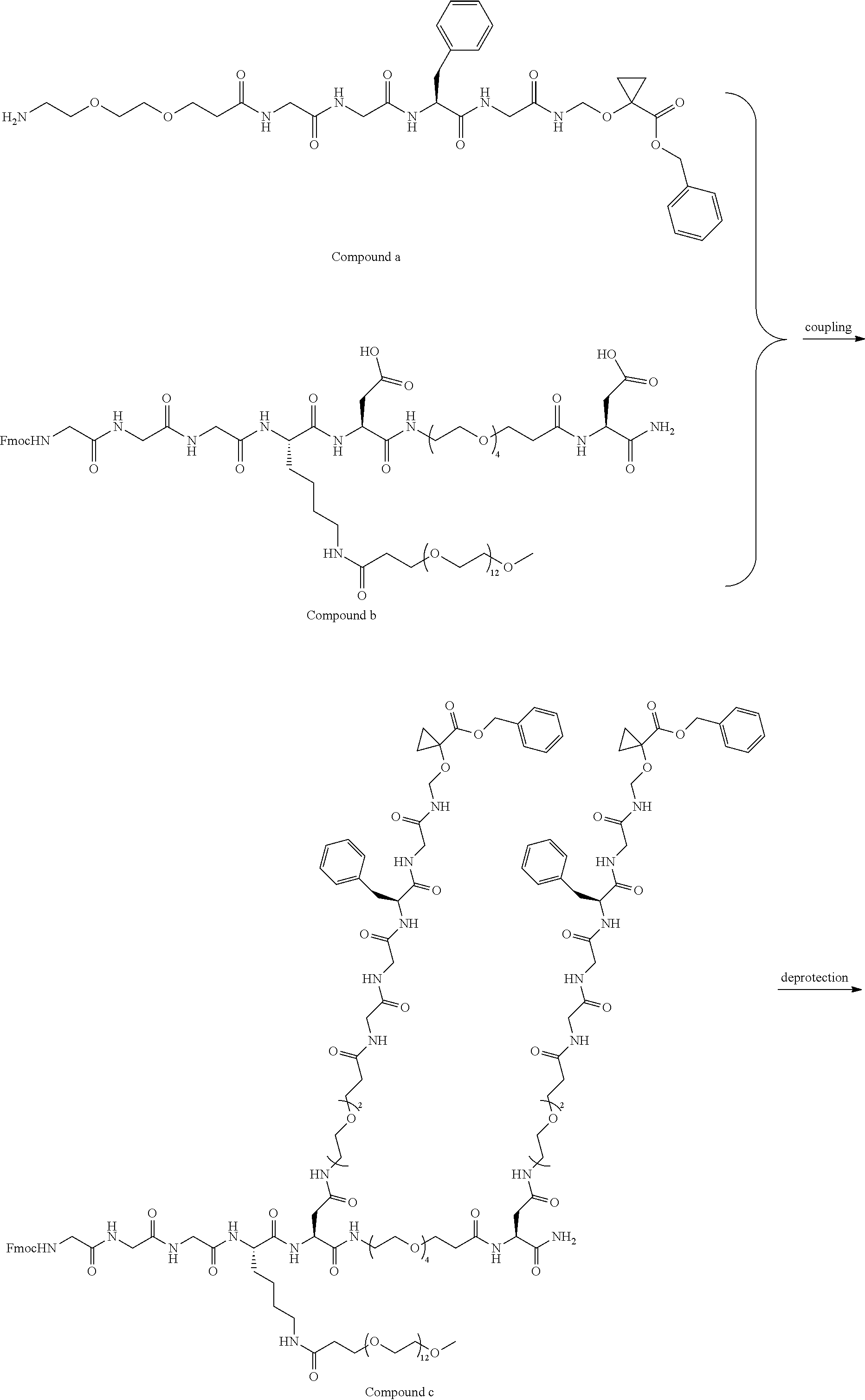

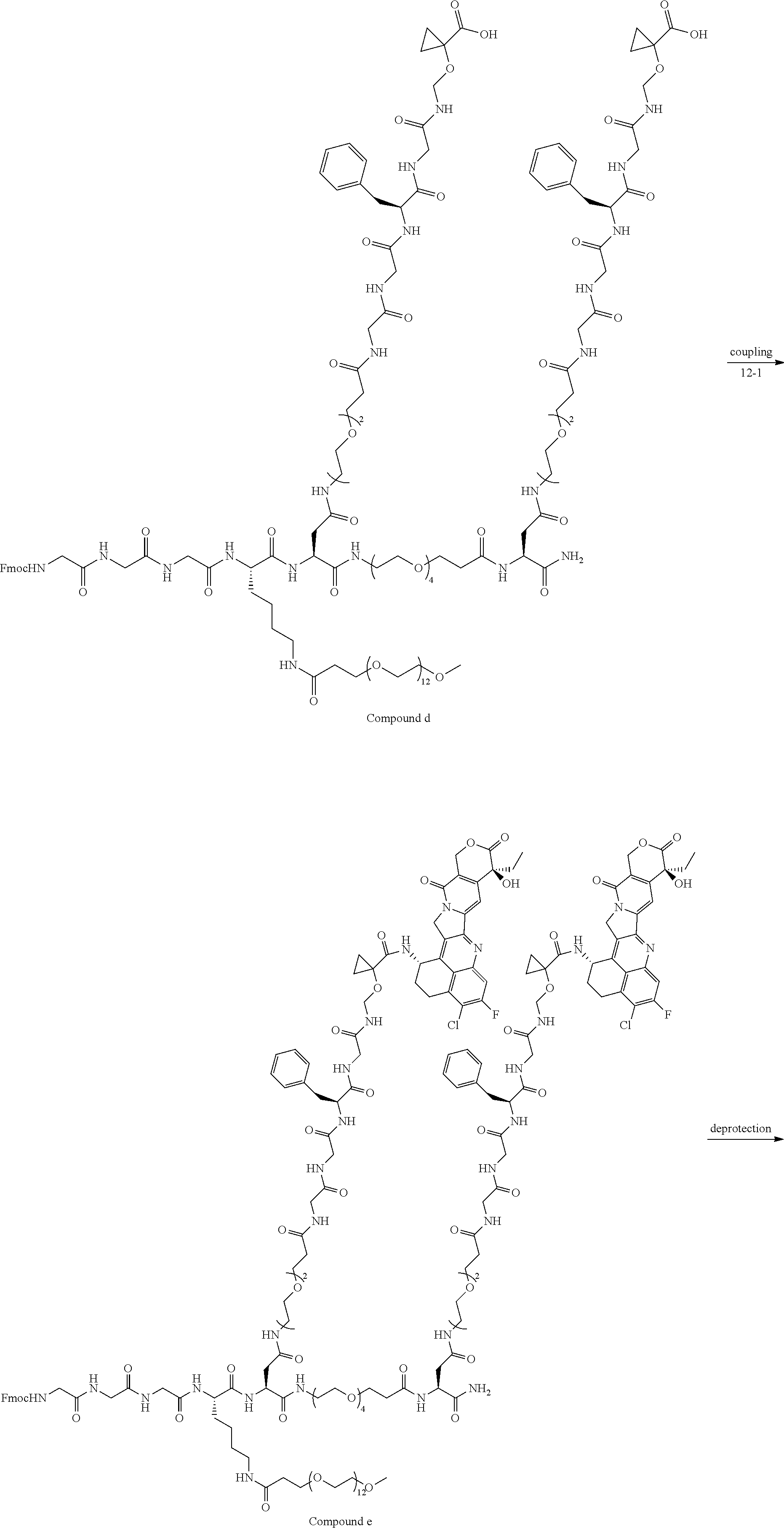

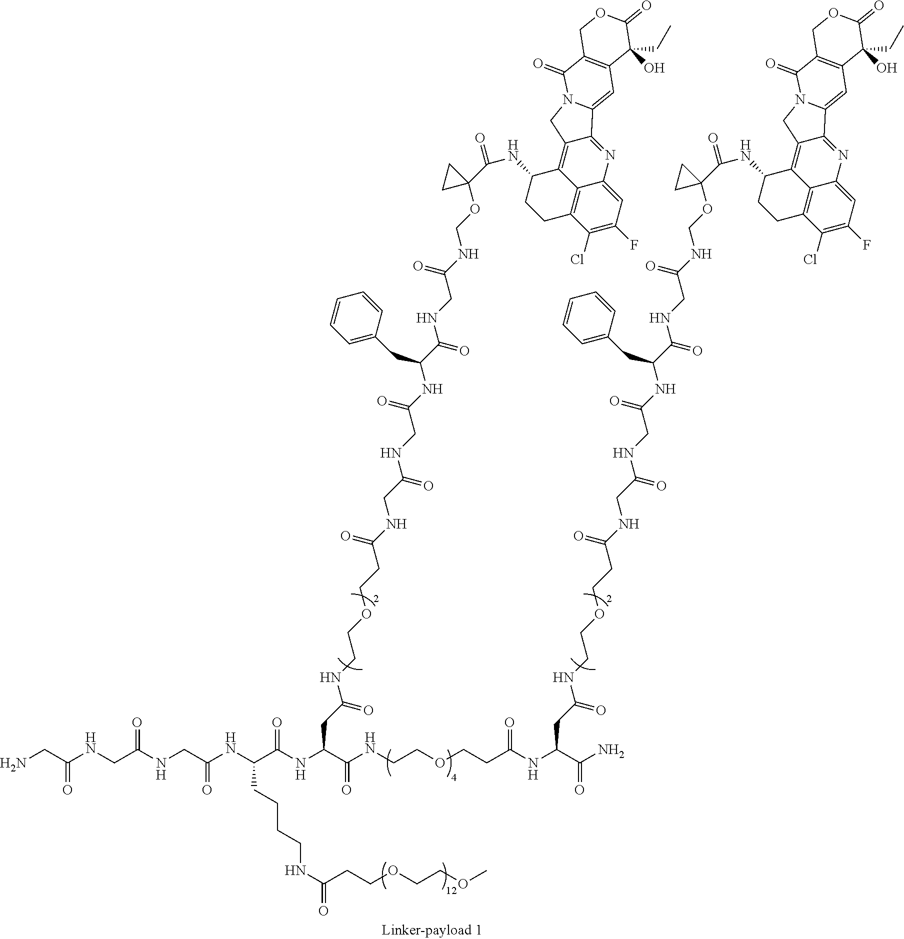

### Step 1: Preparation of Intermediate Compound b

### Step 1.1 Preparation of NH₂-Asp(OtBu)-Rink Amide Resin

400 g of Rink amide resin was weighed and fully swelled by 2400 mL of DCM. 2400 mL of deprotection reagent was added to remove Fmoc completely and then washed several times with DMF and DCM in room temperature. In the subsequent ninhydrin test, the resin showed blue color.

88.87 g Fmoc-Asp(OtBu)-OH and 29.19 g HOBT were weighed and dissolved in 2000 mL DMF and 80 mL DIC solution. After being placed in an ice bath at −10° C. for 0.5 h, it was slowly added into the reaction kettle with resin, and the reaction was stirred at room temperature for 2-5 h with nitrogen, and then filtered. The resin was washed with DMF and DCM successively and showed colorless or light yellow in the subsequent ninhydrin test.

2400 mL of deprotection reagent was added to remove Fmoc completely and then washed several times with DMF and DCM in room temperature. In the subsequent ninhydrin test, the resin showed blue color.

### Step 1.2 Preparation of NH₂-PEG4-Asp(OtBu)-Rink Amide Resin

131.64 g Fmoc-PEG4-OH and 48.64 g HOBT were weighed and dissolved in 2000 mL DMF and 80.0 mL DIC solution. After being placed in an ice bath at −10° C. for 0.5 h, it was slowly added to the reaction kettle with resin, and the reaction was stirred at room temperature for 2-4 h with nitrogen, and then filtered. The resin was washed with DMF and DCM successively and showed colorless or light yellow in the subsequent ninhydrin test.

2400 mL of deprotection reagent was added to remove Fmoc completely and then washed several times with DMF and DCM in room temperature. In the subsequent ninhydrin test, the resin showed blue color.

### Step 1.3 Preparation of NH₂-Asp(OtBu)-PEG4-Asp(OtBu)-Rink Amide Resin

222.18 g Fmoc-Asp(OtBu)-OH and 72.96 g HOBT were weighed and dissolved in 2000 mL DMF and 80 mL DIC solution. After being placed in an ice bath at −10° C. for 0.5 h, it was slowly added to the reaction kettle with resin, and the reaction was stirred at room temperature for 2-4 h with nitrogen, and then filtered. The resin was washed with DMF and DCM successively and showed colorless or light yellow in the subsequent ninhydrin test.

2400 mL of deprotection reagent was added to remove Fmoc completely and then washed several times with DMF and DCM in room temperature. In the subsequent ninhydrin test, the resin showed blue color.

Step 1.4 preparation of Dde-Lys(NH₂)-Asp(OtBu)-PEG4-Asp(OtBu)-Rink amide resin

191.75 g Dde-Lys(Fmoc)-OH and 48.64 g HOBT were weighed and dissolved in 2000 mL DMF and 80.0 mL DIC solution. After being placed in an ice bath at −10° C. for 0.5 h, it was slowly added to the reaction kettle with resin, and the reaction was stirred at room temperature for 2-4 h with nitrogen, and then filtered. The resin was washed with DMF and DCM successively and showed colorless or light yellow in the subsequent ninhydrin test.

2400 mL of deprotection reagent was added to remove Fmoc completely and then washed several times with DMF and DCM in room temperature. In the subsequent ninhydrin test, the resin showed blue color.

### Step 1.5 Preparation of Dde-Lys(mPEG12)-Asp(OtBu)-PEG4-Asp(OtBu)-Rink Amide Resin

170.84 g m-PEG12-CH₂CH₂COOH and 48.64 g HOBT were weighed and dissolved in 2000 mL DMF and 80.0 mL DIC solution. After being placed in an ice bath at −10° C. for 0.5 h, it was slowly added to the reaction kettle with resin, and the reaction was stirred at room temperature for 2-4 h with nitrogen, and then filtered. The resin was washed with DMF and DCM successively and showed colorless or light yellow in the subsequent ninhydrin test.

### Step 1.6 Preparation of NH₂-Lys(PEG12)-Asp(OtBu)-PEG4-Asp(OtBu)-Rink Amide Resin

2400 mL of de-Dde reagent was added, and the reaction was stirred at room temperature under nitrogen for 0.5 h, and then filtered. After repeating the operation 3 times, the resin was washed with DMF and DCM successively and showed blue color in the subsequent ninhydrin test.

### Step 1.7 Preparation of Fmoc-Gly-Gly-Gly-Lys(PEG12)-Asp(OtBu)-PEG4-Asp(OtBu)-Rink Amide Resin

111.08 g Fmoc-Gly-Gly-Gly-OH and 48.64 g HOBT were weighed and dissolved in 2000 mL DMF and 80.0 mL DIC solution. After being placed in an ice bath at −10° C. for 0.5 h, it was slowly added to the reaction kettle with resin, and the reaction was stirred at room temperature for 2-4 h with nitrogen, and then filtered. The resin was washed with DMF and DCM successively and showed colorless or light yellow in the subsequent ninhydrin test. The resin peptide was washed three times with anhydrous ethanol, filtered and waited for cleavage.

### Step 1.8 Preparation of Intermediate Compound b

10000 mL cleavage reagent (TFA:TIS:H₂O=95:2.5:2.5) was added to the 10L reactor and cooled to −10±2° C. The dried and weighed resin was added. The reaction was warmed to room temperature and stirred under nitrogen for 2-3 h. After that, the resin was filtered and washed once with 100 mL of TFA. The filtrate and washing solution were combined.

40 L of pre-cooled (below −10° C.) cold ether was added into product solution. The mixture was stirred for 10 minutes, and then the precipitate was centrifuged. The supernatant was discarded after centrifugation, and the precipitate was collected and washed with cold ether, and the precipitate was centrifuged again (Each time the centrifugation speed was set to 3600 rpm, the centrifugation time was 5 minutes, and the temperature of the centrifuge cavity was −5° C.).

The precipitate was collected as crude Compound b. The crude product was purified by Prep-HPLC and lyophilized to obtain pure Compound b.

### Step 2: Preparation of Intermediate Compound a

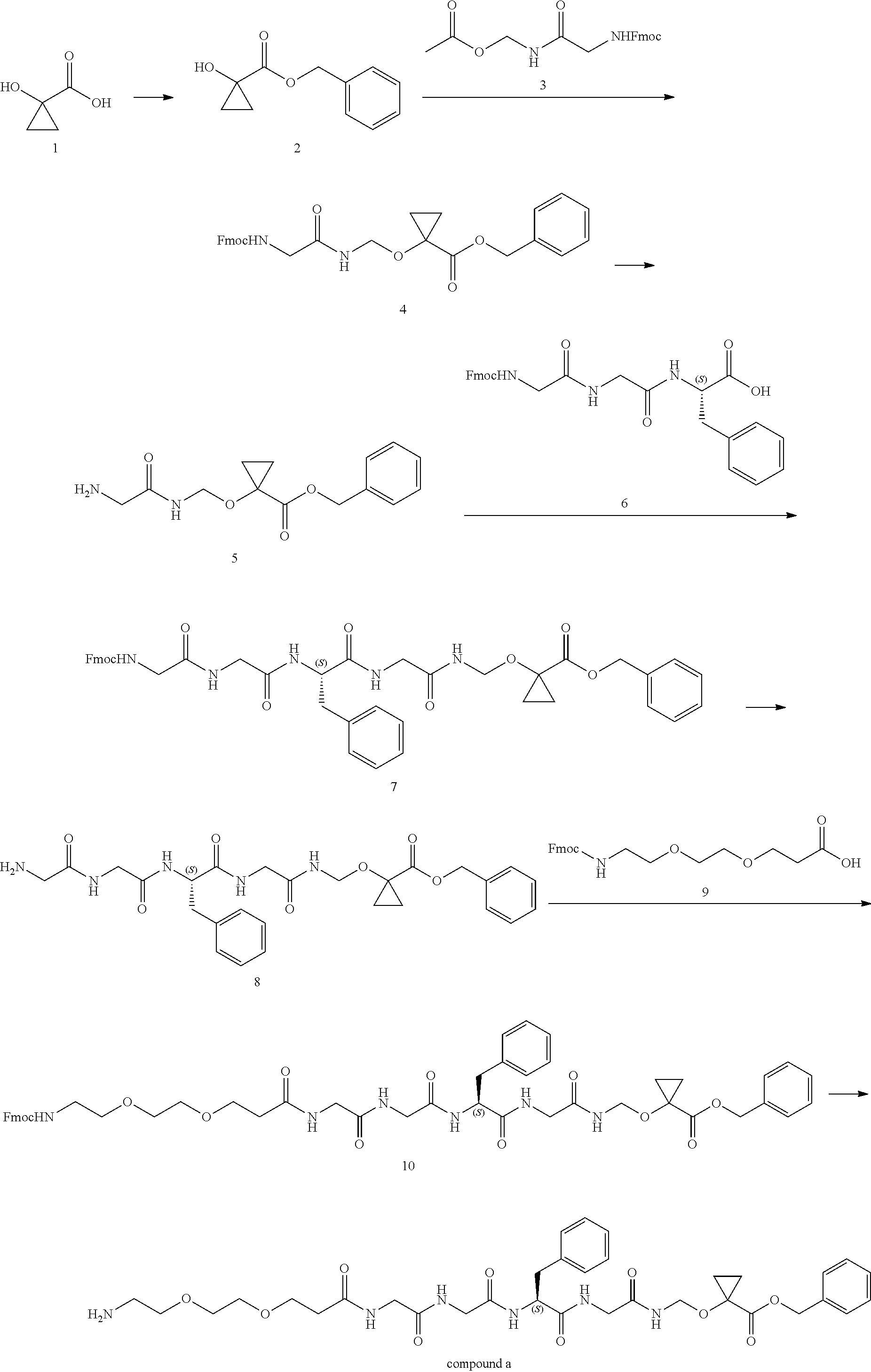

### Step 2.1 Preparation of Compound 2

Compound 1 (1 e.q.) and DMF (5 v/v) were added to the reaction flask, and the mixture was stirred and dissolved under nitrogen protection. After the ice bath was cooled to 0-5° C., DIEA (3 e.q.) was added dropwise, and the mixture was stirred at 5° C. for 10 min after the dropwise addition. Then benzyl bromide (1.3 e.q.) was added dropwise, and after the dropwise addition was completed, it was allowed to naturally rise to room temperature of about 20° C. and stirred for 16 hours.

The reaction solution was slowly poured into ice water, MTBE was added and stirred, and the solution was allowed to stand for separation. The aqueous phase was extracted 4 times with MTBE, the combined organic phases were washed with saturated brine, and then the organic phase was dried over anhydrous sodium sulfate, and the concentrated under vacuum to obtain a crude yellow oil, which was applied to the column by wet method. The light yellow oil was obtained by the elution of PE/EA=6:1, and the yield is 100%.

### Step 2.2 Preparation of Compound 4

Under nitrogen protection, intermediate 2 (2.0 e.q.), compound 3 (1 e.q.) and THF (10 v/v) were added to the reaction flask and stirred to dissolve, TsOH (0.1 e.q.) was weighed and added to the reaction, and the reaction was kept at 20-22° C. for 4 h. The reaction solution was slowly poured into ice water, extracted 3 times with EA, the combined organic phase was washed with saturated aqueous sodium bicarbonate solution, water and saturated brine successively, the organic phase was dried with anhydrous sodium sulfate, filtered and concentrated under vacuum to obtain the crude product. The product was collected by mixing silica gel sample through column by the elution of PE/EA=1:1, and concentrated to obtain a white solid with a yield of 40%.

### Step 2.3 Preparation of Compound 7

Under nitrogen protection, compound 4 and DMAc (10 v/v) were added to the reaction flask and stirred to dissolve. The reaction was cooled down to 14-18° C., DBU (0.5 e.q.) was added dropwise, and the reaction was stirred at this temperature for 1.5 h, the completion of reaction of the raw materials was monitored by TLC. The reaction was cooled down to 0-5′T, PPTS (0.5 e.q.), EDCI (1 e.q.), HOBT (1 e.q.) and compound 6 (0.85 eq) were added and reacted at 0-10° C. for 3-4 h, and the reaction was monitored by LCMS.

The reaction solution was added to ice water, 2-methyltetrahydrofuran was added to extract once, and the aqueous phase was extracted twice with 2-methyltetrahydrofuran. The organic phases were combined, washed with 0.5 M hydrochloric acid, washed with saturated aqueous NaHCO₃, water, and saturated brine, dried over anhydrous sodium sulfate, filtered, concentrated, evaporated to dryness, mixed with silica gel, and purified by column. The product was collected by the elution of DCM/MeOH and concentrated under vacuum to obtain a white solid with a yield of 78%.

### Step 2.4 Preparation of Compound 10

Under nitrogen protection, intermediate 7 and DMAc (10 v/v) were added to the reaction flask and stirred to dissolve. The reaction was cooled down to 14-18° C., DBU (0.5 e.q.) was added dropwise, and the reaction was stirred at this temperature for 1.5 h, the completion of reaction was monitored by TLC. The reaction was cooled down to 0-5° C., PPTS (0.5 e.q.), EDCI (1 e.q.), HOBT (1 e.q.) and compound 9 (0.85 e.q.) were added and reacted at 0-109° C. for 3-4 h, and the reaction was monitored by LCMS.

The reaction solution was added to ice water, 2-methyltetrahydrofuran was added to extract once, and the aqueous phase was extracted twice with 2-methyltetrahydrofuran. The organic phases were combined, washed with 0.5 M hydrochloric acid, saturated aqueous NaHCO₃, water, and saturated brine, dried over anhydrous sodium sulfate, filtered, concentrated, evaporated to dryness, mixed with silica gel, and purified by column. The product was collected by the elution of DCM/MeOH and concentrated under vacuum to obtain a white solid with a yield of 50%.

### Step 2.5 Preparation of Compound a

Under nitrogen protection, intermediate 10 was dissolved in DCM (15 v/v), DBU (0.5 e.q.) was added dropwise at 20° C., and the reaction was stirred at 18-22° C. for 5 h. The complete reaction was monitored by LCMS. The reaction solution was diluted with DCM and purified by the column by wet method, and the product was collected by the elution of DCM:MeOH to obtain a white solid with a yield of 82%.

### Step 3: Preparation of Compound c

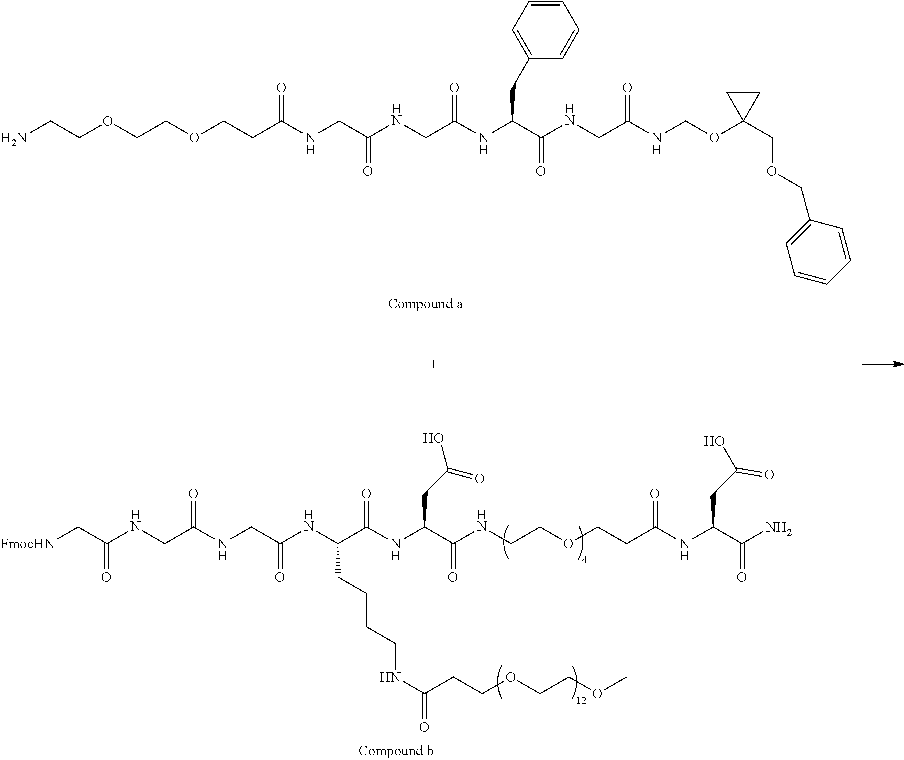

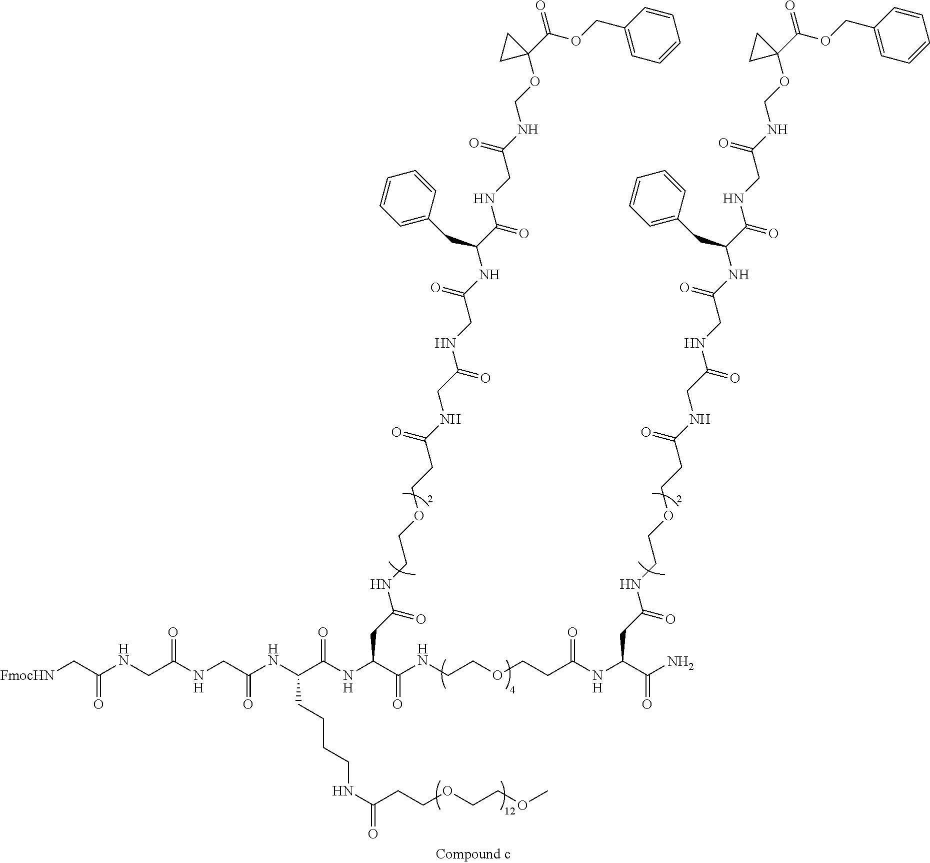

Compound b (400 mg, 0.245 mmol) and Compound a (377 mg, 0.539 mmol) were dissolved in DMF (6 ml), then DIPEA (159 mg, 1.23 mmol) and HATU (233 mg, 0.613 mmol) were added into the reaction solution, and the reaction was stirred at room temperature for 2h. After the disappearance of the Compound b, it was purified by prep-HPLC, and the preparation solution was lyophilized to obtain 380 mg of the product with a yield of 52%. Calcd for C₁₄₂H₂₀₇O₄₉N₂₁ [(M+3H)/3]⁺: 997.8, found: 875.9 (fragmented mass).

### Step 4: Preparation of Intermediate Compound d

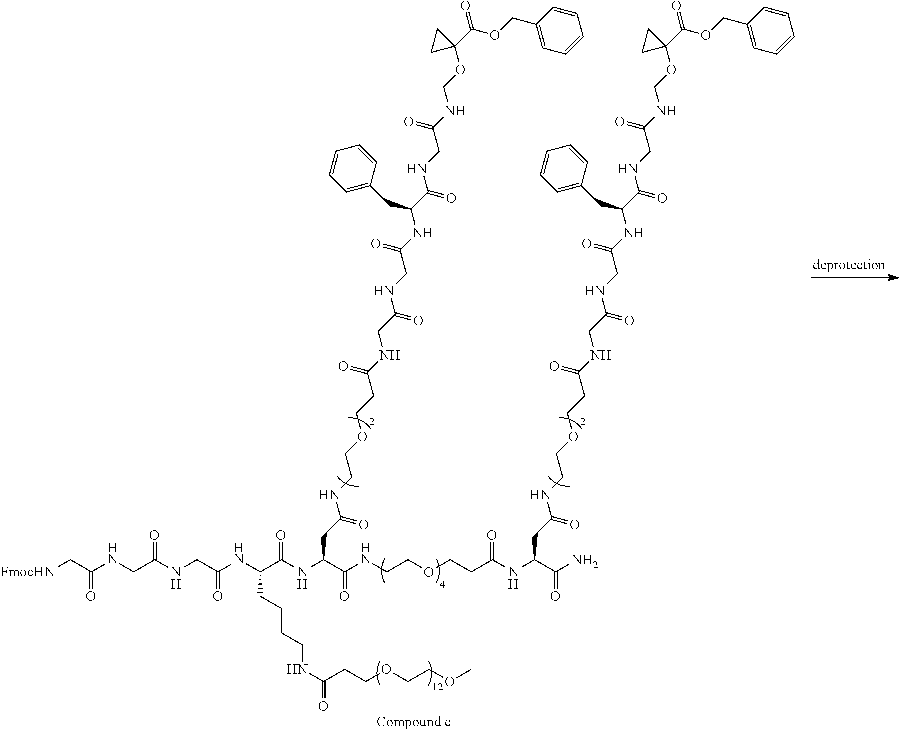

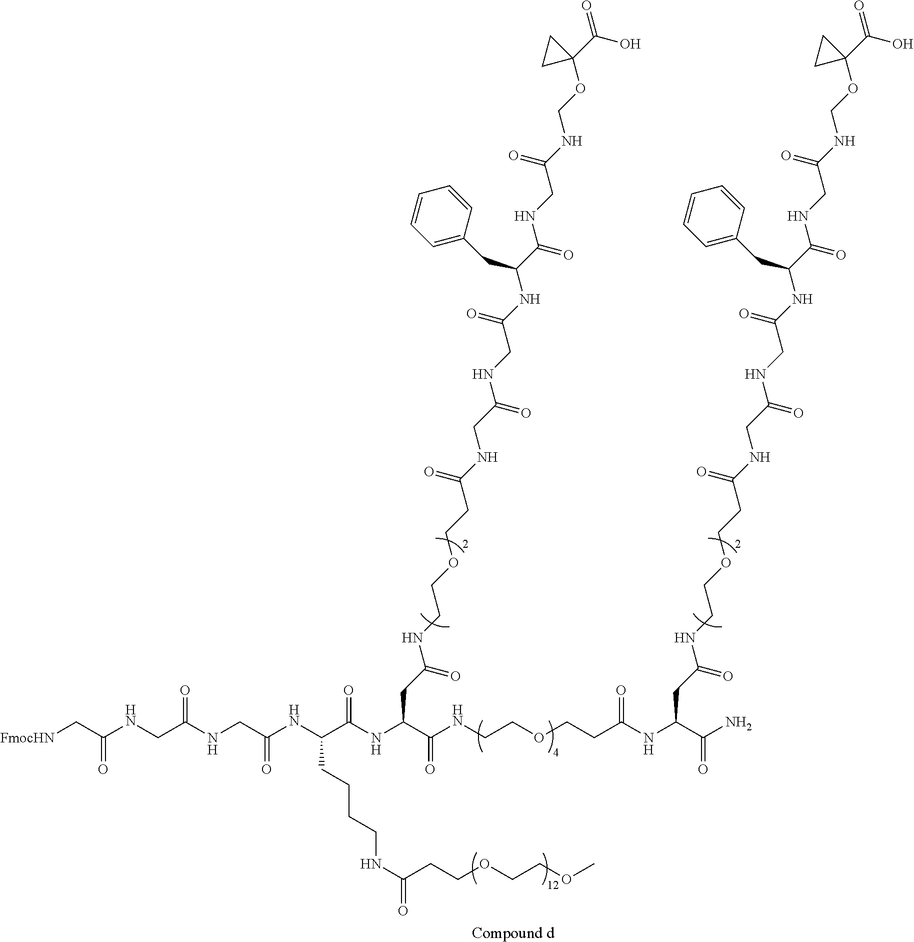

Compound c (380 mg, 0.245 mmol) was dissolved in purified water (80 ml), and palladium hydroxide (38 mg) was added. The system replaced with hydrogen for 3 times, the reaction was stirred at room temperature. The reaction progress was monitored by HPLC, the reaction was stopped immediately after the disappearance of the raw materials to prevent the increase of de-Fmoc products. The reaction solution was filtered and purified by prep-HPLC to obtain 270 mg of the product with a yield of 76%. Calcd for C₁₂₈H₁₉₅O₄₉N₂₁ [(M+3H)/3]⁺: 937.8, found: 875.9 (fragmented mass).

### Step 5: Preparation of Intermediate Compound e

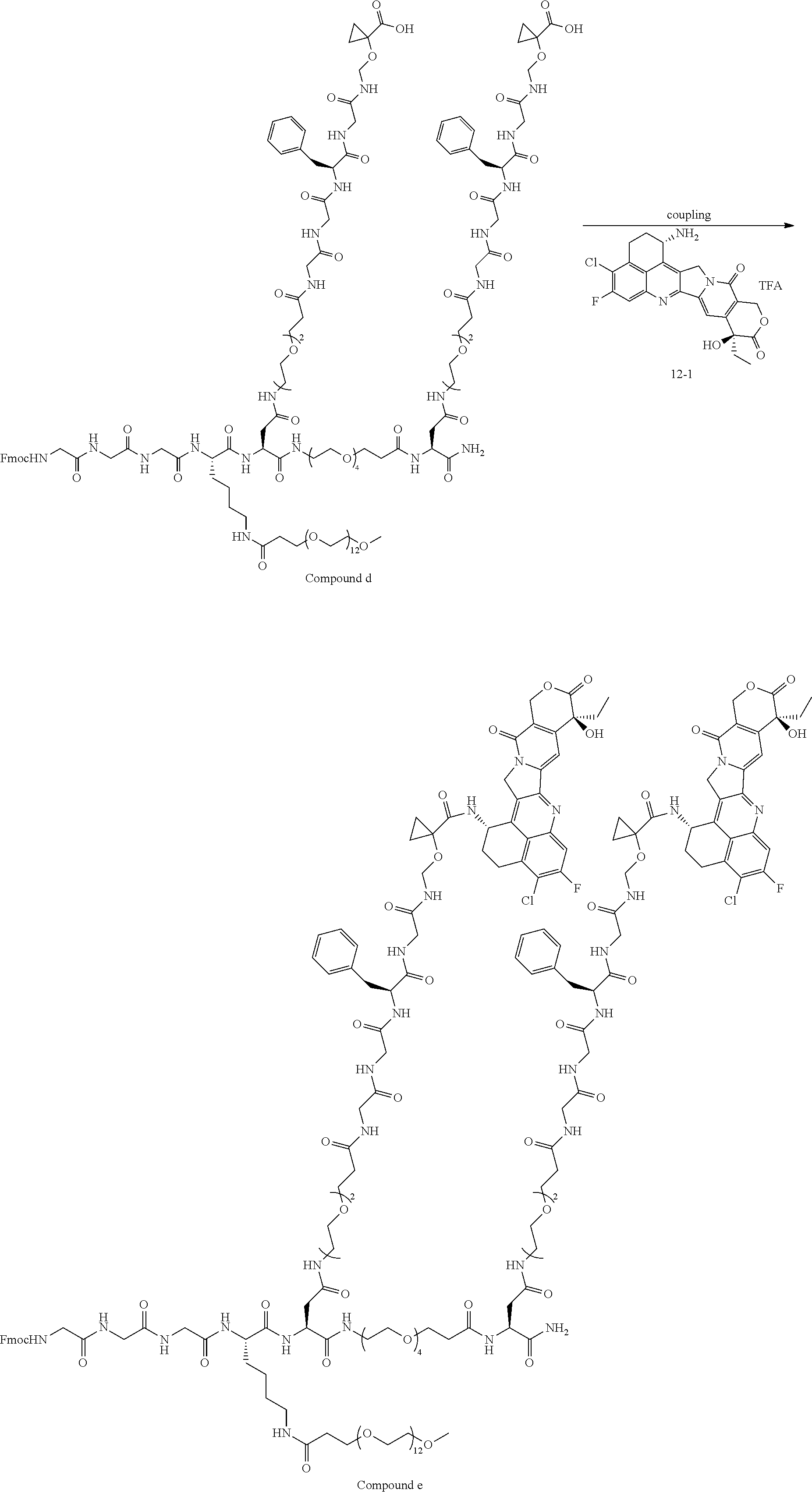

Compound d (270 mg, 0.096 mmol) and 12-1 (120 mg, 0.211 mmol) were dissolved in DMF (5 ml), then DIPEA (62 mg, 0.48 mmol) and HATU (92 mg, 0.24 mmol) were added into the reaction solution and stirred at room temperature for 2-16h. After the completion of reaction monitored by HPLC. The reaction mixture was directly purified by prep-HPLC, and the collected eluents were lyophilized to obtain 235 mg of the product with a yield of 66%. Calcd for C₁₇₄H₂₂₉O₅₅Cl₂F₂N₂₇ [(M+3H)/3]⁺: 1229.2, found: 1229.3.

Preparation of 12-1 is showed in 2.3.1 and 2.3.2.

### Step 6: Preparation of Linker-Payload 1

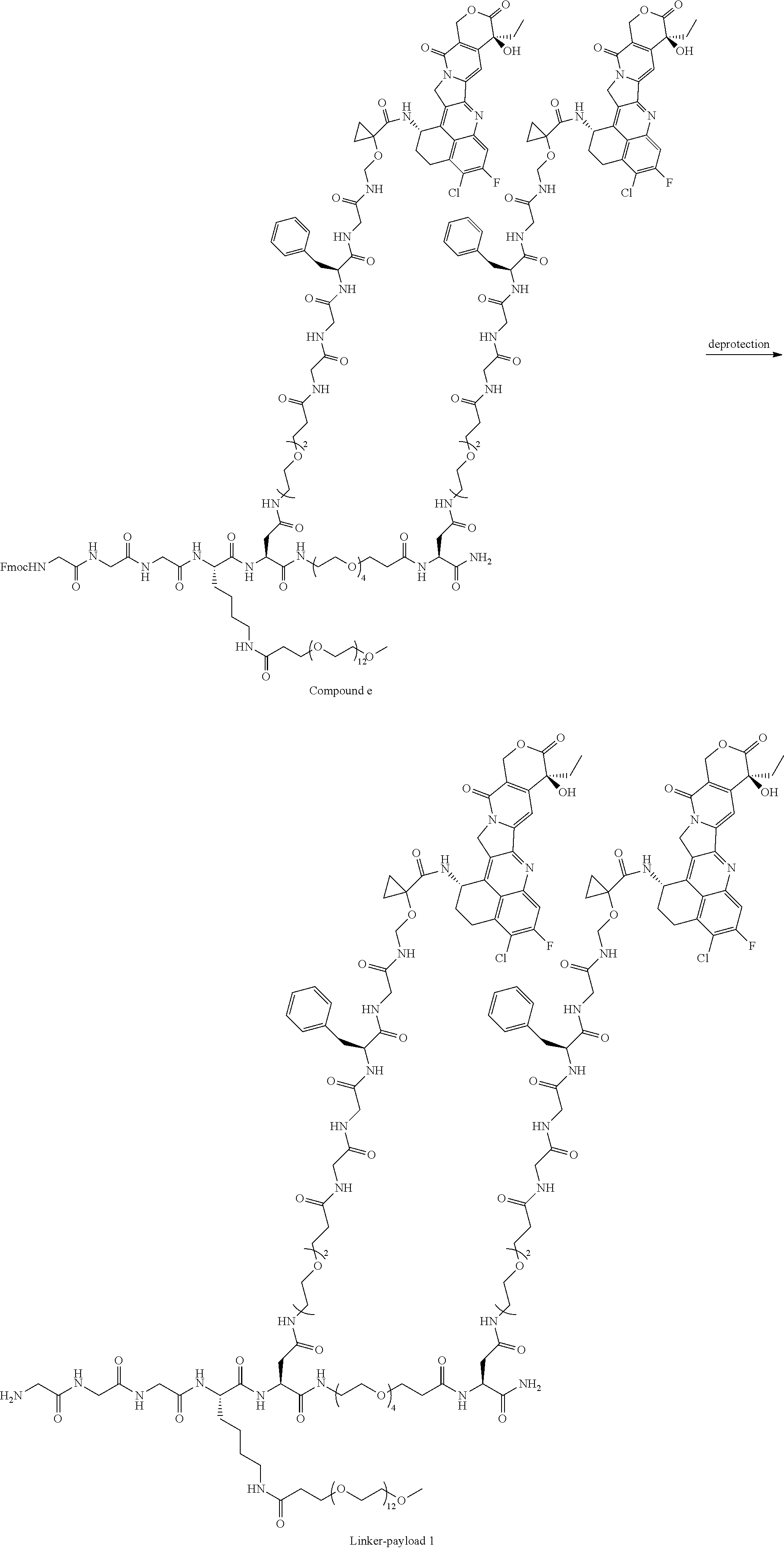

Compound e (210 mg, 0.057 mmol) was dissolved in DMF (5 ml), then diethylamine (0.5 ml) was added and the reaction was reacted at room temperature for 15 min, the reaction end point was monitored by HPLC. After the reaction was completed, it was adjusted to neutrality with 10% TFA aqueous solution under ice bath, and the reaction was purified by prep-HPLC, and 145 mg of product was obtained after lyophilization with a yield of 73%. Calcd for C₁₅₉H₂₁₉O₅₃Cl₂F₂N₂₇ [(M+3H)/3]⁺: 1155.2, found: 1155.3.

### 2.3 Preparation of Linker-Payload 2

### 2.3.1 Preparation of Payload Small Molecules

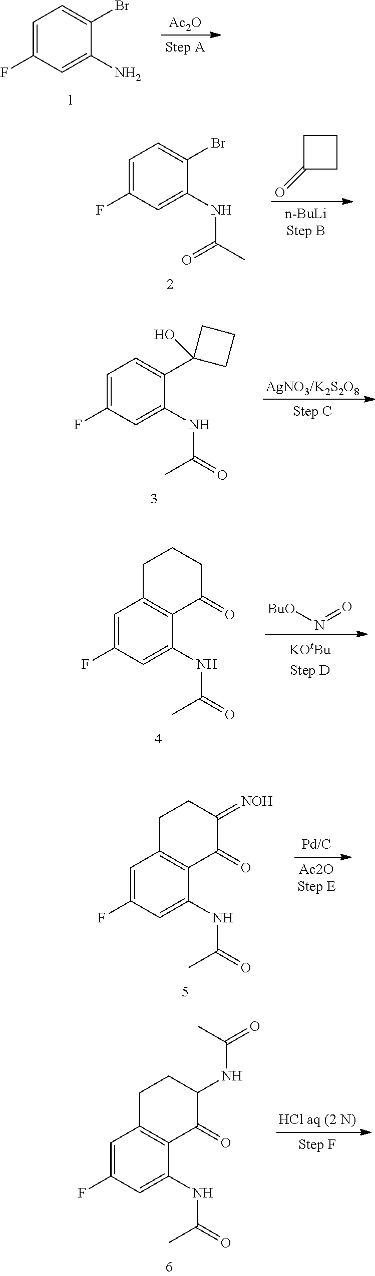

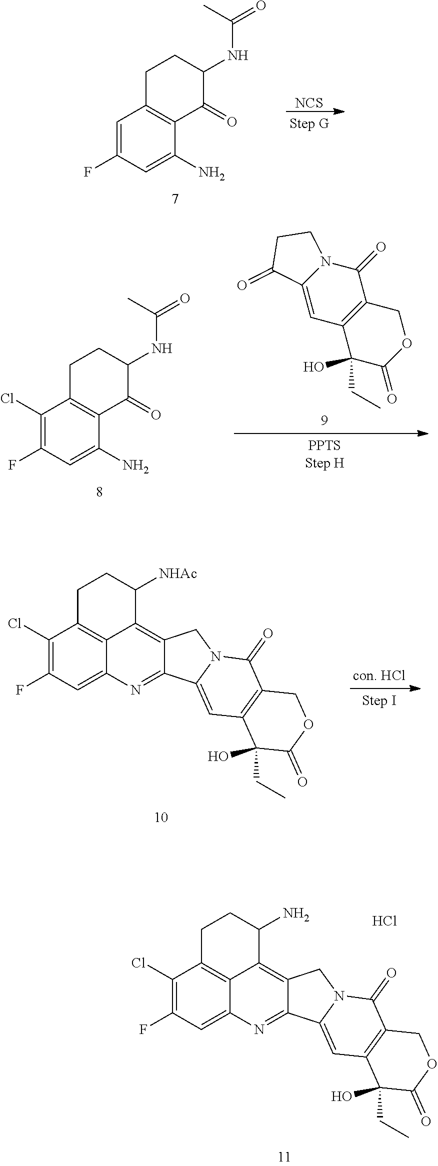

Step A: N-(2-bromo-5-fluorophenyl)acetamide: To a stirred solution of acetic anhydride (214 g, 2.10 mol) in acetic acid (500 mL) was added con. H₂SO₄ (3 mL), followed with 2-bromo-5-fluoroaniline (100 g, 526.27 mmol) in portions at room temperature. The mixture was stirred for 3 h, then poured into 2000 mL ice-water. A precipitate was formed, which was collected by filtration and dried in vacuo at room temperature to afford N-(2-bromo-5-fluorophenyl)acetamide (105 g) as a yellow solid. ¹H NMR (400 MHz, DMSO-d₆) δ 7.68 (dd, J=8.9, 6.0 Hz, 1H), 7.61 (ddd, J=10.7, 5.3, 3.1 Hz, 1H), 7.02 (ddd, J=8.9, 8.0, 3.1 Hz, 1H), 2.11 (s, 3H). MS m/z 232.0(M+H).

Step B: N-(5-fluoro-2-(1-hydroxycyclobutyl)phenyl)acetamide: To a stirred solution of N-(2-bromo-5-fluorophenyl)acetamide (105 g, 452.48 mmol) in THF (1000 mL) was added n-BuLi (594 mL, 1.6 M in n-hexane, 950.22 mmol) dropwise over 1 h at −78° C. After completion, the mixture was stirred for 0.5 h under N₂. Then a solution of cyclobutanone (38.06 g, 542.98 mmol) in THF (50 mL) was added dropwise at −78° C. over 0.5 h, the mixture was stirred at -78° C. to room temperature for 6 h. The mixture was poured into 500 mL saturated NH₄Cl aq at 0° C. Extracted with ethyl acetate (500 mL×3), washed with brine (250 mL×2), dried over Na₂SO₄ and concentrated. The mixture was triturated with (PE/EA=1:1, 100 mL) for 10 mins, filtered and the cake was collected and dried in vacuo to afford N-(5-fluoro-2-(1-hydroxycyclobutyl)phenyl)acetamide (24 g) as a yellow solid. MS m/z 206.1(M-18+H), 246.1(M+Na).

Step C: N-(3-fluoro-8-oxo-5,6,7,8-tetrahydronaphthalen-1-yl)acetamide: To a stirred mixture of N-(5-fluoro-2-(1-hydroxycyclobutyl)phenyl)acetamide (24 g, 107.50 mmol) in CH₂Cl₂(170 mL) and water (170 mL) was added silver nitrate (AgNO₃) (5.48 g, 32.25 mmol) and potassium persulfate (K₂S₂O₈) (58.12 g, 215.01 mmol), the mixture was stirred at 30° C. for 6 h. The mixture was filtered on Celite and washed with CH₂Cl₂ (100 mL), the filtrate was concentrated and purified by FCC(EA/PE=0-40%) to afford N-(3-fluoro-8-oxo-5,6,7,8-tetrahydronaphthalen-1-yl)acetamide (14 g) as a light yellow solid. MS m/z 222.1(M+H).

Step D: N-(3-fluoro-7-(hydroxyimino)-8-oxo-5,6,7,8-tetrahydronaphthalen-1-yl)acetamide: To a stirring mixture of N-(3-fluoro-8-oxo-5,6,7,8-tetrahydronaphthalen-1-yl)acetamide (14 g, 63.28 mmol) in THF (500 mL) at 0° C. was added 1-butyl nitrite (8.48 g, 63.28 mmol), followed with t-BuOK (8.52 g, 75.94 mmol). The mixture was stirred at 0° C. for 2 h. After completion, the mixture was acidified by HCl (2 N) to adjust pH=3. The mixture was extracted by ethyl acetate (200 mL×3), washed by brine (100 mL×2), dried over Na₂SO₄ and concentrated under reduced pressure. The crude mixture was triturated with tert-butyl methyl ether (200 mL) for 10 mins, filtered and the cake was collected and dried in vacuo to afford N-(3-fluoro-7-(hydroxyimino)-8-oxo-5,6,7,8-tetrahydronaphthalen-1-yl)acetamide (12 g) as a yellow solid. MS m/z 251.1(M+H).

Step E: N,N′-(3-fluoro-8-oxo-5,6,7,8-tetrahydronaphthalene-1,7-diyl)diacetamide: To a solution of N-(3-fluoro-7-(hydroxyimino)-8-oxo-5,6,7,8-tetrahydronaphthalen-1-yl)acetamide (12 g, 47.96 mmol) in acetic anhydride (90 mL) and THF (90 mL) was added 10% Pd/C (1 g), the mixture was stirred at 25° C. under H₂ atmosphere for 16 h. After cooling to 0° C., Et₃N (20 mL) was added dropwise, the mixture was stirred at 0° C. for 1 h. Filtered on Celite, the filtrate was poured into ice-water (500 mL). Extracted with ethyl acetate (500 mL×3), washed with brine (250 mL×2), dried over Na₂SO₄ and concentrated. The residue was triturated with tert-butyl methyl ether (120 mL) for 10 mins, filtered and the cake was collected and dried in vacuo to give N,N′-(3-fluoro-8-oxo-5,6,7,8-tetrahydronaphthalene-1,7-diyl)diacetamide (7.9 g) as a yellow solid. MS m/z 279.1(M+H).

Step F: N,N′-(3-fluoro-8-oxo-5,6,7,8-tetrahydronaphthalene-1,7-diyl)diacetamide: To a solution of N,N′-(3-fluoro-8-oxo-5,6,7,8-tetrahydronaphthalene-1,7-diyl)diacetamide (7.9 g, 28.39 mmol) in MeOH (150 mL) was added HCl aq (2 N, 150 mL), the mixture was stirred at 50° C. for 7 h. After cooling to 0° C., Sat. NaHCO₃ aq was added dropwise to adjust pH=8. Extracted with ethyl acetate (200 mL×3), washed with brine (200 mL×2), dried over Na₂SO₄ and concentrated under reduced pressure to give N,N′-(3-fluoro-8-oxo-5,6,7,8-tetrahydronaphthalene-1,7-diyl)diacetamide (6.0 g) as a yellow solid. ¹H NMR (400 MHz, Chloroform-d) δ 6.57 (s, 3H), 6.18 (td, J=11.1, 2.4 Hz, 2H), 4.52 (dt, J=13.3, 5.0 Hz, 1H), 3.13 (ddd, J=17.5, 13.0, 4.6 Hz, 1H), 3.00-2.81 (m, 1H), 2.69 (dtd, J=9.4, 4.6, 2.5 Hz, 1H), 2.09 (s, 3H), 1.79 (qd, J=13.0, 4.3 Hz, 1H). MS m/z 237.1(M+H).

Step G: N-(8-amino-5-chloro-6-fluoro-1-oxo-1,2,3,4-tetrahydronaphthalen-2-yl)acetamide: To a solution of N,N′-(3-fluoro-8-oxo-5,6,7,8-tetrahydronaphthalene-1,7-diyl)diacetamide (4.0 g, 16.93 mmol) in DMF (80 mL) was added NCS (2.26 g, 16.93 mmol) in portions at 0° C., the mixture was stirred at room temperature for 16 h. The mixture was poured into 200 mL ice-water. A precipitate was formed, which was collected by filtration and dried in vacuo at room temperature to afford N-(8-amino-5-chloro-6-fluoro-1-oxo-1,2,3,4-tetrahydronaphthalen-2-yl)acetamide (4.0 g) as a yellow solid. ¹H NMR (400 MHz, DMSO-d₆) δ 8.11 (d, J=8.0 Hz, 1H), 7.71 (s, 2H), 6.62 (d, J=11.9 Hz, 1H), 4.53 (ddd, J=13.0, 8.0, 4.7 Hz, 1H), 3.18-3.04 (m, 1H), 2.91 (ddd, J=17.5, 12.4, 4.8 Hz, 1H), 2.21-2.08 (m, 1H), 1.99-1.83 (m, 4H). MS m/z 271.0(M+H).

Step H: N-(9S)-4-chloro-9-ethyl-5-fluoro-9-hydroxy-10,13-dioxo-2,3,9,10,13,15-hexahydro-1H,12H-benzo[de]pyrano[3′,4′:6,7]indolizino[1,2-b]quinolin-1-yl)acetamide: To a mixture of N-(8-amino-5-chloro-6-fluoro-1-oxo-1,2,3,4-tetrahydronaphthalen-2-yl)acetamide (4.0 g, 14.78 mmol) in toluene (400 mL) was added (S)-4-ethyl-4-hydroxy-7,8-dihydro-1H-pyrano[3,4-f]indolizine-3,6,10(4H)-trione (4.28 g, 16.25 mmol), pyridinium p-Toluenesulfonate (1.11 g, 4.43 mmol) and o-cresol (10 mL), the mixture was heated to reflux under N₂ for 24 h. The solvent was removed by reduced pressure and the mixture was purified by FCC(THF/CH₂Cl₂=0-60%) to afford N-(9S)-4-chloro-9-ethyl-5-fluoro-9-hydroxy-10,13-dioxo-2,3,9,10,13,15-hexahydro-1H,12H-benzo[de]pyrano[3′,4′:6,7]indolizino[1,2-b]quinolin-1-yl)acetamide (4.1 g) as a brown solid. MS m/z 498.1(M+H).

Step I: (9S)-1-amino-4-chloro-9-ethyl-5-fluoro-9-hydroxy-1,2,3,9,12,15-hexahydro-10H,13H-benzo[de]pyrano[3′,4′:6,7]indolizino[1,2-b]quinoline-10,13-dione: A mixture of N-(9S)-4-chloro-9-ethyl-5-fluoro-9-hydroxy-10,13-dioxo-2,3,9,10,13,15-hexahydro-1H,12H-benzo[de]pyrano[3′,4′:6,7]indolizino[1,2-b]quinolin-1-yl)acetamide (2.0 g, 4.02 mmol) in 20 mL con. HCl aq was stirred at 70° C. under N₂ for 36 h. The mixture was concentrated under reduced pressure to give crude (9S)-1-amino-4-chloro-9-ethyl-5-fluoro-9-hydroxy-1,2,3,9,12,15-hexahydro-10H,13H-benzo[de]pyrano[3′,4′:6,7]indolizino[1,2-b]quinoline-10,13-dione hydrochloride (2 g) as a brown solid. MS (ESI) m/z 456.1 (M+H).

### 2.3.2 Preparation of Intermediate 12 (12-1, 12-2)

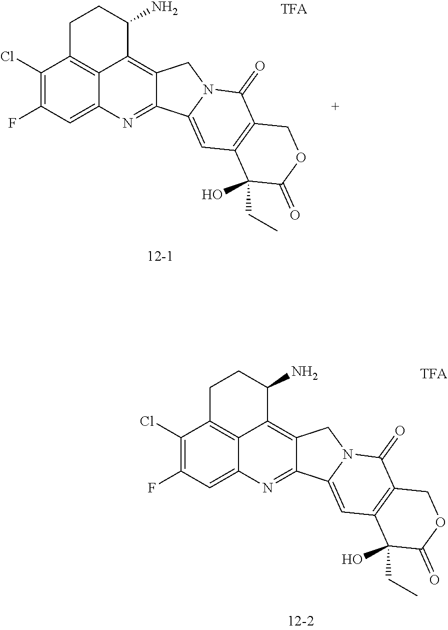 [SMILES: CC[C@@]1(O)C(=O)OCc2c1cc1n(c2=O)Cc2c-1nc1cc(F)c(Cl)c3c1c2[C@@H](N)CC3 ; CC[C@@]1(O)C(=O)OCc2c1cc1n(c2=O)Cc2c-1nc1cc(F)c(Cl)c3c1c2[C@H](N)CC3]

12-1 and 12-2 were prepared by prep-HPLC from (9S)-1-amino-4-chloro-9-ethyl-5-fluoro-9-hydroxy-1,2,3,9,12,15-hexahydro-10H,13H-benzo[de]pyrano[3′,4′:6,7]indolizino[1,2-b]quinoline-10,13-dione hydrochloride (intermediate 11) as TFA salt.

TABLE 2

| | | | | Retention |
|---|---|---|---|---|
| | | | | time on |
| | | | MS | HPLC |
| Number | Structure | ¹HNMR | (M + H) | (min) |
| 12-1 | 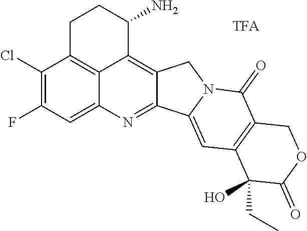 [SMILES: CC[C@@]1(O)C(=O)OCc2c1cc1n(c2=O)Cc2c-1nc1cc(F)c(Cl)c3c1c2[C@@H](N)CC3] | ¹H NMR (400 MHz, DMSO-d6) δ8.51 (d, J = 4.8 Hz, 3H), 8.17 (d, J = 10.2 Hz, 1H), 7.38 (s, 1H), 6.56 (s, 1H), 5.74 (d, J = 19.4 Hz, 1H), 5.52 - 5.40 (m, 3H), 5.16 (s, 1H), 3.44 (dd, J = 16.3, 4.1 Hz, 1H), 3.19 (t, J = 13.9 Hz, 1H), 2.57 (d, J = 14.0 Hz, 1H), 2.26 (t, J = 14.3 Hz, 1H), 1.89 (hept, J = 7.0 Hz, 2H), 0.89 (t, J = 7.3 Hz, 3H). | 456.0 | 1.395 |
| 12-2 | 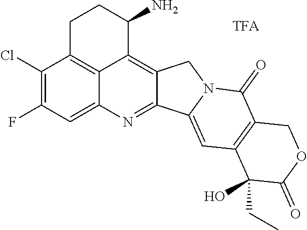 [SMILES: CC[C@@]1(O)C(=O)OCc2c1cc1n(c2=O)Cc2c-1nc1cc(F)c(Cl)c3c1c2[C@H](N)CC3] | ¹H NMR (400 MHz, DMSO-d6) δ 8.56 (s, 2H), 8.17 (d, J = 10.1 Hz, 1H), 7.38 (s, 1H), 6.57 (s, 1H), 5.74 (d, J = 19.4 Hz, 1H), 5.53 - 5.28 (m, 3H), 5.16 (s, 1H), 3.59 - 3.01 (m, 2H), 2.35 − 2.19 (m, 2H), 2.00 − 1.78 (m, 2H), 0.88 (t, J = 7.3 Hz, 3H). | 456.0 | 1.541 |

Conditions of HPLC above: Equipment: Agilent 1200;
Chromatographic column: Waters XBridge C18 4.6*50 mm,3.5 um;
Flow: 2.0 mL/min;
Gradient elute: 5.0%-95.0%-95.0%-5.0%-5.0%, 0.00 min-1.50 min-2.50 min-2.52 min-3.00 min;
Temperature: 40° C.;
Phase: A: Acetonitrile, B: H₂O (0.05% TFA);
Wavelength: 214 nm/254 nm.

### 2.3.3 Preparation of Linker-Payload 2

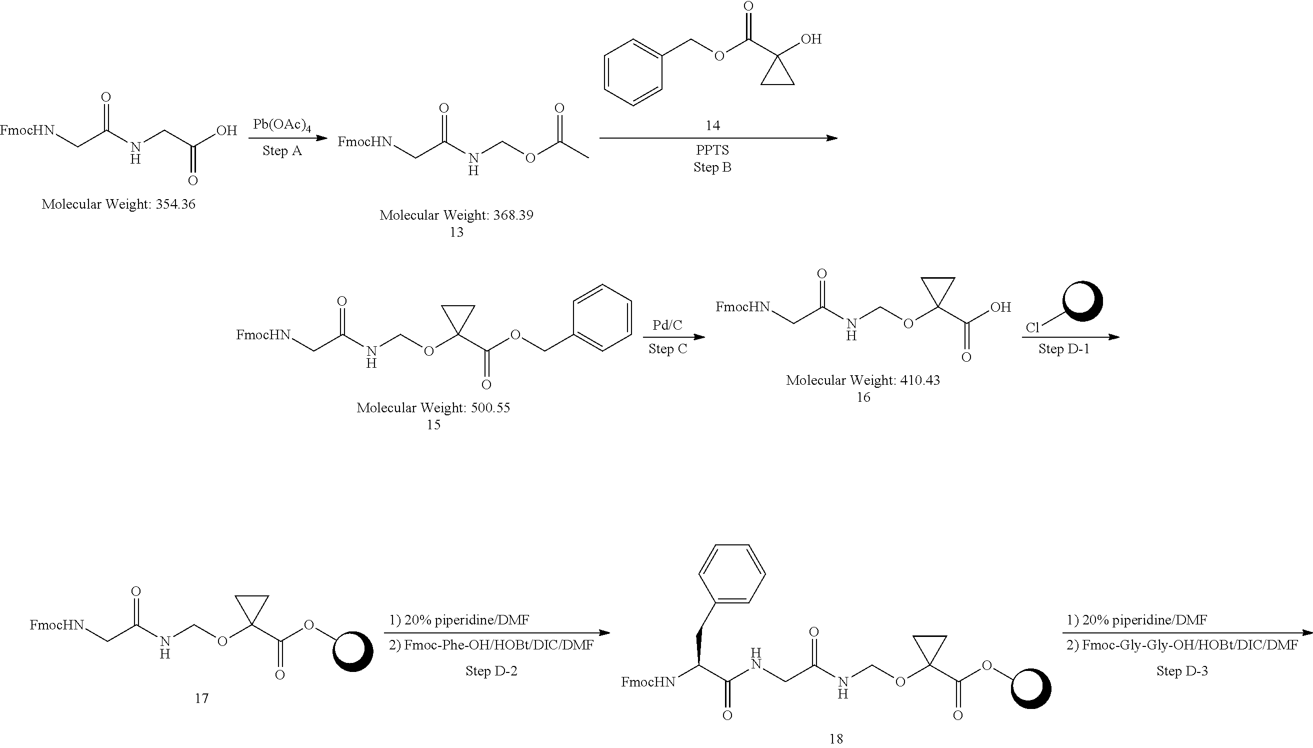

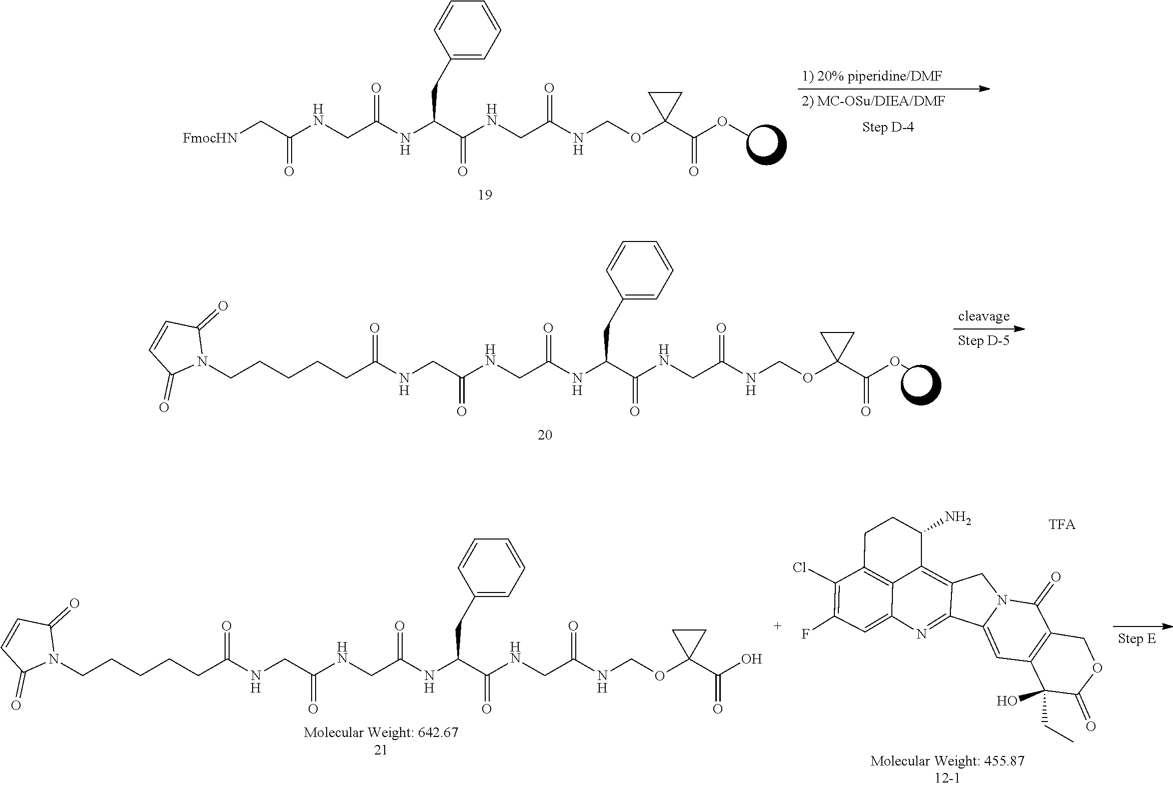

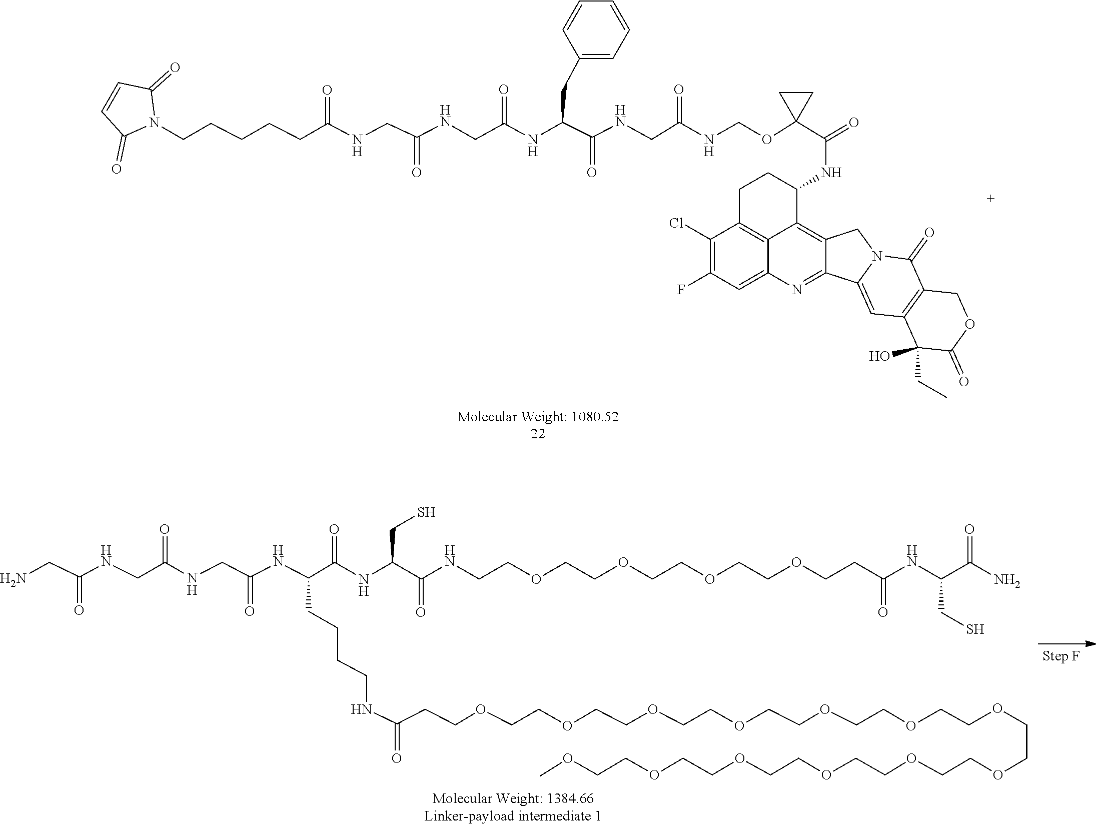 [SMILES: CC[C@@]1(O)C(=O)OCc2c1cc1n(c2=O)Cc2c-1nc1cc(F)c(Cl)c3c1c2[C@@H](NC(=O)C1(OCNC(=O)CNC(=O)[C@H](Cc2ccccc2)NC(=O)CNC(=O)CNC(=O)CCCCCN2C(=O)C=CC2=O)CC1)CC3 ; COCCOCCOCCOCCOCCOCCOCCOCCOCCOCCOCCOCCOCCC(=O)NCCCC[C@H](NC(=O)CNC(=O)CNC(=O)CN)C(=O)N[C@@H](CS)C(=O)NCCOCCOCCOCCOCCC(=O)N[C@@H](CS)C(N)=O]

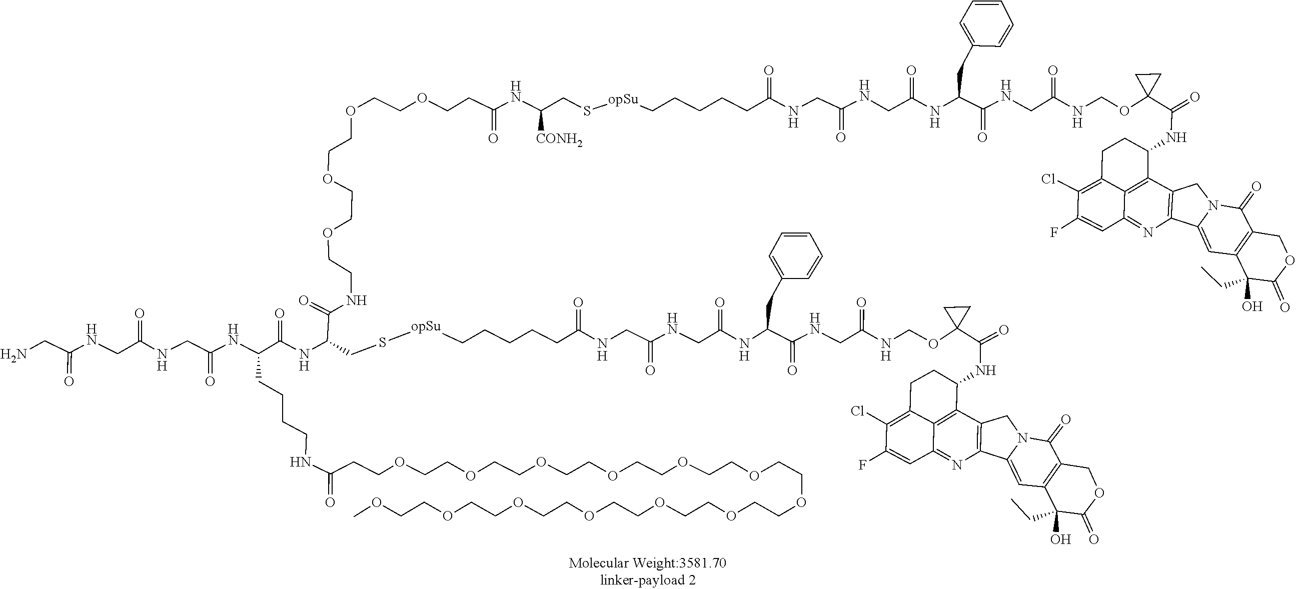

opSu is a mixture of

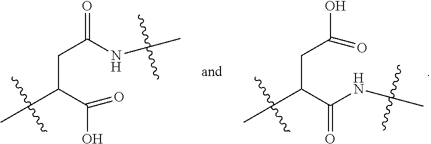 [SMILES: *NC(=O)CC(*)C(=O)O ; *NC(=O)C(*)CC(=O)O]

### 2.3.3.1 Preparation of Compound 13 (Step A)

4.33 g Fmoc-Gly-Gly-OH and 6.84 g Pb(OAc)₄ were weighed and added into a 500 ml single-neck round bottom flask. Anhydrous THF/Toluene (120/40 ml) was added under nitrogen atmosphere and stirred for dissolving. Then 1.16 mL of pyridine was added to the reaction system. The reaction system was heated to 80° C. and refluxed for 5 hr under nitrogen atmosphere. Samples were taken and detected by HPLC to monitor the reaction.

The reaction system was cooled to room temperature, filtered, and the filter cake was washed with EA for 3 times. The filtrates were combined and concentrated to dryness. Column chromatography was performed (PE:EA=100:0-50:100) to give about 2000 mg of the target product in white solid with a yield of 44%.

### 2.3.3.2 Preparation of Compound 15 (Step B)

200 mg Compound 13 was weighed and added into a 100 ml single-neck round bottom flask. Then 15 ml THF was added and stirred for dissolving. Then Compound 14 (312 mg, 3.0 e.q.) and TsOH H₂O (15 mg, 0.15 e.q.) were added to the reaction system. The reaction system was reacted overnight at room temperature. Samples were taken and detected by TLC (PE/EA=1:1) to monitor the reaction. The raw material basically disappeared, and a new point was detected.

Saturated sodium bicarbonate solution was added to quench reaction. Extraction was conducted with EA for 3 times. The organic phase was combined and washed with saline, dried with anhydrous magnesium sulfate and concentrated. The crude product was purified by column chromatography (PE:EA=5:1-1:1) to give about 80 mg of the target product in colorless oil with a yield of 29%. MS: [M+H]⁺=501.1.

### 2.3.3.3 Preparation of Compound 16 (Step C)

200 mg of Compound 15 was weighed and added into a 100 ml single-neck round bottom flask. Then 10 ml of EtOH and 5 ml of EA were added with complete dissolution. Then 40 mg of palladium carbon was added to the reaction system under nitrogen atmosphere, and the reaction system was purged with hydrogen gas for three times. The reaction system was kept under hydrogen atmosphere and stirred for 0.5 hour at room temperature. Samples were taken and detected by TLC (DCM/MeOH=10:1) to monitor the reaction. The raw material basically disappeared, and a new point was detected.

The reaction system was filtered, and the filter cake was washed with EA for 3 times. The filtrates were combined and concentrated to dryness to give 200 mg product in white solid with 100% yield. The product can be directly used in the next reaction without purification. MS: [M−H]⁻=409.4.

### 2.3.3.4 Preparation of Compound 21 (Step D)

### Step D-1

2.0 g of dichlororesin was weighed and placed in a polypeptide synthesis tube. DCM (10 ml) was added and swelled at room temperature for 30 minutes. The solvent was removed by vacuum suction. The resin was washed twice with DCM, with a volume of 7 mL and a time length of 1 minute for each wash. The solvent was removed by vacuum suction. Then Compound 16 (200 mg) was weighed and added into a 50 ml centrifuge tube. DCM (about 10 ml) was added. the solid was dissolved by shaking. Added to the above resin. Stirring was conducted to soak all the resin in the solution (if there was resin attached to the tube wall, a small amount of DCM was used to wash the tube wall). Stirring was conducted for 4-5 hours. After the reaction was complete, an appropriate amount of methanol was added. Stirring was conducted for 30 min. The solvent was removed by vacuum suction. The resin was washed with DMF once, methanol once, DMF once, methanol once and DMF twice in sequence, with a volume of 10 mL and a time length of 1 minute for each wash. The solvent was removed by vacuum suction. A small amount of dry resin was taken for ninhydrin detection. The resin was colorless and transparent, and the solution was yellowish, indicating qualified for the next coupling step.

### Step D-2

The deprotection was conducted twice by adding 10 mL readymade 20% piperidine/DMF solution and reacting for 10 minutes for each time. After the reaction was complete, the solution was removed by vacuum suction. The resin was washed with DMF twice, methanol once, DMF once, methanol once and DMF twice in sequence, with a volume of 10 mL and a time length of 1 minute for each wash. The solvent was removed by vacuum suction. A small amount of dry resin was taken for ninhydrin detection. Both the resin and solution were dark blue.

To a 50 mL centrifuge tube was added 563 mg Fmoc-Phe-OH, 197 mg HOBt. Then about 7 mL DMF was added. The solid was dissolved by shaking. Then 0.24 mL DIC was added. Activated for 10-30 minutes to give the activated reaction solution.

3 molar equivalent of activated reaction solution added to the resin. Stirring was conducted to soak the resin completely in the solution (if there was resin attached to the tube wall, a small amount of DCM was used to wash the tube wall). Stirring was conducted for 2-3 hours. After the reaction was complete, the solvent was removed by vacuum suction. The resin was washed with DMF twice, methanol once, DMF once, methanol once and DMF twice in sequence, with a volume of 10 mL and a time length of 1 minute for each wash. The solvent was removed by vacuum suction. A small amount of dry resin was taken for ninhydrin detection. The resin was colorless and transparent, and the solution was yellowish, indicating qualified for the next coupling step.

### Step D-3

The deprotection was conducted twice by adding 10 mL readymade 20% piperidine/DMF solution and reacting for 10 minutes for each time. After the reaction was complete, the solution was removed by vacuum suction. The resin was washed with DMF twice, methanol once, DMF once, methanol once and DMF twice in sequence, with a volume of 10 mL and a time length of 1 minute for each wash. The solvent was removed by vacuum suction. A small amount of dry resin was taken for ninhydrin detection. Both the resin and solution were dark blue.

To a 50 mL centrifuge tube was added 531 mg Fmoc-GG-OH, 197 mg HOBt. Then about 10 mL DMF was added. The solid was dissolved by shaking. Then 0.24 mL DIC was added. Activated for 10-30 minutes to give the activated reaction solution.

3 molar equivalent of activated reaction solution was added to the resin. Stirring was conducted to soak the resin completely in the solution (if there was resin attached to the tube wall, a small amount of DCM was used to wash the tube wall). Stirring was conducted for 2-3 hours. After the reaction was complete, the reaction solution was removed by vacuum suction. The resin was washed with DMF twice, methanol once, DMF once, methanol once and DMF twice in sequence, with a volume of 10 mL and a time length of 1 minute for each wash. The solvent was removed by vacuum suction. A small amount of dry resin was taken for ninhydrin detection. The resin was colorless and transparent, and the solution was yellowish, indicating qualified for the next coupling step.

### Step D-4

The deprotection was conducted twice by adding 10 mL readymade 20% piperidine/DMF solution and reacting for 10 minutes for each time. After the reaction was complete, the solution was removed by vacuum suction. The resin was washed with DMF twice, methanol once, DMF once, methanol once and DMF twice in sequence, with a volume of 10 mL and a time length of 1 minute for each wash. The solvent was removed by vacuum suction. A small amount of dry resin was taken for ninhydrin detection. Both the resin and solution were dark blue. Then, 462 mg MC-OSu was placed in a 50 mL centrifuge tube, about 10 mL DMF was added. The solid was dissolved by shaking. Then 0.24 mL DIEA was added to the resin. Stirring was conducted to soak the resin completely in the solution (if there was resin attached to the tube wall, a small amount of DCM was used to wash the tube wall). Stirring was conducted for 2-3 hours. After the reaction was complete, the reaction solution was removed by vacuum suction. The resin was washed with DMF twice, methanol once, DMF once, methanol once and DMF twice in sequence, with a volume of 10 mL and a time length of 1 minute for each wash. The solvent was removed by vacuum suction. A small amount of dry resin was taken for ninhydrin detection. The resin was colorless and transparent, and the solution was yellowish, indicating qualified for the next coupling step.

### Step D-5

The resin was washed twice with 10 mL of methanol. Then the solvent was removed thoroughly by vacuum suction. The resin was poured out and weighed. The lysis buffer was prepared in a 250 mL conical flask, wherein: the ratio of TFE/DCM was 80%/20%, and the volume was 7-8 times of the weight of peptide resin. The lysis buffer was added into the peptide resin, shaken well. The resin was fully soaked in the lysis buffer, and lysis was carried out at room temperature for 2-3 hours. The lysis buffer was then filtered out using a simple filter made of a syringe, and the resin was washed with 1-2 ml DCM and discarded. Then 150 mL precooled anhydrous ether was added to the lysis buffer, shaken well and then stood for 20-30 minutes. Using a 50 mL centrifuge tube, the above system was centrifuged in a centrifuge at 3500 rpm for 3 minutes, and the supernatant was poured out and discarded. The solid was shaken with precooled anhydrous ether, washed once under ultrasound, centrifuged at 3500 rpm for 3 minutes, and the supernatant was poured out and discarded. The solid was placed in a centrifuge tube and allowed to air dry overnight, and then subjected to preparative purification to give 125 mg of product in white solid with a yield of 40%. MS: [M−H]⁻=641.5.

### 2.3.3.5 Preparation of Compound 22 (Step E)

150 mg of raw material Compound 21 and 55 mg of TSTU were weighed and added into a 10 mL single-neck round bottom flask, and anhydrous DMF (3 mL) was added under nitrogen atmosphere and stirred for 20 min. Then 18 mg Compound 12-1 and 20 μl DIEA were added in sequence to the reaction system. Stirring was conducted at room temperature for 2 hours under nitrogen atmosphere. Samples were taken and detected by HPLC to monitor the reaction. The raw material peak completely disappeared, and new peaks were detected.

The reaction system was subjected to preparative purification, and the target product was collected and lyophilized to give about 22 mg of product in yellowish solid. MS: [M+H]⁺=1081.0.

### 2.3.3.6 Preparation of Linker-Payload 2 (Step F)

Compound 22 (30 mg) was weighed and added into a 10 ml single-neck round bottom flask, purified water (2 ml) was added. Stirring was conducted for dissolving. DMF solution (2 ml) containing Linker-payload intermediate 1 (19.5 mg) was added to the reaction system and stirred. After reacting overnight, HPLC was used to monitor the reaction until all of the raw material had converted into intermediates. The reaction mixture was directly added with an appropriate amount of Tris Base solution or other solution that promotes the ring-opening reaction, and the reaction was performed at 0-40° C. for another 0.2-20 h. The reaction was monitored by HPLC until all the intermediates were consumed and then quenched by acetic acid solution.

The reaction system was subjected to preparative purification, and the target product was collected and lyophilized to give about 25 mg of linker-payload 2 with yellowish solid. MS: [(M+3H)/3]⁺=1194.4.

### 2.4 Preparation of Linker-Payload 3

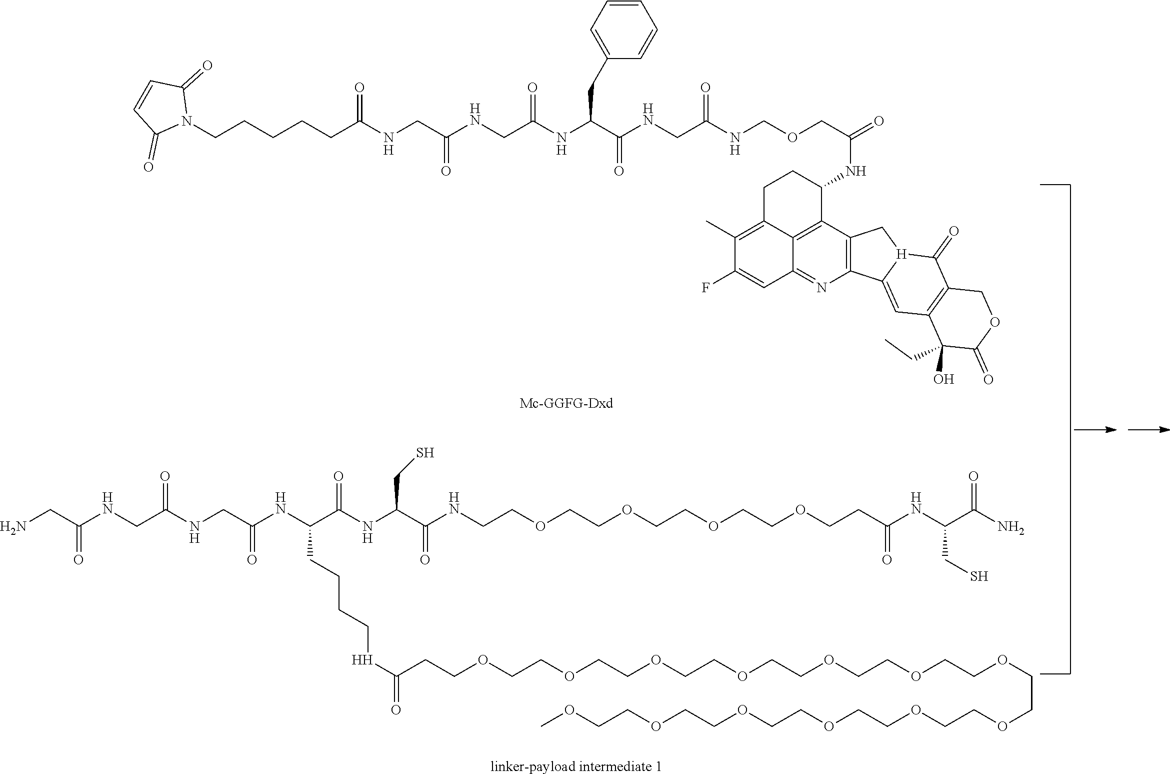

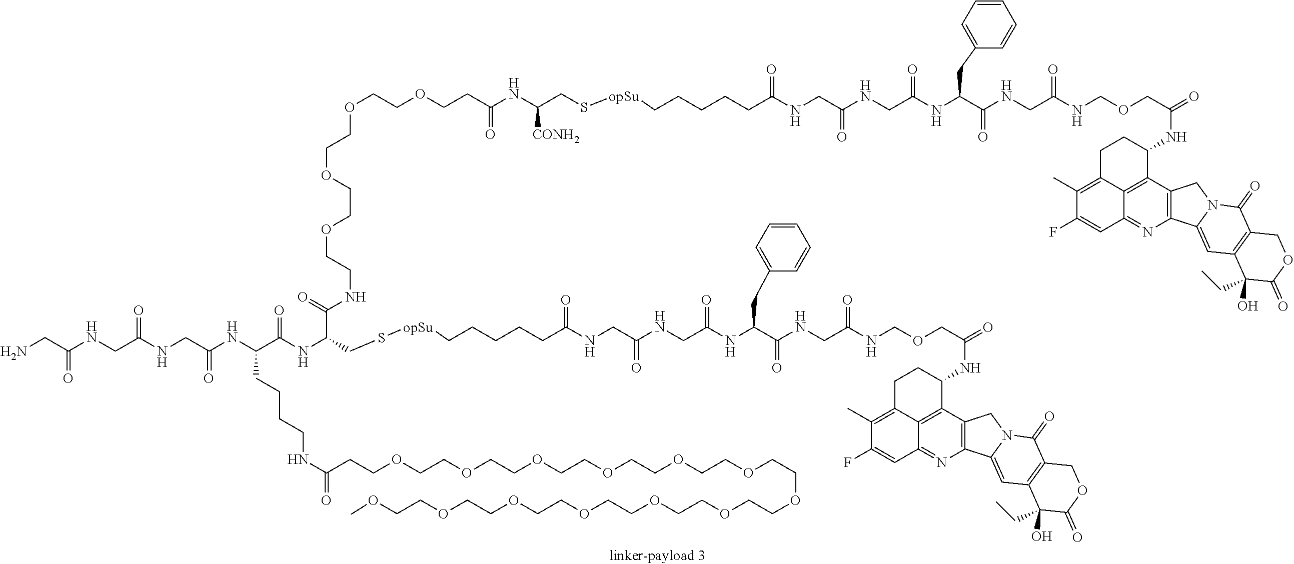

opSu is a mixture of

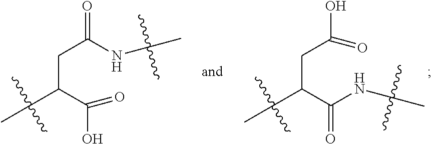 [SMILES: *NC(=O)CC(*)C(=O)O ; *NC(=O)C(*)CC(=O)O]

### Step 2.4.1: Preparation of Linker-Payload Intermediate 1

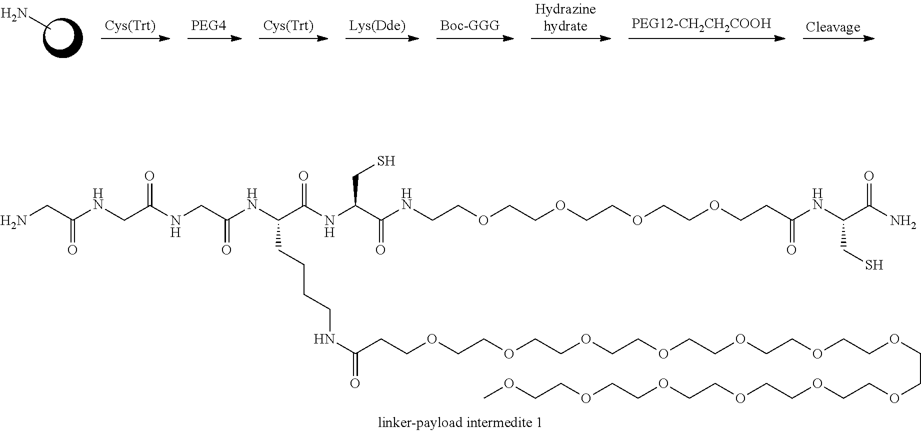

Linker-payload intermediate 1 is synthesized by a conventional solid phase polypeptide synthesis using Rink-amide-MBHA-resin. Fmoc was used to protect the amino acid in the linking unit. The conjugation reagent was selected from HOBT, HOAt/DIC, DCC, EDCI or HATU. After synthesis, the resin was cleaved using trifluoroacetic acid. The product was purified by HPLC, lyophilized and stored for use. Theoretical Mass: 1383.70, measured: [M−H]⁻=1382.6.

### Step 2.4.2: Preparation of Linker-Payload 3

Linker-payload intermediate 1 and intermediate MC-GGFG-Dxd (commercial purchased) with molar ratio -1:2 were weighed and dissolved in water and DMF, respectively, and then thoroughly mixed to give a mixture, which was reacted at 0-40° C. for 0.5-30 h. Once the reaction was completed, the reaction mixture was directly added with an appropriate amount of Tris Base solution or other solution that promotes the ring-opening reaction, and the reaction was performed at 0-40° C. for another 0.2-20 h. After the reaction was completed, the product was purified by semi-preparative/preparative HPLC and lyophilized to obtain linker-payload 3. Theoretical Mass: 3486.52, measured: [(M+3H)/3]⁺=1163.3.

### 2.5 Preparation of Linker-Payload 4

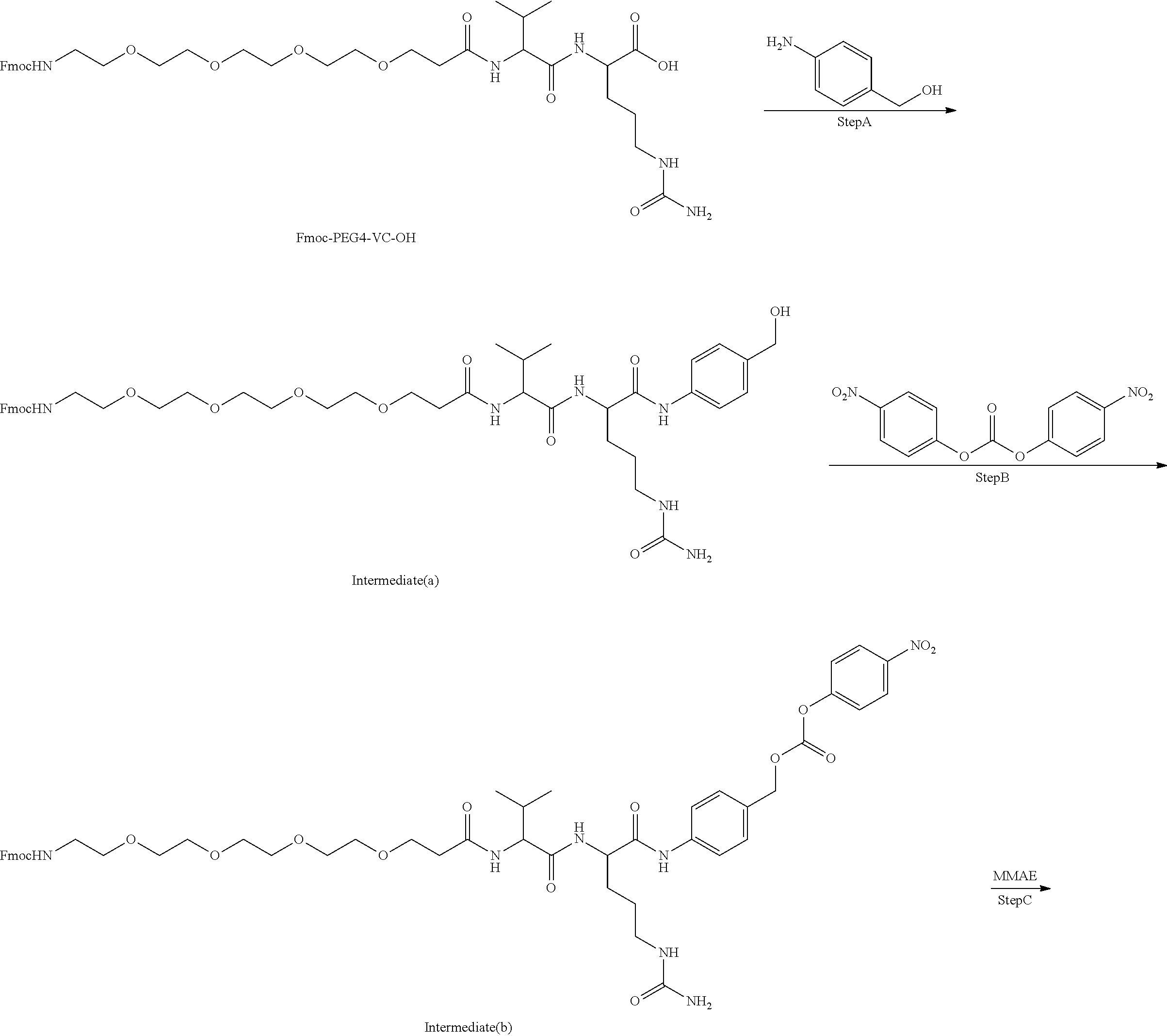

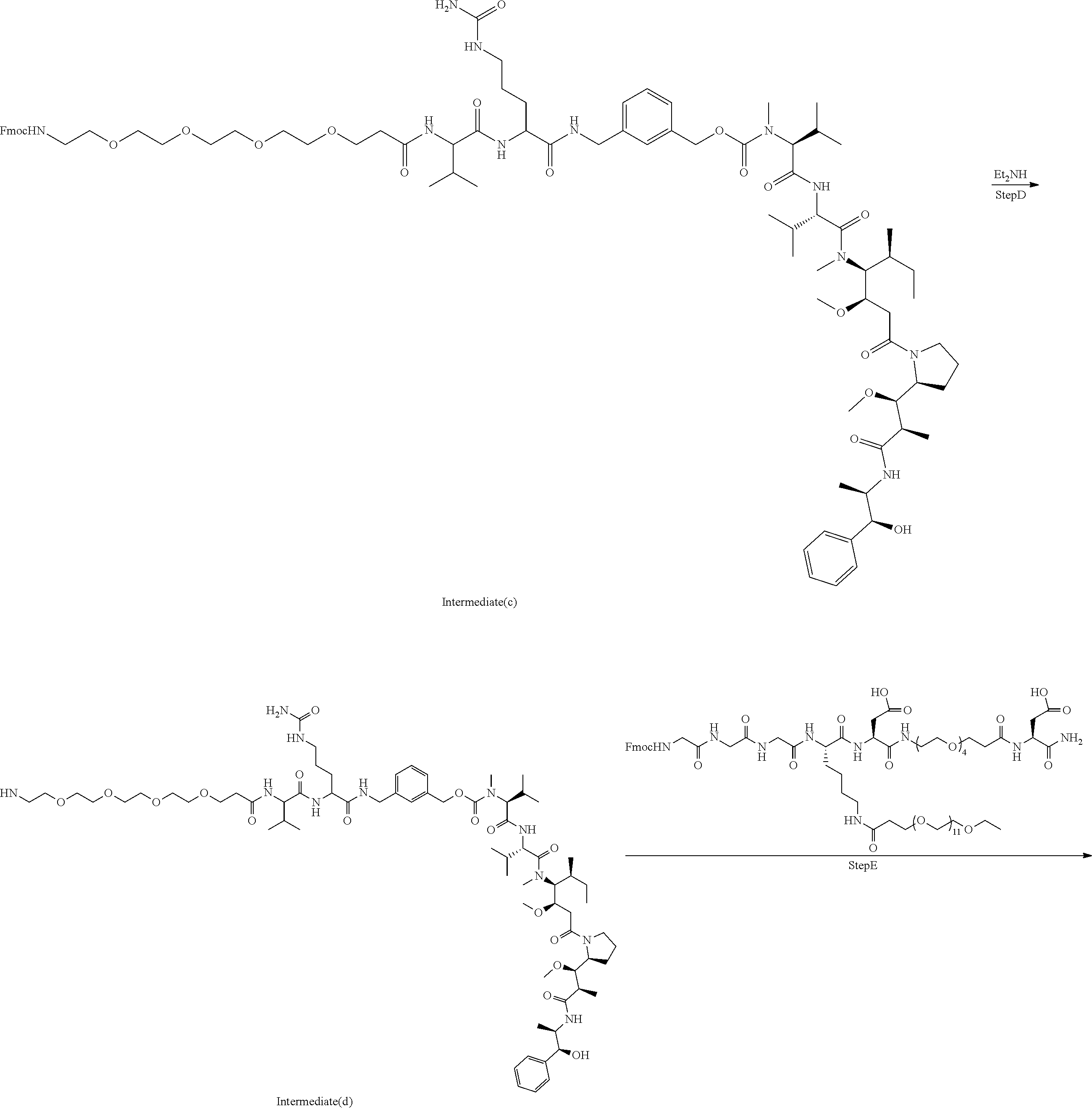

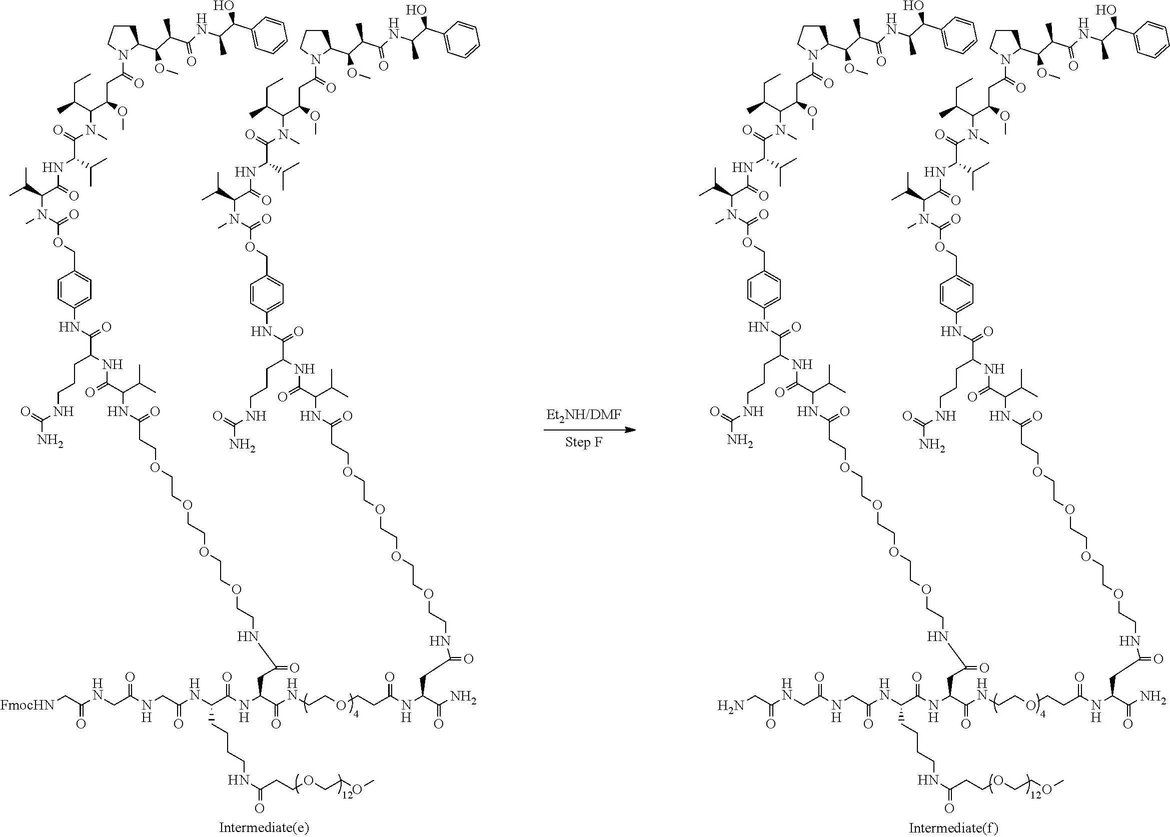

### Step A: Synthesis of Intermediate (a)

Fmoc-PEG4-VC-OH (1 equivalent) and p-hydroxybenzyl alcohol (5.0 equivalent) were added to the reaction flask and dissolved in a solvent of DCM:MeOH=2:1. After adding EEDQ (5.0 equivalent) under nitrogen atmosphere and stirring uniformly, the reaction solution was reacted in the dark at room temperature. The reaction was monitored by HPLC until the reaction was complete (about 3 h). The reaction system was directly prepared by prep-HPLC, and the compound Intermediate (a) (white solid, yield 41%) was obtained by freeze-drying the preparation solution. MS (ESI) Calcd for C₄₃H₆₀O₁₁N₆ [M+H]⁺: 849.4, found: 849.8.

### Step B: Synthesis of Intermediate (b)

The compound Intermediate (a)(1.0 equivalent) was dissolved in DMF and the mixture was cooled to 0° C. P-(dinitrobenzene) carbonate (4.0 equivalent) and DIPEA (6.0 equivalent) were added to the reaction. The reaction system was stirred at 0° C. for 0.5 hour, and then was resumed at room temperature for 2 hours. The reaction was monitored by HPLC until the reaction was complete. The reaction liquid system was concentrated and purified by column chromatography (eluting with 0-10% MeOH in DCM) to obtain the compound Intermediate (b) (white solid, yield 75%). MS (ESI) Calcd for C₅₁H₆₄O₁₅N₇ [M+H]⁺: 1015.4, found: 1015.0.

### Step C: Synthesis of Intermediate (c)

The compound Intermediate (b)(1.2 equivalents) and HOBT (2.0 equivalents) were weighed and dissolved in DMF, then MMAE (1.0 equivalents) and DIPEA (10.0 equivalents) were added and the reaction was stirred uniformly. The mixture was reacted overnight at room temperature. The reaction was monitored by HPLC until the reaction was complete (about 16 h) and the result mixture was directly used for the next reaction.

### Step D: Synthesis of Intermediate (d)

Diethylamine (10% v/v) was added into the reaction solution of the previous step C, and the reaction solution was stirred at room temperature. The reaction was monitored by HPLC until the reaction was complete (about 1.5 h). The reaction system was directly prepared by prep-HPLC, and the compound H0152 (yellow solid, yield 62%) was obtained by freeze-drying the preparation solution. MS (ESI) Calcd for C₆₉H₁₁₆O₁₇N₁₁ [M+H]+: 1370.8, found: 1370.6.

### Step E: Synthesis of Intermediate (e)

The compound Intermediate (d)(2.4 equivalents) and compound b (1.0 equivalent) were weighed and dissolved in DMF, then DIPEA (4.0 equivalents) were added and the reaction was stirred uniformly at 0° C. HATU (2.5 equivalents) was added, and the reaction system was stirred at 0° C. The reaction was monitored by HPLC until the reaction was complete (about 2 h). The reaction system was directly prepared by prep-HPLC, and the compound Intermediate (e) (yellow solid, yield 50%) was obtained by freeze-drying the preparation solution. MS (ESI) Calcd for C₂₁₂H₃₁₉O₆₃N₃₁ [M+4H+]⁴⁺/4: 1084.4, found: 1085.0.

### Step F: Synthesis of Intermediate (f)

The compound Intermediate (e) was weighed and dissolved in DMF, then diethylamine (10% v/w) was added and the reaction solution was stirred at room temperature. The reaction was monitored by HPLC until the reaction was complete (about 0.5 h). The reaction system was directly prepared by prep-HPLC, and the compound Intermediate (f) (yellow solid, yield 90%) was obtained by freeze-drying the preparation solution. MS (ESI) Calcd for C₁₉₇H₃₀₉O₆₁N₃₁ [M+4H+]⁴⁺/4: 1028.9, found: 1029.4.

### Example 3 Preparation of Targeting Molecule-Pharmaceutical Conjugates

3.1 The Linker-payload intermediates were respectively conjugated to an antibody in a site-specific manner by a ligase to form an ADC. The method for conjugation reaction can be found in WO2015165413A1. ADC-4 was prepared using the same method of example 3 in WO2015165413A1. The resulting ADCs are as listed in the following table:

| | Linker- | | |
|---|---|---|---|
| Name of ADC | Payload | Antibody | DAR value |
| ADC-1 | Linker- | Ab0001-LCCT_{L}-HC | 3.7 |
| | Payload 1 | | |
| ADC-2 | Linker- | Ab0001-LCCT_{L}-HC | 3.6 |
| | Payload 2 | | |
| ADC-3 | Linker- | Ab0001-LCCT_{L}-HC | 3.6 |
| | Payload 3 | | |
| ADC-4 | — | Ab0001-LCCT_{L}-HC | 1.8 |
| Enhertu | — | Ab0001 | 7.5 |
| ADC-5 | Linker- | Ab2 | 3.5 |
| | Payload 1 | | |
| ADC-6 | Linker- | Ab2 | 3.6 |
| | Payload 2 | | |
| ADC-7 | Linker- | Ab2 | 3.6 |
| | Payload 3 | | |
| ADC-8 | Linker- | Ab3 | 3.6 |
| | Payload 1 | | |
| ADC-9 | Linker- | Ab3 | 3.7 |
| | Payload 2 | | |
| ADC-10 | Linker- | Ab3 | 3.6 |
| | Payload 3 | | |

3.2 The reference ADC Enhertu was prepared by directly connecting the intermediate MC-GGFG-Dxd to the (optionally modified) antibody (Cys conjugation, i.e. conjugation through connections formed by maleimide structure(s) with thiol group(s) of Cys). The method for conjugation reaction is known in the art.

### Effect Example 1 Bystander Killing Effect of Conjugates in SK-BR-3 and MDA-MB-468 Co-Culture

The SK-BR-3 tumor cells (ATCC, HTB-30) were cultured in McCoy'5A medium supplemented with 10% fetal bovine serum at 37° C. in an atmosphere of 5% CO₂. The MDA-MB-468 tumor cells (ATCC, HTB-132) were cultured in Leibovitz's L-15 medium supplemented with 10% fetal bovine serum at 37° C. in an atmosphere of 0% CO₂. MDA-MB-468-Luc-GFP cell line was constructed by lentiviral infection method and sorted by FACS. Co-culture of SK-1BR-3 and MDA-MB-468-Luc-GFP was seeded with 4:1 ratio (1×10⁴ cells/well) in Corning®s 96-well black/clear bottom polystyrene microplates for 24 h. The test articles (ADC-1, ADC-2, ADC-3, Enhertu, and ADC-4 were administrated according to the experimental design, and incubated for 120 h. The MDA-MB-468-Luc-GFP cell count was performed on BioTek Cytation3 (BioTek, LAB 14002). And then luciferase activity was detected using Firefly Luciferase Reporter Gene Assay Kit (Beyotime, RG006) on BioTek Synergy HTX (BioTek, MAB 16038).

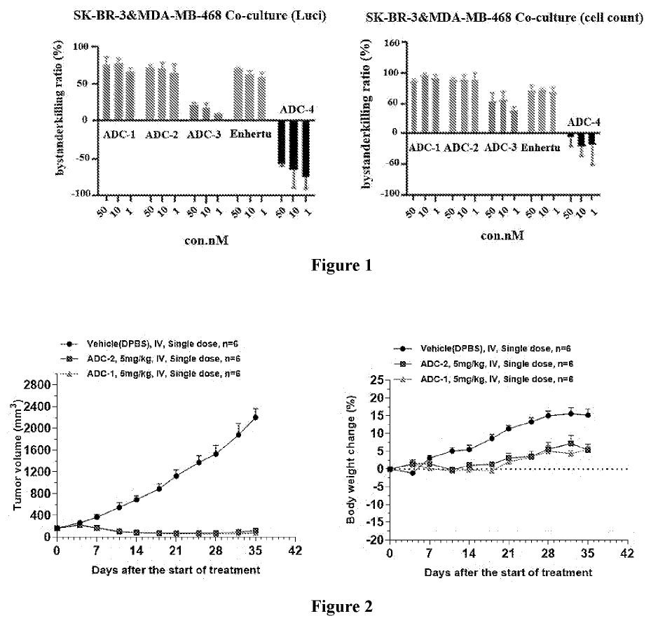

In SK-B3R-3 and MDA-MB-468-Luc-GFP co-culture (FIG. 1), the ADC-1, ADC-2 exhibited similar efficacy. And both of ADC-1 and ADC-2 exhibited the comparable or even better efficacy than Enhertu.

### Effect Example 2: Effect of ADC-2 and ADC-1 in JIMT-1 CDX Model In Vivo

The JIMT-1 tumor cells (DSMZ-ACC 589) were maintained in vitro as a monolayer culture in RPMI 1640 medium supplemented with 10% fetal bovine serum, 1% Antibiotic-Antimycotic, at 37° C. in an atmosphere of 5% CO₂ in air. The tumor cells were routinely subcultured twice weekly by trypsin-EDTA treatment.

SCID Beige, female, 6-8 weeks, weighing approximately 18-20 g. A total of 27 (18 plus 50% spare) was needed for the study, which were purchased from Shanghai Lingchang biotechnology co. LTD. or other certified vendors.

Each mouse was inoculated subcutaneously at the right flank with JIMT-1 tumor cells (5×10⁶) in 0.2 mL of PBS with Matrigel (1:1) for tumor development. The animals were randomized and treatment was started when the average tumor volume reached approximately 100-200 mm³ for the efficacy study. The test articles (ADC-2, ADC-1) administration and the animal numbers in each group were in accordance with the experimental design.

After inoculation, the animals were checked daily for morbidity and mortality. At the time of routine monitoring, the animals were checked for any effects of tumor growth and treatments on normal behavior such as mobility, food and water consumption, body weight gain/loss (body weights will be measured twice weekly), eye/hair matting and any other abnormal effect. The major endpoint was to see if the tumor growth can be delayed or mice can be cured.

Statistical Analysis: For comparison between two groups, an independent sample t-test was used. For comparison among three or more groups, a one-way ANOVA was performed. If a significant F -statistics (a ratio of treatment variance to the error variance) was obtained, multiple comparison procedures was applied after ANOVA. All data was analyzed using SPSS 17.0. p<0.05 was considered to be statistically significant.

According to the results (FIG. 2), ADC-1 exhibited the comparable or even better efficacy than ADC-2.

### Effect Example 3: Effect of ADC-2, ADC-3 and Enhertu in JIMT-1 CDX Model In Vivo

Each mouse was inoculated subcutaneously at the right flank with JIMT-1 tumor cells (5×10⁶) in 0.2 mL mixture of PBS and Matrigel (PBS: Matrigel=1:1) for tumor development. Treatments were started on day 10 after tumor inoculation when the average tumor volume reached 200 mm³. The mean tumor volume of the vehicle control group reached 1,474 mm³ on day 35 after administration. T/C and TGI values were calculated using tumor volume. The calculation formula is as follows: T/C %=T_{RTV}/C_{RTV}×100% (T_{RTV}: RTV of the treatment group; C_{RTV}: RTV of the vehicle control group). The relative tumor volume (RTV) was calculated based on the results of tumor measurement, and the calculation formula was RTV=Vₜ/V₀, where V₀ was the average tumor volume measured at the time of grouping (i.e., D0), Vₜ was the average tumor volume at one measurement, and T_{RTV} and C_{RTV} took the same day of data. Calculation of TGI (%): TGI (%)=[1−(average tumor volume at the end of administration of a treatment group−average tumor volume at the beginning of administration of the treatment group)/(average tumor volume at the end of treatment of the vehicle control group−the average tumor volume at the beginning of treatment of the vehicle control group)]×100%. Treatments with ADC-2 (T/C=6.23%, TGI=108.47%, p=0.011) and Enhertu (T/C=33.51%, TGI=76.90%, p=0.041) at 5 mg/kg produced significant antitumor activity with mean tumor volume of 92 mm³ and 494 mm³. Treatment with ADC-3 (T/C=43.16%, TGI=63.73%, p=0.089) at 5 mg/kg also produced some antitumor activity with mean tumor volume of 636 mm³ on day 35.

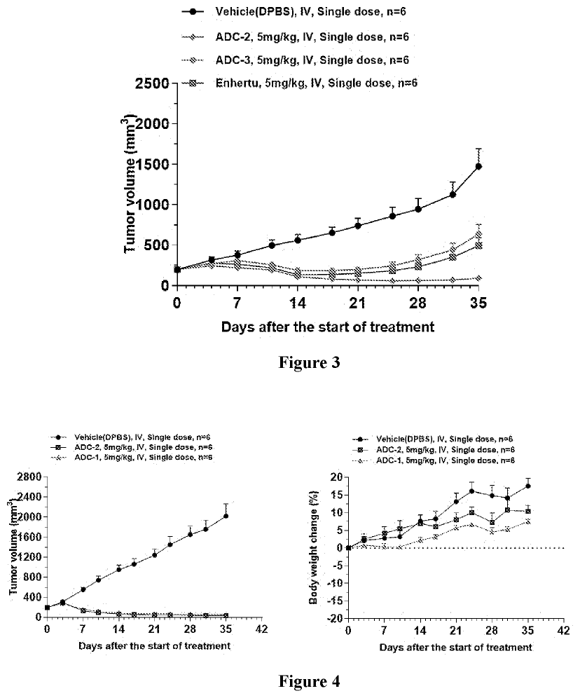

According to the results (FIG. 3) showed that the efficacy of ADC-3 and Enhertu were not statistically significant difference. And these data also suggested that ADC-2 represented better antitumor efficacy than Enhertu in JIMT-1 model.

From the results of effect example 2 (FIG. 2) and effect example 3 (FIG. 3), the conjugate of ADC-1 exhibited the comparable or even better efficacy than ADC-2 and further better efficacy than Enhertu.

### Effect Example 4: Effect of Conjugates in NCI-N87 CDX Model In Vivo

The NCI-N87 tumor cells (ATCC, Manassas, VA, cat #CRL-5822) were maintained in vitro as a monolayer culture in RPMI 1640 medium supplemented with 10% fetal bovine serum, 1% Antibiotic-Antimycotic, at 37° C. in an atmosphere of 5% CO₂ in air. The tumor cells were routinely subcultured twice weekly by trypsin-EDTA treatment.

BALB/c Nude, female, 6-8 weeks, weighing approximately 18-20 g. A total of 25 (18 plus 39% spare) was needed for the study, which were purchased from Shanghai Lingchang biotechnology co. LTD. or other certified vendors.

Each mouse was inoculated subcutaneously at the right flank with NCI-N87 tumor cells (10×10⁶) in 0.2 mL of PBS with Matrigel (1:1) for tumor development. The animals were randomized and treatment was started when the average tumor volume reached approximately 150-200 mm³ for the efficacy study. The test articles (ADC-2, ADC-1) administration and the animal numbers in each group followed the experimental design.

After inoculation, the animals were checked daily for morbidity and mortality. At the time of routine monitoring, the animals were checked for any effects of tumor growth and treatments on normal behavior such as mobility, food and water consumption, body weight gain/loss (body weights will be measured twice weekly), eye/hair matting and any other abnormal effect. The major endpoint was to see if the tumor growth can be delayed or mice can be cured.

According to the results (FIG. 4), the conjugate of ADC-1 exhibited the comparable efficacy to ADC-2.

### Effect Example 5: Effect of ADC-5 on the Proliferation of TROP2-Positive Tumor Cells BxPC-3

1) TROP2 positive human pancreatic cancer cell BxPC-3 was inoculated into 96-well cell plates at 100 μl per well (containing 1000˜5000 cells), and cultured overnight in a cell incubator at 37° C., 5% CO₂, 95% air and 100% humidity.

2) ADCs with different concentrations (200, 40, 8, 1.6, 0.32, 0.064, 0.013, 0.0026, 0.00051 and 0.00010 nM) were added to BxPC-3 cells cultured overnight. Puromycin with a 5 μM final concentration was added into the control group. Incubation was continued at 37° C. for 120 h.

3) The cell plate was removed from the cell incubator at 37° C. and equilibrated for about 30 minutes to room temperature. 100 μl CellTiterGlo reagent was added to each well. After oscillating 2 min, the oscillator was placed 10 min in the dark at room temperature, and the relative light unit (RLU) was measured by Cytation3 microplate analyzer.

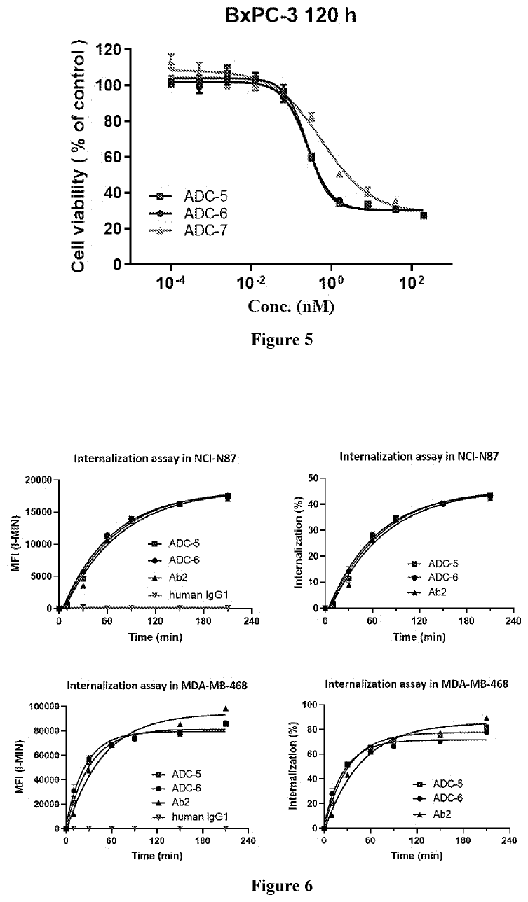

4) The results of the inhibitory effects of different drugs on the proliferation of tumor cells are shown in Table 3 and FIG. 5. ADCs have obvious inhibitory effects on TROP2 positive cells, wherein the effects of ADC-5 and ADC-6 are essentially the same, which are obviously better than ADC-7.

TABLE 3
Inhibitory effects of different drugs
on tumor cell proliferation (IC₅₀, nM)

| | ADC-5 | ADC-6 | ADC-7 |
|---|---|---|---|
| IC₅₀, nM | 0.2491 | 0.2543 | 0.6019 |

### Effect Example 6: Detection of the Endocytosis of ADC-5 by TROP2-Positive Tumor Cells

1) TROP2 positive human gastric cancer cell NCI-N87 and human breast cancer cell MDA-MB-468 were inoculated into a 96-well cell plate with 1100 μl (containing 10,000˜50,000 cells) per well, and incubated with 50 μg/mL fluorescent labeled ADCs at 4° C. for 30 min in the dark.

2) After the incubation, PBS was used to clean the unbound ADC, and the cells were incubated in a cell incubator at 37° C., 5% CO₂, 95% air and 100% humidity for 10 min, 30 min, 1 h, 1.5h, 2.5h and 3.5h respectively, and then the cells were removed, and the pre-cooled PBS was added to terminate endocytosis. The acid buffer with pH value of 2.5 was used for ice bath for 3 minutes, and the cells were precipitated by centrifugation at 2000 rpm for 3 minutes, and then the cells were resuspended with 100 μl/well FACS buffer, which was ready for flow cytometry detection.

3) The results of the endocytosis of TROP2 positive tumor cells on the detected ADCs are shown in Table 4 and FIG. 6. The endocytosis of ADC ADC-5 is essentially the same as that of ADC-6 and monoclonal antibody Ab2. Under the same conditions, the negative control of human IgG1 does not induce the endocytosis of cells, indicating that the preparation of ADCs by monoclonal antibody conjugated with payload does not affect the intracellular endocytosis induced by monoclonal antibody, and the endocytosis of ADCs is target-dependent.

TABLE 4
Endocytosis of ADC by tumor cells

| | | Maximum | Maximum |
|---|---|---|---|
| | Half | endocytosis | endocytosis |
| ADC | Life(min) | (MFI) | rate (%) |
| ADC-5 | 45.65 | 18554 | 45.97 |
| ADC-6 | 42.76 | 18228 | 45.04 |
| Ab2 | 48.17 | 18549 | 45.95 |
| Human lgG1 | — | — | — |

Note:
“—” means not determined.

### Effect Example 7: Effect of ADC-5 on Tumor Growth in TROP2 Positive NCI-N87 CDX Mouse Model

1) Cell culture: NCI-N87 tumor cells (ATCC, cat #CRL-5822) were maintained in vitro as a monolayer culture in RPMI-1640 medium supplemented with 10% fetal bovine serum and 1% antibiotics-antimycotic at 37° C. in an atmosphere of 5% CO₂ in air. The tumor cells were routinely subcultured twice weekly by trypsin-EDTA treatment. Cells growing in exponential growth period were collected and counted for tumor inoculation.

2) Animals: BALB/c female nude mice, 7-9 weeks, weighing approximately 18-22 g. A total of 24 mice were needed for the study, which were purchased from Shanghai Lingchang Biotechnology Co., Ltd.

3) Tumor inoculation and administration: Each mouse was inoculated subcutaneously at the right flank with 0.2 mL (10×10⁶) NCI-N87 tumor cells (the ratio of PBS to Matrigel was 1:1). When the average tumor volume reached approximately 150-200 mm³, the animals were randomized according to the size of tumor, 6 mice in each group, and were treated by intravenous injection at a dose of 3 mg/kg. The whole process of animal breeding and experiment met the requirements of animal welfare.

4) Effect evaluation: after administration, the tumor volume and animal weight were measured every week, and the tumor volume was calculated by the formula V=0.5×a×b², wherein a and b are the long and short diameters of the tumor respectively. Then TGI (%) and tumor relative proliferation rate T/C (%) were calculated using tumor volume. The calculation formula of TGI for each group is as follows: TGI(%)=[1−(Tᵢ−T₀)/(Vᵢ−V₀)]×100, wherein Tᵢ is the average tumor volume of the treatment group on a certain day, T₀ is the average tumor volume of the treatment group on the first day of treatment, Vᵢ is the average tumor volume of the vehicle control group on the same day as Tᵢ, and V₀ is the average tumor volume of the vehicle control group on the first day of treatment. The calculation formula of T/C (%) value is as follows: T/C %=T_{RTV}/C_{RTV}×100% (T_{RTV}: relative average tumor volume (RTV) in the treatment group; C_{RTV}: relative average tumor volume (RTV) of vehicle control group on the same day as T_{RTV}. The relative tumor volume (RTV) of each group was calculated as follows: RTV=Vₜ/V₀, wherein V₀ is the tumor volume on the first day of treatment and Vₜ is the tumor volume on a certain day.

5) Statistical analysis: For comparison between the two groups, an independent sample T test was used. For comparison among three or more groups, a one-way ANOVA was performed. If a significant F-statistics (a ratio of treatment variance to error variance) was obtained, multiple comparison procedures were applied after variance analysis. All data was analyzed using SPSS 17.0. P<0.05 was considered the difference to be statistically significant.

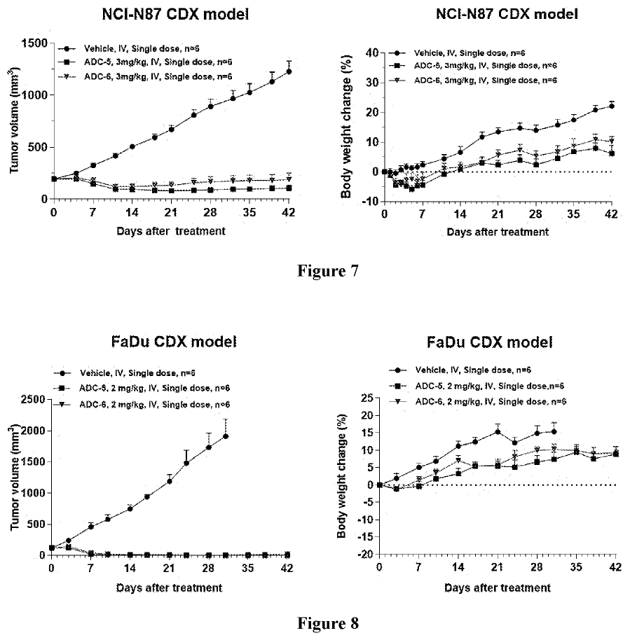

6) Changes in tumor volume and animal weight are detailed in table 5 and FIG. 7. The inhibitory effects of ADC-5 and ADC-6 on NCI-N87 tumor volume are comparable, and all of above groups are extremely significant compared to the control group. No significant body weight loss was observed in each ADC-administration group, indicating that the mice have good tolerance to ADC-5 and ADC-6.

TABLE 5
Statistical analysis of experimental results

| | | Tumor | | | |
|---|---|---|---|---|---|
| | | volume(mm³)¹ | T/C² | TGI³ | |
| Treatment | N | on day 42 | (%) | (%) | pvalue⁴ |
| Vehicle | 6 | 1,226 ± 102 | — | — | — |
| ADC-5, 3 mg/kg | 6 | 106 ± 28 | 8.69 | 108.67 | <0.001 |
| ADC-6, 3 mg/kg | 6 | 189 ± 63 | 15.41 | 100.68 | <0.001 |

Note:
¹Mean ± SEM;
²The calculation formula of T/C (%) is as follows: T/C % = T_{RTV}/C_{RTV} × 100%. The calculation formula of the relative tumor volume (RTV) of each group is as follows: RTV = V_{t/}V₀; V₀ is the average tumor volume on the first day of treatment, and Vₜ is the average tumor volume on a certain day;
³The calculation formula of TGI for each group is as follows: TGI (%) = [1 − (T₄₂ − T₀)/(V₄₂ − V₀)] × 100;
⁴P value is calculated according to tumor volume.

### Effect Example 8: Effect of ADC-5 on Tumor Growth in TROP2 Positive FaDu CDX Mouse Model

The experiment was carried out with reference to the method described in Effect Example 7, wherein the administration concentration was set at 2 mg/kg. The changes of tumor volume and animal weight are shown in Table 6 and FIG. 8. The inhibitory effect of ADC-5 test group (CR: 6 mice) on the tumor volume of FaDu is slightly better than that of ADC-6 (CR: 2 mice), and both of above groups are significant differences compared to the control group. No significant body weight loss of mice was observed in each ADC-administration group, indicating that the mice have good tolerance to ADC-5 and ADC-6.

TABLE 6
Statistical analysis of experimental results

| | | Tumor | | | | |
|---|---|---|---|---|---|---|
| | | volume(mm³)¹ | T/C² | TGI³ | | |
| Treatment | N | on day 31 | (%) | (%) | pvalue⁴ | CR |
| Vehicle | 6 | 1,911 ± 279 | — | — | — | 0 |
| ADC-5, 2 mg/kg | 6 | 1 ± 0 | 0.05 | 106.60 | 0.007 | 6 |
| ADC-6, 2 mg/kg | 6 | 4 ± 1 | 0.22 | 106.42 | 0.007 | 2 |

Note:
¹Mean ± SEM;
²The calculation formula of T/C (%) is as follows: T/C % = T_{RTV}/C_{RTV} × 100%. The calculation formula of the relative tumor volume (RTV) of each group is as follows: RTV = Vₜ/V₀; V₀ is the average tumor volume on the first day of treatment, and Vₜ is the average tumor volume on a certain day;
³The calculation formula of TGI for each group is as follows: TGI (%) = [1 − (T₃₁ − T₀)/(V₃₁ − V₀)] × 100;
⁴P value is calculated according to tumor volume.

### Effect Example 9: Effect of ADC-8 on Tumor Growth in FGFR3 Positive RT112/84 CDX Mouse Model

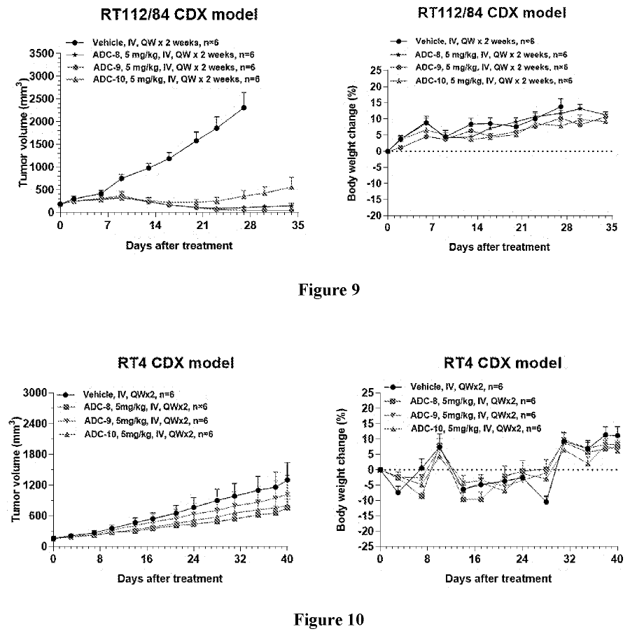

The experiment was carried out with reference to the method described in Effect Example 7, wherein the administration concentration was set at 5 mg/kg. The changes of tumor volume and animal weight are shown in Table 7 and FIG. 9. The inhibitory effects of ADC-8 and ADC-9 on RT112/84 tumor volume were comparable and significantly superior to that of ADC-10, and all of above groups are extremely significant compared to the control group. There is no difference in body weight between each ADC administration group and the control group, indicating that the mice have good tolerance to ADC-8, ADC-9, and ADC-10.

TABLE 7
Statistical analysis of experimental results

| | | Tumor | | | |
|---|---|---|---|---|---|
| | | volume(mm³)¹ | T/C² | TGI³ | |
| Treatment | N | on day 27 | (%) | (%) | pvalue⁴ |
| Vehicle | 6 | 2,307 ± 336 | — | — | — |
| ADC-10, 5 mg/kg | 6 | 357 ± 121 | 15.53 | 91.80 | 0.013 |
| ADC-9, 5 mg/kg | 6 | 48 ± 6 | 2.09 | 106.36 | 0.009 |
| ADC-8, 5 mg/kg | 6 | 106 ± 43 | 4.60 | 103.63 | 0.010 |

Note:
¹Mean ± SEM;
²The calculation formula of T/C (%) is as follows: T/C % = T_{RTV}/C_{RTV} × 100%. The calculation formula of the relative tumor volume (RTV) of each group is as follows: RTV = V_{t/}V₀; V₀ is the average tumor volume on the first day of treatment, and Vₜ is the average tumor volume on a certain day;
³The calculation formula of TGI for each group is as follows: TGI (%) = [1 − (T₂₇ − T₀)/(V₂₇ − V₀)] × 100;
⁴P value is calculated according to tumor volume.

### Effect Example 10: Effect of ADC-8 on Tumor Growth in FGFR3 Positive RT4 CDX Mouse Model

The experiment was carried out with reference to the method described in Effect Example 7, wherein the administration concentration was set at 5 mg/kg. The changes of tumor volume and animal weight are shown in Table 8 and FIG. 10. ADC-8, ADC-9 and ADC-10 all have certain inhibitory effects on RT4 tumor volume, and ADC-8 and ADC-10 are slightly better than ADC-9. There is no difference in body weight between each ADC administration group and the control group, indicating that the mice have good tolerance to ADC-8, ADC-9, and ADC-10.

TABLE 8
Statistical analysis of experimental results

| | | Tumor | | |
|---|---|---|---|---|
| | | volume(mm³)¹ | T/C² | TGI³ |
| Treatment | N | on day 40 | (%) | (%) |
| Vehicle | 6 | 1,300 ± 339 | — | — |
| ADC-10, 5 mg/kg | 6 | 789 ± 196 | 60.70 | 44.92 |
| ADC-9, 5 mg/kg | 6 | 1,023 ± 370 | 78.69 | 24.36 |
| ADC-8, 5 mg/kg | 6 | 758 ± 169 | 58.24 | 47.62 |

Note:
¹Mean ± SEM;
²The calculation formula of T/C (%) is as follows: T/C % = T_{RTV}/C_{RTV} × 100%. The calculation formula of the relative tumor volume (RTV) of each group is as follows: RTV = V_{t/}V₀; V₀ is the average tumor volume on the first day of treatment, and Vₜ is the average tumor volume on a certain day;
³The calculation formula of TGI for each group is as follows: TGI (%) = [1 − (T₄₀ − T₀)/(V₄₀ − V₀)] × 100;
⁴P value is calculated according to tumor volume.

Sequence Listing

SEQ ID No. 1: Ab0001-LCCT_{L}-HC Light chain:
DIQMTQSPSSLSASVGDRVTITCRASQDVNTAVAWYQQKPGKAPKLLIYSASFLY
SGVPSRFSGSRSGTDFTLTISSLQPEDFATYYCQQHYTTPPTFGQGTKVEIKRTVAA
PSVFIFPPSDEQLKSGTASVVCLLNNFYPREAKVQWKVDNALQSGNSQESVTEQ
DSKDSTYSLSSTLTLSKADYEKHKVYACEVTHQGLSSPVTKSFNRGECGALPETG
G
SEQ ID No. 2: Ab0001-LCCT_{L}-HC Heavy chain:
EVQLVESGGGLVQPGGSLRLSCAASGFNIKDTYIHWVRQAPGKGLEWVARIYPT
NGYTRYADSVKGRFTISADTSKNTAYLQMNSLRAEDTAVYYCSRWGGDGFYAM
DYWGQGTLVTVSSASTKGPSVFPLAPSSKSTSGGTAALGCLVKDYFPEPVTVSW
NSGALTSGVHTFPAVLQSSGLYSLSSVVTVPSSSLGTQTYICNVNHKPSNTKVDK
KVEPKSCDKTHTCPPCPAPELLGGPSVFLFPPKPKDTLMISRTPEVTCVVVDVSHE
DPEVKFNWYVDGVEVHNAKTKPREEQYNSTYRVVSVLTVLHQDWLNGKEYKC
KVSNKALPAPIEKTISKAKGQPREPQVYTLPPSREEMTKNQVSLTCLVKGFYPSDI
AVEWESNGQPENNYKTTPPVLDSDGSFFLYSKLTVDKSRWQQGNVFSCSVMHE
ALHNHYTQKSLSLSPGK
SEQ ID No. 3: Ab0001-LCCT_{L}-HCCT_{L} Light chain:
DIQMTQSPSSLSASVGDRVTITCRASQDVNTAVAWYQQKPGKAPKLLIYSASFLY
SGVPSRFSGSRSGTDFTLTISSLQPEDFATYYCQQHYTTPPTFGQGTKVEIKRTVAA
PSVFIFPPSDEQLKSGTASVVCLLNNFYPREAKVQWKVDNALQSGNSQESVTEQ
DSKDSTYSLSSTLTLSKADYEKHKVYACEVTHQGLSSPVTKSFNRGECGALPETG
G
SEQ ID No. 4: Ab0001-LCCT_{L}-HCCT_{L} Heavy chain:
EVQLVESGGGLVQPGGSLRLSCAASGFNIKDTYIHWVRQAPGKGLEWVARIYPT
NGYTRYADSVKGRFTISADTSKNTAYLQMNSLRAEDTAVYYCSRWGGDGFYAM
DYWGQGTLVTVSSASTKGPSVFPLAPSSKSTSGGTAALGCLVKDYFPEPVTVSW
NSGALTSGVHTFPAVLQSSGLYSLSSVVTVPSSSLGTQTYICNVNHKPSNTKVDK
KVEPKSCDKTHTCPPCPAPELLGGPSVFLFPPKPKDTLMISRTPEVTCVVVDVSHE
DPEVKFNWYVDGVEVHNAKTKPREEQYNSTYRVVSVLTVLHQDWLNGKEYKC
KVSNKALPAPIEKTISKAKGQPREPQVYTLPPSREEMTKNQVSLTCLVKGFYPSDI
AVEWESNGQPENNYKTTPPVLDSDGSFFLYSKLTVDKSRWQQGNVFSCSVMHE
ALHNHYTQKSLSLSPGKGALPETGG
SEQ ID No. 5: Ab2 Light chain:
DIQLTQSPSSLSASVGDRVSITCKASQDVSIAVAWYQQKPGKAPKLLIYSASYRYT
GVPDRFSGSGSGTDFTLTISSLQPEDFAVYYCQQHYITPLTFGAGTKVEIKRTVAAP
SVFIFPPSDEQLKSGTASVVCLLNNFYPREAKVQWKVDNALQSGNSQESVTEQD
SKDSTYSLSSTLTLSKADYEKHKVYACEVTHQGLSSPVTKSFNRGECGALPETGG
SEQ ID No. 6: Ab2 heavy chain:
QVQLQQSGSELKKPGASVKVSCKASGYTFTNYGMNWVKQAPGQGLKWMGWI
NTYTGEPTYTDDFKGRFAFSLDTSVSTAYLQISSLKADDTAVYFCARGGFGSSYW
YFDVWGQGSLVTVSSASTKGPSVFPLAPSSKSTSGGTAALGCLVKDYFPEPVTVS
WNSGALTSGVHTFPAVLQSSGLYSLSSVVTVPSSSLGTQTYICNVNHKPSNTKVD
KRVEPKSCDKTHTCPPCPAPELLGGPSVFLFPPKPKDTLMISRTPEVTCVVVDVSH
EDPEVKFNWYVDGVEVHNAKTKPREEQYNSTYRVVSVLTVLHQDWLNGKEYK
CKVSNKALPAPIEKTISKAKGQPREPQVYTLPPSREEMTKNQVSLTCLVKGFYPS
DIAVEWESNGQPENNYKTTPPVLDSDGSFFLYSKLTVDKSRWQQGNVFSCSVMH
EALHNHYTQKSLSLSPGK
SEQ ID No. 7: Ab3 light chain:
QSVLTQPPSLSVAPGKTATFTCGGNNIGDKSVHWYRQKPGQAPVLVMYLDTERP
SGIPERMSGSNFGNTATLTITRVEAEDEADYYCQVWDSGSDHVVFGGGTKLTVL
GQPKAAPSVTLFPPSSEELQANKATLVCLISDFYPGAVTVAWKADSSPVKAGVET
TTPSKQSNNKYAASSYLSLTPEQWKSHRSYSCQVTHEGSTVEKTVAPAECSGALP
ETGG
SEQ ID No. 8: Ab3 heavy chain:
EVQLVQSGAEVKKPGASVKVSCKASGYMFTSYGISWVRQAPGQGLEWMGWVS
TYNGDTNYAQKFQGRVTVTTDTSTSTAYMELRSLRSEDTAVYYCARVLGYYDSI
DGYYYGMDVWGQGTTVTVSSASTKGPSVFPLAPSSKSTSGGTAALGCLVKDYF
PEPVTVSWNSGALTSGVHTFPAVLQSSGLYSLSSVVTVPSSSLGTQTYICNVNHKP
SNTKVDKKVEPKSCDKTHTCPPCPAPELLGGPSVFLFPPKPKDTLMISRTPEVTCV
VVDVSHEDPEVKFNWYVDGVEVHNAKTKPREEQYNSTYRVVSVLTVLHQDWL
NGKEYKCKVSNKALPAPIEKTISKAKGQPREPQVYTLPPSREEMTKNQVSLTCLV
KGFYPSDIAVEWESNGQPENNYKTTPPVLDSDGSFFLYSKLTVDKSRWQQGNVF
SCSVMHEALHNHYTQKSLSLSPGK

1. A compound of formula (I): 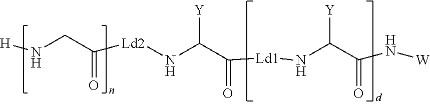
wherein,
W is hydrogen, LKb or —C₂H₄-(PEG)ₜ-(CO)NH₂;
Y is LKa-LKb;
each LKa is independently 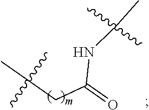 [SMILES: *CC(=O)N*]
each LKb is independently L²-L¹-B;
each B is independently a terminal group R¹⁰, or a combination of 1) a self-immolative spacer Sp1; 2) a bond, or one of or a combination of two or more of the bivalent groups selected from: -CR¹R²—, C₄₋₁₀ alkylene, C₄₋₁₀ cycloalkylene, C₄₋₁₀ heterocyclylene and —(CO)—; and 3) a terminal group R¹⁰;
R¹⁰ is hydrogen, or a group which can leave when reacting with a group in the payload;
each L¹ is independently Cleavable sequence 1 comprising an amino acid sequence which can be cleaved by enzyme, and Cleavable sequence 1 comprises 1-10 amino acids;
each L² is independently a bond; or a C₂₋₂₀ alkylene wherein one or more —CH₂— structures in the alkylene is optionally replaced by —CR³R⁴—, —O—, —(CO)—, —S(═O)₂—, —NR⁵—, -N_{R}⁶R⁷—, C₄₋₁₀ cycloalkylene, C₄₋₁₀ heterocyclylene, phenylene; wherein the cycloalkylene, heterocyclylene and phenylene are each independently unsubstituted or substituted with at least one substituent selected from halogen, —C₁₋₁₀ alkyl, —C₁₋₁₀ haloalkyl, —C₁₋₁₀ alkylene—NH—R⁸ and -C₁₋₁₀ alkylene-O—R⁹;
Ld2 and each Ld1 are independently a bond; or selected from —NH—C₁₋₂₀ alkylene-(CO)—, -NH-(PEG)ᵢ-(CO)—, or are a natural amino acid or oligomeric natural amino acids having a degree of polymerization of 2-10 independently unsubstituted or substituted with —(CO)-(PEG)ⱼ-OR¹¹ on the side chain;
-(PEG)ₜ-, -(PEG)ᵢ- and -(PEG)ⱼ- are each a PEG fragment, which comprises the denoted number of consecutive —(O—C₂H₄)- structure units or consecutive —(C₂H₄—O)— structure units, with an optional additional C₁₋₁₀ alkylene at one terminal;
R¹, R², R³, R⁴, R⁵, R⁶, R⁷, R⁸, R⁹ are each independently selected from hydrogen, halogen, —C₁₋₁₀ alkyl, —C₁₋₁₀ haloalkyl, C₄₋₁₀ cycloalkylene; or
R¹ and R² together with the carbon atom to which they are attached form a 3-6 membered cycloalkyl group; or
R³ and R⁴ together with the carbon atom to which they are attached form a 3-6 membered cycloalkyl group;
R¹¹ is C₁₋₁₀ alkyl;
m is any integer of 1 to 5;
n is any integer of 2 to 20;
d is any integer of 0 to 6, particularly 1, 2, 3;
each i is independently an integer of 0-100, preferably 0 to 20; preferably each i is independently an integer of 0 to 12; more preferably 0 to 8; particularly 4;
each j is independently an integer of 1-100, preferably 1 to 20; preferably each j is independently an integer of 1 to 12; more preferably 8 to 12; particularly 8 or 12;
each t is independently an integer of 1-100, preferably 1 to 20; preferably each t is independently an integer of 1 to 12; more preferably 8 to 12; particularly 8 or 12.

2. The compound of claim 1, selected from

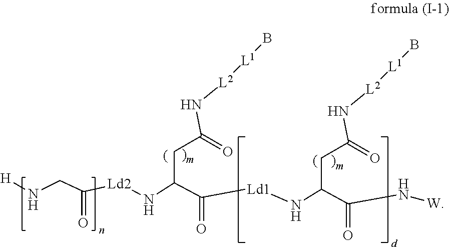

3. The compound of claim 1, selected from 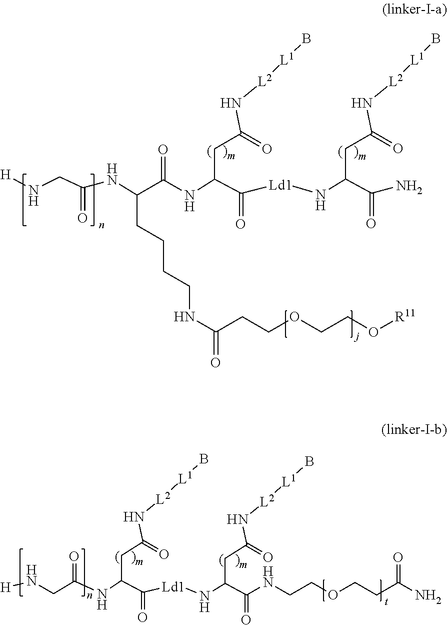
wherein,
m is any integer of 1 to 5;
each t is independently an integer of 1-100, preferably 1 to 20; preferably each t is independently an integer of 1 to 12; more preferably 8 to 12; particularly 8 or 12;
each j is independently an integer of 1-100, preferably 1 to 20; preferably each j is independently an integer of 1 to 12; more preferably 8 to 12; particularly 8 or 12.

4. The compound of claim 1, wherein
Ld2 and each Ld1 are independently a bond or 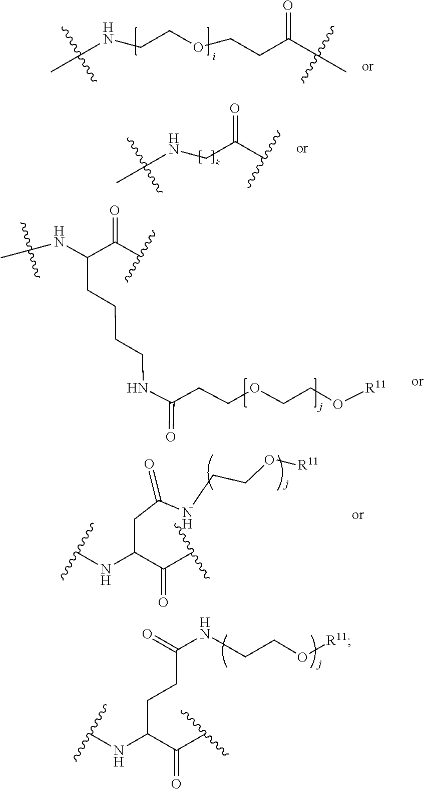
each k is independently an integer of 1-100, preferably 1 to 20; preferably each k is independently an integer of 1 to 12; more preferably 1 to 7; particularly 1, or 3 or 5;
preferably, Ld1 is 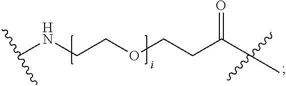
Ld2 is independently selected from a bond or 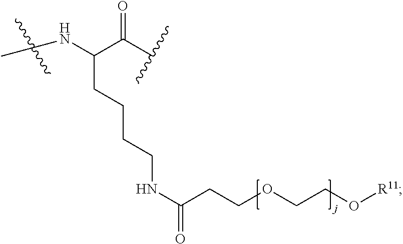
each i is independently an integer of 0-5, preferably 0-4; particularly 0, 2 or 4;
each j is independently an integer of 1-100, preferably 1 to 20; preferably each j is independently an integer of 1 to 12; more preferably 8 to 12; particularly 8 or 12.

5. The compound of claim 1, wherein
Cleavable sequence 1 is selected from Gly-Gly-Phe-Gly, Phe-Lys, Val-Cit, Val-Lys, Gly-Phe-Leu-Gly, Ala-Leu-Ala-Leu, Ala-Ala-Ala and the combination thereof, preferably, Cleavable sequence 1 is Gly-Gly-Phe-Gly or Val-Cit;
and/or
Sp1 is selected from PABC, acetal, heteroacetal and the combination thereof, preferably, Sp1 is acetal, heteroacetal or PABC; preferably, the heteroacetal is selected from N,O-heteroacetal; preferably, Sp1 is —O—CH₂—U- or —NH—CH₂—U—; wherein the —O— or the —NH— is connected to Cleavable sequence 1, and U is absent or U is O, S or NH, preferably O or S; more preferably Sp1 is PABC.

6. The compound of claim 1, wherein
W is hydrogen; and/or R¹⁰ is hydrogen, hydroxy, or 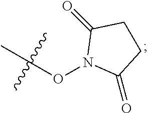 [SMILES: *ON1C(=O)CCC1=O]
R¹¹ is C₁₋₆ alkyl, preferably methyl; and/or
m is an integer of 1-3, preferably 1 or 2;
n is an integer of 2 to 5, especially 3; and/or
d is 0, or is any integer of 1 to 4; preferably, 1, 2 or 3; more preferably 1.

7. The compound of claim 1, selected from 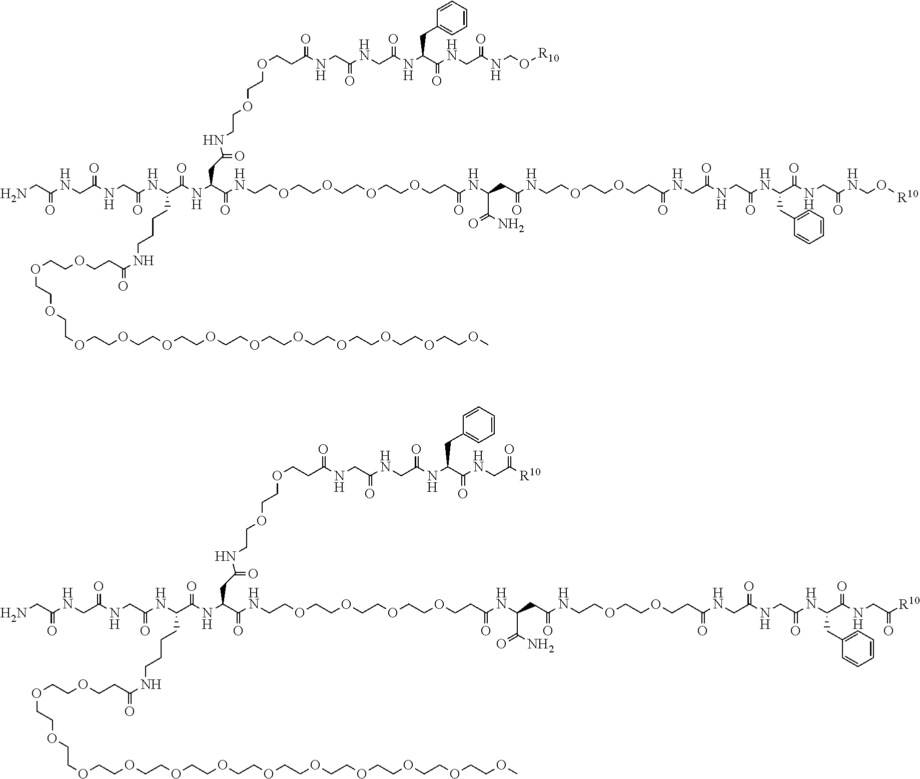 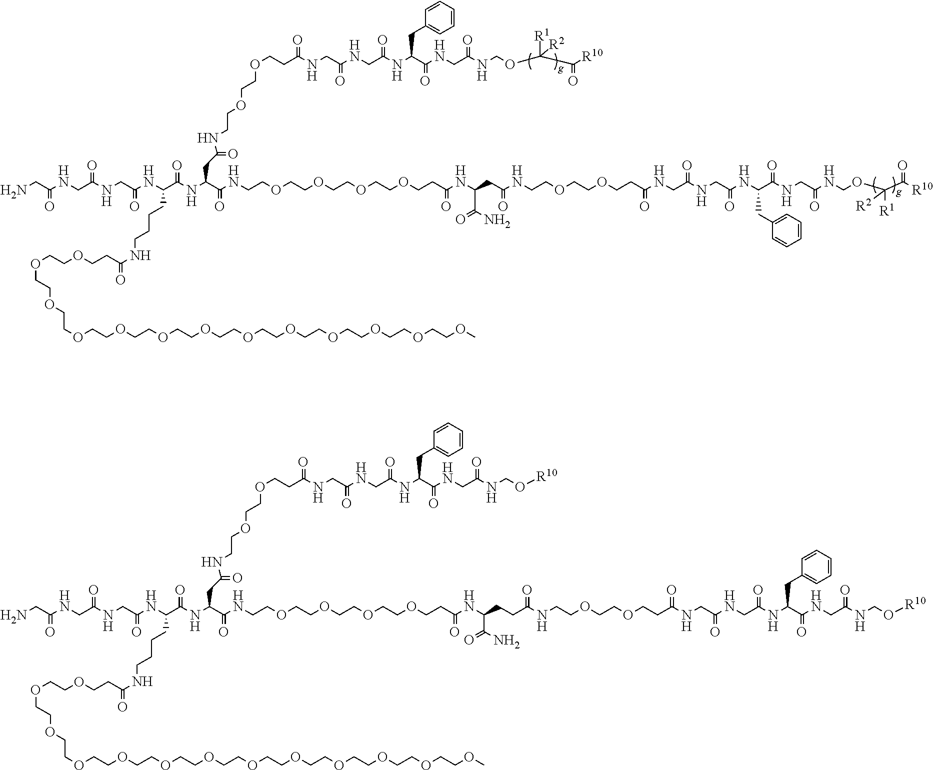 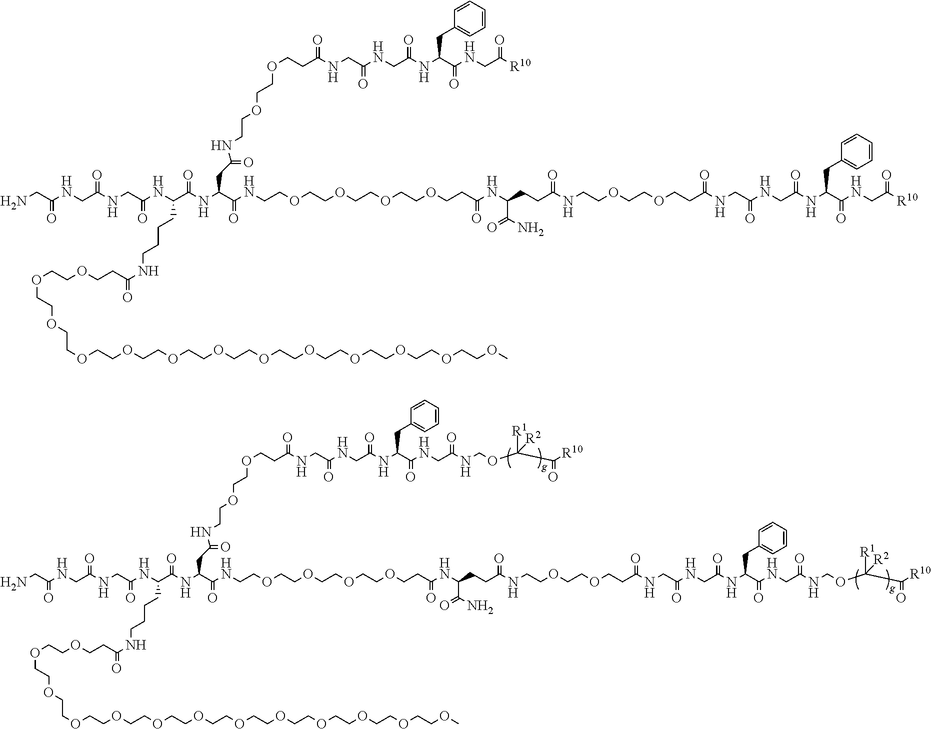 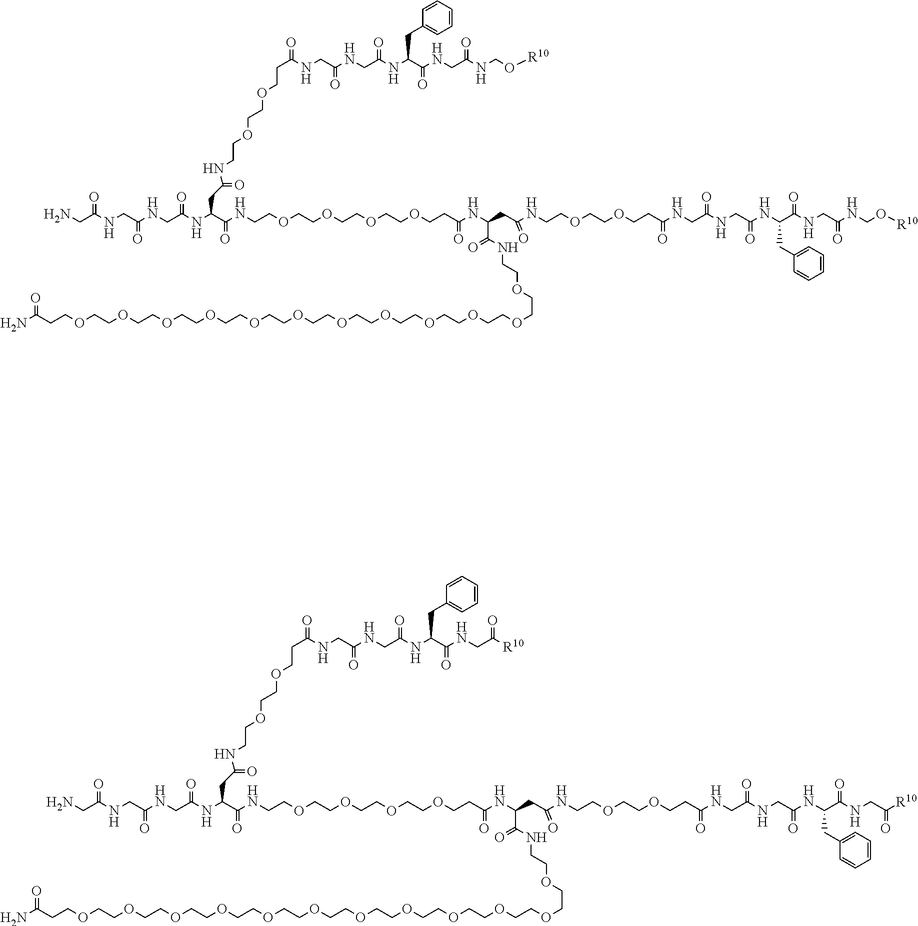 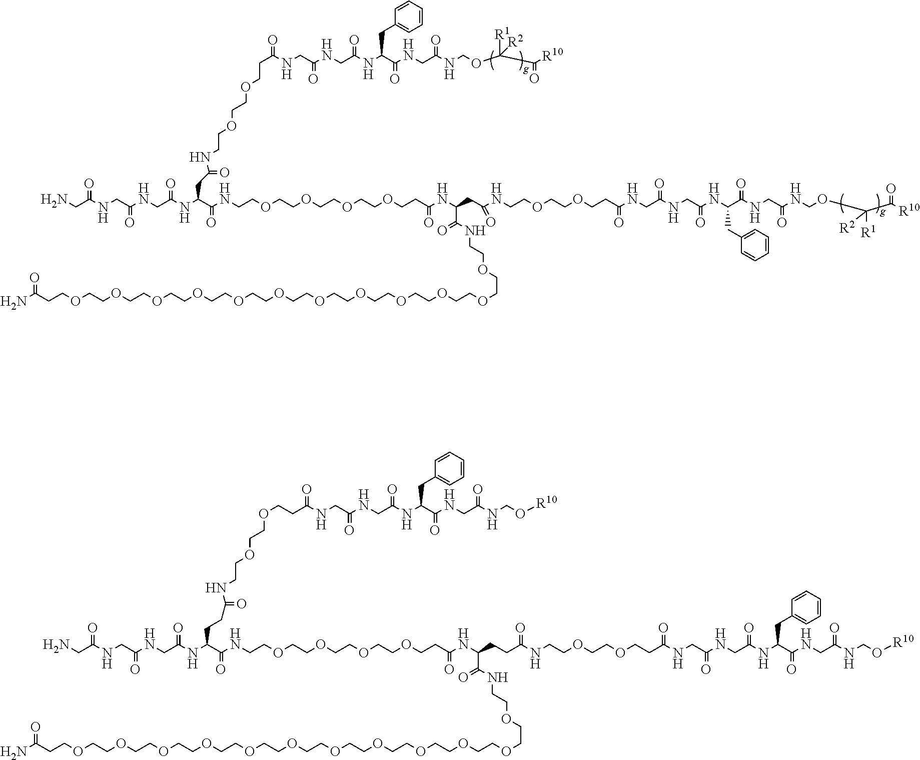 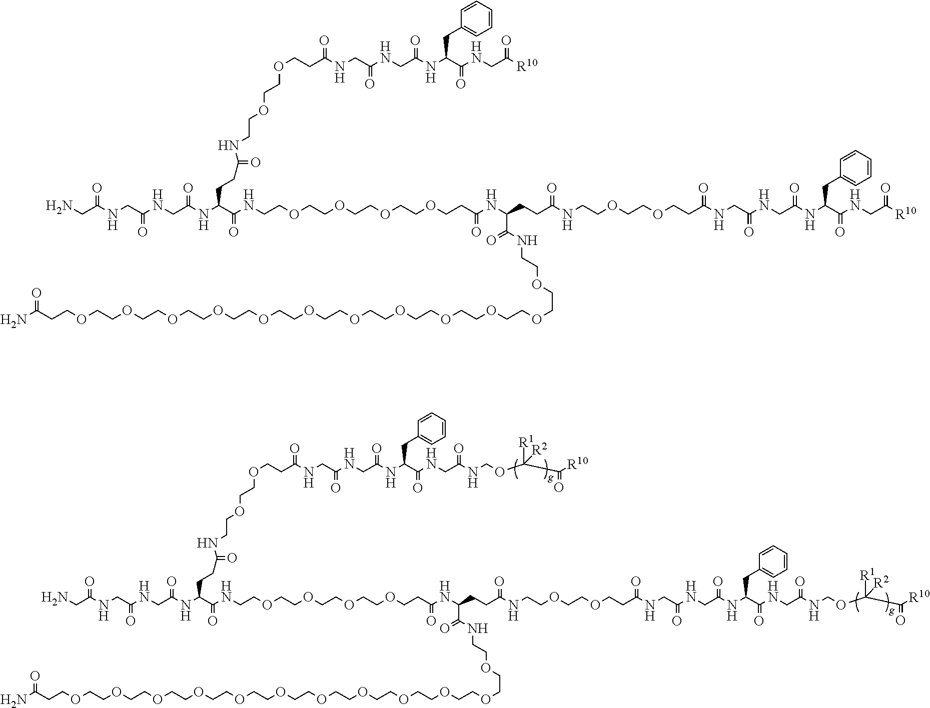 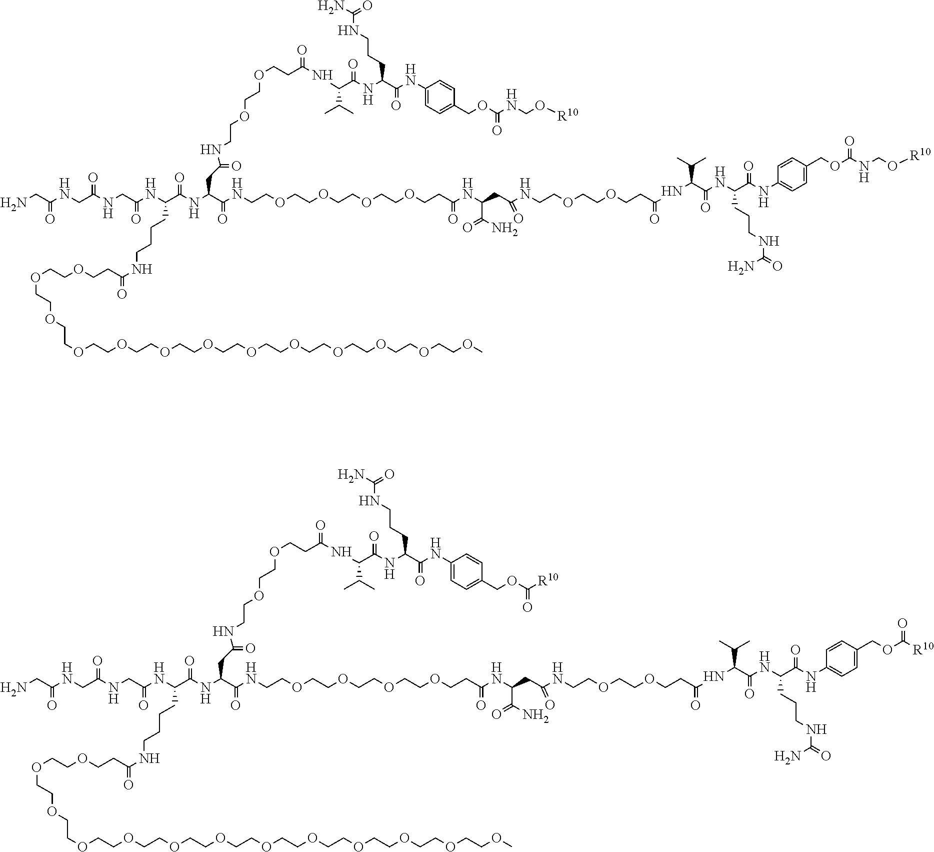 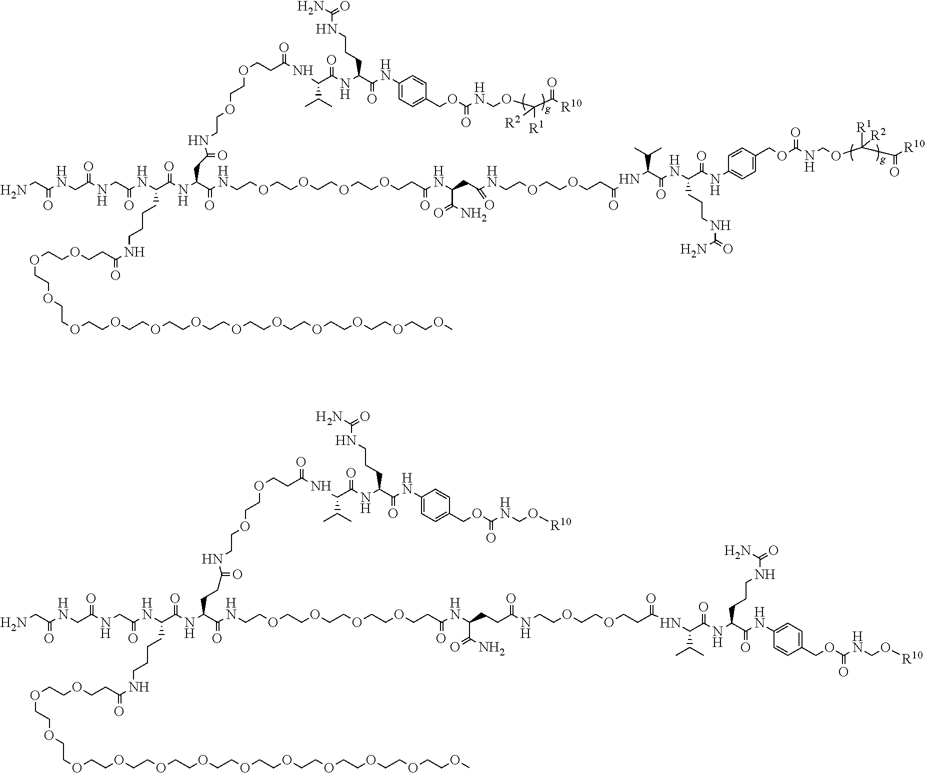 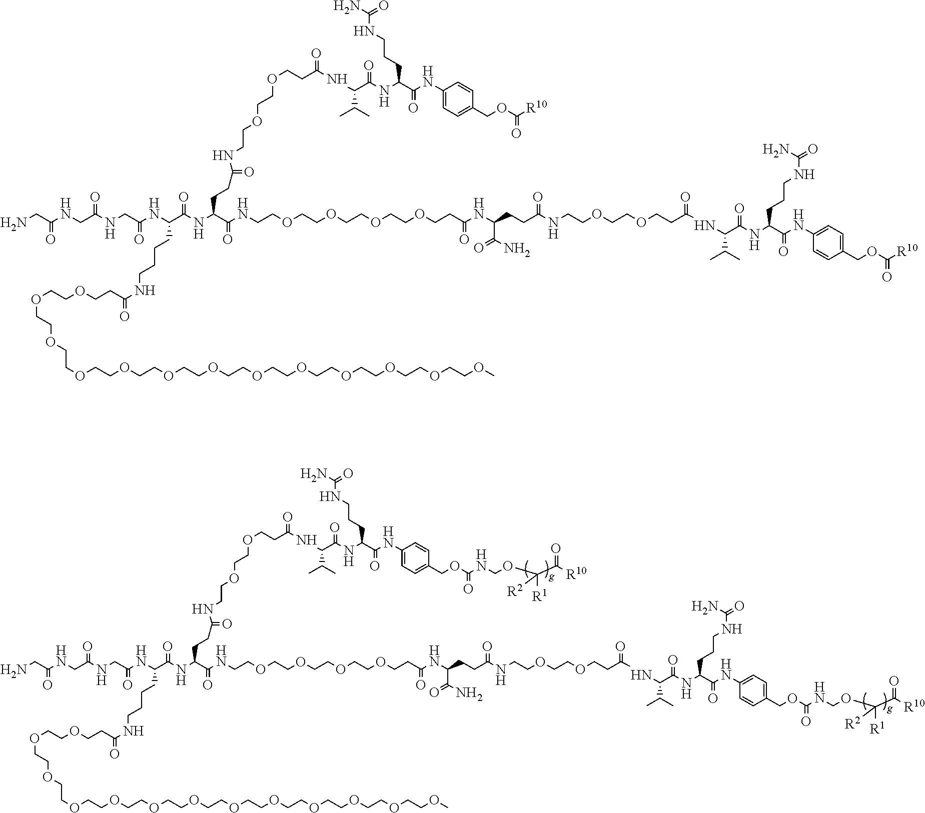 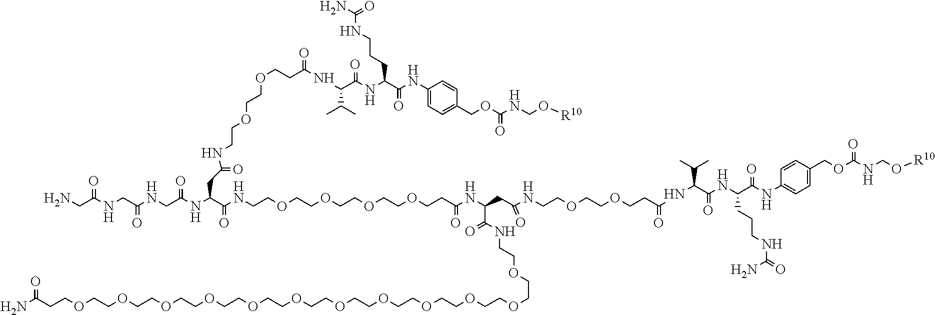 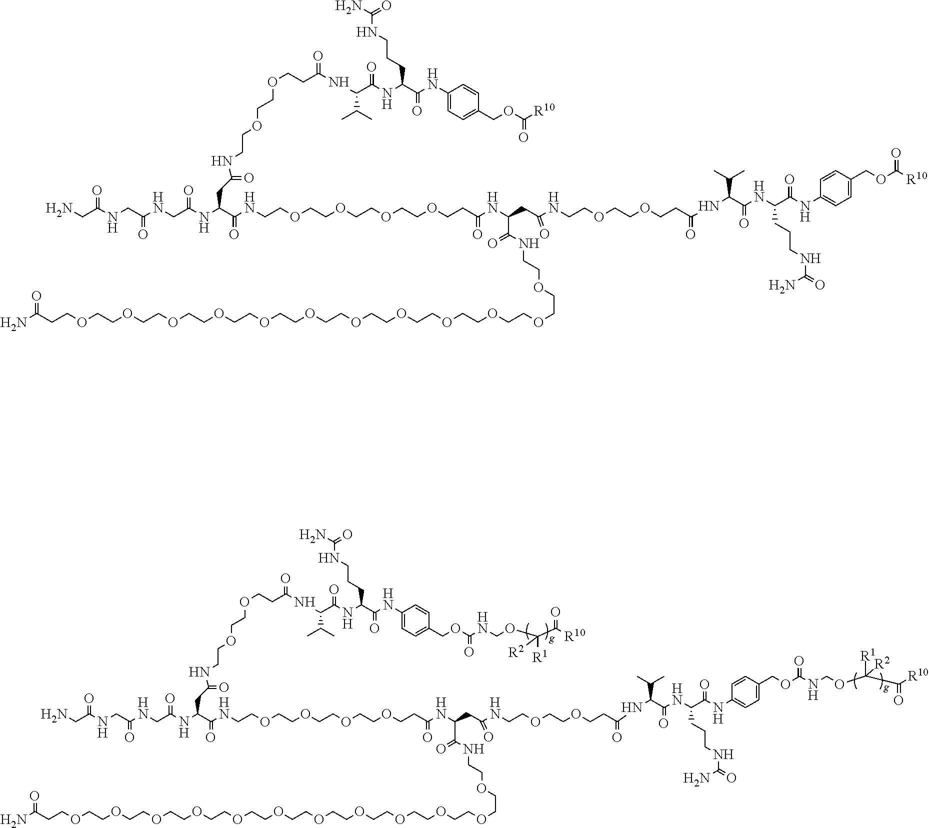 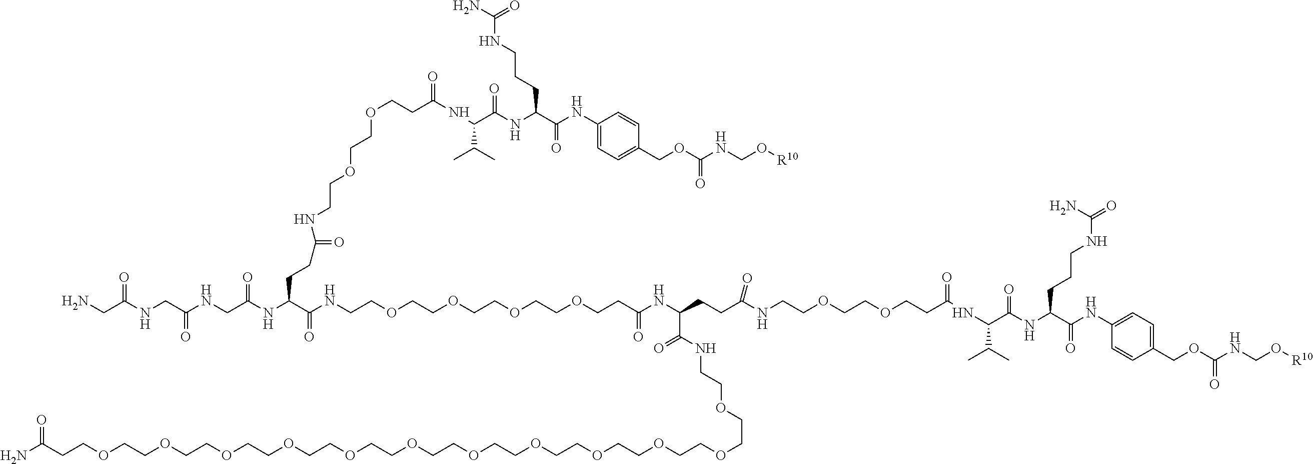 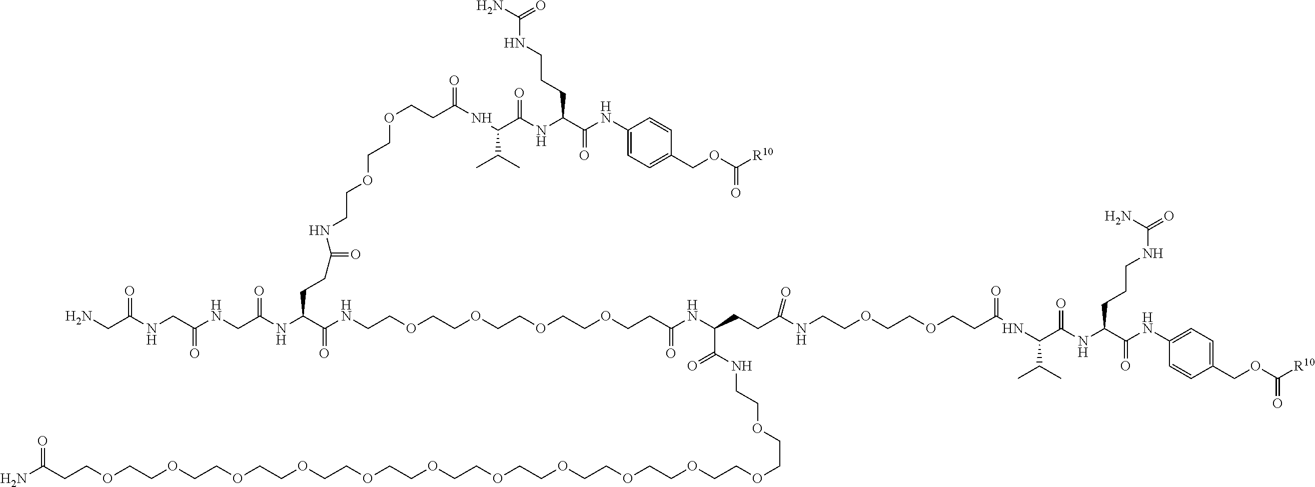 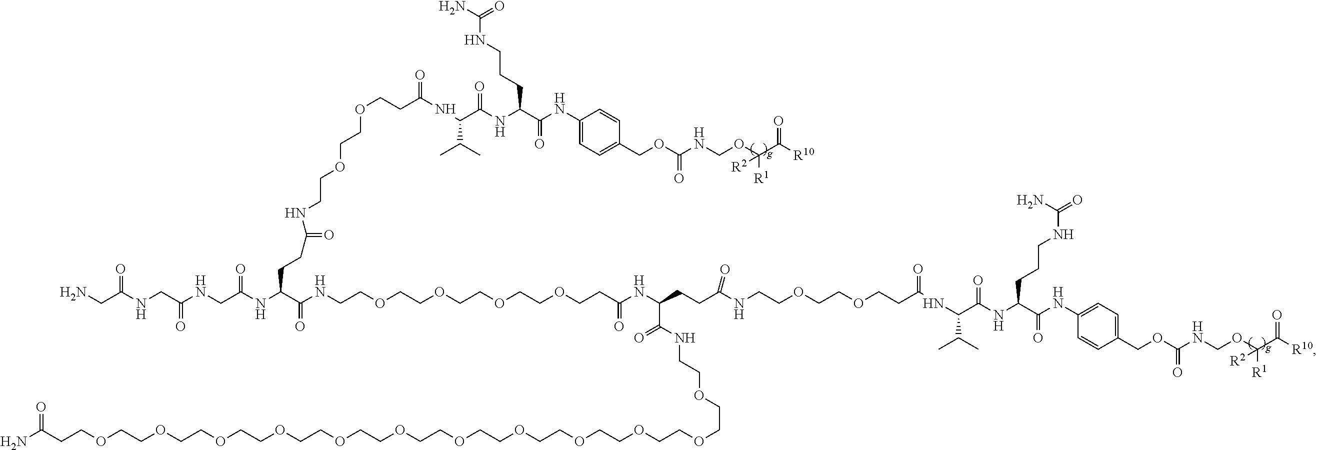
wherein R¹, R² and R¹⁰ are as defined in claim 1, and g is an integer of 1 to 10, preferably 1.

8. A compound having the structure of formula (II) 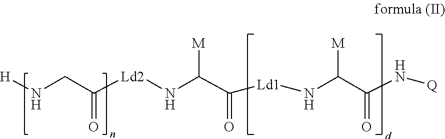
Wherein
Q is hydrogen, —C₂H₄-(PEG)ₜ-(CO)NH₂ or LKb-P;
M is LKa-LKb-P;
P is a payload which is linked to the B moiety or L¹ moiety of the compound of formula (I) as defined in claim 1;
n, d, Ld1, Ld2, t, LKa and LKb are as defined in claim 1;
preferably, M is LKa-L²-L¹-B-P; wherein each B is independently absent, or is a combination of the following 1) and 2): 1) a self-immolative spacer Sp1; and 2) a bond, or one of or a combination of two or more of the bivalent groups selected from: —CR¹R²—, C₁₋₁₀ alkylene, C₄₋₁₀ cycloalkylene, C₄₋₁₀ heterocyclylene and —(CO)—;
preferably, Sp1 is selected from PABC, acetal, heteroacetal and the combination thereof, more preferably, Sp1 is acetal, heteroacetal or PABC; further preferably, the heteroacetal is selected from N,O-heteroacetal; more preferably, Sp1 is —O—CH₂—U- or —NH—CH₂—U—; wherein the —O— or the —NH— is connected to Cleavable sequence 1, and U is absent or U is O, S or NH, preferably O or S.

9. A conjugate having the structure of formula (III): 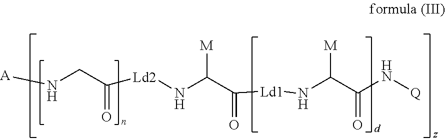
wherein,
Q is hydrogen, —C₂H₄-(PEG)ₜ-(CO)NH₂ or LKb-P;
n, d, t, Ld1 and Ld2 are as defined in claim 1;
M is LKa-LKb-P;
preferably, M is LKa-L²-L¹-B-P; wherein
each B is independently absent, or is a combination of the following 1) and 2): 1) a self-immolative spacer Sp1; and 2) a bond, or one of or a combination of two or more of the bivalent groups selected from: -CR¹R²—, C₁₋₁₀ alkylene, C₄₋₁₀ cycloalkylene, C₄₋₁₀ heterocyclylene and —(CO)—; preferably, B is —NH—CH₂—U— or absent or -NH-CH₂-U—(CR¹R²)_{g}—(CO)—, g is an integer of 1 to 10, preferably 1; wherein R¹ and R² are each independently selected from hydrogen, halogen, -C₁₋₁₀ alkyl, —C₁₋₁₀ haloalkyl, C₄₋₁₀ cycloalkylene; or
R¹ and R² together with the carbon atom to which they are attached form a 3-6 membered cycloalkyl group;
P is a payload which is linked to the B moiety or L¹ moiety of the compound of formula (I) as defined in claim 1;
A is a targeting molecule;
z is an integer of 1 to 20.

10. The conjugate of claim 9, wherein
the conjugate has the structure of the following formula (III-1):

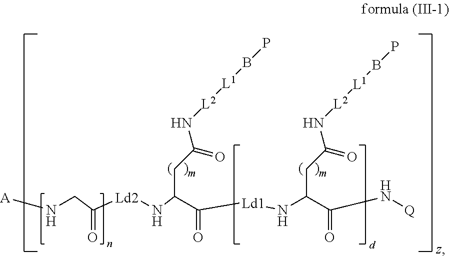

11. The conjugate of claim 9, wherein
the conjugate has the structure of the following: 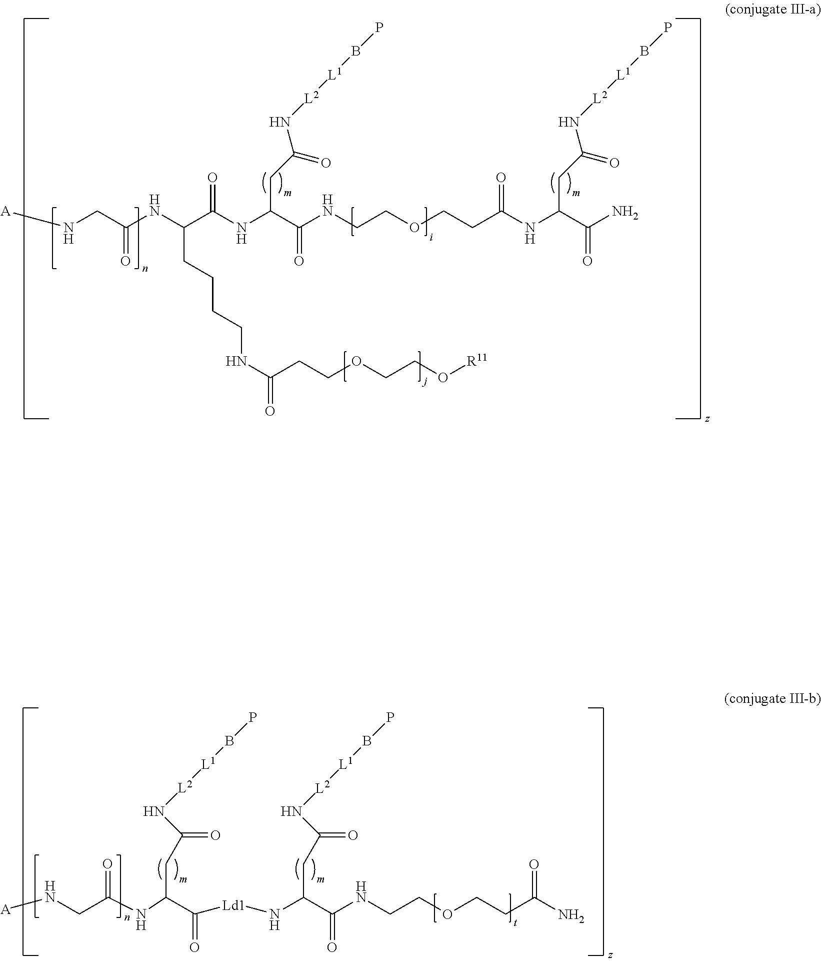
preferably, z is 1 to 4; preferably 2;
n is an integer of 2 to 5, preferably n is 3, L² is —(C₂H₄—O)ₚ—(CH₂)₂(CO)—, p is 2 to 4, L¹ is Gly-Gly-Phe-Gly, B is —NH—CH₂—U- or absent or -NH-CH2-U—(CR¹R²)_{g}—(CO)—, U is absent, or U is O, g is 1; wherein R¹ and R² are each independently selected from hydrogen, halogen, —C₁₋₁₀ alkyl, —C₁₋₁₀ haloalkyl, C₄₋₁₀ cycloalkylene; or
R¹ and R² together with the carbon atom to which they are attached form a 3-6 membered cycloalkyl group;
each i is independently an integer of 0-100, preferably 0 to 20; preferably each i is independently an integer of 0 to 12; more preferably 0 to 8; particularly 4;
each j is independently an integer of 1-100, preferably 1 to 20; preferably each j is independently an integer of 1 to 12; more preferably 8 to 12; particularly 8 or 12;
each t is independently an integer of 1-100, preferably 1 to 20; preferably each t is independently an integer of 1 to 12; more preferably 8 to 12; particularly 8 or 12;
m is any integer of 1 to 3; particularly 1 or 2.

12. The conjugate of claim 9, wherein
the targeting molecule is an antibody or an antigen binding fragment thereof; the antibody or antigen binding fragment is preferably modified to connect with the Gₙ moiety in the compound of formula (I);
preferably, A is an anti-human monoclonal antibody connected to the rest of the conjugate through a modified heavy chain and/or light chain C-terminal, wherein the modified heavy chain and/or light chain C-terminal is modified to comprise Leu-Pro-Xaa-Thr, wherein Xaa is any natural or unnatural single amino acid, and z is 2;
preferably, the antibody is an anti-human HER2 antibody, an anti-human TROP2 antibody or anti-FGFR3 antibody.

13. The conjugate of claim 9, wherein the payload is a cytotoxin or a fragment thereof, with an optional derivatization in order to connect to the B moiety or L¹ moiety in the compound of formula (I);
preferably, the cytotoxin is selected from the group consisting of taxanes, maytansinoids, auristatins, epothilones, combretastatin A-4 phosphate, combretastatin A-4 and derivatives thereof, indol-sulfonamides, vinblastines such as vinblastine, vincristine, vindesine, vinorelbine, vinflunine, vinglycinate, anhydrovinblastine, dolastatin 10 and analogues, halichondrin B, eribulin, indole-3-oxoacetamide, podophyllotoxins, 7-diethylamino-3-(2′-benzoxazolyl)-coumarin (DBC), discodermolide, laulimalide, camptothecins and derivatives thereof, mitoxantrone, mitoguazone, nitrogen mustards, nitrosoureasm, aziridines, benzodopa, carboquone, meturedepa, uredepa, dynemicin, esperamicin, neocarzinostatin, aclacinomycin, actinomycin, antramycin, bleomycins, actinomycin C, carabicin, carminomycin, cardinophyllin, carminomycin, actinomycin D, daunorubicin, detorubicin, adriamycin, epirubicin, esorubicin, idarubicin, marcellomycin, mitomycins, nogalamycin, olivomycin, peplomycin, porfiromycin, puromycin, ferric adriamycin, rodorubicin, rufocromomycin, streptozocin, zinostatin, zorubicin, trichothecene, T-2 toxin, verracurin A, bacillocporin A, anguidine, ubenimex, azaserine, 6-diazo-5-oxo-L-norleucine, dimethyl folic acid, methotrexate, pteropterin, trimetrexate, edatrexate, fludarabine, 6-mercaptopurine, tiamiprine, thioguanine, ancitabine, gemcitabine, enocitabine, azacitidine, 6-azauridine, carmofur, cytarabine, dideoxyuridine, doxifluridine, floxuridine, calusterone, dromostanolone propionate, epitiostanol, mepitiostane, testolactone, aminoglutethimide, mitotane, trilostane, flutamide, nilutamide, bicalutamide, leuprorelin acetate, protein kinase inhibitors and a proteasome inhibitors; and/or
selected from vinblastines, colchicines, taxanes, auristatins, maytansinoids, calicheamicin, doxonubicin, duocarmucin, SN-38, cryptophycin analogue, deruxtecan, duocarmazine, calicheamicin, centanamycin, dolastansine, pyrrolobenzodiazepine, exatecan and derivatives thereof, and/or
selected from auristatins, especially MMAE, MMAF or MMAD; and/or
selected from exatecan and derivatives thereof, such as DX8951f; and/or
selected from DXd-(1) and DXd-(2); preferably DXd-(1).

14. The conjugate of claim 9, wherein the payload is selected from 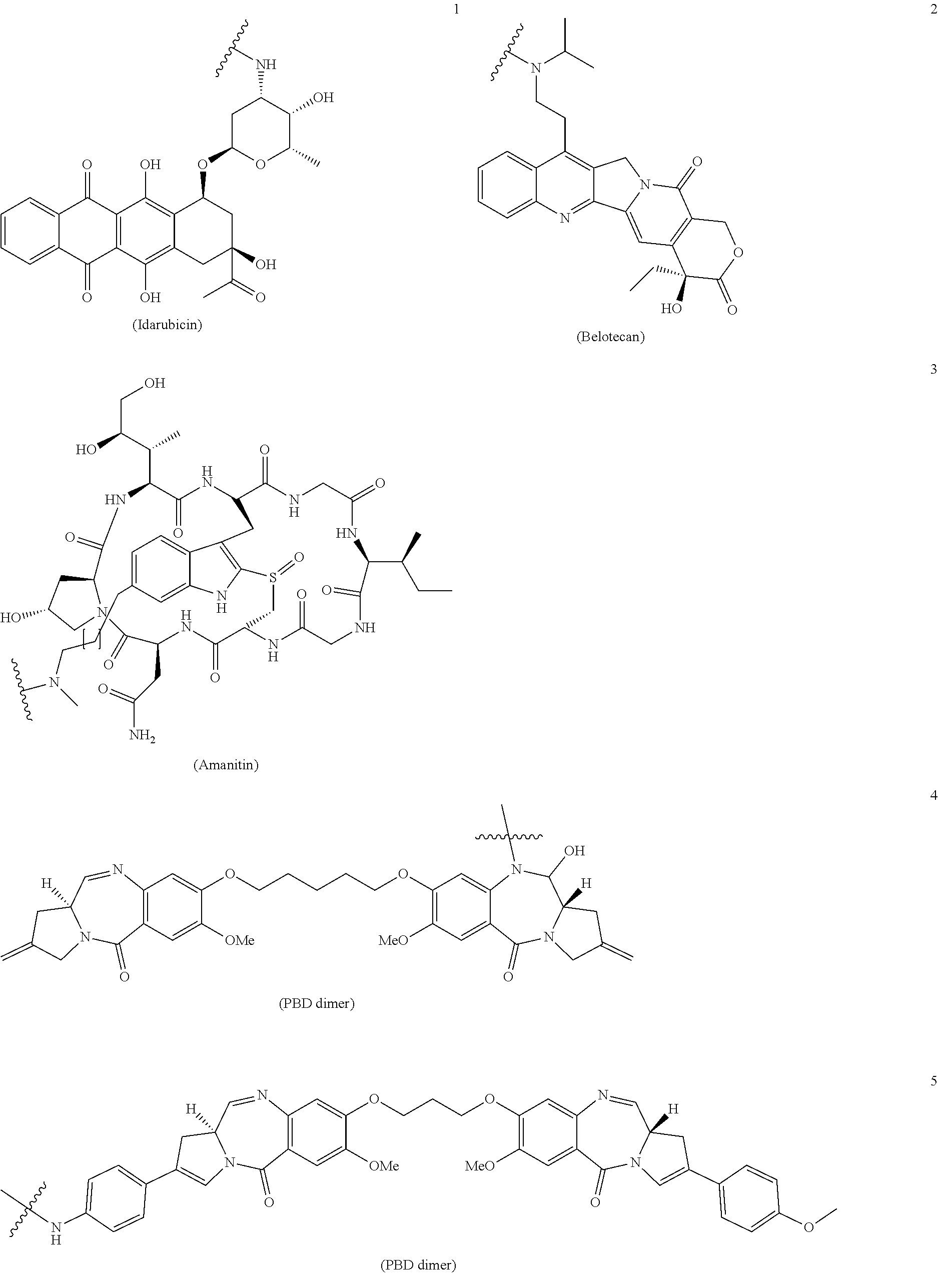 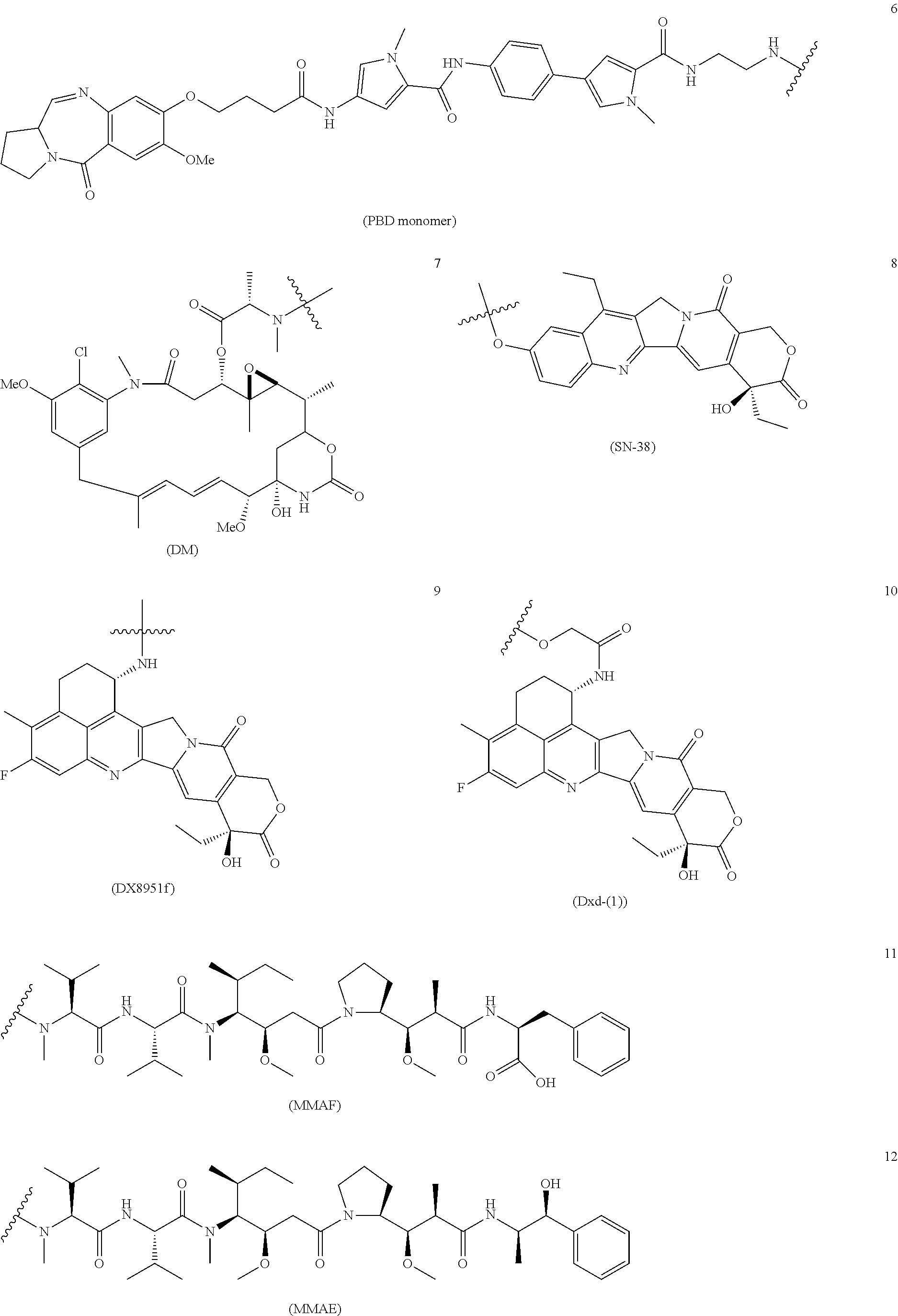 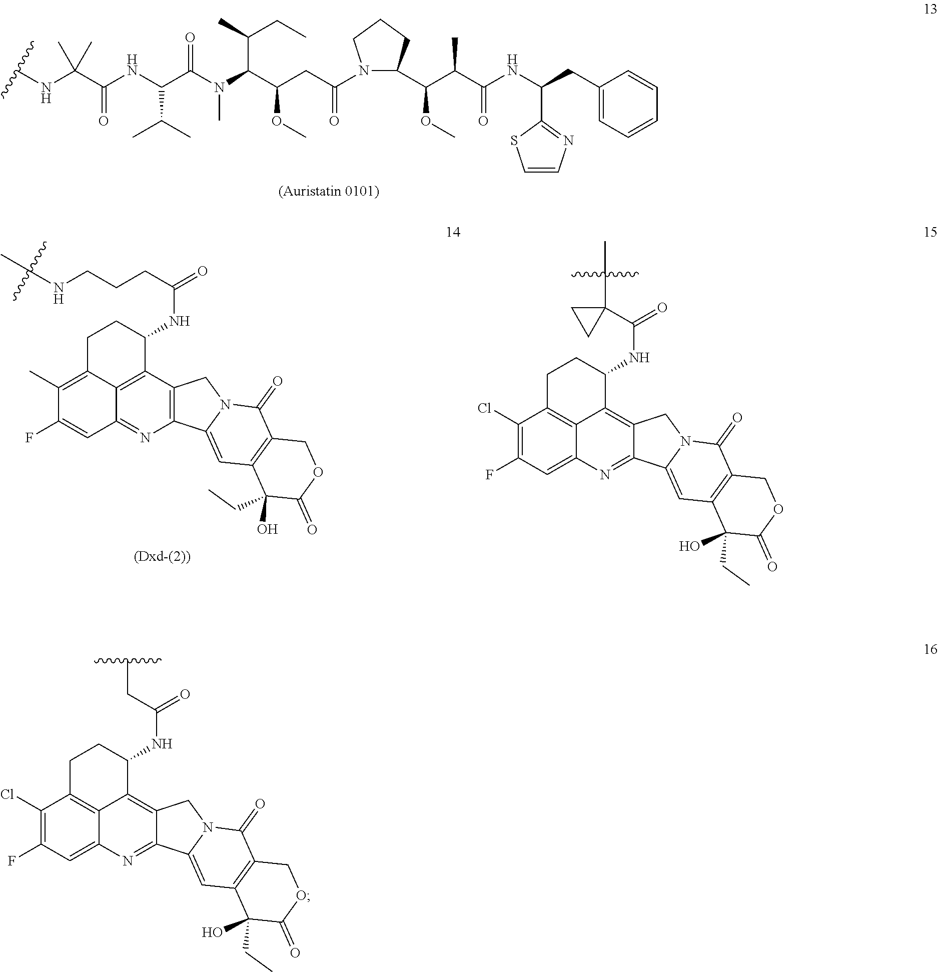
especially selected from

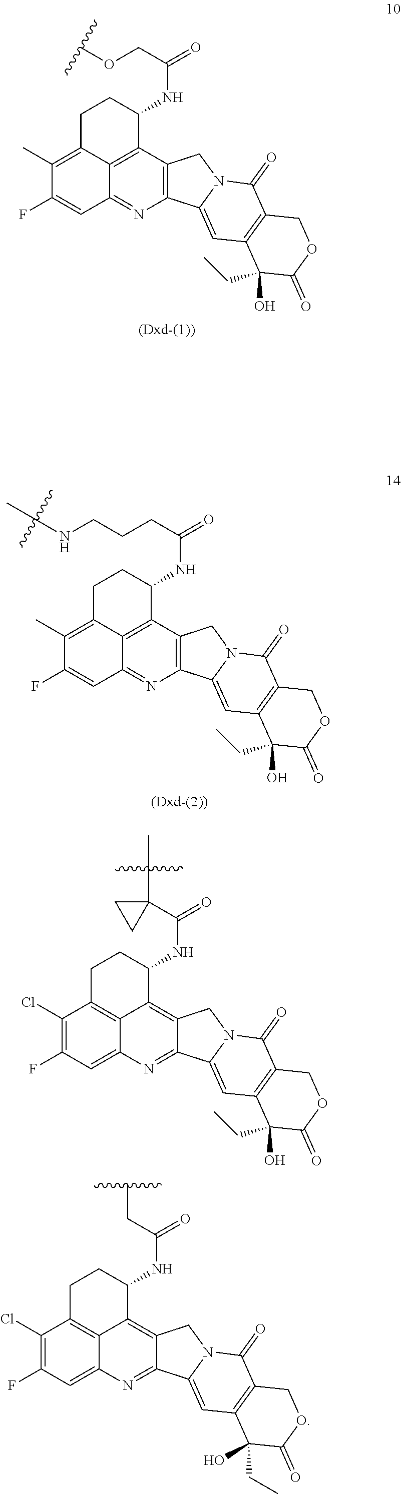 [SMILES: *OCC(=O)N[C@H]1CCc2c(C)c(F)cc3nc4c(c1c23)Cn1c-4cc2c(c1=O)COC(=O)[C@]2(O)CC ; *NCCCC(=O)N[C@H]1CCc2c(C)c(F)cc3nc4c(c1c23)Cn1c-4cc2c(c1=O)COC(=O)[C@]2(O)CC ; *C1(C(=O)N[C@H]2CCc3c(Cl)c(F)cc4nc5c(c2c34)Cn2c-5cc3c(c2=O)COC(=O)[C@]3(O)CC)CC1 ; *CC(=O)N[C@H]1CCc2c(Cl)c(F)cc3nc4c(c1c23)Cn1c-4cc2c(c1=O)COC(=O)[C@]2(O)CC]

15. The conjugate of claim 9, wherein formula (III) having the following structure: 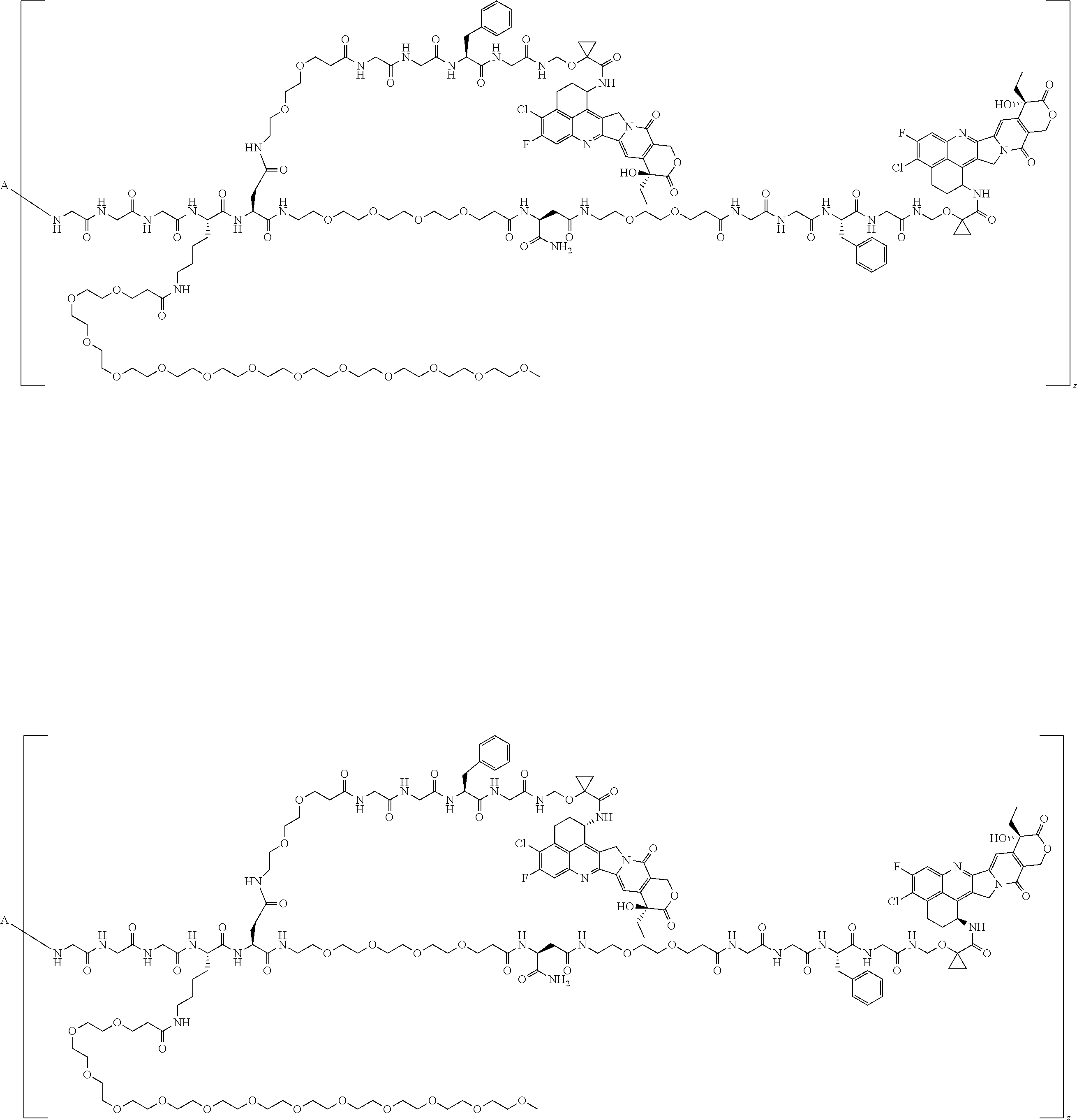 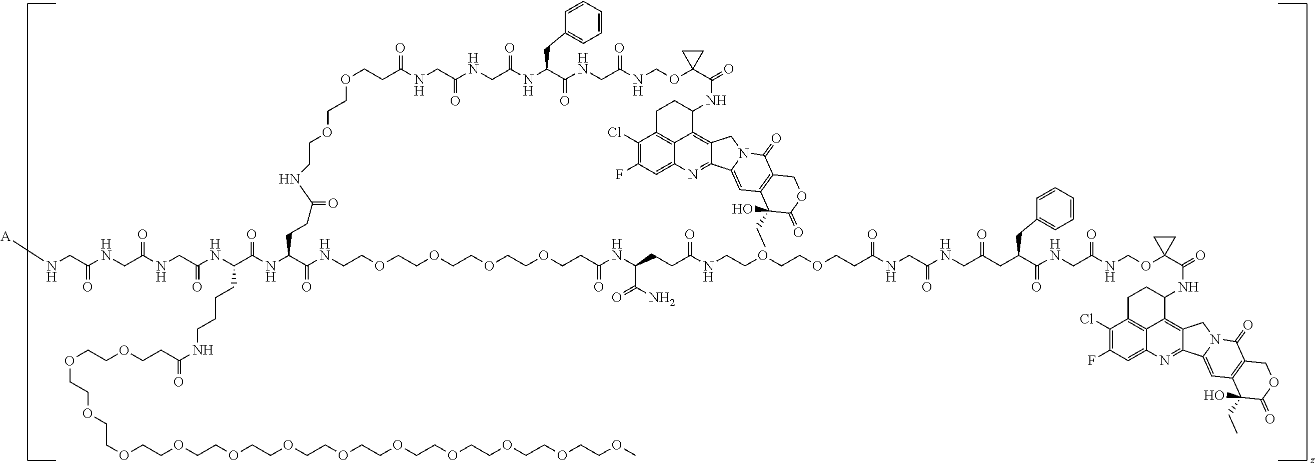 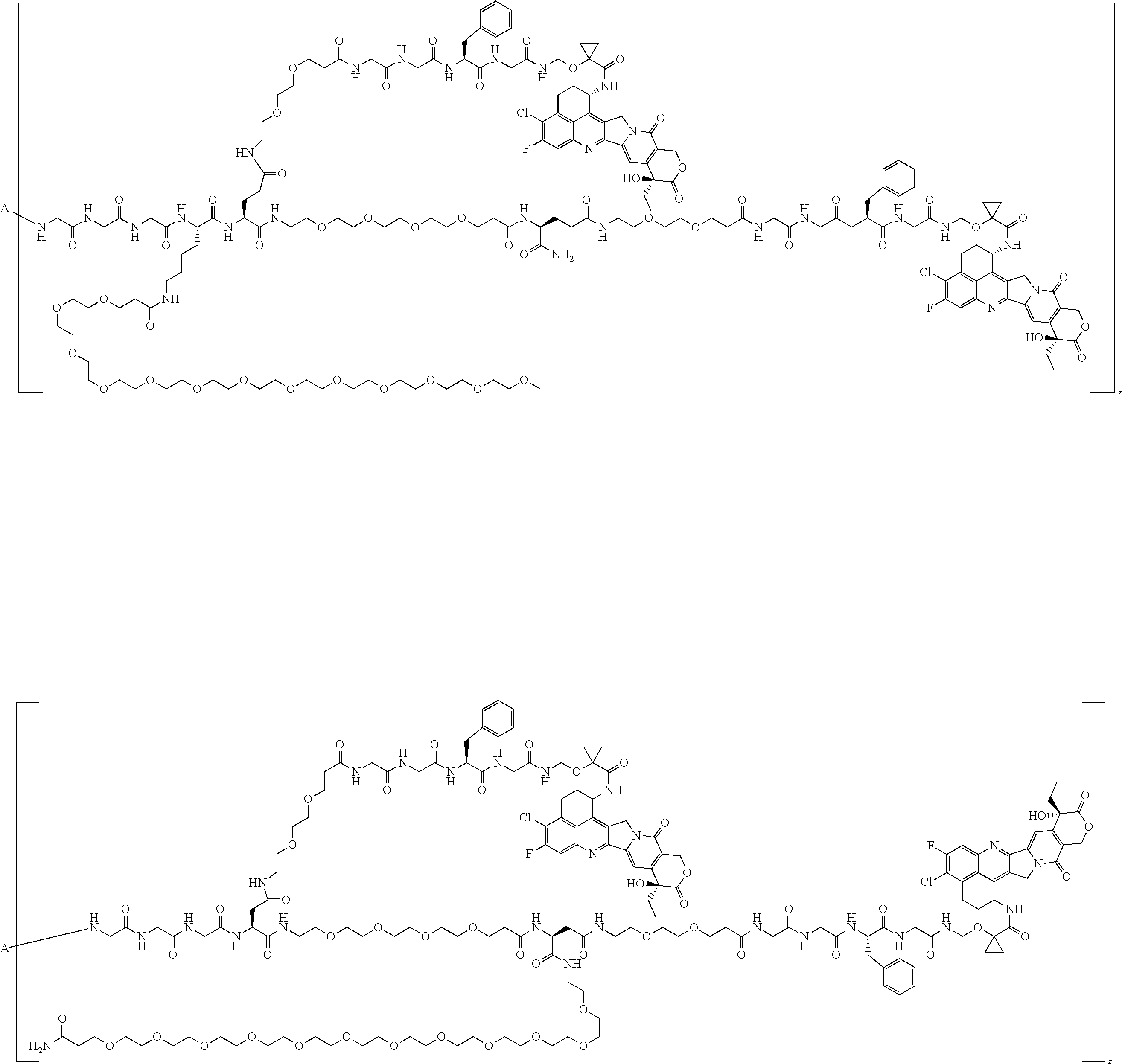 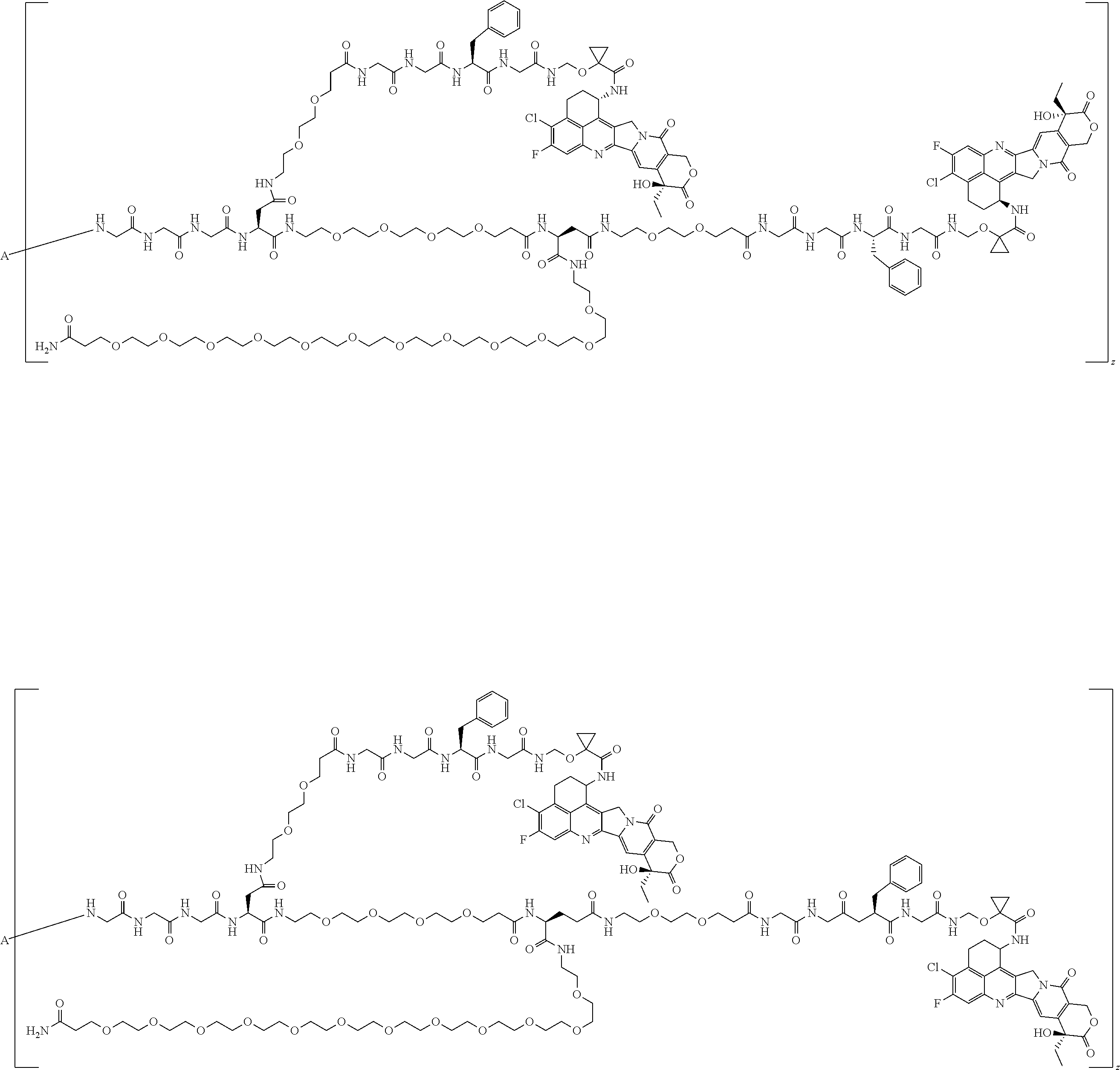 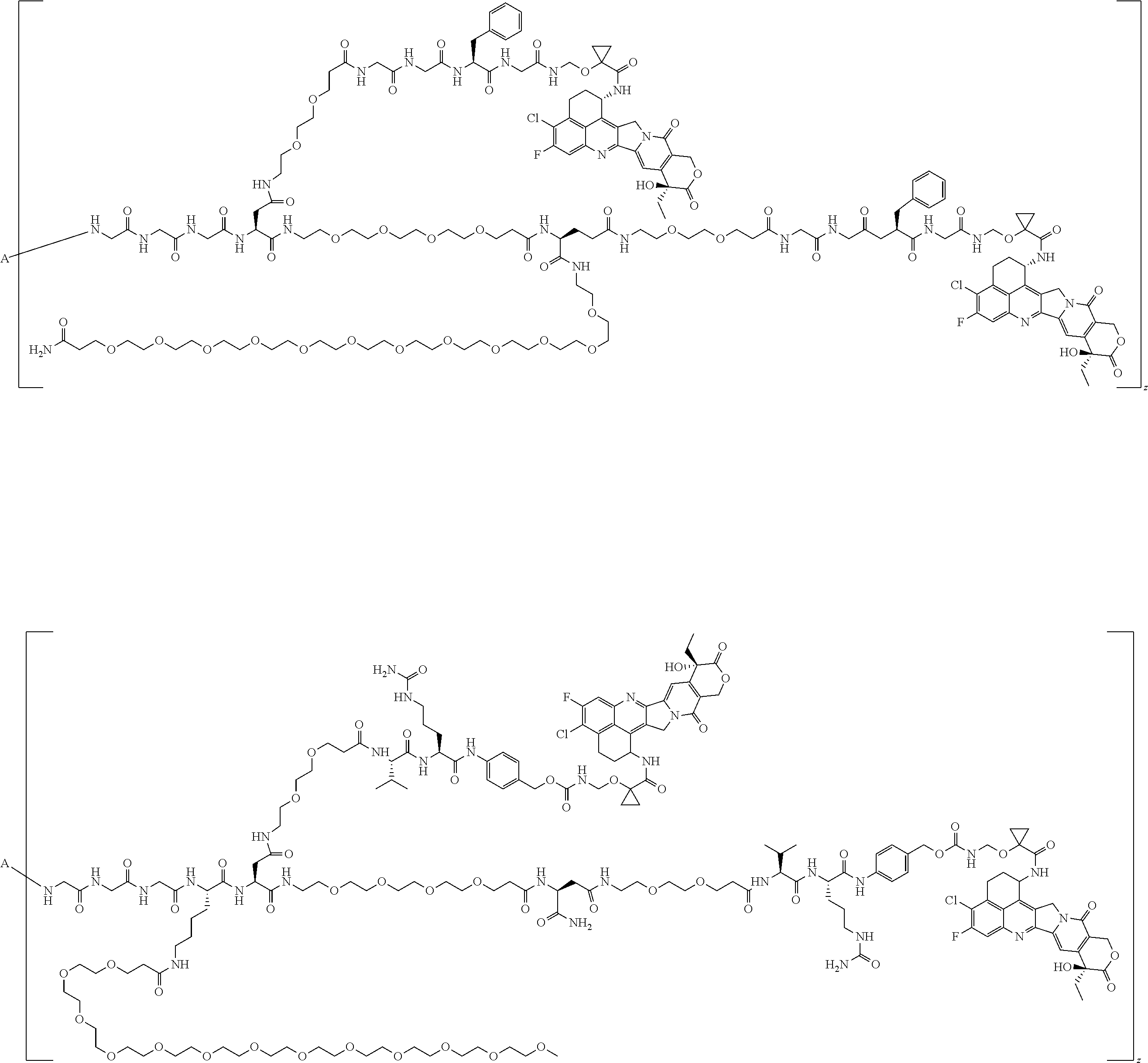 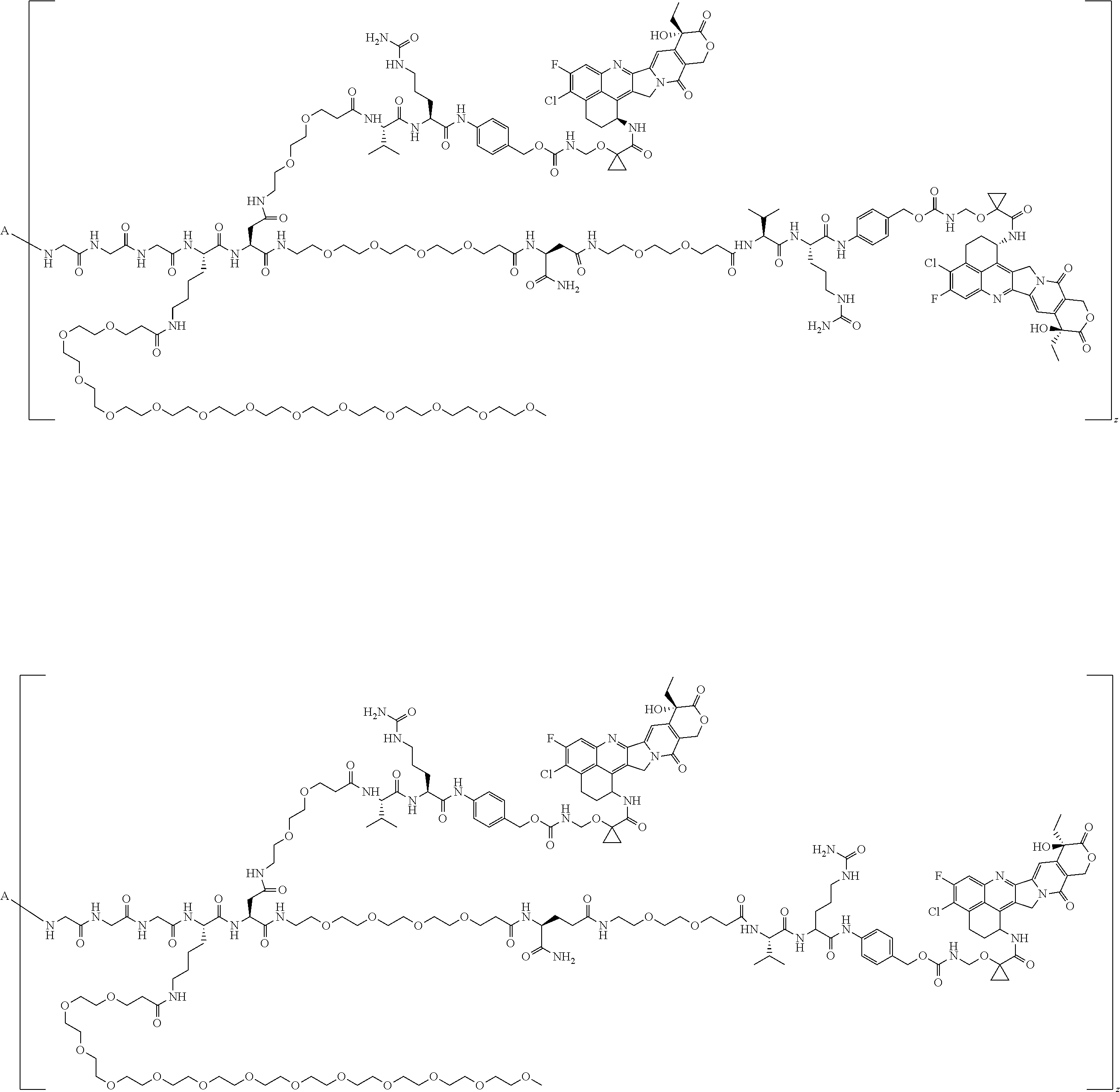 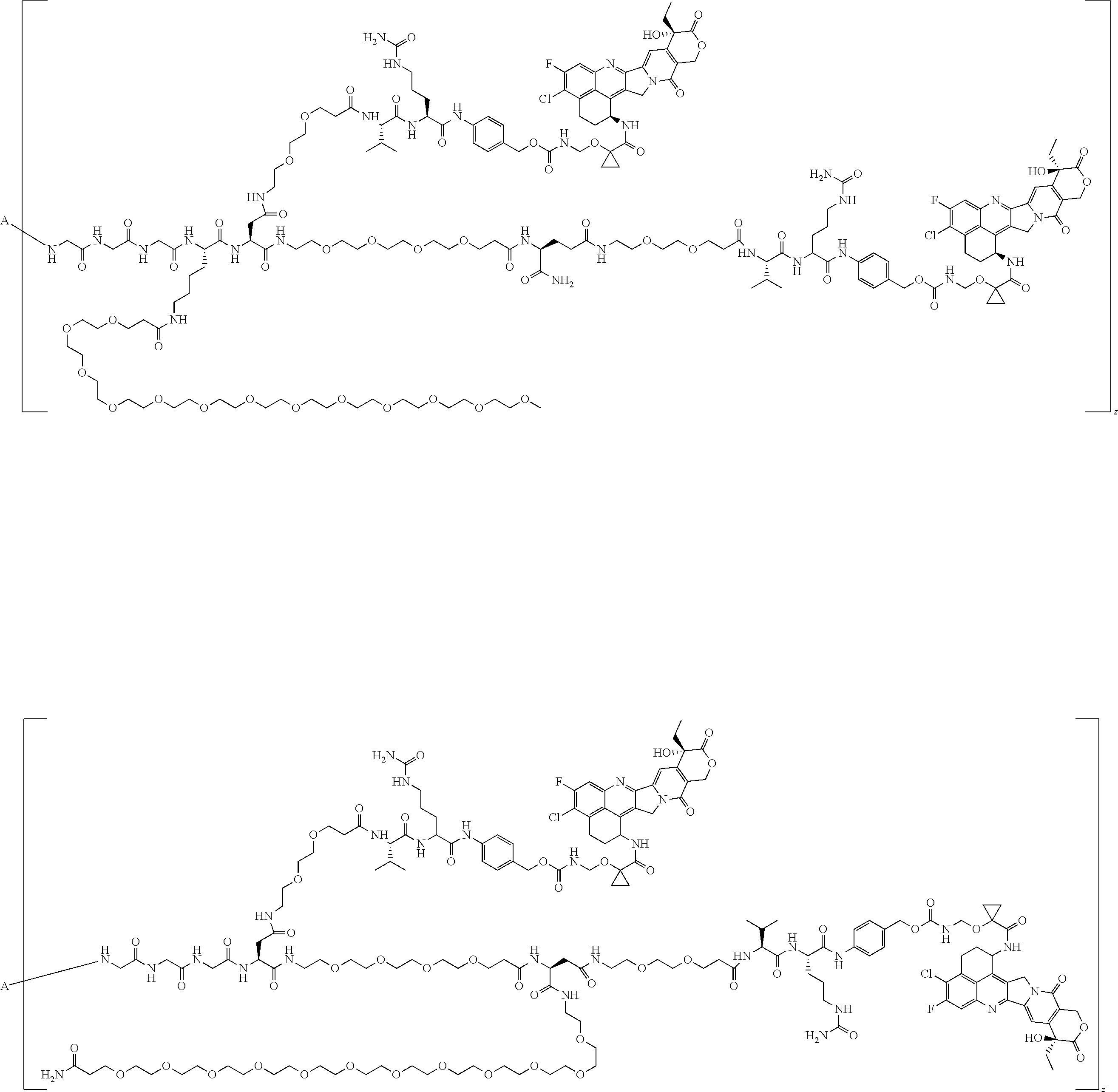 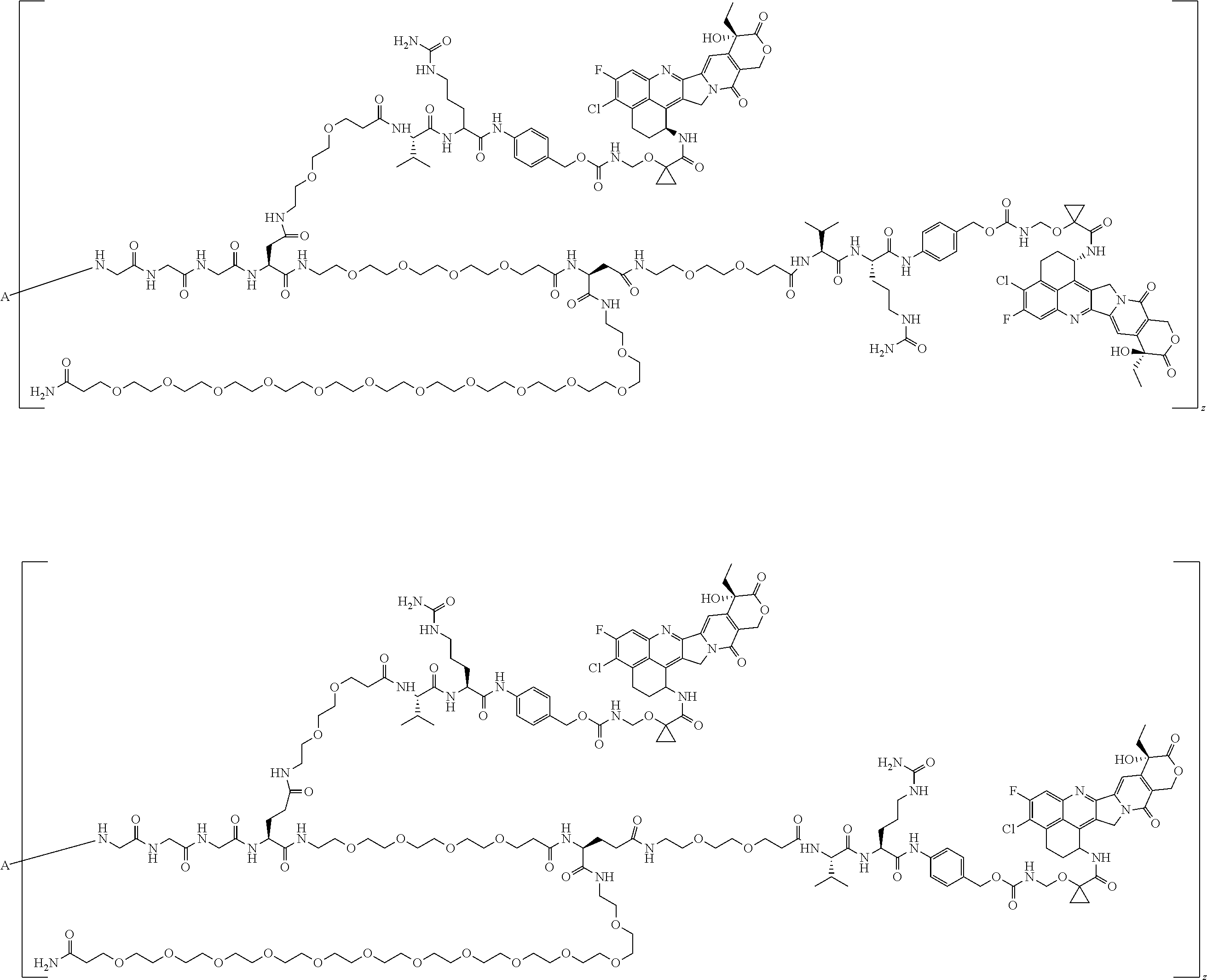 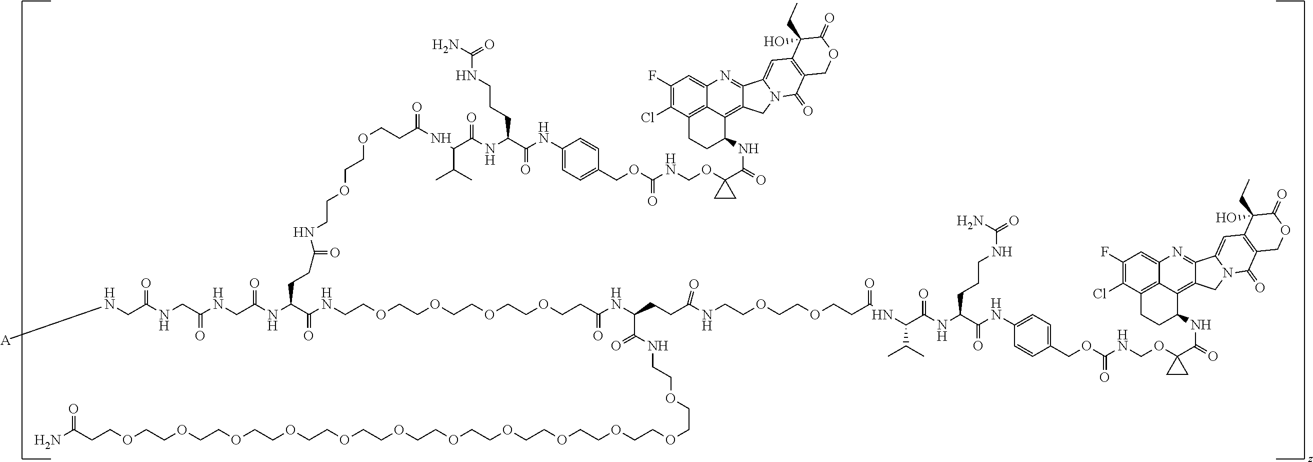
each z is independently an integer of 1 to 20.

16. A pharmaceutical composition comprising a prophylactically or therapeutically effective amount of a conjugate of claim 9, and at least one pharmaceutically acceptable carrier.

17. A method treating a disease, comprising administering the conjugate of claim 9 to a subject in need thereof; wherein the disease is a tumor or an autoimmune disease; preferably a HER2-positive tumor, a TROP2-positive tumor or a FGFR3-positive tumor;
preferably,
the HER2-positive tumor is selected from breast cancer, gastric cancer, lung cancer, ovarian cancer, and urothelial cancer;
the TROP2-positive tumor is selected from breast cancer, gastric cancer, lung cancer, ovarian cancer, and urothelial cancer;
the FGFR3-positive tumor is selected from brain cancer, bladder cancer, urothelial cancer, cervical cancer, multiple myeloma or intrahepatic cholangiocarcinoma.

18. A compound of formula (IV): 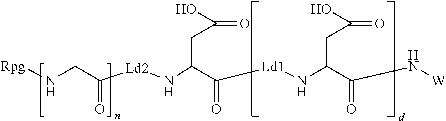
wherein,
Rpg is selected from hydrogen, or a protecting group, preferably selected from acetyl, trifluoroacetyl, t-butoxycarbonyl (Boc), benzyloxycarbonyl (Cbz) and 9-fluorenylmethylenoxycarbonyl (Fmoc);
W, Ld1, Ld2, n and d are as defined in claim 1.

19. The compound of claim 18, wherein the formula (IV) having the following structure: [chemical structure]


SEQUENCE LISTING (8 sequences)

SEQ ID NO: 1 - protein, 222 aa, synthetic construct
DIQMTQSPSSLSASVGDRVTITCRASQDVNTAVAWYQQKPGKAPKLLIYSASFLYSGVPSRFSGSRSGTDFTLTISSLQPEDFATYYCQQHYTTPPTFGQGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGECGALPETGG

SEQ ID NO: 2 - protein, 450 aa, synthetic construct
EVQLVESGGGLVQPGGSLRLSCAASGFNIKDTYIHWVRQAPGKGLEWVARIYPTNGYTRYADSVKGRFTISADTSKNTAYLQMNSLRAEDTAVYYCSRWGGDGFYAMDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 3 - protein, 222 aa, synthetic construct; identical to SEQ ID NO: 1

SEQ ID NO: 4 - protein, 458 aa, synthetic construct
EVQLVESGGGLVQPGGSLRLSCAASGFNIKDTYIHWVRQAPGKGLEWVARIYPTNGYTRYADSVKGRFTISADTSKNTAYLQMNSLRAEDTAVYYCSRWGGDGFYAMDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGKGALPETGG

SEQ ID NO: 5 - protein, 222 aa, synthetic construct
DIQLTQSPSSLSASVGDRVSITCKASQDVSIAVAWYQQKPGKAPKLLIYSASYRYTGVPDRFSGSGSGTDFTLTISSLQPEDFAVYYCQQHYITPLTFGAGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGECGALPETGG

SEQ ID NO: 6 - protein, 451 aa, synthetic construct
QVQLQQSGSELKKPGASVKVSCKASGYTFTNYGMNWVKQAPGQGLKWMGWINTYTGEPTYTDDFKGRFAFSLDTSVSTAYLQISSLKADDTAVYFCARGGFGSSYWYFDVWGQGSLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 7 - protein, 222 aa, synthetic construct
QSVLTQPPSLSVAPGKTATFTCGGNNIGDKSVHWYRQKPGQAPVLVMYLDTERPSGIPERMSGSNFGNTATLTITRVEAEDEADYYCQVWDSGSDHVVFGGGTKLTVLGQPKAAPSVTLFPPSSEELQANKATLVCLISDFYPGAVTVAWKADSSPVKAGVETTTPSKQSNNKYAASSYLSLTPEQWKSHRSYSCQVTHEGSTVEKTVAPAECSGALPETGG

SEQ ID NO: 8 - protein, 456 aa, synthetic construct
EVQLVQSGAEVKKPGASVKVSCKASGYMFTSYGISWVRQAPGQGLEWMGWVSTYNGDTNYAQKFQGRVTVTTDTSTSTAYMELRSLRSEDTAVYYCARVLGYYDSIDGYYYGMDVWGQGTTVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK